(19) United States
(12) Patent Application Publication
(10) Pub. No.: US 2023/0227426 A1
(43) Pub. Date: Jul. 20, 2023

(54) PHD INHIBITOR COMPOUNDS, COMPOSITIONS, AND USE

(71) Applicant: Akebia Therapeutics, Inc., Cambridge, MA (US)

(72) Inventors: Paul E. FLEMING, Cambridge, MA (US); Thomas P. BLAISDELL, Cambridge, MA (US); Senkara Rao ALLU, Cambridge, MA (US)

(73) Assignee: Akebia Therapeutics, Inc., Cambridge, MA (US)

(21) Appl. No.: 17/906,650

(22) PCT Filed: Mar. 19, 2021
(86) PCT No.: PCT/US2021/023219

§ 371 Date: Sep. 19, 2022

Related U.S. Application Data
(60) Provisional application No. 62/992,585, filed on Mar. 20, 2020.

(51) Int. Cl. C07D 401/04; C07D 401/14

(52) U.S. Cl. CPC C07D 401/04; C07D 401/14

(57) ABSTRACT

The present invention provides, in part, novel small molecule inhibitors of PHD, having a structure according to Formula (A), and sub-formulas thereof, or a pharmaceutically acceptable salt thereof. The compounds provided herein can be useful for treatment of diseases including heart (e.g. ischemic heart disease, congestive heart failure, and valvular heart disease), lung (e.g., acute lung injury, pulmonary hypertension, pulmonary fibrosis, and chronic obstructive pulmonary disease), liver (e.g. acute liver failure and liver fibrosis and cirrhosis), and kidney (e.g. acute kidney injury and chronic kidney disease) disease.

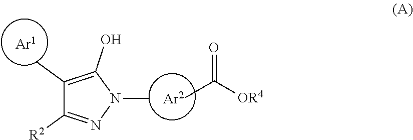

### CROSS-REFERENCE TO RELATED APPLICATION

This application claims priority to U.S. Provisional Patent Application No. 62/992,585, filed Mar. 20, 2020, which is hereby incorporated by reference in its entirety.

### BACKGROUND

Hypoxia is a condition or state in which the supply of oxygen is insufficient for normal life function, for example, where there is low arterial oxygen supply. Hypoxia can lead to functional impairment of cells and structural tissue damage. The activation of cellular defense mechanisms during hypoxia is mediated by HIF (Hypoxia-inducible factor) protein. In response to hypoxic conditions, levels of HIFα are elevated in most cells because of a decrease in HIFα prolyl hydroxylation. Prolyl hydroxylation of HIFα is accomplished by a family of proteins variously termed the prolyl hydroxylase domain-containing proteins (PHD1, 2, and 3), also known as HIF prolyl hydroxylases (HPH-3, 2, and 1) or EGLN-2, 1, and 3. The PHD proteins are oxygen sensors and regulate the stability of HIF in an oxygen dependent manner. The three PHD isoforms function differently in their regulation of HIF and may have other non-HIF related regulatory roles.

In fact, many studies demonstrate that stabilization of HIF can dampen tissue inflammation and promote its repair. Accordingly, compounds that can inhibit the activity of PHD proteins may be particularly beneficial in new therapies (Lee et al. (2019) Exp. Mol. Med. 51:68)

Described herein are novel small molecule PHD inhibitors that have utility for the treatment of disease including heart (e.g. ischemic heart disease, congestive heart failure, and valvular heart disease), lung (e.g., acute lung injury, pulmonary hypertension, pulmonary fibrosis, and chronic obstructive pulmonary disease), liver (e.g. acute liver failure and liver fibrosis and cirrhosis), and kidney (e.g. acute kidney injury and chronic kidney disease) disease.

### SUMMARY

The present invention provides, among other things, novel small molecule inhibitors of PHD and have utility for the treatment of diseases including, but not limited to heart (e.g. ischemic heart disease, congestive heart failure, and valvular heart disease), lung (e.g., acute lung injury, pulmonary hypertension, pulmonary fibrosis, and chronic obstructive pulmonary disease), liver (e.g. acute liver failure and liver fibrosis and cirrhosis), and kidney (e.g. acute kidney injury and chronic kidney disease) disease.

In an aspect, provided herein are compounds having a structure according to Formula (A),

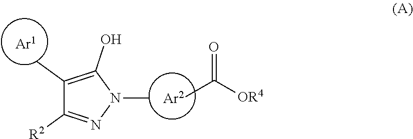

or a pharmaceutically acceptable salt thereof, wherein:

Ar¹ is phenyl or a six-membered nitrogen-containing heteroaryl, wherein said phenyl or heteroaryl is optionally substituted with halogen, CN, OH, C₁₋₃ alkyl optionally substituted with one or more halogens, or C₁₋₃ alkoxy;

R² is H or C₁₋₃ alkyl;

Ar² is a six-membered nitrogen-containing heteroaryl, optionally substituted with halogen, OH, amine, or C₁₋₃ alkyl;

R⁴ is hydrogen or C₁₋₄ alkyl; and

wherein Formula (A) excludes the following compounds:

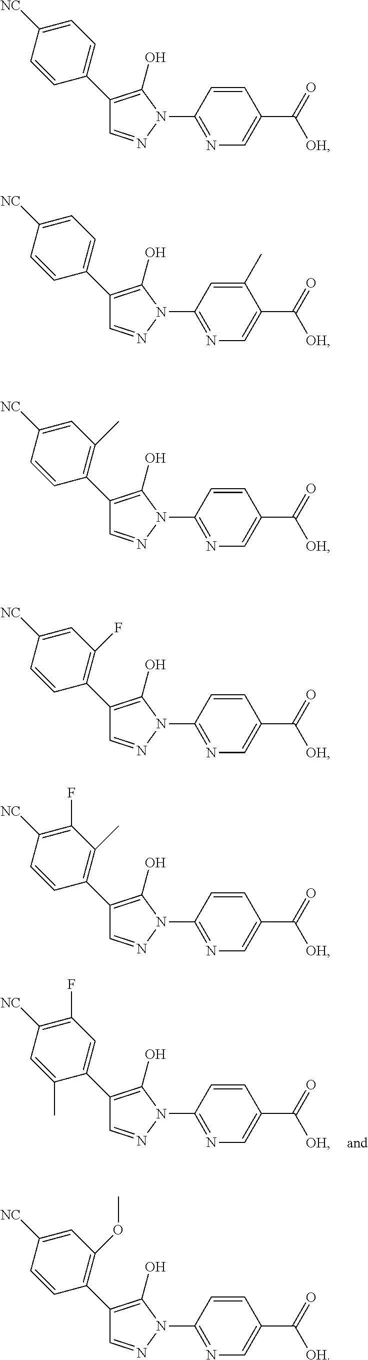

In embodiments, R² is H.

In embodiments, R² is C₁₋₃ alkyl;

In embodiments, Ar¹ is

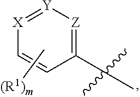

wherein

X, Y, and Z are independently CH or N, wherein N is optionally oxidized;

each R¹ is independently selected from the group consisting of hydrogen, halogen, CN, OH, C₁₋₃ alkyl optionally substituted with one or more halogens, and C₁₋₃ alkoxy; and

m is 1, 2, 3, or 4.

In embodiments, Ar¹ is a substituted phenyl. In embodiments, Ar¹ is a substituted by at least one R¹, wherein R¹ is CN or halogen.

In embodiments, Ar¹ is substituted by one or two R¹ groups independently selected from C₁₋₃ alkyl optionally substituted with one or more halogens, halogen, CN or OH.

In embodiments, Ar¹ is a pyridyl N-oxide or is a pyridyl optionally substituted by at least one R¹ that is C₁₋₃ alkoxy or halogen.

In embodiments, each R¹ is independently selected from the group consisting of hydrogen, halogen, CN, OH, C₁₋₃ alkyl optionally substituted with one or more halogens, and C₁₋₃ alkoxy;

In embodiments, Ar² is

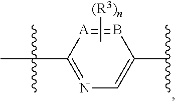

wherein

A and B are independently CH or N, wherein N is optionally oxidized;

each R³ is independently selected from the group consisting of hydrogen, halogen, OH, amine, and C₁₋₃ alkyl; and

n is 0, 1, or 2.

In embodiments, Ar² is a group that is pyridyl or pyrazinyl, and wherein said group is unsubstituted or comprises a substituent that is halogen, C₁₋₃ alkyl, or OH.

In embodiments, each R³ is independently selected from the group consisting of hydrogen, halogen, OH, amine, and C₁₋₃ alkyl.

In embodiments, R⁴ is H. In embodiments, R⁴ is C₁₋₄ alkyl.

In embodiments, a compound of Formula (A) is not

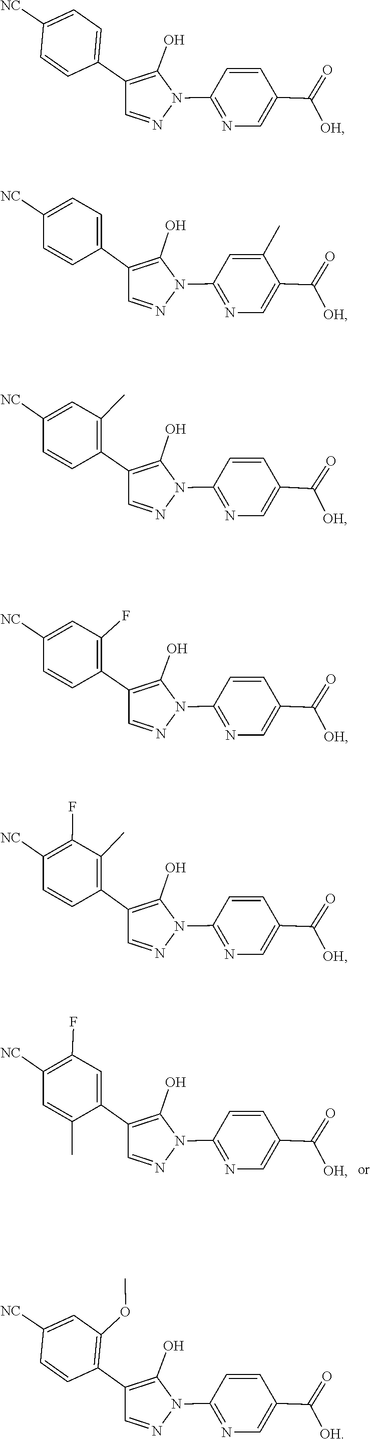

In embodiments, a compound of Formula (A) excludes the following compounds

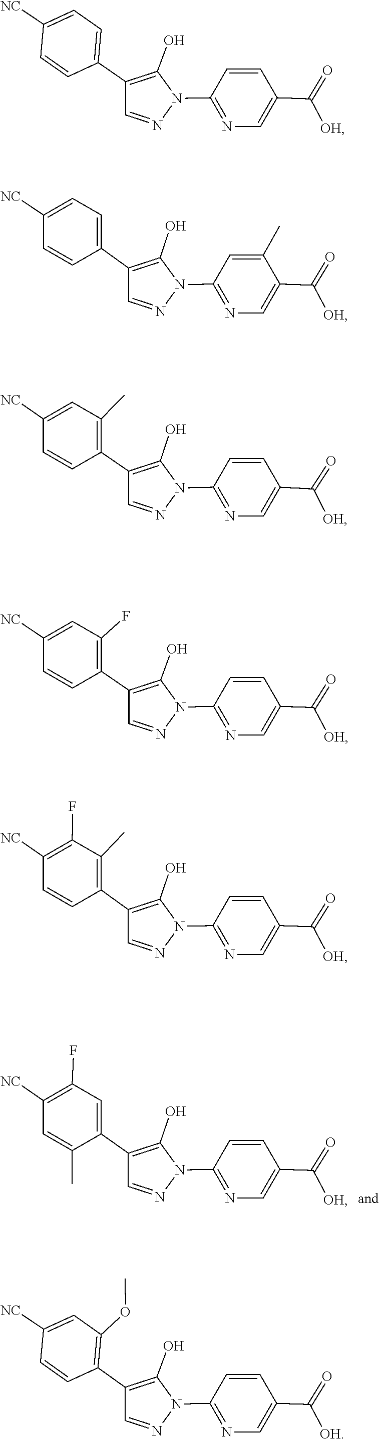

In embodiments, a compound of Formula (A) has the following structure,

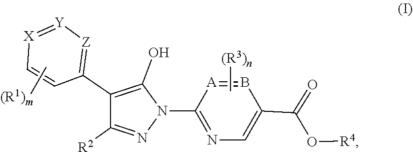

or a pharmaceutically acceptable salt thereof.

In embodiments X, Y, Z, A and B are independently CH or N, wherein N is optionally oxidized; m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; each R³ is independently selected from the group consisting of hydrogen, halogen, OH, amine, and C₁₋₃ alkyl; and R⁴ is hydrogen or C₁₋₄ alkyl.

In embodiments, a compound of Formula (A) or Formula (I) has the following

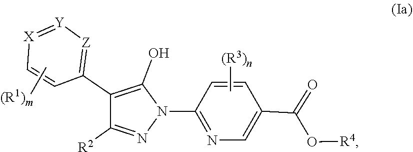

or a pharmaceutically acceptable salt thereof.

In embodiments X, Y, and Z are independently CH or N, wherein N is optionally oxidized; m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; each R³ is independently selected from the group consisting of hydrogen, halogen, OH, amine, and C₁₋₃ alkyl; and R⁴ is hydrogen or C₁₋₄ alkyl.

In embodiments, a compound of Formula (I) is not

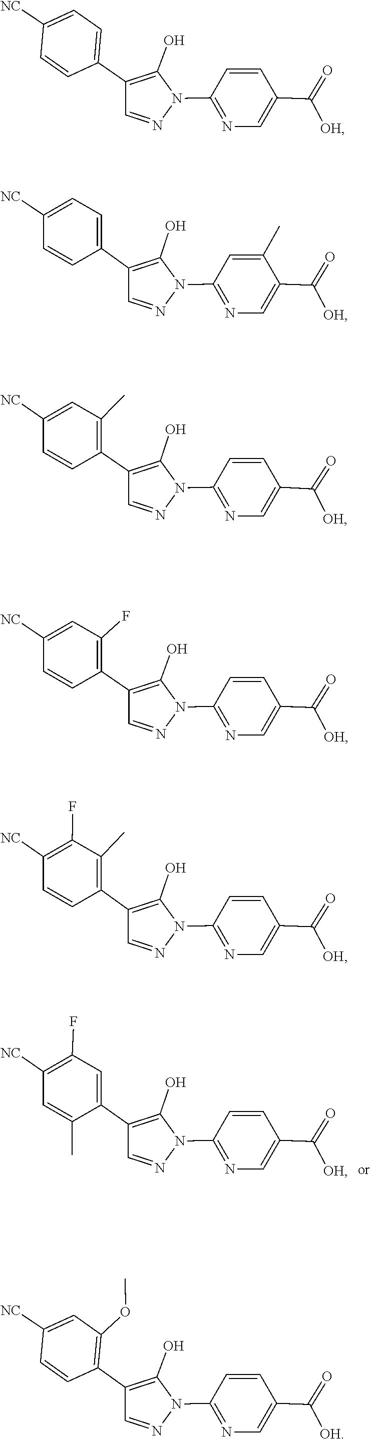

In embodiments, a compound of Formula (I) excludes the following compounds

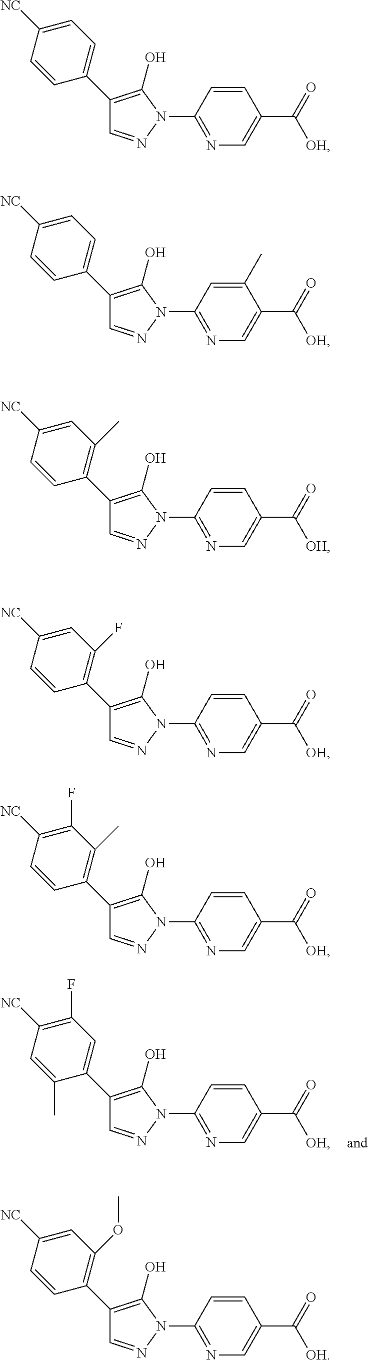

In embodiments, a compound of Formula (A) or Formula (I) has the following structure,

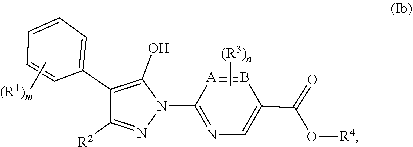

or a pharmaceutically acceptable salt thereof.

In embodiments A and B are independently CH or N, wherein N is optionally oxidized; m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; each R³ is independently selected from the group consisting of hydrogen, halogen, OH, amine, and C₁₋₃ alkyl; and R⁴ is hydrogen or C₁₋₄ alkyl.

In embodiments, a compound of Formula (Ib) is not

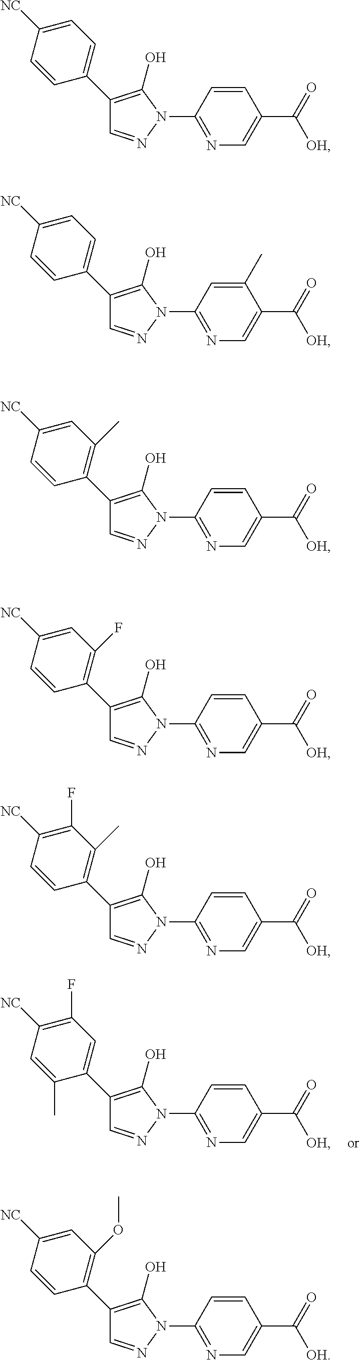

In embodiments, a compound of Formula (Ib) excludes the following compounds

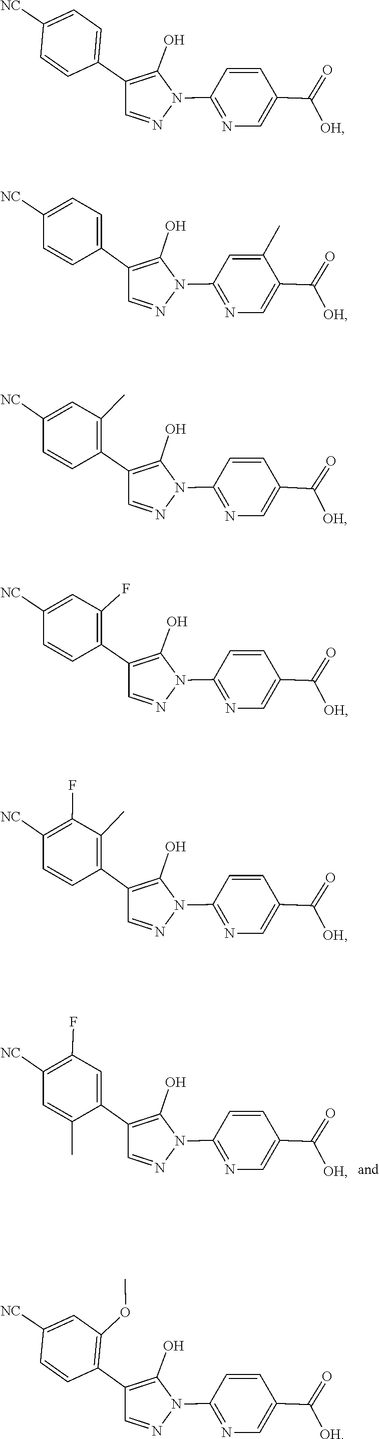

In embodiments, a compound of Formula (A) or Formula (I) has the following structure,

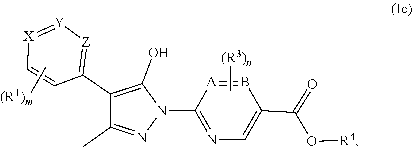

or a pharmaceutically acceptable salt thereof.

In embodiments X, Y, Z, A and B are independently CH or N, wherein N is optionally oxidized; m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; each R³ is independently selected from the group consisting of hydrogen, halogen, OH, amine, and C₁₋₃ alkyl; and R⁴ is hydrogen or C₁₋₄ alkyl.

In embodiments, a compound of Formula (A) or Formula (I) has the following structure,

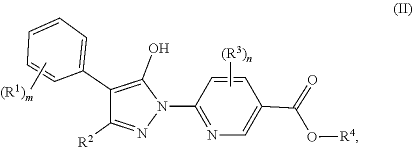

or a pharmaceutically acceptable salt thereof.

In embodiments m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; each R³ is independently selected from the group consisting of hydrogen, halogen, OH, amine, and C₁₋₃ alkyl; and R⁴ is hydrogen or C₁₋₄ alkyl.

In embodiments, a compound of Formula (II) is not

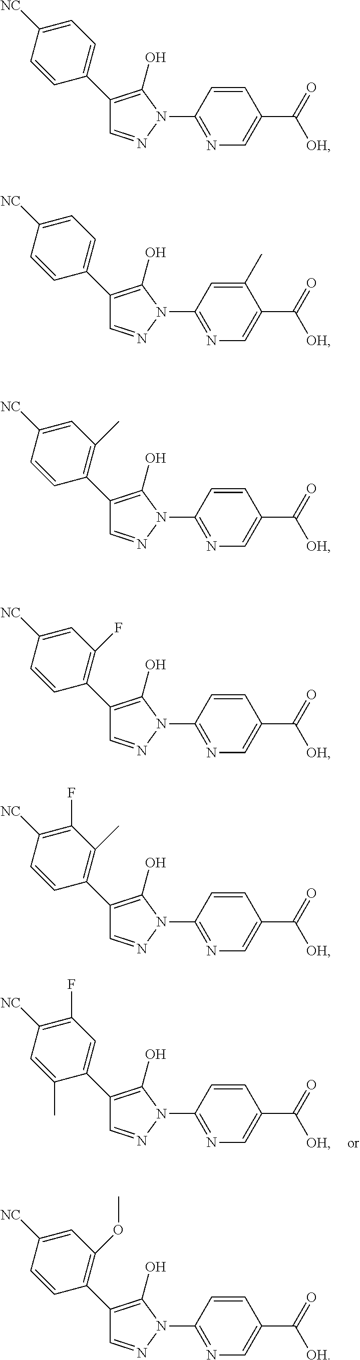

In embodiments, a compound of Formula (II) excludes the following compounds

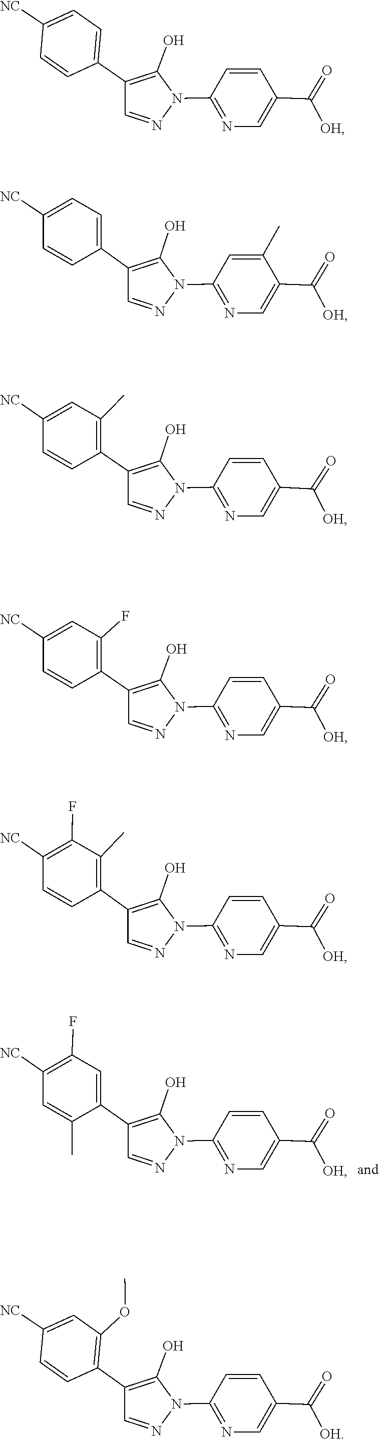

In embodiments, a compound of Formula (A), Formula (I) or Formula (II) has the following structure,

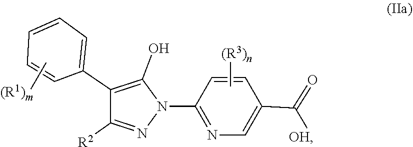

or a pharmaceutically acceptable salt thereof.

In embodiments m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; and each R³ is independently selected from the group consisting of hydrogen, halogen, OH, amine, and C₁₋₃ alkyl.

In embodiments, a compound of Formula (IIa) is not

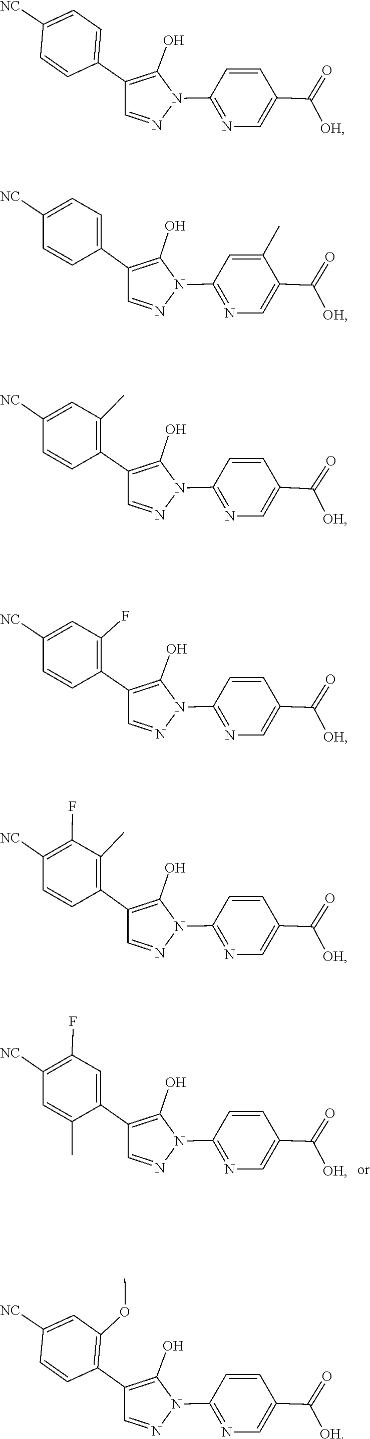

In embodiments, a compound of Formula (IIa) excludes the following compounds

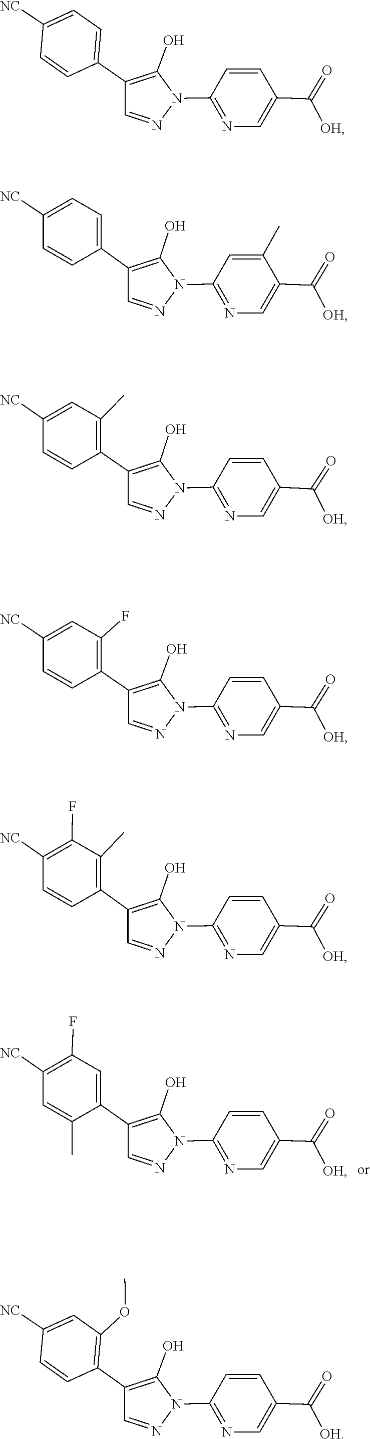

In embodiments, a compound of Formula (A), Formula (I) or Formula (II), has the following structure,

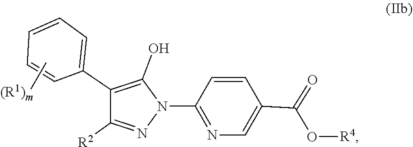

or a pharmaceutically acceptable salt thereof.

In embodiments m is 1, 2, 3, or 4; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; and R⁴ is hydrogen or C₁₋₄ alkyl.

In embodiments, a compound of Formula (IIb) is not

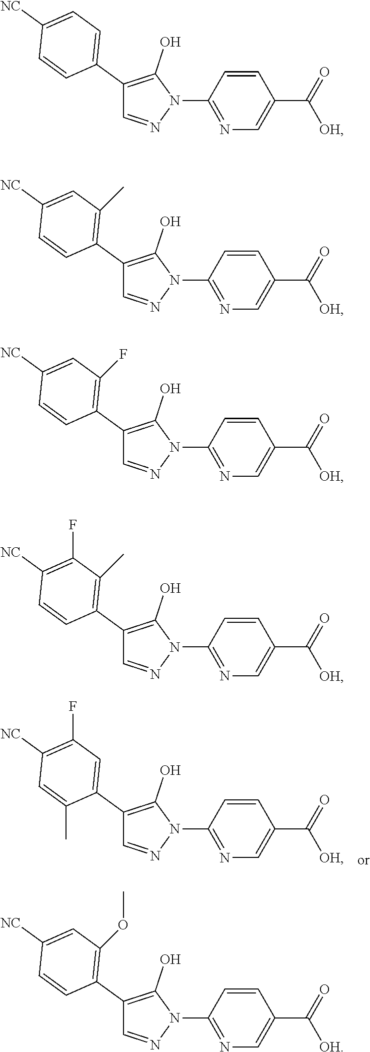

In embodiments, a compound of Formula (IIb) excludes the following compounds

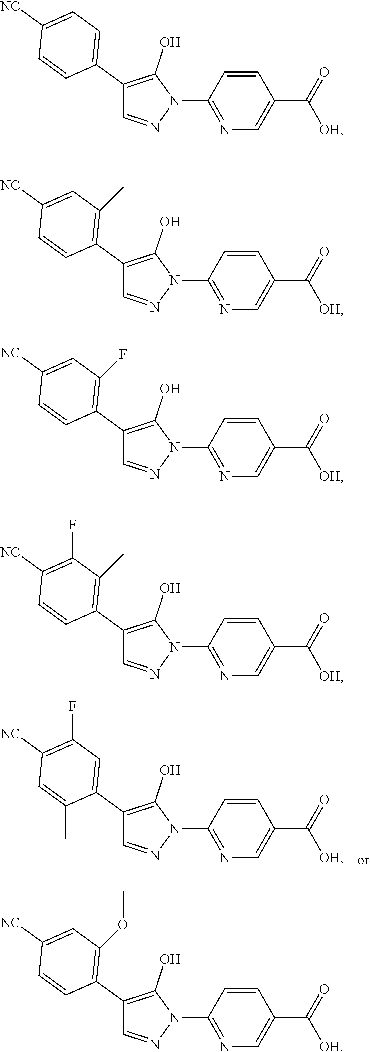

In embodiments, a compound of Formula (A), Formula (I) or Formula (II), has the following structure,

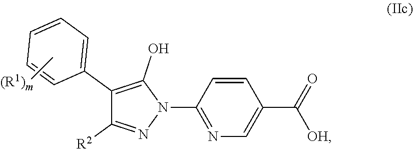

or a pharmaceutically acceptable salt thereof.

In embodiments m is 1, 2, 3, or 4; each R¹ is independently selected from the group consisting of hydrogen, halogen, CN, OH, C₁₋₃ alkyl optionally substituted with one or more halogens, and C₁₋₃ alkoxy; and R² is hydrogen or C₁₋₃ alkyl.

In embodiments, a compound of Formula (IIc) is not

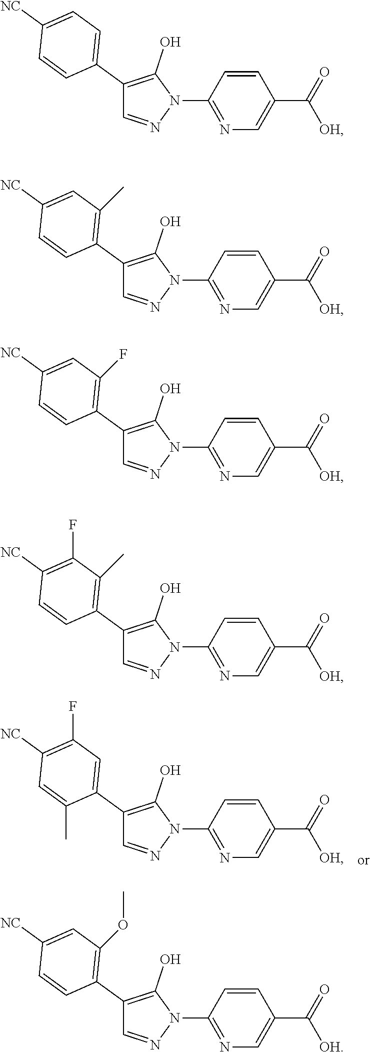

In embodiments, a compound of Formula (IIc) excludes the following compounds

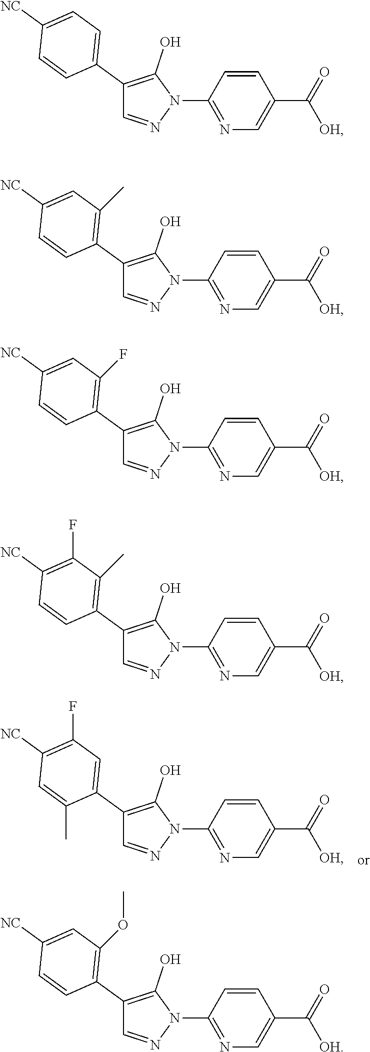

In embodiments, a compound of Formula (A), Formula (I) or Formula (II), has the following structure,

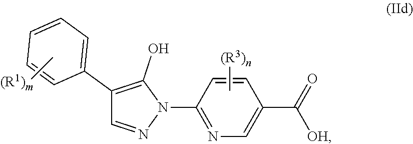

or a pharmaceutically acceptable salt thereof.

In embodiments m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; and each R³ is independently selected from the group consisting of hydrogen, halogen, OH, amine, and C₁₋₃ alkyl.

In embodiments, a compound of Formula (IId) is not

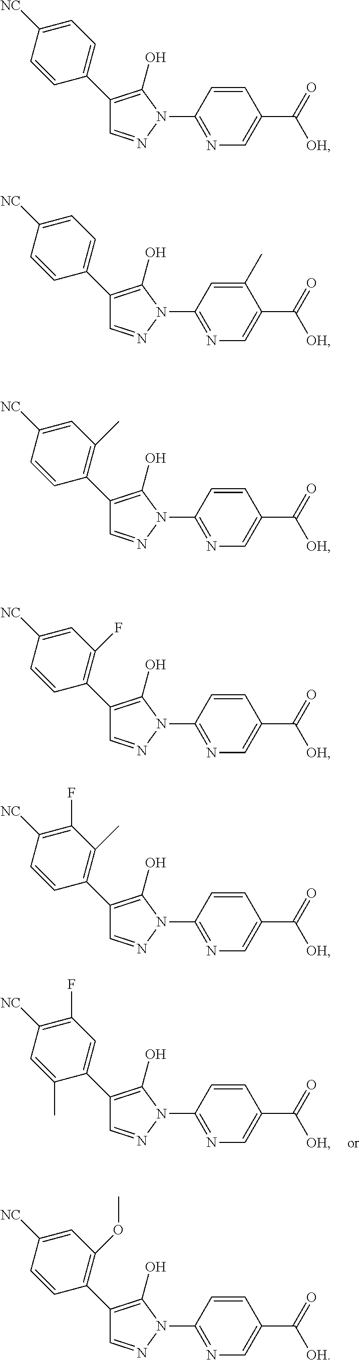

In embodiments, a compound of Formula (IId) excludes the following compounds

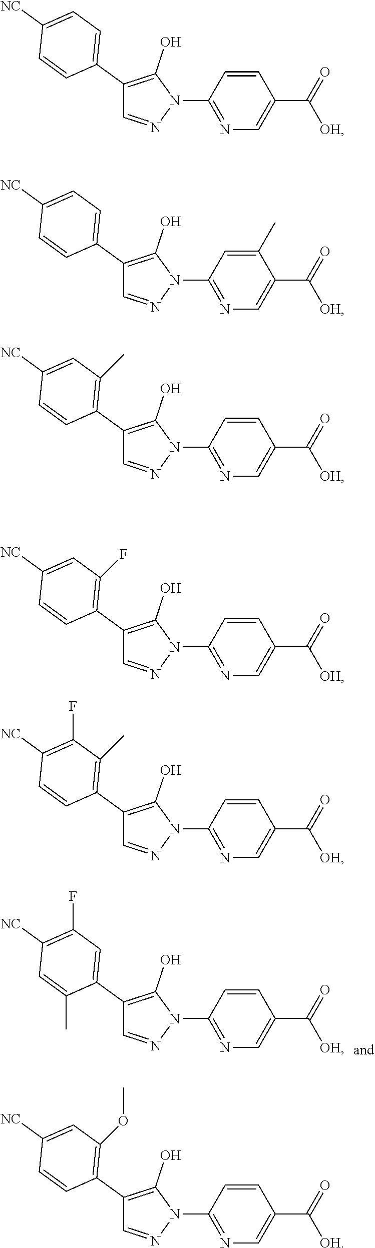

In embodiments, a compound of Formula (A), Formula (I) or Formula (II), has the following structure,

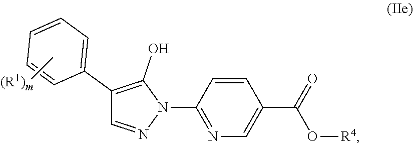

or a pharmaceutically acceptable salt thereof.

In embodiments m is 1, 2, 3, or 4; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; each R³ is independently selected from the group consisting of hydrogen, halogen, OH, amine, and C₁₋₃ alkyl; and R⁴ is hydrogen or C₁₋₄ alkyl.

In embodiments, a compound of Formula (IIe) is not

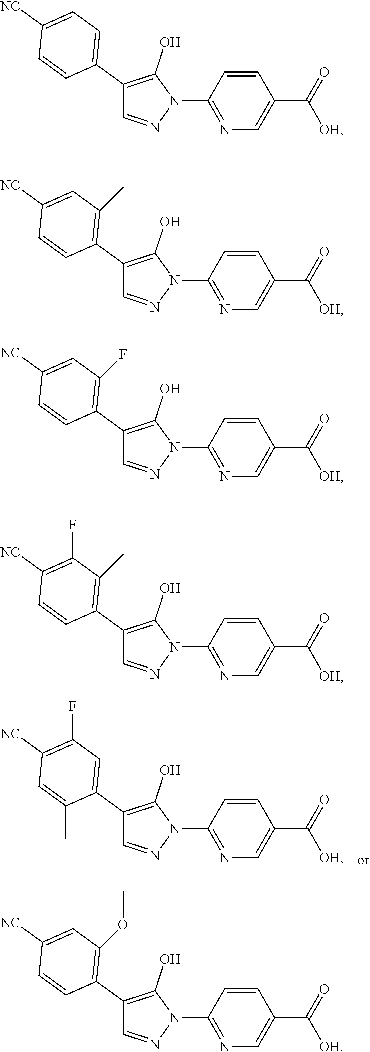

In embodiments, a compound of Formula (IIe) excludes the following compounds

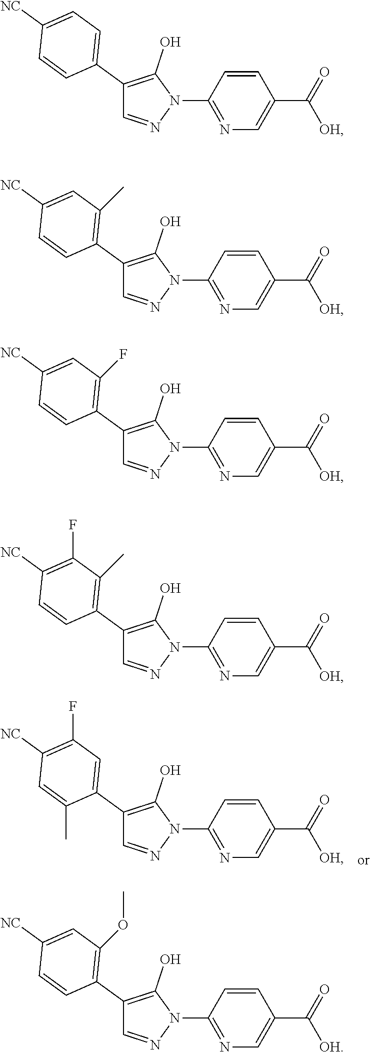

In embodiments, a compound of Formula (A), Formula (I) or Formula (II), has the following structure,

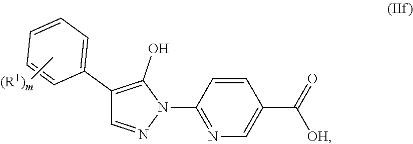

or a pharmaceutically acceptable salt thereof.

In embodiments m is 1, 2, 3, or 4; and each R¹ is independently selected from the group consisting of hydrogen, halogen, CN, OH, C₁₋₃ alkyl optionally substituted with one or more halogens, and C₁₋₃ alkoxy.

In embodiments, a compound of Formula (IIf) is not

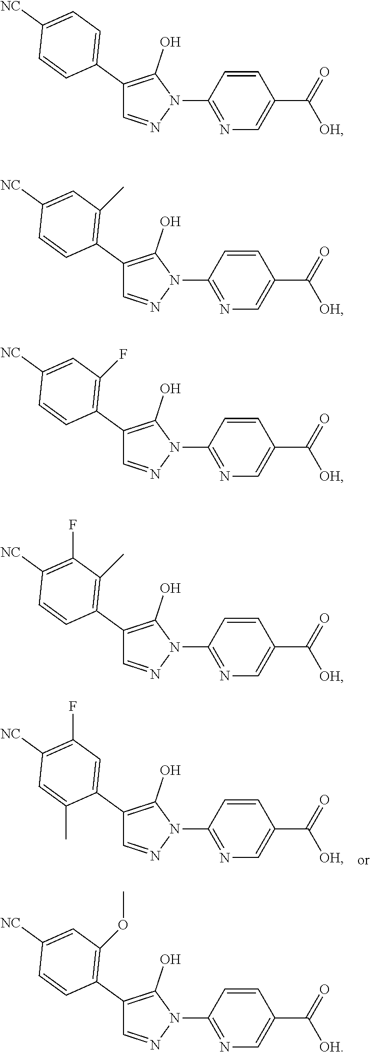

In embodiments, a compound of Formula (IIf) excludes the following compounds

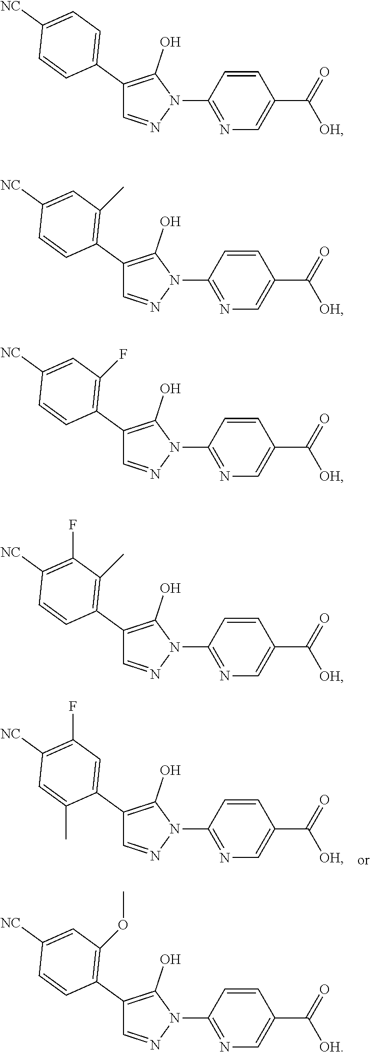

In embodiments, a compound of Formula (A), Formula (I) or Formula (II), has the following structure,

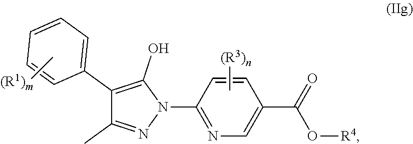

or a pharmaceutically acceptable salt thereof.

In embodiments m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; each R³ is independently selected from the group consisting of hydrogen, halogen, OH, amine, and C₁₋₃ alkyl; and R⁴ is hydrogen or C₁₋₄ alkyl.

In embodiments, a compound of Formula (A), Formula (I) or Formula (II), has the following structure,

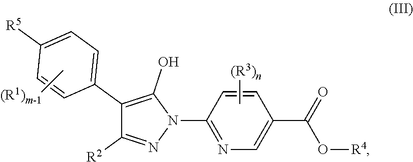

or a pharmaceutically acceptable salt thereof.

In embodiments m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; each R³ is independently selected from the group consisting of hydrogen, halogen, OH, amine, and C₁₋₃ alkyl; R⁴ is hydrogen or C₁₋₄ alkyl; and R⁵ is CN or halogen.

In embodiments, a compound of Formula (III) is not

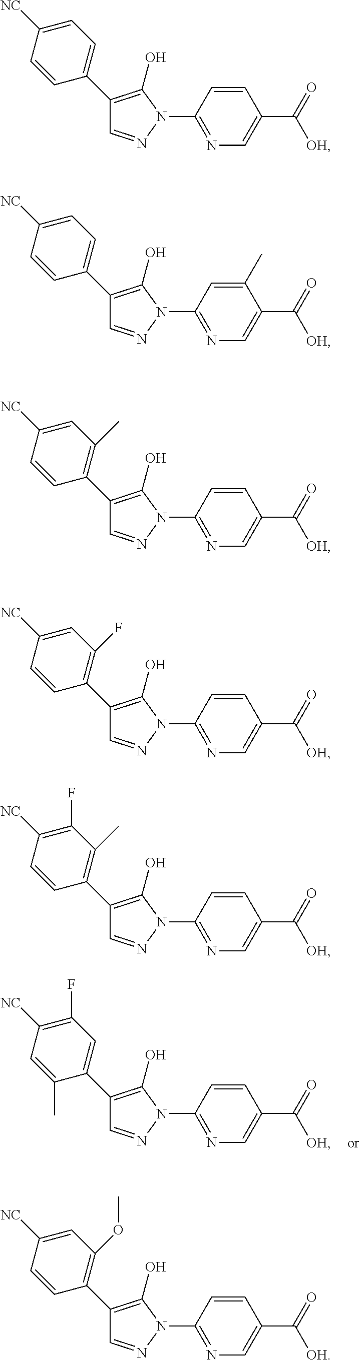

In embodiments, a compound of Formula (III) excludes the following compounds

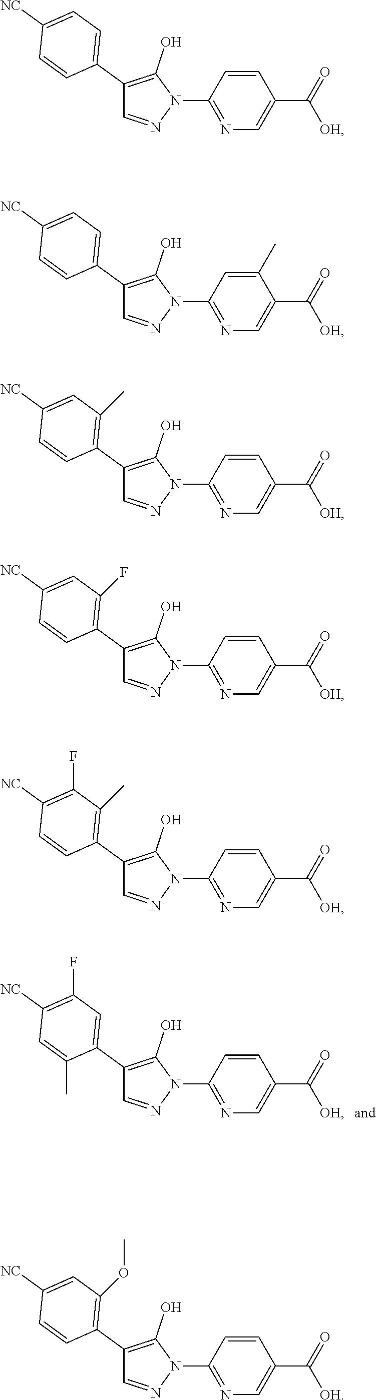

In embodiments, a compound of Formula (A), Formula (I), Formula (II), or Formula (III), has the following structure,

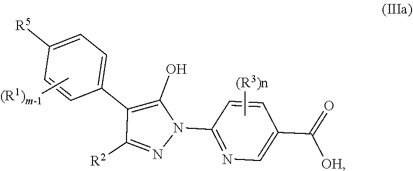

or a pharmaceutically acceptable salt thereof.

In embodiments m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; each R³ is independently selected from the group consisting of hydrogen, halogen, OH, amine, and C₁₋₃ alkyl; and R⁵ is CN or halogen.

In embodiments, a compound of Formula (IIIa) is not

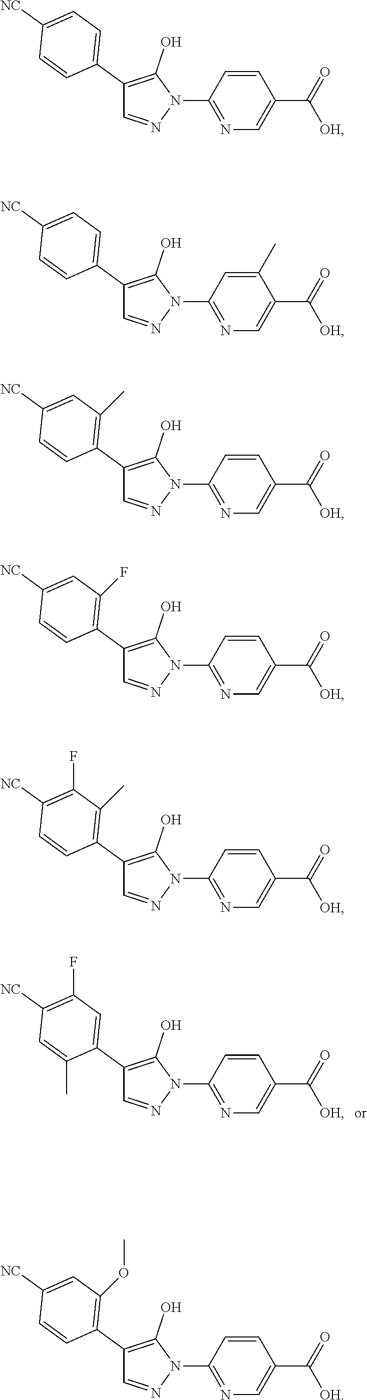

In embodiments, a compound of Formula (IIIa) excludes the following compounds

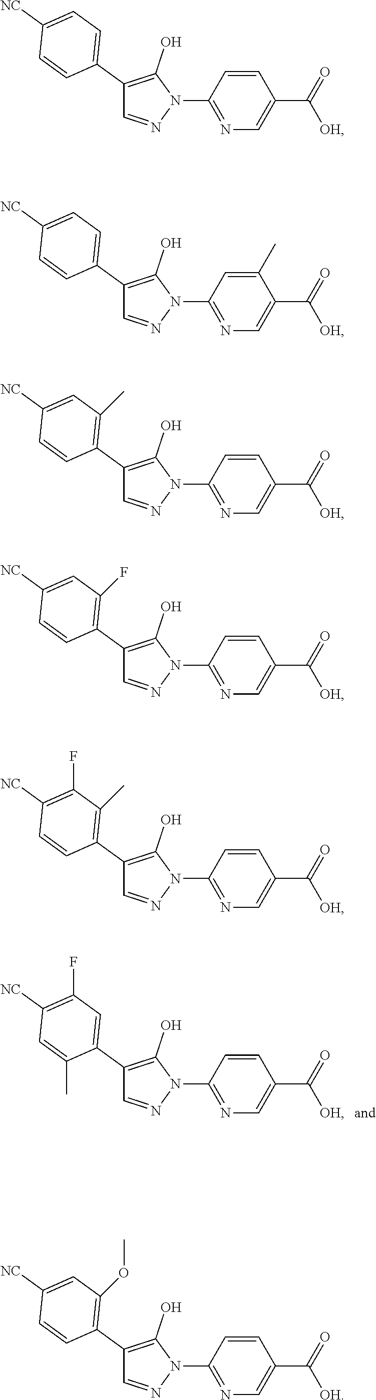

In embodiments, a compound of Formula (A), Formula (I), Formula (II), or Formula (III), has the following structure,

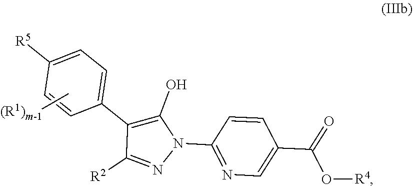

or a pharmaceutically acceptable salt thereof.

In embodiments m is 1, 2, 3, or 4; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; R⁴ is hydrogen or C₁₋₄ alkyl; and R⁵ is CN or halogen.

In embodiments, a compound of Formula (IIIb) is not

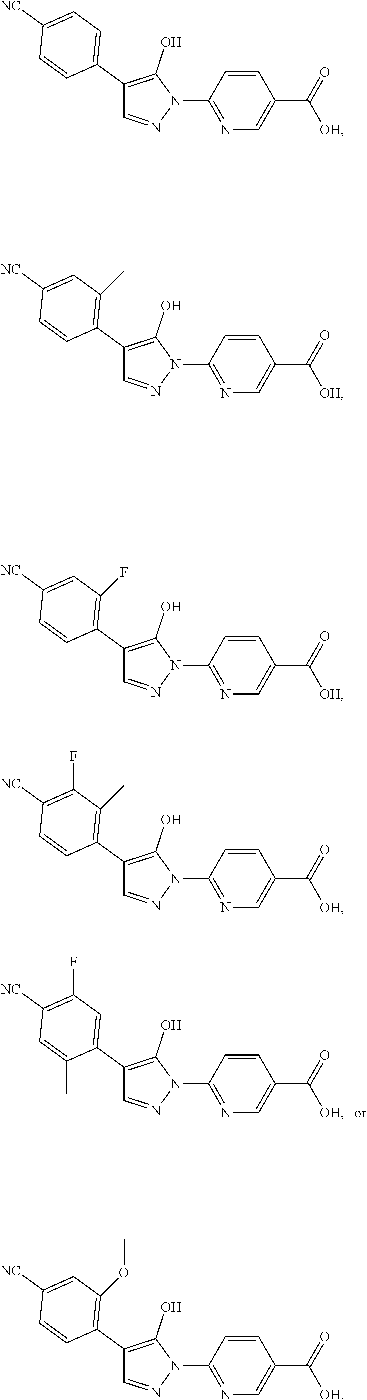 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (IIIb) excludes the following compounds

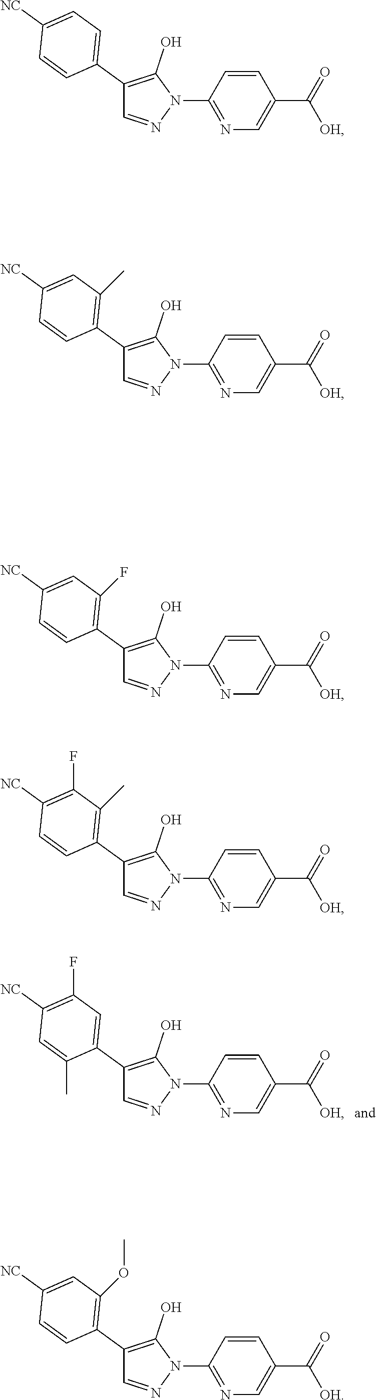 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (A), Formula (I), Formula (II), or Formula (III), has the following structure,

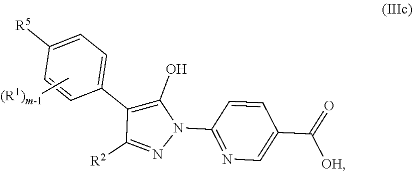

or a pharmaceutically acceptable salt thereof.

In embodiments m is 1, 2, 3, or 4; each R¹ is independently selected from the group consisting of hydrogen, halogen, CN, OH, C₁₋₃ alkyl optionally substituted with one or more halogens, and C₁₋₃ alkoxy; R² is hydrogen or C₁₋₃ alkyl; and R⁵ is CN or halogen.

In embodiments, a compound of Formula (IIIc) is not

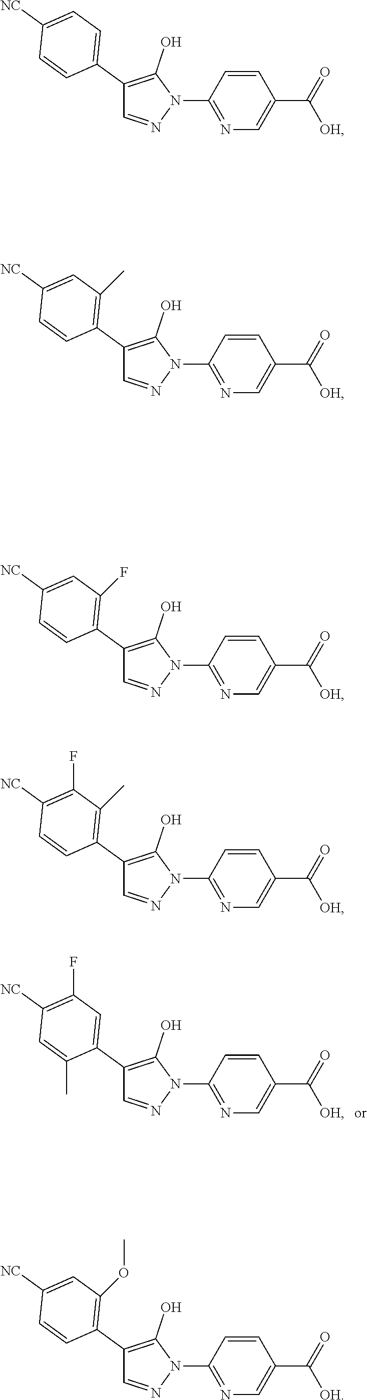 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (IIIc) excludes the following compounds

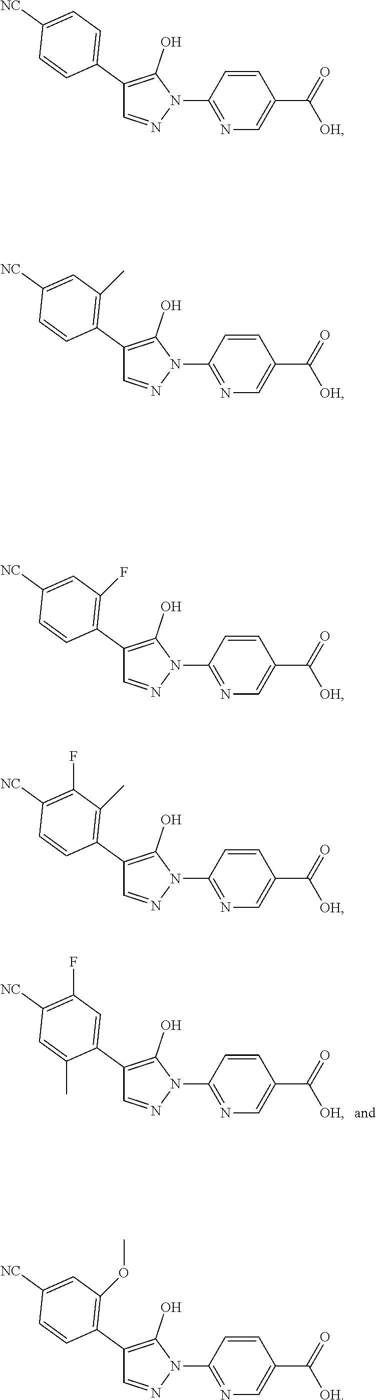 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (A), Formula (I), Formula (II), or Formula (III), has the following structure,

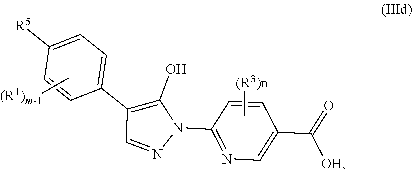

or a pharmaceutically acceptable salt thereof.

In embodiments m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; each R³ is independently selected from the group consisting of hydrogen, halogen, OH, amine, and C₁₋₃ alkyl; and R⁵ is CN or halogen.

In embodiments, a compound of Formula (IIId) is not

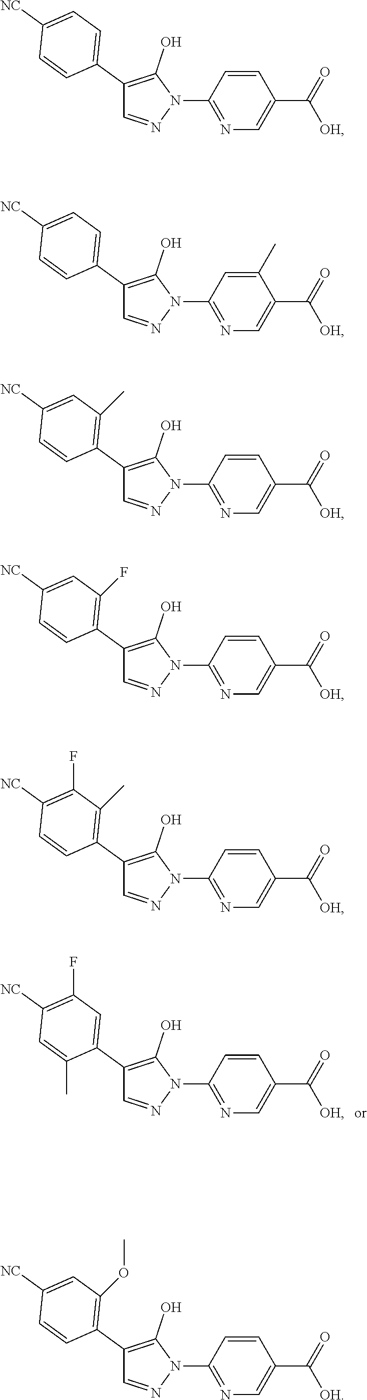

In embodiments, a compound of Formula (IIId) excludes the following compounds

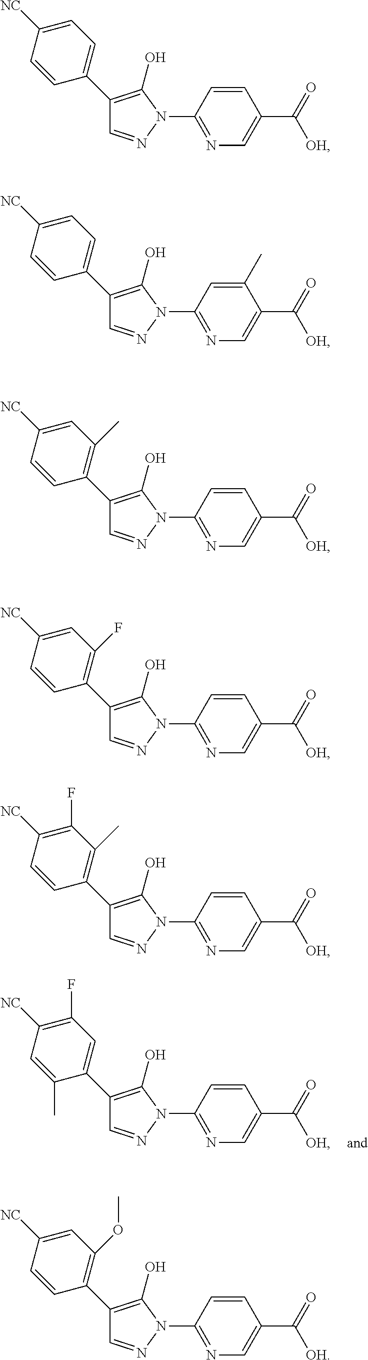

In embodiment, a compound of Formula (A), Formula (I), Formula (II), or Formula (III), has the following structure,

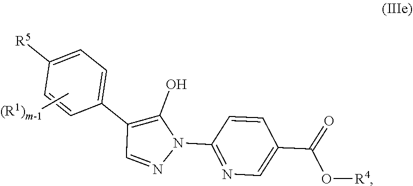

or a pharmaceutically acceptable salt thereof.

In embodiments m is 1, 2, 3, or 4; each R¹ is independently selected from the group consisting of hydrogen, halogen, CN, OH, C₁₋₃ alkyl optionally substituted with one or more halogens, and C₁₋₃ alkoxy; R⁴ is hydrogen or C₁₋₄ alkyl; and R⁵ is CN or halogen.

In embodiments, a compound of Formula (IIIe) is not

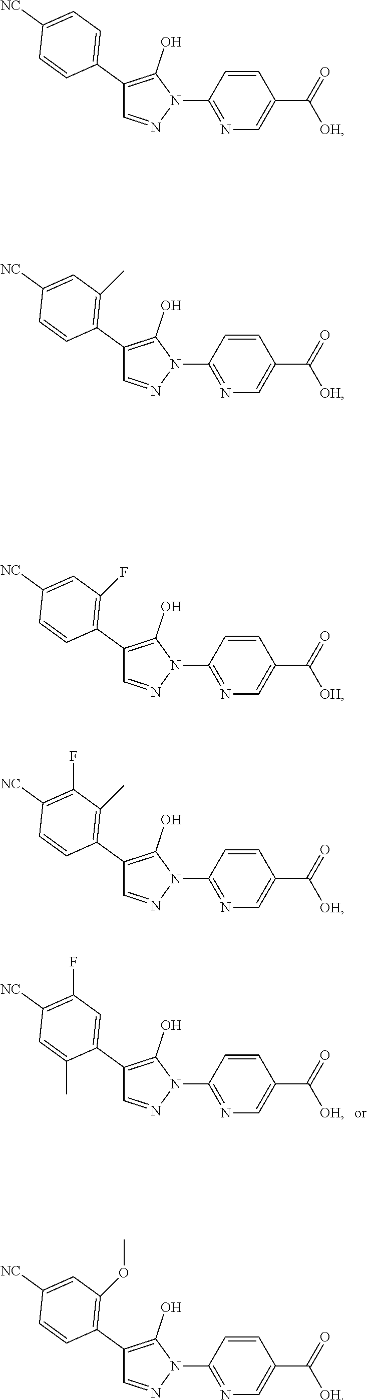 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (IIIe) excludes the following compounds

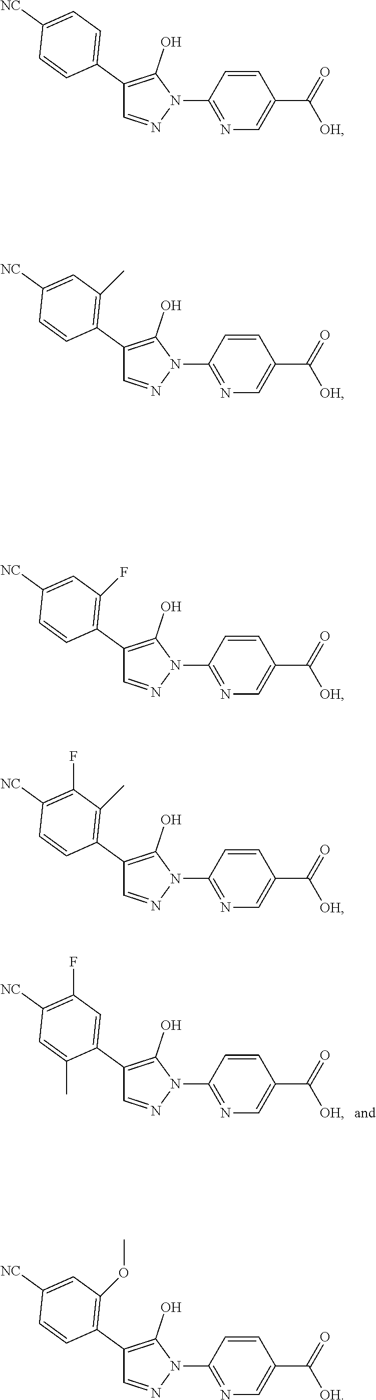 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (A), Formula (I), Formula (II), or Formula (III), has the following structure,

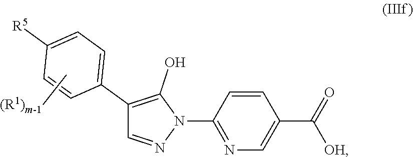

or a pharmaceutically acceptable salt thereof.

In embodiments m is 1, 2, 3, or 4; each R¹ is independently selected from the group consisting of hydrogen, halogen, CN, OH, C₁₋₃ alkyl optionally substituted with one or more halogens, and C₁₋₃ alkoxy; and R⁵ is CN or halogen.

In embodiments, a compound of Formula (IIIf) is not

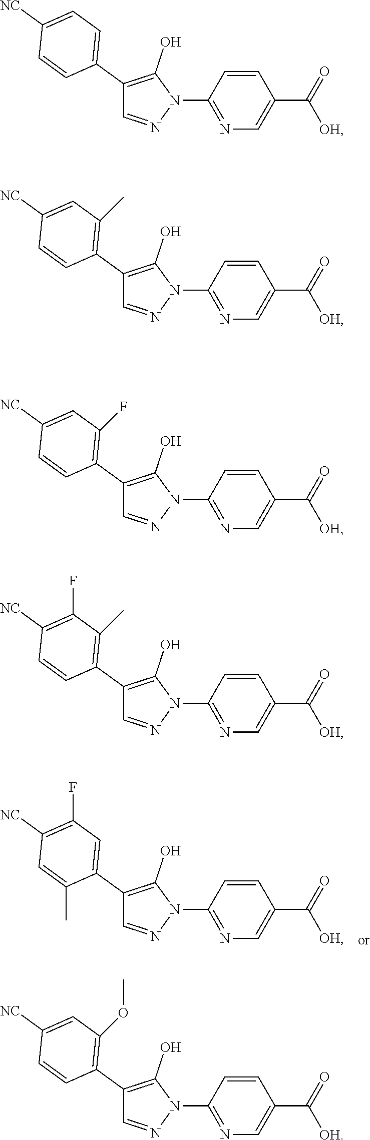 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (IIIf) excludes the following compounds

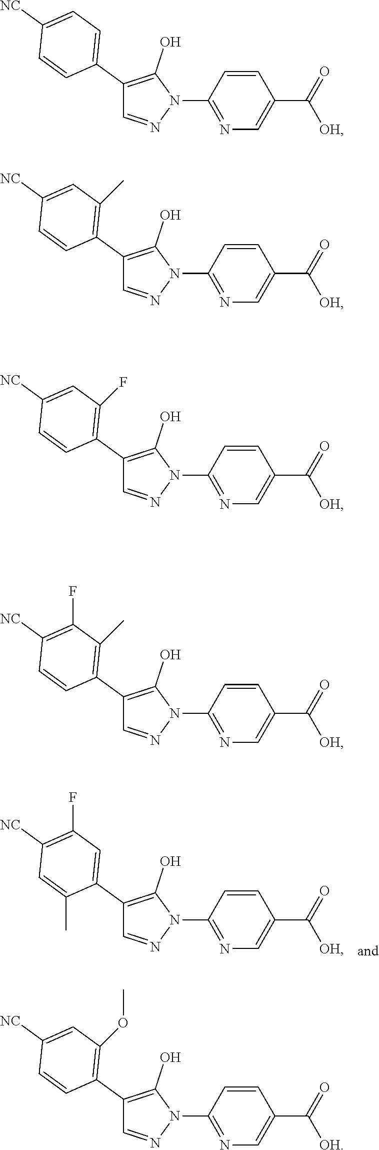 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (A), Formula (I), Formula (II), or Formula (III), has the following structure,

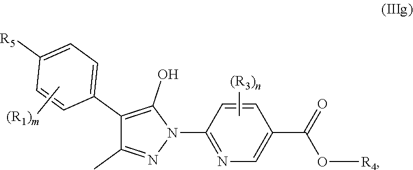

or a pharmaceutically acceptable salt thereof.

In embodiments m is 1, 2, 3, or 4; n is 0, 1, or 2; each R¹ is independently selected from the group consisting of hydrogen, halogen, CN, OH, C₁₋₃ alkyl optionally substituted with one or more halogens, and C₁₋₃ alkoxy; each R³ is independently selected from the group consisting of hydrogen, halogen, OH, amine, and C₁₋₃ alkyl; R⁴ is hydrogen or C₁₋₄ alkyl; and R⁵ is CN or halogen.

In embodiments, X is CH. In embodiments, X is N, wherein N is optionally oxidized.

In embodiments, Y is CH. In embodiments, Y is N.

In embodiments, Z is CH, In embodiments, Z is N.

In embodiments, A is CH, In embodiments, A is N.

In embodiments, B is CH, In embodiments, B is N.

In embodiments, m is 1. In embodiments, m is 2. In embodiments, m is 3. In embodiments, m is 4.

In embodiments, n is 0. In embodiments, n is 1. In embodiments, n is 2.

In embodiments, R¹ is hydrogen. In embodiments, R¹ is CN. In embodiments, R¹ is OH.

In embodiments, R¹ is halogen. In embodiments, R¹ is F. In embodiments, R¹ is Cl. In embodiments, R¹ is Br.

In embodiments, R¹ is C₁₋₃ alkyl optionally substituted with one or more halogens. In embodiments, R¹ is C₁₋₃ alkyl. In embodiments, R¹ is methyl. In embodiments, R¹ is ethyl. In embodiments, R¹ is CF₃.

In embodiments, R¹ is C₁₋₃ alkoxy. In embodiments, R¹ is methoxy.

In embodiments, R² is hydrogen.

In embodiments, R² is C₁₋₃ alkyl. In embodiments, R² is methyl.

In embodiments, R³ is hydrogen.

In embodiments, R³ is halogen. In embodiments, R³ is F.

In embodiments, R³ is OH.

In embodiments, R³ is amine. In embodiments, R³ is NH₂.

In embodiments, R³ is C₁₋₃ alkyl. In embodiments, R³ is methyl.

In embodiments, R⁴ is hydrogen.

In embodiments, R⁴ is C₁₋₄ alkyl. In embodiments, R⁴ is methyl. In embodiments, R⁴ is ethyl. In embodiments, R⁴ is isopropyl. In embodiments, R⁴ is tert-butyl.

In embodiments, R⁵ is F. In embodiments, R⁵ is Cl. In embodiments, R⁵ is Br.

In embodiments, R⁵ is CN.

In some embodiments, the compound is any one of Compounds 1-44, or a pharmaceutically acceptable salt thereof:

| Compd. | |
|---|---|
| No. | Structure |
| 1 | 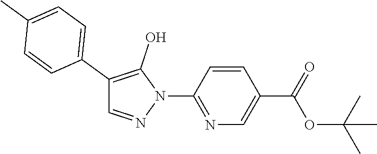 [SMILES: Cc1ccc(-c2cnn(-c3ccc(C(=O)OC(C)(C)C)cn3)c2O)cc1] |
| 2 | 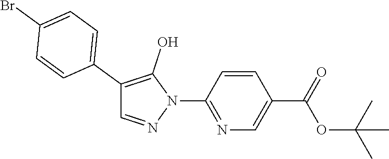 [SMILES: CC(C)(C)OC(=O)c1ccc(-n2ncc(-c3ccc(Br)cc3)c2O)nc1] |
| 3 | 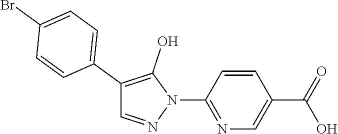 [SMILES: O=C(O)c1ccc(-n2ncc(-c3ccc(Br)cc3)c2O)nc1] |
| 4 | 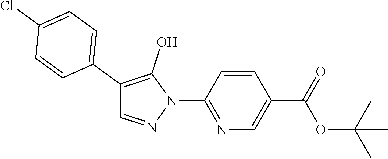 [SMILES: CC(C)(C)OC(=O)c1ccc(-n2ncc(-c3ccc(Cl)cc3)c2O)nc1] |
| 5 | 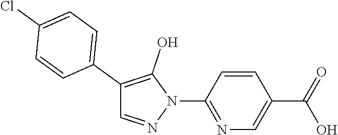 [SMILES: O=C(O)c1ccc(-n2ncc(-c3ccc(Cl)cc3)c2O)nc1] |
| 6 | 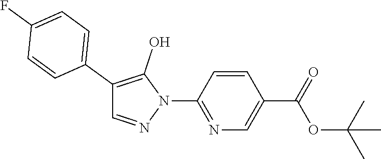 [SMILES: CC(C)(C)OC(=O)c1ccc(-n2ncc(-c3ccc(F)cc3)c2O)nc1] |
| 7 | 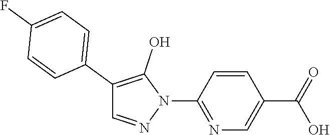 [SMILES: O=C(O)c1ccc(-n2ncc(-c3ccc(F)cc3)c2O)nc1] |
| 8 | 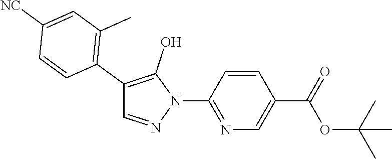 [SMILES: Cc1cc(C#N)ccc1-c1cnn(-c2ccc(C(=O)OC(C)(C)C)cn2)c1O] |
| 9 | 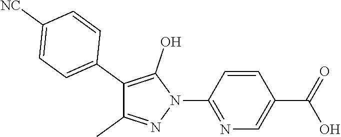 [SMILES: Cc1nn(-c2ccc(C(=O)O)cn2)c(O)c1-c1ccc(C#N)cc1] |
| 10 | 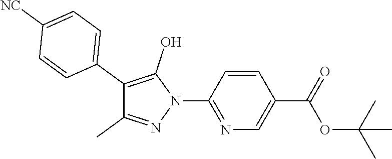 [SMILES: Cc1nn(-c2ccc(C(=O)OC(C)(C)C)cn2)c(O)c1-c1ccc(C#N)cc1] |
| 11 | 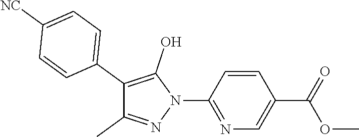 [SMILES: COC(=O)c1ccc(-n2nc(C)c(-c3ccc(C#N)cc3)c2O)nc1] |
| 12 | 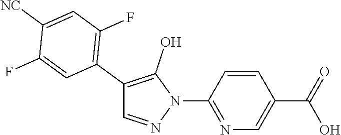 [SMILES: N#Cc1cc(F)c(-c2cnn(-c3ccc(C(=O)O)cn3)c2O)cc1F] |
| 13 | 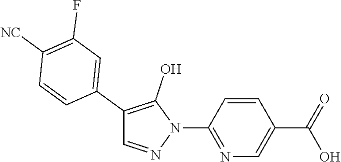 [SMILES: N#Cc1ccc(-c2cnn(-c3ccc(C(=O)O)cn3)c2O)cc1F] |
| 14 | 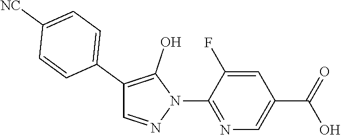 [SMILES: N#Cc1ccc(-c2cnn(-c3ncc(C(=O)O)cc3F)c2O)cc1] |
| 15 | 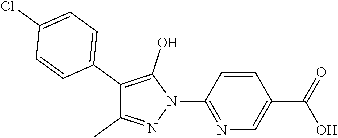 [SMILES: Cc1nn(-c2ccc(C(=O)O)cn2)c(O)c1-c1ccc(Cl)cc1] |
| 16 | 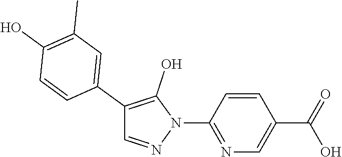 [SMILES: Cc1cc(-c2cnn(-c3ccc(C(=O)O)cn3)c2O)ccc1O] |
| 17 | 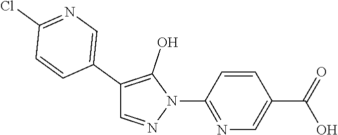 [SMILES: O=C(O)c1ccc(-n2ncc(-c3ccc(Cl)nc3)c2O)nc1] |
| 18 | 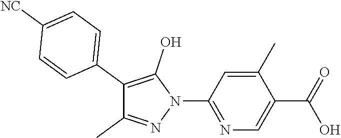 [SMILES: Cc1cc(-n2nc(C)c(-c3ccc(C#N)cc3)c2O)ncc1C(=O)O] |
| 19 | 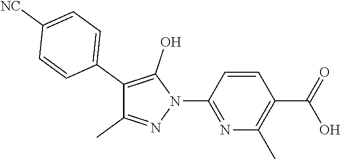 [SMILES: Cc1nc(-n2nc(C)c(-c3ccc(C#N)cc3)c2O)ccc1C(=O)O] |
| 20 | 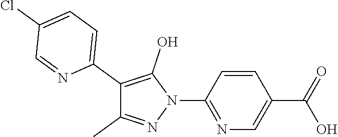 [SMILES: Cc1nn(-c2ccc(C(=O)O)cn2)c(O)c1-c1ccc(Cl)cn1] |
| 21 | 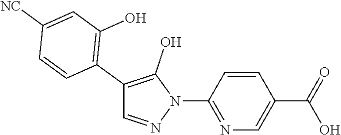 [SMILES: N#Cc1ccc(-c2cnn(-c3ccc(C(=O)O)cn3)c2O)c(O)c1] |
| 22 | 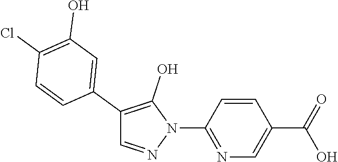 [SMILES: O=C(O)c1ccc(-n2ncc(-c3ccc(Cl)c(O)c3)c2O)nc1] |
| 23 | 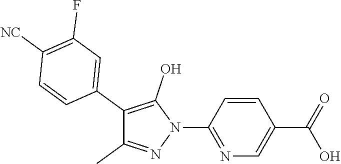 [SMILES: Cc1nn(-c2ccc(C(=O)O)cn2)c(O)c1-c1ccc(C#N)c(F)c1] |
| 24 | 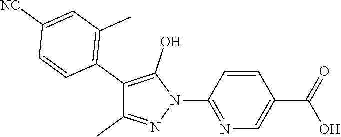 [SMILES: Cc1cc(C#N)ccc1-c1c(C)nn(-c2ccc(C(=O)O)cn2)c1O] |
| 25 | 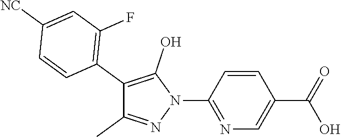 [SMILES: Cc1nn(-c2ccc(C(=O)O)cn2)c(O)c1-c1ccc(C#N)cc1F] |
| 26 | 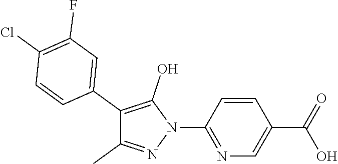 [SMILES: Cc1nn(-c2ccc(C(=O)O)cn2)c(O)c1-c1ccc(Cl)c(F)c1] |
| 27 | 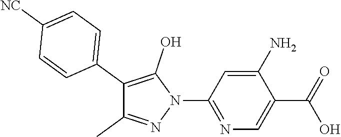 [SMILES: Cc1nn(-c2cc(N)c(C(=O)O)cn2)c(O)c1-c1ccc(C#N)cc1] |
| 28 | 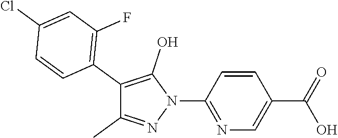 [SMILES: Cc1nn(-c2ccc(C(=O)O)cn2)c(O)c1-c1ccc(Cl)cc1F] |
| 29 | 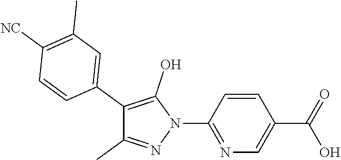 [SMILES: Cc1cc(-c2c(C)nn(-c3ccc(C(=O)O)cn3)c2O)ccc1C#N] |
| 30 | 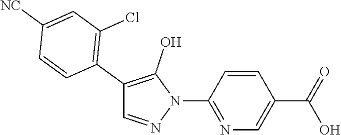 [SMILES: N#Cc1ccc(-c2cnn(-c3ccc(C(=O)O)cn3)c2O)c(Cl)c1] |
| 31 | 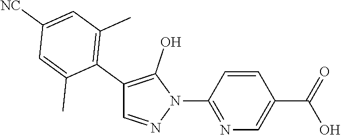 [SMILES: Cc1cc(C#N)cc(C)c1-c1cnn(-c2ccc(C(=O)O)cn2)c1O] |
| 32 | 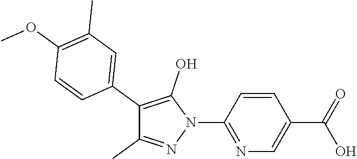 [SMILES: COc1ccc(-c2c(C)nn(-c3ccc(C(=O)O)cn3)c2O)cc1C] |
| 33 | 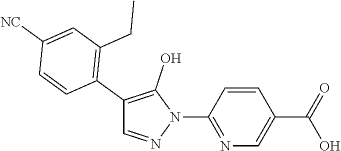 [SMILES: CCc1cc(C#N)ccc1-c1cnn(-c2ccc(C(=O)O)cn2)c1O] |
| 34 | 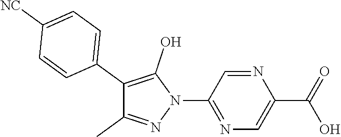 [SMILES: Cc1nn(-c2cnc(C(=O)O)cn2)c(O)c1-c1ccc(C#N)cc1] |
| 35 | 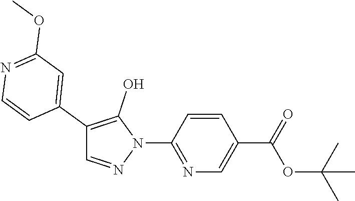 [SMILES: COc1cc(-c2cnn(-c3ccc(C(=O)OC(C)(C)C)cn3)c2O)ccn1] |
| 36 | 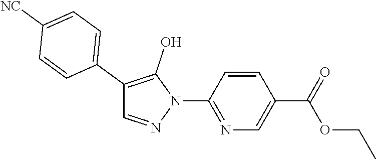 [SMILES: CCOC(=O)c1ccc(-n2ncc(-c3ccc(C#N)cc3)c2O)nc1] |
| 37 | 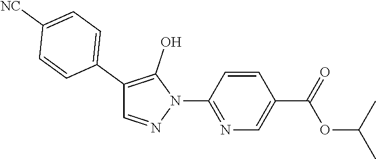 [SMILES: CC(C)OC(=O)c1ccc(-n2ncc(-c3ccc(C#N)cc3)c2O)nc1] |
| 38 | 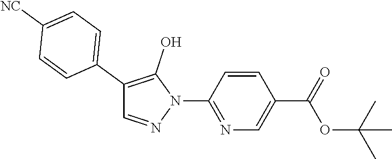 [SMILES: CC(C)(C)OC(=O)c1ccc(-n2ncc(-c3ccc(C#N)cc3)c2O)nc1] |
| 39 | 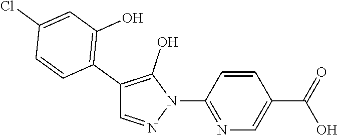 [SMILES: O=C(O)c1ccc(-n2ncc(-c3ccc(Cl)cc3O)c2O)nc1] |
| 40 | 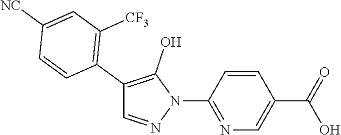 [SMILES: N#Cc1ccc(-c2cnn(-c3ccc(C(=O)O)cn3)c2O)c(C(F)(F)F)c1] |
| 41 | 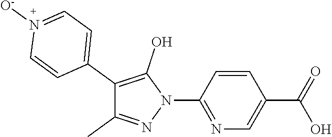 |
| 42 | 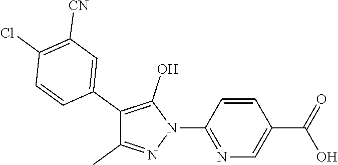 [SMILES: Cc1nn(-c2ccc(C(=O)O)cn2)c(O)c1-c1ccc(Cl)c(C#N)c1] |
| 43 | 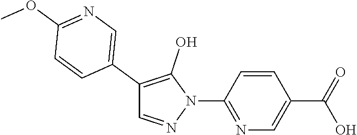 [SMILES: COc1ccc(-c2cnn(-c3ccc(C(=O)O)cn3)c2O)cn1] |
| 44 | 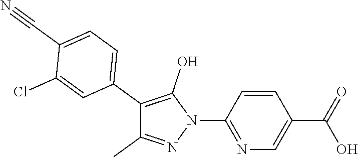 [SMILES: Cc1nn(-c2ccc(C(=O)O)cn2)c(O)c1-c1ccc(C#N)c(Cl)c1] |

In embodiments, in the compound of Formulas (A) and (I)-(III) such as any one of Compounds 1-44, or a pharmaceutically acceptable salt thereof, at least one hydrogen atom is replaced with a deuterium atom.

In another aspect, the invention features a pharmaceutical composition comprising any compound described herein (e.g., a compound of Formulas (A) and (I)-(III) such as any one of Compounds 1-44), or a pharmaceutically acceptable salt thereof, and a pharmaceutically acceptable excipient.

In another aspect, the invention features a method for treating a disease mediated by PHD activity comprising administering to a subject any compound described herein (e.g., a compound of Formulas (A) and (I)-(III) such as any one of Compounds 1-44), or a pharmaceutically acceptable salt thereof.

In embodiments, a disease mediated by PHD activity is an ischemic reperfusion injury. (e.g., stroke, myocardial infarction, or acute kidney injury).

In embodiments, a disease mediated by PHD activity is inflammatory bowel disease (e.g., ulcerative colitis or Crohn's disease).

In embodiments, a disease mediated by PHD activity is cancer (e.g., colorectal cancer).

In embodiments, a disease mediated by PHD activity is liver disease.

In embodiments, a disease mediated by PHD activity is atherosclerosis.

In embodiments, a disease mediated by PHD activity is cardiovascular disease

In embodiments, a disease mediated by PHD activity is a disease or condition of the eye (e.g., radiation retinopathy, retinopathy of prematurity, diabetic retinopathy, age-related macular degeneration, and ocular ischemia).

In embodiments, a disease mediated by PHD activity is anemia (e.g., anemia associated with chronic kidney disease).

In embodiments, a disease mediated by PHD activity is associated with hyperoxia

In embodiments, a disease mediated by PHD activity is retinopathy of prematurity.

In embodiments, a disease mediated by PHD activity is bronchopulmonary dysplasia (BPD).

In embodiments, a disease mediated by PHD activity is ischemic heart disease, valvular heart disease, congestive heart failure, acute lung injury, pulmonary fibrosis, pulmonary hypertension, chronic obstructive pulmonary disease (COPD), acute liver failure, liver fibrosis, or cirrhosis.

### BRIEF DESCRIPTION OF DRAWINGS

FIG. 1 is an exemplary schematic illustration demonstrating the principle of the TR-FRET Assay for PHD enzymes (PHD1, PHD2, and PHD3). In the presence of 2-oxoglutarate and O₂, PHD enzyme hydroxylates proline 564 of biotin-tagged HIF-1α peptide resulting in generation of biotin-tagged HIF-1α-hydroxyproline, succinate and CO₂. The resulting proximity of the donor fluorophore complex, monoclonal antibody anti-6His-Terbium (Tb)-cryptate Gold, bound to the His-tagged VHL protein, EloB, EloC complex (His-VBC) and the acceptor fluorophore, SA-D2 complex, bound to HIF-1α-hydroxyproline results in a fluorescence resonance energy transfer signal that can be detected and quantified

### DETAILED DESCRIPTION OF THE DISCLOSURE

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, l2%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in the description and the appended claims, the singular forms “a,” “an,” and “the” include plural referents unless the context clearly dictates otherwise. Thus, for example, reference to “a composition” includes mixtures of two or more such compositions.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance may or may not occur, and that the description includes instances where the event or circumstance occurs and instances where it does not.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Aliphatic: As used herein, the term aliphatic refers to C₁-C₄₀ hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls), C₂-C₂₀ alkenyls (e.g., linear or branched C₄-C₂₀ dienyls, linear, or branched C₆-C₂₀ trienyls, and the like), and C₂-C₂₀ alkynyls (e.g., linear or branched C₂-C₂₀ alkynyls). C₁-C₂₀ aliphatics can include C₃-C₂₀ cyclic aliphatics (e.g., C₃-C₂₀ cycloalkyls, C₄-C₂₀ cycloalkenyls, or C5-C20 cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the aliphatic is unsubstituted. In some embodiments, the aliphatic does not include any heteroatoms.

Alkyl: As used herein, the term “alkyl” means acyclic linear and branched hydrocarbon groups, e.g. “C₁-C₂₀ alkyl” refers to alkyl groups having 1-20 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C1-Cis alkyl, C₁-C₁₀ alkyl, or C₁-C₃ alkyl). In some embodiments, R′ independently is unsubstituted C₁-C₃ alkyl. In some embodiments, the alkyl is substituted (e.g., with 1, 2, 3, 4, 5, or 6 substituent groups as described herein). In some embodiments, an alkyl group is substituted with a-OH group and may also be referred to herein as a “hydroxyalkyl” group, where the prefix denotes the —OH group and “alkyl” is as described herein. In some embodiments, the alkyl is substituted with a —OR′ group and may also be referred to herein as “alkoxy” group.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR¹ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “C₂-C₂₀ alkenyl” refers to an alkenyl group having 2-20 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In some embodiments, the alkenyl comprises 1, 2, or 3 carbon-carbon double bond. In some embodiments, the alkenyl comprises a single carbon-carbon double bond. In some embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R¹ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkenyl is unsubstituted. In some embodiments, the alkenyl is substituted (e.g., with 1, 2, 3, 4, 5, or 6 substituent groups as described herein). In some embodiments, an alkenyl group is substituted with a-OH group and may also be referred to herein as a “hydroxyalkenyl” group, where the prefix denotes the —OH group and “alkenyl” is as described herein.

Alkynyl: As used herein, “alkynyl” means any hydrocarbon chain of either linear or branched configuration, having one or more carbon-carbon triple bonds occurring in any stable point along the chain, e.g. “C₂-C₂₀ alkynyl” refers to an alkynyl group having 2-20 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In some embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkynyl is unsubstituted. In some embodiments, the alkynyl is substituted (e.g., with 1, 2, 3, 4, 5, or 6 substituent groups as described herein).

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In some embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

Arylene: The term “arylene” as used herein refers to an aryl group that is divalent (that is, having two points of attachment to the molecule). Exemplary arylenes include phenylene (e.g., unsubstituted phenylene or substituted phenylene).

Halogen or Halo: As used herein, the term “halogen” or “halo” means fluorine, chlorine, bromine, or iodine.

Amide: The term “amide” or “amido” refers to a chemical moiety with formula —C(O)N(R′)₂, —C(O)N(R′)—, —NR′C(O)R′, —NR′C(O)N(R′)₂—, or —NR′C(O)—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Amino: The term “amino” or “amine” refers to a —N(R′)₂ group, where each R¹ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, heterocycloalkyl (bonded through a ring carbon), sulfonyl, or carbonyl group, unless stated other-wise in the specification, each of which moiety can itself be optionally substituted as described herein, or two R′ can combine with the nitrogen atom to form a 3-, 4-, 5-, 6-, or 7-membered ring. In embodiments, an amino group is —NHR′, where R¹ is aryl (“arylamino”), heteroaryl (“heteroarylamino”), or alkyl (“alkylamino”).

Sulfonyl: The term “sulfonyl” refers to a —S(═O)₂R′, or —S(═O)₂— group, where R¹ is selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, heterocycloalkyl (bonded through a ring carbon), unless stated other-wise in the specification, each of which moiety can itself be optionally substituted as described herein.

Sulfinyl: The term “sulfinyl” refers to a chemical moiety with formula —S(═O)R′, —S(═O)—, or —S(═O)(═NR′)—, where R¹ is selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, heterocycloalkyl (bonded through a ring carbon), unless stated other-wise in the specification, each of which moiety can itself be optionally substituted as described herein.

Carbonyl: The term “carbonyl” refers to a —C(═O)R′, or —C(═O)— group, where R¹ is selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, heterocycloalkyl (bonded through a ring carbon), unless stated other-wise in the specification, each of which moiety can itself be optionally substituted as described herein.

Phosphoryl: The term “phosphoryl” refers to a —P(═O)(R′)₂, or —P(═O)(R′)— group, where R′ is selected from hydrogen, alkyl, alkenyl, alkynyl, heteroalkyl (bonded through a chain carbon or through the heteroatom), cycloalkyl, aryl, arylalkyl, heteroaryl (bonded through a ring carbon), heteroarylalkyl, or heterocycloalkyl (bonded through a ring carbon) group, unless stated other-wise in the specification, each of which moiety can itself be optionally substituted as described herein, or two R′ can combine with the nitrogen atom to form a 3-, 4-, 5-, 6-, or 7-membered ring.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P.

Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

Heteroaryl: The term “heteroaryl,” as used herein, refers to a monocyclic, bicyclic, or tricyclic carbocyclic ring system having a total of six to fourteen ring members, wherein said ring system has a single point of attachment to the rest of the molecule, wherein at least one ring in the system is aromatic, wherein each ring in the system contains 4 to 7 ring members, and wherein at least one ring atom is a heteroatom such as, but not limited to, nitrogen and oxygen.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. The heterocycloalkyl group can be substituted or unsubstituted.

Deuterium: The term “deuterium” (“D” or “²H”) is also called heavy hydrogen. Deuterium is isotope of hydrogen with a nucleus consisting of one proton and one neutron, which is double the mass of the nucleus of ordinary hydrogen (one proton).

Isotope: The term “isotope” refers to a variant of a particular chemical element which differs in neutron number, and consequently in nucleon number. All isotopes of a given element have the same number of protons but different numbers of neutrons in each atom.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system, e.g., the substitution results in a stable compound (e.g., a compound which does not spontaneously undergo transformation such as by rearrangement, cyclization, elimination, or other reaction). In cases where a specified moiety or group is not expressly noted as being optionally substituted or substituted with any specified substituent, it is understood that such a moiety or group is intended to be unsubstituted.

When a ring system (e.g., cycloalkyl, heterocyclyl, aryl, or heteroaryl) is substituted with a number of substituents varying within an expressly defined range, it is understood that the total number of substituents does not exceed the normal available valencies under the existing conditions. It is also understood that hydrogen atoms are presumed present to fill the remaining valence of a ring system. The substituted group encompasses only those combinations of substituents and variables that result in a stable or chemically feasible compound. A stable compound or chemically feasible compound is one that, among other factors, has stability sufficient to permit its preparation and detection.

A wide variety of substituents are well known, and methods for their formation and introduction into a variety of parent groups are also well known. Representative substituents include but are not limited to alkyl, cycloalkyl, alkenyl, cycloalkenyl, alkynyl, arylalkyl, alkylaryl, aryl, arylalkoxy, arylamino, heteroarylamino, heteroaryl, heteroarylalkoxy, heterocycloalkyl, hydroxyalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, amide, cyano, alkoxy, hydroxy, sulfonamide, halo (e.g., —Cl and —Br), nitro, oximino, —COOR⁵⁰, —COR⁵⁰, —S₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO(R⁵⁰), —N(R⁵²)COOR⁵⁰ and —N(R⁵²)CON(R⁵⁰(R⁵¹), wherein R⁵⁰, R⁵¹ and R⁵² may be independently selected from the following: a hydrogen atom and a branched or straight-chain, C₁₋₆-alkyl, C₃₋₆-cycloalkyl, C₄₋₆-heterocycloalkyl, heteroaryl and aryl group, with or without substituents. When permissible, R⁵⁰ and R⁵¹ can be joined together to form a carbocyclic or heterocyclic ring system.

In preferred embodiments, the substituent is selected from halogen, —COR′, —CO₂H, —CO₂R′, —CN, —OH, —OR′, —OCOR′, —OCO₂R′, —NH₂, —NHR′, —N(R′)₂, —SR′, and —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In certain embodiments thereof, R′ independently is an unsubstituted alkyl (e.g., unsubstituted C₁-C₂₀ alkyl, C₁-C₁₅ alkyl, C₁-C₁₀ alkyl, or C₁-C₃ alkyl). Preferably, R′ independently is unsubstituted C₁-C₃ alkyl.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

### Compound of the Invention

Disclosed herein are compounds that are potent inhibitors of PHD. In some embodiments, the compounds of the present invention have enzymatic half maximal inhibitory concentration (IC₅₀) values of less than 100 μM against any one of PHD1, PHD2, and PHD3. In some embodiments, the compounds of the present invention have an IC₅₀ value of less than 50 μM against any one of PHD1, PHD2, and PHD3. In some embodiments, the compounds of the present invention have an IC₅₀ value of less than 25 μM against any one of PHD1, PHD2, and PHD3. In some embodiments, the compounds of the present invention have an IC₅₀ value of less than 20 μM against any one of PHD1, PHD2, and PHD3. In some embodiments, the compounds of the present invention have an IC₅₀ value of less than 15 μM against any one of PHD1, PHD2, and PHD3. In some embodiments, the compounds of the present invention have an IC₅₀ value of less than 10 μM against any one of PHD1, PHD2, and PHD3. In some embodiments, the compounds of the present invention have an IC₅₀ value of less than 5 μM against any one of PHD1, PHD2, and PHD3. In some embodiments, the compounds of the present invention have an IC₅₀ value of less than 1p M against any one of PHD1, PHD2, and PHD3. In some embodiments, the compounds of the present invention have an IC₅₀ value of about 3 nM to about 5 nM against any one of PHD1, PHD2, and PHD3. In some embodiments, the compounds of the present invention have an IC₅₀ value of about 5 nM to about 10 nM against any one of PHD1, PHD2, and PHD3. In some embodiments, the compounds of the present invention have an IC₅₀ value of about 10 nM to about 20 nM against any one of PHD1, PHD2, and PHD3. In some embodiments, the compounds of the present invention have an IC₅₀ value of about 20 nM to about 50 nM against any one of PHD1, PHD2, and PHD3. In some embodiments, the compounds of the present invention have an IC₅₀ value of about 50 nM to about 100 nM against any one of PHD1, PHD2, and PHD3. In some embodiments, the compounds of the present invention have an IC₅₀ value of about 100 nM to about 200 nM against any one of PHD1, PHD2, and PHD3. In some embodiments, the compounds of the present invention have an IC₅₀ value of about 200 nM to about 500 nM against any one of PHD1, PHD2, and PHD3. In some embodiments, the compounds of the present invention have an IC₅₀ value of about 500 nM to about 1000 nM against any one of PHD1, PHD2, and PHD3.

Representative examples from this class show inhibitory activity for PHD1, PHD2 and PHD3 in vitro.

Exemplary compounds are described herein. In particular, these selective inhibitors can feature a pyrazole moiety (e.g., a 5-hydroxy substituted pyrazole) linking the two aromatic moieties.

Compounds of Formulas (A) and (I)-(III)

In an aspect, provided herein are compounds having a structure according to Formula (A):

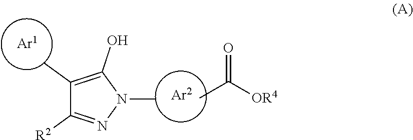

or a pharmaceutically acceptable salt thereof, wherein:

Ar¹ is phenyl or a six-membered nitrogen-containing heteroaryl, wherein said phenyl or heteroaryl is optionally substituted with halogen, CN, OH, C₁₋₃ alkyl optionally substituted with one or more halogens, or C₁₋₃ alkoxy;

R² is H or C₁₋₃ alkyl;

Ar² is a six-membered nitrogen-containing heteroaryl, optionally substituted with halogen, OH, amine, or C₁₋₃ alkyl; and

R⁴ is hydrogen or C₁₋₄ alkyl, and

In embodiments of formulas described herein (e.g., Formula (A)), a compound (e.g., a compound of Formula (A)) is not

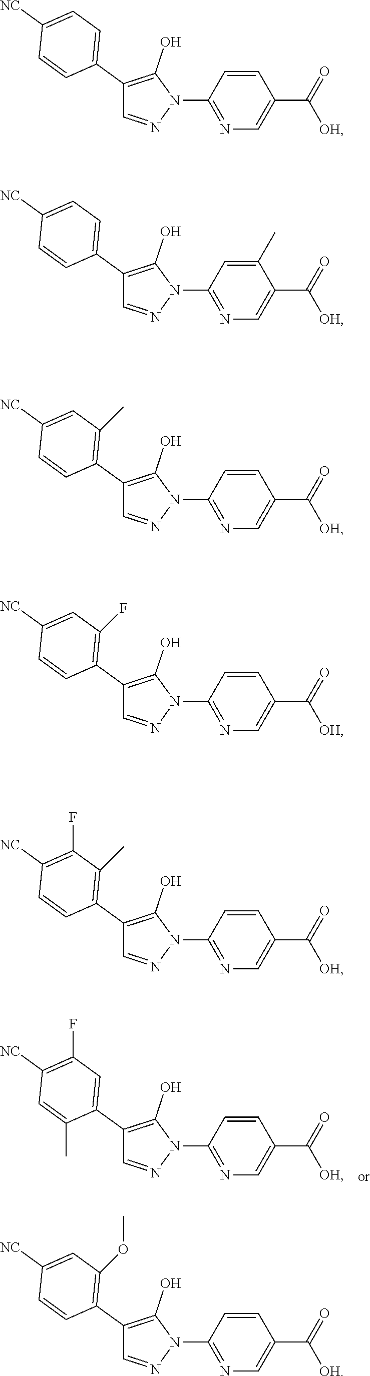

In embodiments of formulas described herein (e.g., Formula (A)), a compound (e.g., a compound of Formula (A)) excludes the following compounds

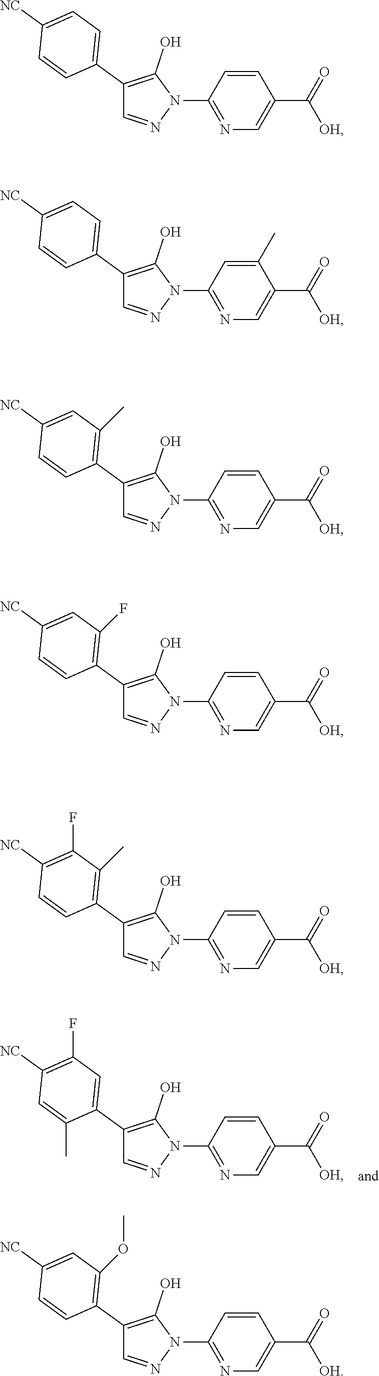

In embodiments, R² is H.

In embodiments, R² is C₁₋₃ alkyl. In embodiments, R² is CH₃. In embodiments, R² is CH₂CH₃. In embodiments, R² is CH₂CH₂CH₃. In embodiments, R² is CH(CH₃)₂.

In embodiments, R⁴ is H.

In embodiments, R⁴ is C₁₋₄ alkyl. In embodiments, R⁴ is CH₃. In embodiments, R⁴ is CH₂CH₃. In embodiments, R⁴ is CH₂CH₂CH₃. In embodiments, R⁴ is CH(CH₃)₂. In embodiments, R⁴ is CH₂CH₂CH₂CH₃. In embodiments, R⁴ is CH(CH₃)(CH₂CH₃). In embodiments, R⁴ is C(CH₃)₃.

In embodiments, Ar¹ is an unsubstituted aryl. In embodiments, Ar¹ is a substituted aryl. In embodiments, Ar¹ is an unsubstituted phenyl. In embodiments, Ar¹ is a substituted phenyl.

In embodiments, Ar¹ is an unsubstituted 6-membered nitrogen-containing heteroaryl. In embodiments, Ar¹ is a substituted 6-membered nitrogen-containing heteroaryl.

In embodiments, Ar¹ is substituted with one or more groups selected from halogen, CN, OH, C₁₋₃ alkyl optionally substituted with one or more halogens, and C₁₋₃ alkoxy. In some embodiments, Ar¹ is substituted with 1 substituent group. In some embodiments, Ar¹ is substituted with 2 substituent groups. In some embodiments, Ar¹ is substituted with 3 substituent groups. In some embodiments, Ar¹ is substituted with 4 substituent groups.

In embodiments, Ar¹ comprises one or more R¹ groups, wherein each R¹ is selected independently from hydrogen, halogen, CN, OH, C₁₋₃ alkyl optionally substituted with one or more halogens, and C₁₋₃ alkoxy. In embodiments, Ar¹ comprises a quantity of R¹ groups that is represented by m, wherein m is 1, 2, 3, or 4. When R¹ is present, R¹ can replace a hydrogen in the parent molecular structure. In embodiments, when R¹ is present and is a non-hydrogen moiety, R¹ represents a substituent group. In embodiments, R¹ is selected independently from halogen, CN, OH, C₁₋₃ alkyl optionally substituted with CN or one or more halogens, and C₁₋₃ alkoxy.

Accordingly, it is also understood that for any value of m described herein, hydrogens are present as appropriate in order to complete valency requirements at constituent atoms of Ar¹ such that the molecule is a stable compound (e.g., the molecule is a compound which does not spontaneously undergo transformation such as by rearrangement, cyclization, elimination, or other reaction). Exemplary embodiments of Ar¹, R¹, and m are described herein.

In embodiments, Ar¹ is

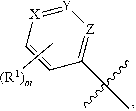

wherein

X, Y, and Z are independently CH or N, wherein N is optionally oxidized;

each R¹ is independently selected from the group consisting of hydrogen, halogen, CN, OH, C₁₋₃ alkyl optionally substituted with one or more halogens, and C₁₋₃ alkoxy; and

m is 1, 2, 3, or 4.

In embodiments, R¹ is not a hydrogen. In embodiments, when R¹ is present and is a non-hydrogen moiety, R¹ represents a substituent group.

In embodiments, the value of m is based on the number of nitrogen atoms present in the ring. In embodiments, when two and only two of X, Y, and Z are N, m is 1, 2, or 3. In embodiments, when each of X, Y, and Z are N, m is 1 or 2.

In embodiments, m is 1. In embodiments, m is 2. In embodiments, m is 3. In embodiments, m is 4.

In embodiments, X, Y and Z are all N, m is 1 or 2. In embodiments, m is 1, and any remaining unsubstituted carbon ring atoms are assumed bonded to hydrogen in order to fill the valence. In embodiments, m is 2.

In embodiments, one of X, Y and Z is CH, and the others are N, wherein N is optionally oxidized, m is 1, 2, or 3. In embodiments, m is 1, and any remaining unsubstituted carbon ring atoms are assumed bonded to hydrogen in order to fill the valence. In embodiments, m is 2, and any remaining unsubstituted carbon ring atoms are assumed bonded to hydrogen in order to fill the valence. In embodiments, m is 3.

In embodiments, two of X, Y and Z are CH and the other is N, wherein N is optionally oxidized, m is 1, 2, 3, or 4. In embodiments, m is 1, and any remaining unsubstituted carbon ring atoms are assumed bonded to hydrogen in order to fill the valence. In embodiments, m is 2, and any remaining unsubstituted carbon ring atoms are assumed bonded to hydrogen in order to fill the valence. In embodiments, m is 3, and any remaining unsubstituted carbon ring atoms are assumed bonded to hydrogen in order to fill the valence. In embodiments, m is 4.

In embodiments, the N atom in Ar¹ is not oxidized.

In embodiments, the N atom in Ar¹ is oxidized.

In embodiments, Ar¹ is a substituted phenyl. In embodiments, Ar¹ is a substituted by at least one R¹, wherein R¹ is CN or halogen.

In embodiments, Ar¹ is substituted by one or two R¹ groups independently selected from C₁₋₃ alkyl optionally substituted with one or more halogens, halogen, CN or OH.

In embodiments, Ar¹ is a pyridyl N-oxide or is a pyridyl optionally substituted by at least one R¹ that is C₁₋₃ alkoxy or halogen.

In embodiments, R¹, each time taken, is hydrogen.

In embodiments, R¹, each time taken, is CN.

In embodiments, R¹, each time taken, is OH.

In embodiments, R¹, each time taken, is halogen. In embodiments, the halogen is Cl.

In embodiments, the halogen is Br. In embodiments, the halogen is I.

In embodiments, R¹, each time taken, is C₁₋₃ alkyl.

In embodiments, R¹, each time taken, is unsubstituted C₁₋₃ alkyl. In embodiments, R¹, each time taken, is CH₃. In embodiments, R¹, each time taken, is CH₂CH₃.

In embodiments, R¹, each time taken, is substituted C₁₋₃ alkyl. In embodiments, R¹, each time taken, is C₁₋₃ alkyl substituted with one or more halogens. In embodiments, a halogen is F. In embodiments, a halogen is Cl. In embodiments, a halogen is Br. In embodiments, a halogen is I.

In embodiments, R¹, each time taken, is CF₃.

In embodiments, R¹, each time taken, is C₁₋₃ alkoxy. In embodiments, R¹, each time taken, is OMe.

In embodiments, Ar¹ is selected from:

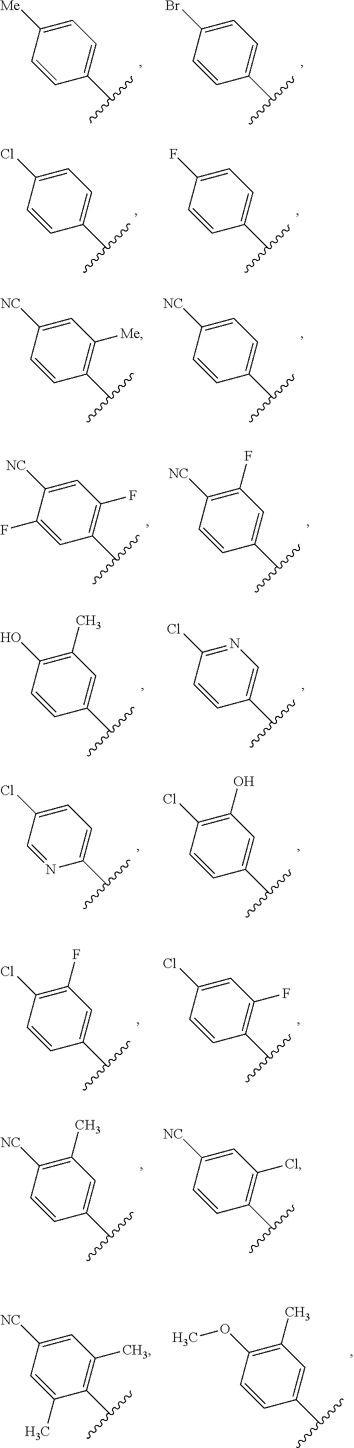

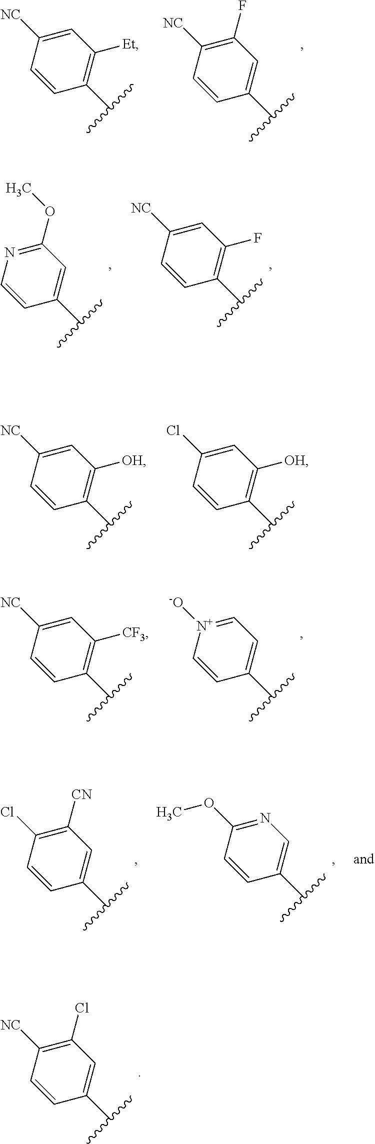

In embodiments, Ar¹ is selected from:

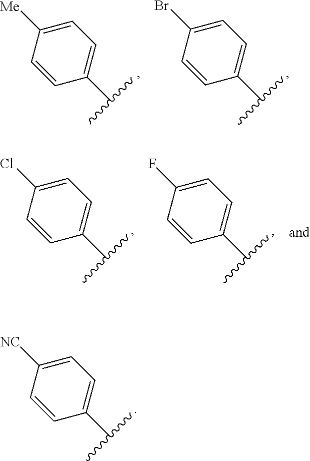 [SMILES: *c1ccc(C)cc1 ; *c1ccc(Br)cc1 ; *c1ccc(Cl)cc1 ; *c1ccc(F)cc1 ; *c1ccc(C#N)cc1]

In embodiments, Ar¹ is selected from:

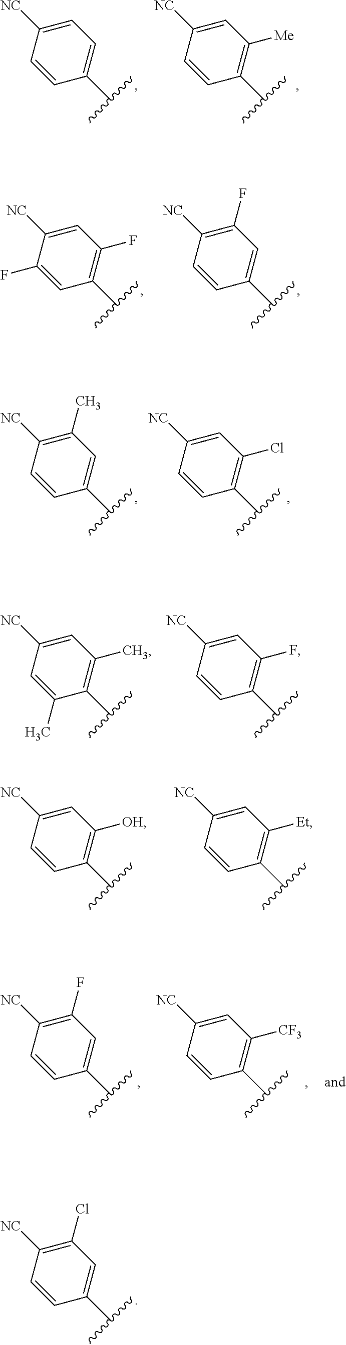

In embodiments, Ar¹ is selected from:

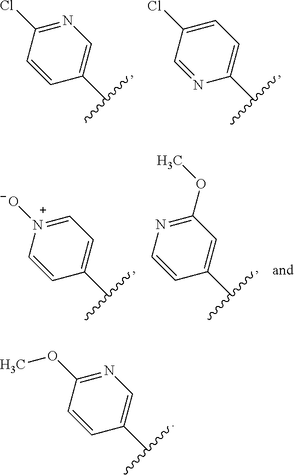

In embodiments, Ar¹ is selected from:

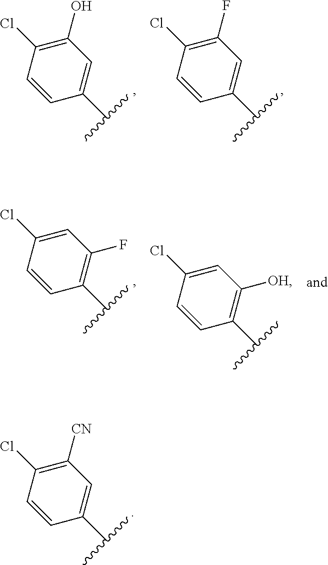

In embodiments, Ar¹ is selected from:

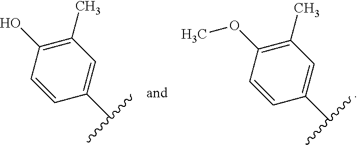

In embodiments, Ar² is an unsubstituted six-membered nitrogen-containing heteroaryl.

In embodiments, Ar² is a six-membered nitrogen-containing heteroaryl substituted with halogen, OH, amine, or C₁₋₃ alkyl. In embodiments, Ar² is substituted by 1 substituent as described herein. In embodiments, Ar² is substituted by 2 substituents as described herein.

In embodiments, Ar² is

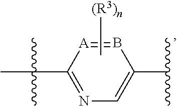

wherein

A and B are independently CH or N, wherein N is optionally oxidized;

each R³ is independently selected from the group consisting of hydrogen, halogen, OH, amine, and C₁₋₃ alkyl; and

n is 0, 1, or 2.

In embodiments, the value of n is based on the number of nitrogen atoms present in the ring. In embodiments, when one and only one of A and B are N, n is 0, 1, or 2. In embodiments, when both A and B are N, n is 0 or 1.

In embodiments, n is 0. In embodiments, n is 1. In embodiments, n is 2.

In embodiments, A and B are both N, wherein N is optionally oxidized, n is 0 or 1. In embodiments, n is 0. In embodiments, n is 1.

In embodiments, one of A and B is CH and the other is N, wherein N is optionally oxidized, n is 0, 1, or 2. In embodiments, n is 0. In embodiments, n is 1. In embodiments, n is 2.

In embodiments, A and B are both CH, n is 0, 1, or 2. In embodiments, n is 0. In embodiments, n is 1. In embodiments, n is 2.

In embodiments, the N atom in Ar² is not oxidized.

In embodiments, the N atom in Ar² is oxidized.

In embodiments, R³, each time taken, is hydrogen.

In embodiments, R³, each time taken, is OH.

In embodiments, R³, each time taken, is halogen. In embodiments, a halogen is F. In embodiments, a halogen is Cl. In embodiments, a halogen is Br. In embodiments, a halogen is I.

In embodiments, R³, each time taken, is amine. In embodiments, R³, each time taken, is NH₂.

In embodiments, R³, each time taken, is C₁₋₃ alkyl.

In embodiments, R³, each time taken, is unsubstituted C₁₋₃ alkyl. In embodiments, R³, each time taken, is CH₃.

In embodiments, Ar² is selected from the group consisting of.

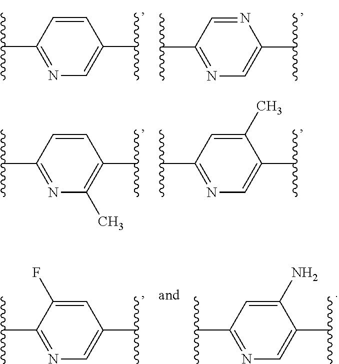

In embodiments, a compound of Formula (A) has the following structure,

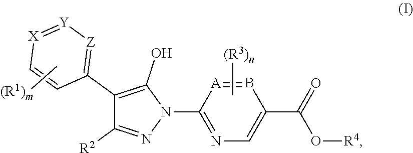

or a pharmaceutically acceptable salt thereof, wherein A, B, X, Y, Z, R¹, R², R³ and R⁴ are as defined anywhere herein.

In embodiments of formulas described herein (e.g., Formula (I)), a compound (e.g., a compound of Formula (I)) is not

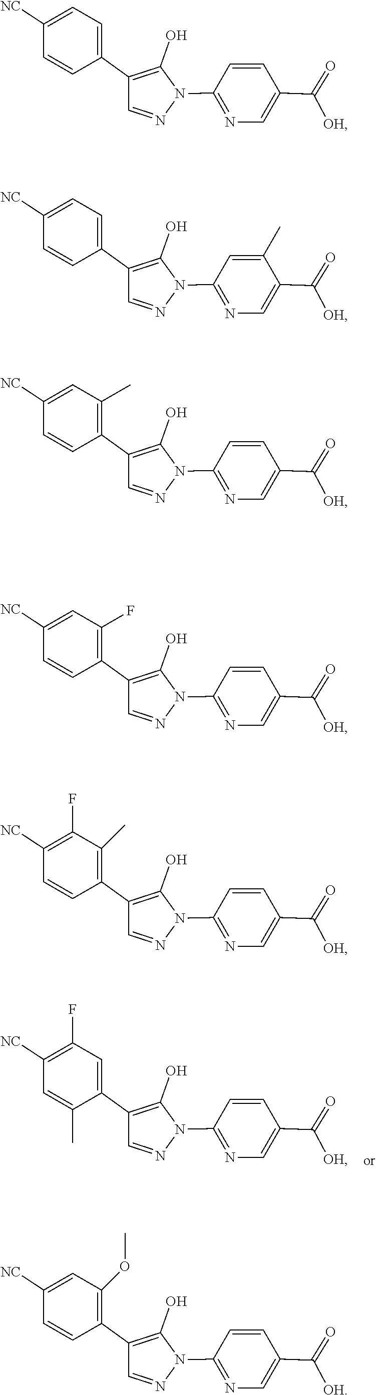

In embodiments of formulas described herein (e.g., Formula (I)), a compound (e.g., a compound of Formula (I)) excludes the following compounds

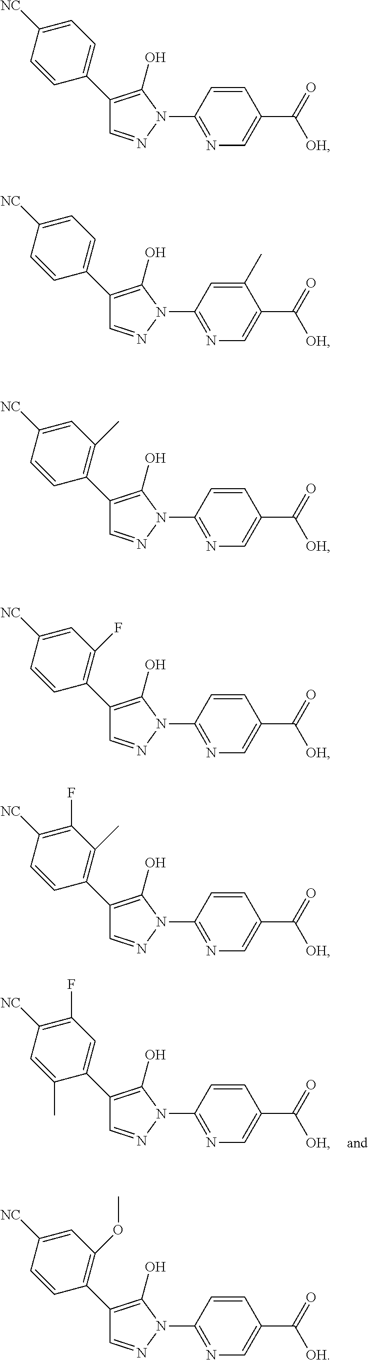

In embodiments, a compound of Formula (A) or Formula (I) has the following structure,

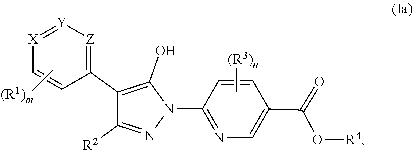

or a pharmaceutically acceptable salt thereof, wherein X, Y, Z, R¹, R², R³ and R⁴ are as defined anywhere herein.

In embodiments of formulas described herein (e.g., Formula (Ia)), a compound (e.g., a compound of Formula (Ia)) is not

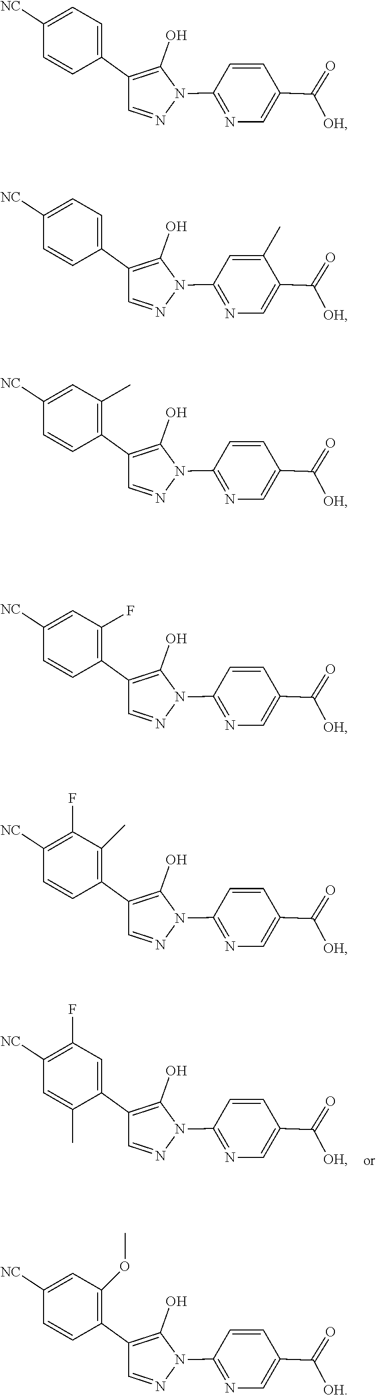

In embodiments of formulas described herein (e.g., Formula (Ia)), a compound (e.g., a compound of Formula (Ia)) excludes the following compounds

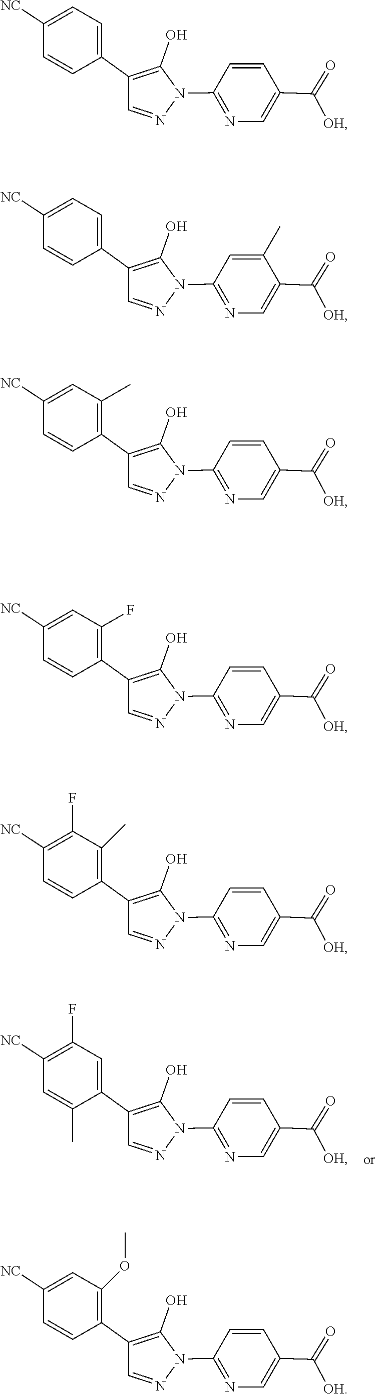

In embodiments, a compound of Formula (A) or Formula (I) has the following structure,

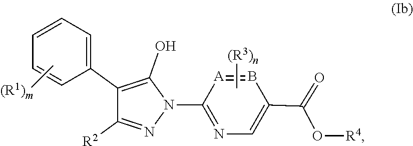

or a pharmaceutically acceptable salt thereof, wherein R¹, R², R³ and R⁴ are as defined anywhere herein.

In embodiments of formulas described herein (e.g., Formula (Ib)), a compound (e.g., a compound of Formula (Ib)) is not

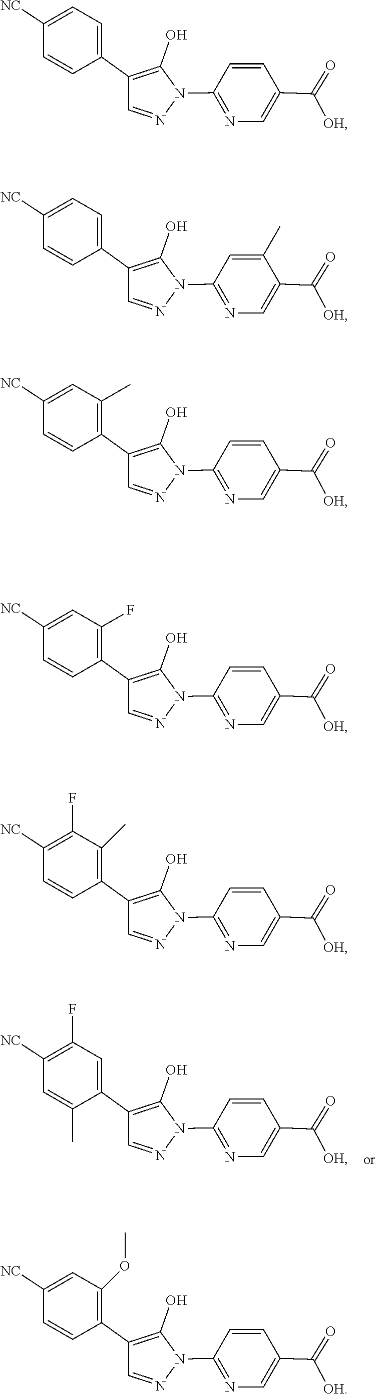

In embodiments of formulas described herein (e.g., Formula (Ib)), a compound (e.g., a compound of Formula (Ib)) excludes the following compounds

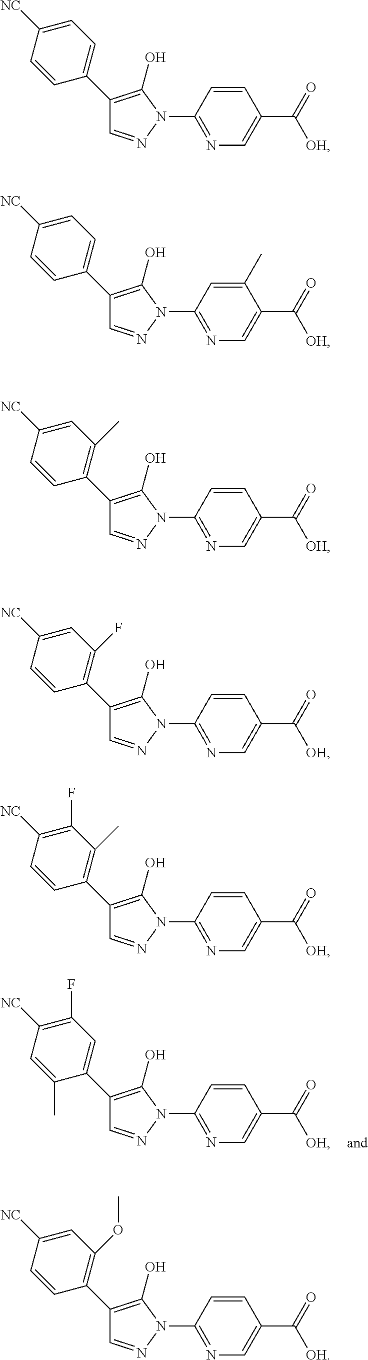

In embodiments, a compound of Formula (A) or Formula (I) has the following structure,

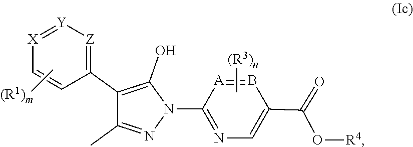

or a pharmaceutically acceptable salt thereof, wherein A, B, X, Y, Z, R¹, R³ and R⁴ are as defined anywhere herein.

In embodiments, a compound of Formula (A) or Formula (I) has the following structure,

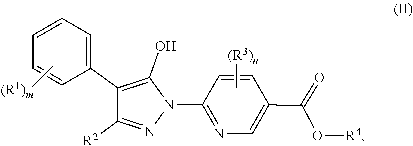

or a pharmaceutically acceptable salt thereof, wherein R¹, R², R³ and R⁴ are as defined anywhere herein.

In embodiments of formulas described herein (e.g., Formula (II)), a compound (e.g., a compound of Formula (II)) is not

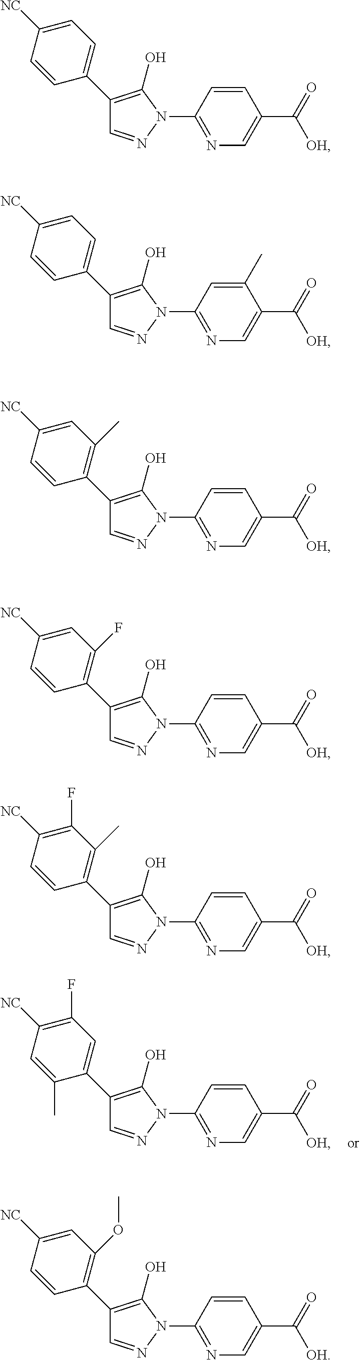

In embodiments of formulas described herein (e.g., Formula (II)), a compound (e.g., a compound of Formula (II)) excludes the following compounds

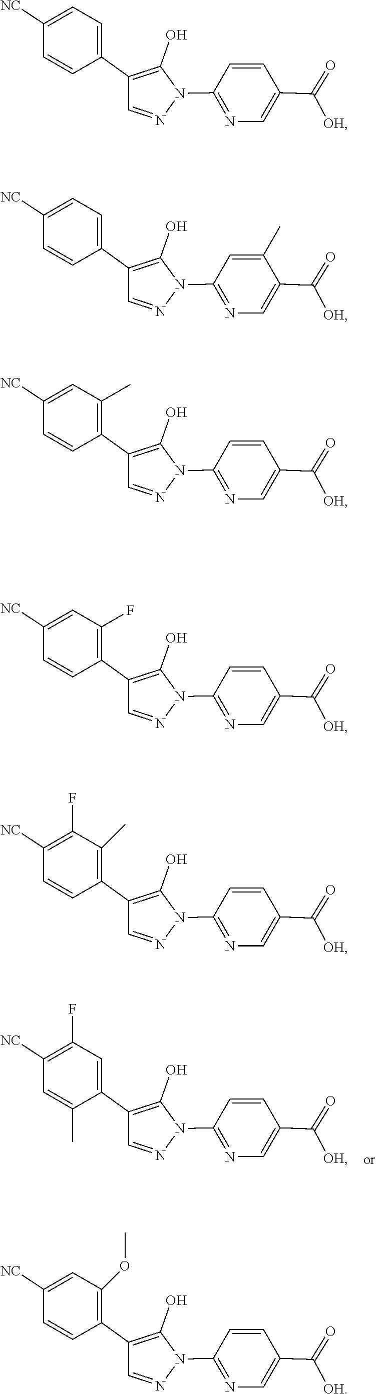

In embodiments, a compound of Formula (A), Formula (I), or Formula (II) has the following structure,

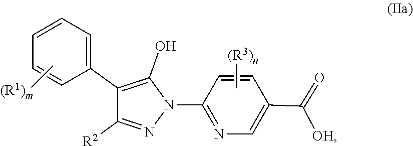

or a pharmaceutically acceptable salt thereof, wherein R¹, R², and R³ are as defined anywhere herein.

In embodiments of formulas described herein (e.g., Formula (IIa)), a compound (e.g., a compound of Formula (IIa)) is not

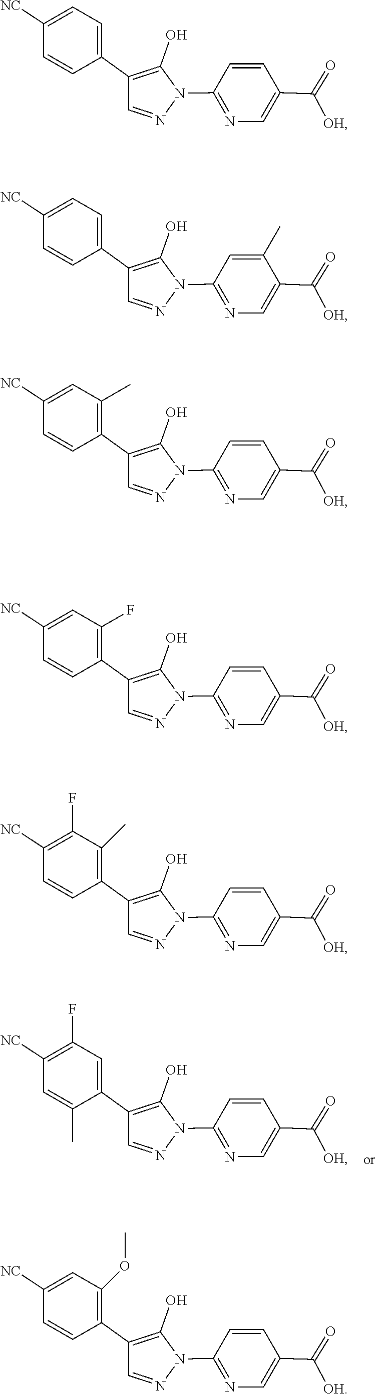

In embodiments of formulas described herein (e.g., Formula (IIa)), a compound (e.g., a compound of Formula (IIa)) excludes the following compounds

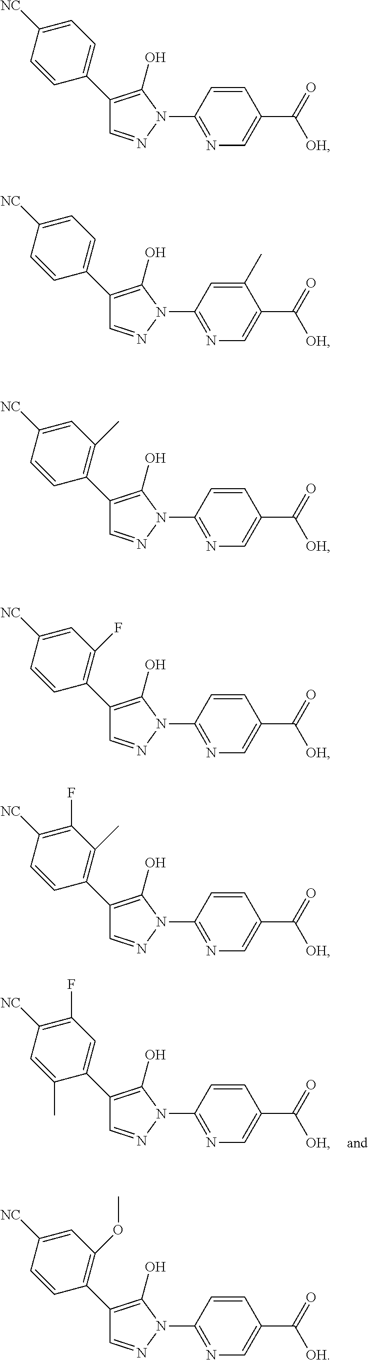

In embodiments, a compound of Formula (A), Formula (I), or Formula (II) has the following structure,

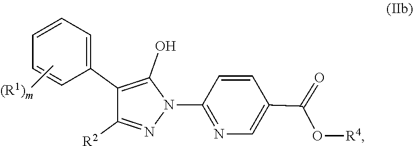

or a pharmaceutically acceptable salt thereof, wherein R¹, R², and R⁴ are as defined anywhere herein.

In embodiments of formulas described herein (e.g., Formula (IIb)), a compound (e.g., a compound of Formula (IIb)) is not

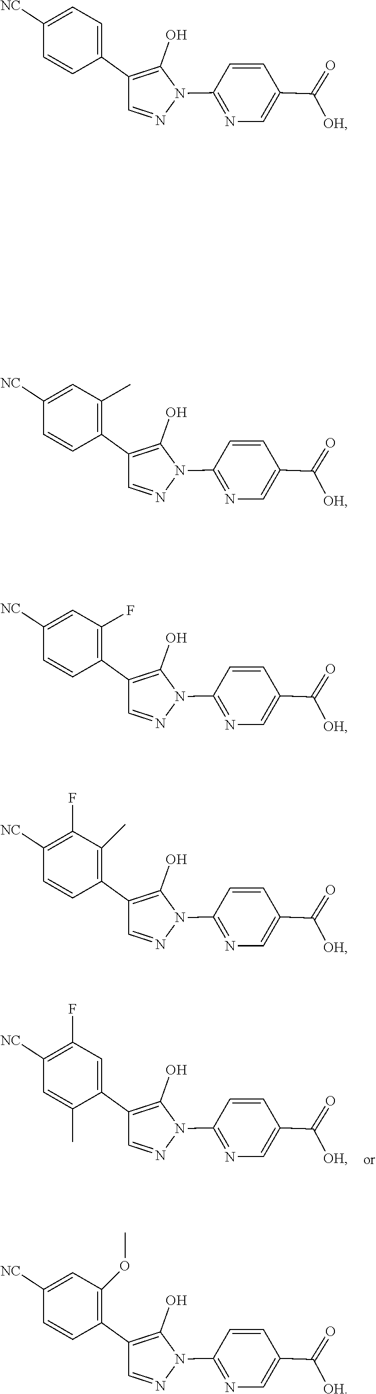 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments of formulas described herein (e.g., Formula (IIb)), a compound (e.g., a compound of Formula (IIb)) excludes the following compounds

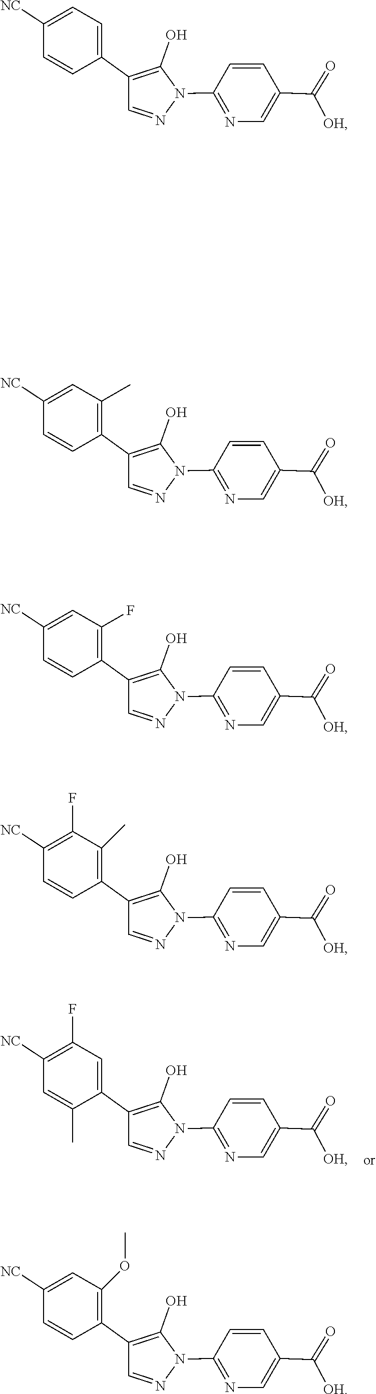 [SMILES: N#Cc1ccc(-c2cnn(-c3ccc(C(=O)O)cn3)c2O)cc1 ; Cc1cc(C#N)ccc1-c1cnn(-c2ccc(C(=O)O)cn2)c1O ; N#Cc1ccc(-c2cnn(-c3ccc(C(=O)O)cn3)c2O)c(F)c1 ; Cc1c(-c2cnn(-c3ccc(C(=O)O)cn3)c2O)ccc(C#N)c1F ; Cc1cc(C#N)c(F)cc1-c1cnn(-c2ccc(C(=O)O)cn2)c1O ; COc1cc(C#N)ccc1-c1cnn(-c2ccc(C(=O)O)cn2)c1O]

In embodiments, a compound of Formula (A), Formula (I), or Formula (II), has the following structure,

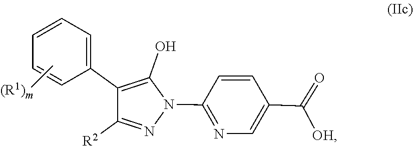

or a pharmaceutically acceptable salt thereof, wherein R¹ and R² are as defined anywhere herein.

In embodiments of formulas described herein (e.g., Formula (IIc)), a compound (e.g., a compound of Formula (IIc)) is not

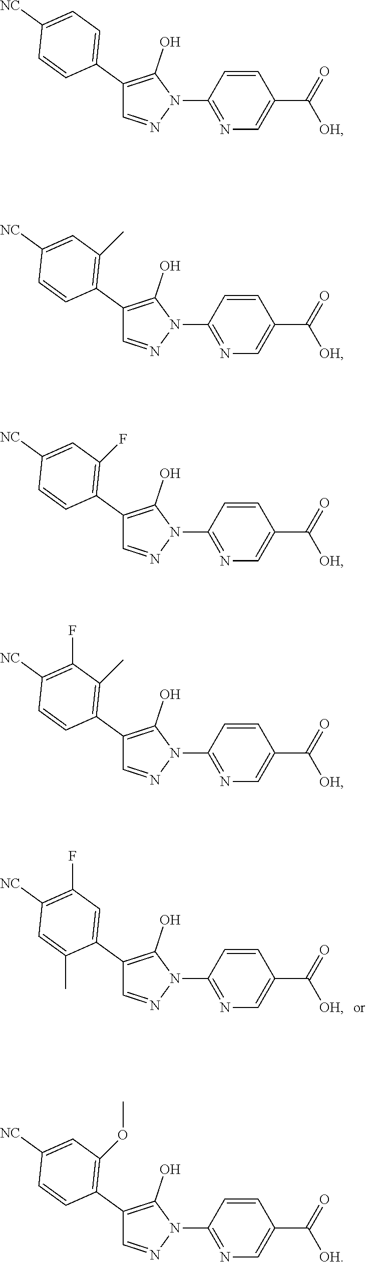

In embodiments of formulas described herein (e.g., Formula (IIc)), a compound (e.g., a compound of Formula (IIc)) excludes the following compounds

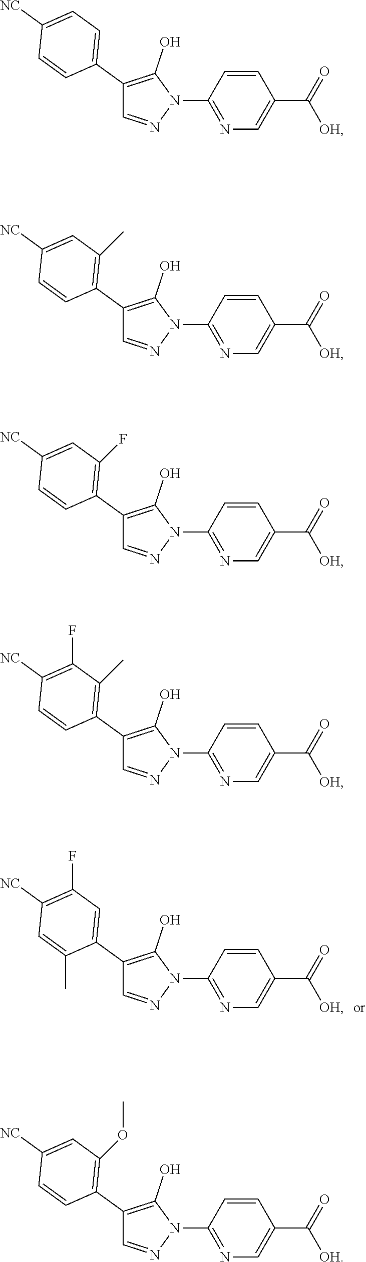

In embodiments, a compound of Formula (A), Formula (I), or Formula (II), has the following structure,

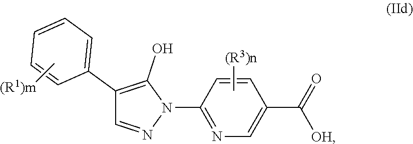

or a pharmaceutically acceptable salt thereof, wherein R¹ and R³ are as defined anywhere herein.

In embodiments of formulas described herein (e.g., Formula (IId)), a compound (e.g., a compound of Formula (IId)) is not

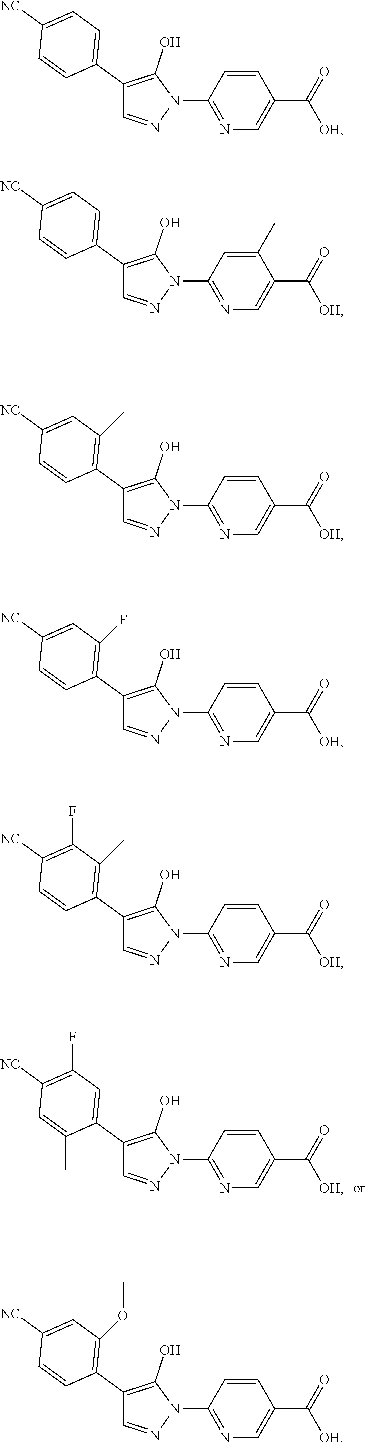

In embodiments of formulas described herein (e.g., Formula (IId)), a compound (e.g., a compound of Formula (IId)) excludes the following compounds

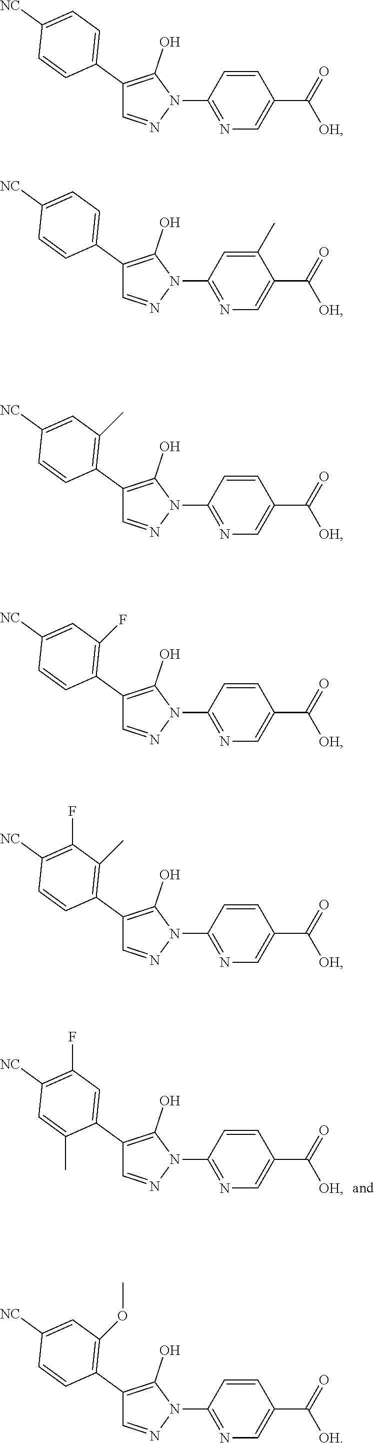

In embodiments, a compound of Formula (A), Formula (I), or Formula (II) has the following structure,

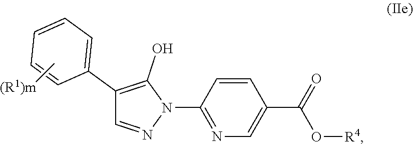

or a pharmaceutically acceptable salt thereof, wherein R¹ and R⁴ are as defined anywhere herein.

In embodiments of formulas described herein (e.g., Formula (IIe)), a compound (e.g., a compound of Formula (IIe)) is not

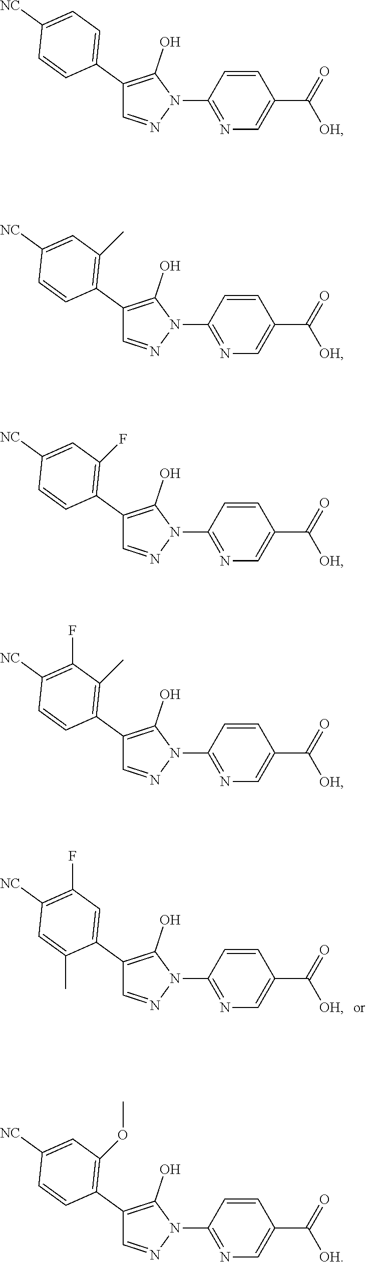

In embodiments of formulas described herein (e.g., Formula (IIe)), a compound (e.g., a compound of Formula (IIe)) excludes the following compounds

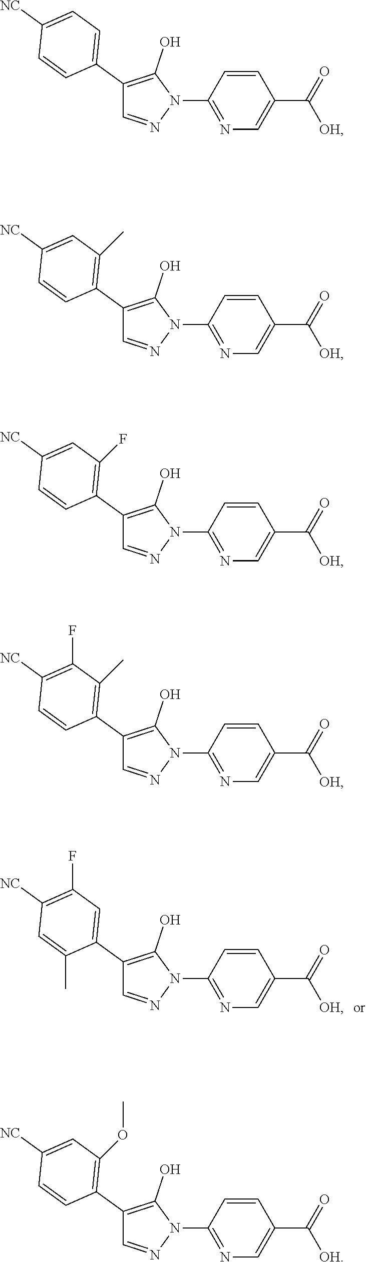

In embodiments, a compound of Formula (A), Formula (I), or Formula (II), has the following structure,

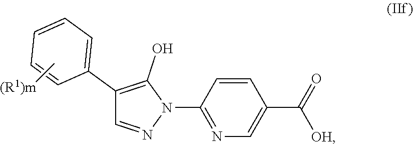

or a pharmaceutically acceptable salt thereof, wherein R¹ is as defined anywhere herein.

In embodiments of formulas described herein (e.g., Formula (IIf)), a compound (e.g., a compound of Formula (IIf)) is not

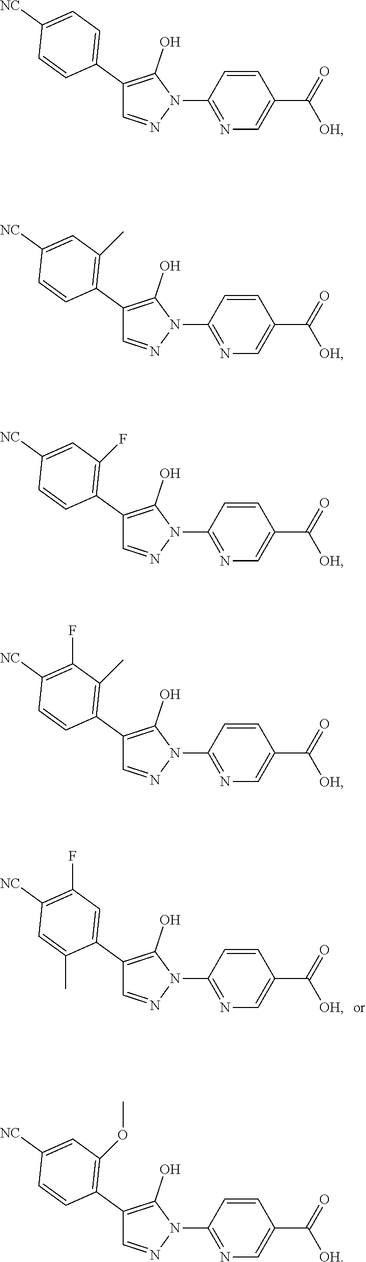

In embodiments of formulas described herein (e.g., Formula (IIf)), a compound (e.g., a compound of Formula (IIf)) excludes the following compounds

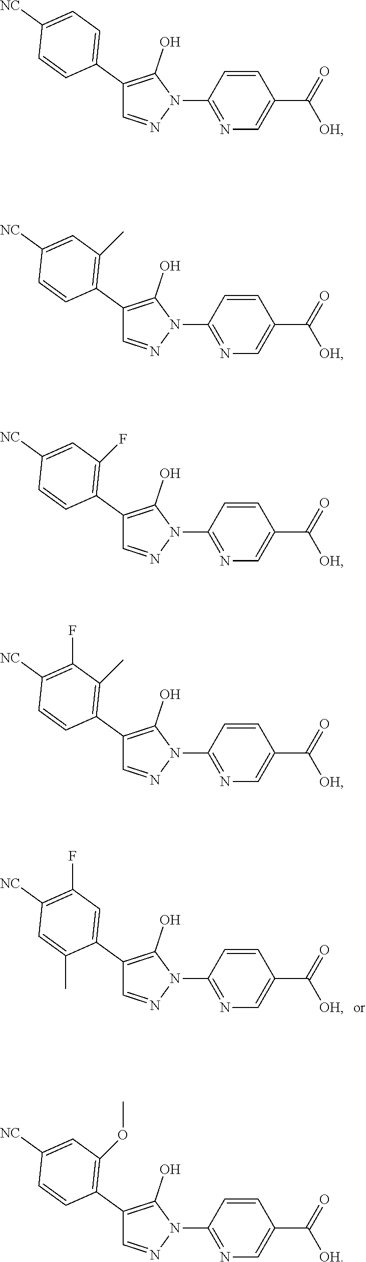

In embodiments, a compound of Formula (A), Formula (I), or Formula (II), has the following structure,

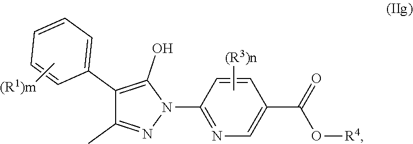

or a pharmaceutically acceptable salt thereof, wherein R¹, R³ and R⁴ are as defined anywhere herein.

In embodiments, a compound of Formula (A), Formula (I), or Formula (II), has the following structure,

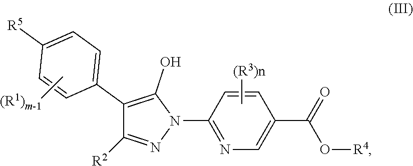

or a pharmaceutically acceptable salt thereof, wherein R¹, R², R³ and R⁴ are as defined anywhere herein, and wherein

R⁵ is CN or halogen.

In embodiments, R⁵ is CN.

In embodiments, R⁵ is halogen. In embodiments, a halogen is F. In embodiments, a halogen is Cl. In embodiments, a halogen is Br. In embodiments, a halogen is I.

In embodiments of formulas described herein (e.g., Formula (III)), a compound (e.g., a compound of Formula (III)) is not

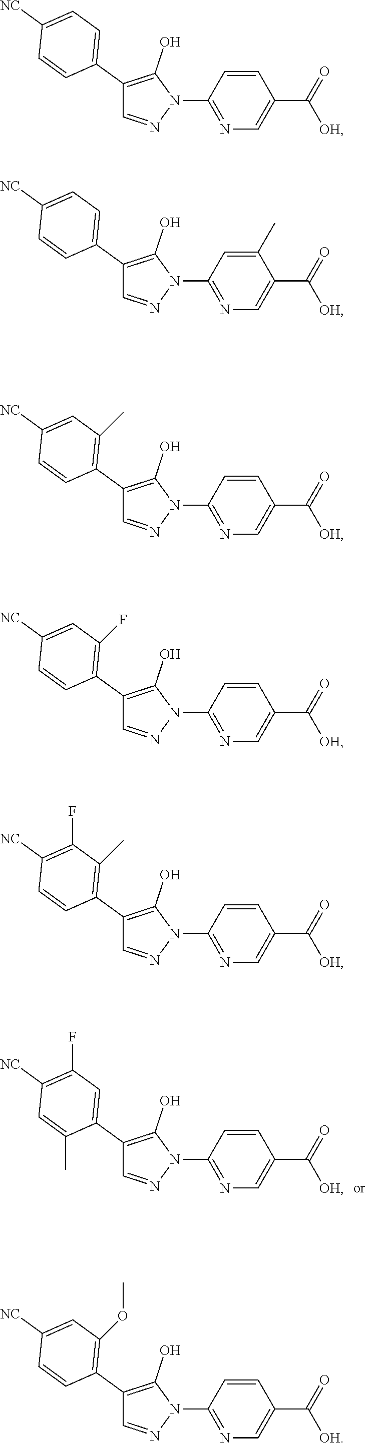

In embodiments of formulas described herein (e.g., Formula (III)), a compound (e.g., a compound of Formula (III)) excludes the following compounds

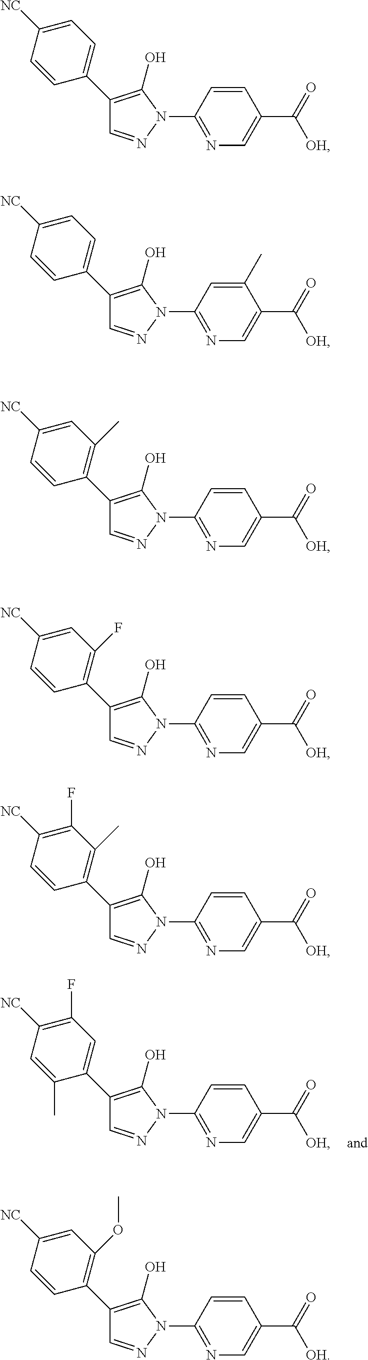

In embodiments, a compound of Formula (A), Formula (I), Formula (II), or Formula (III), has the following structure,

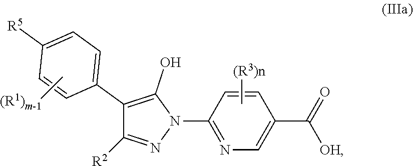

or a pharmaceutically acceptable salt thereof, wherein R¹, R², R³, R⁴ and R⁵ are as defined anywhere herein.

In embodiments of formulas described herein (e.g., Formula (IIIa)), a compound (e.g., a compound of Formula (IIIa)) is not

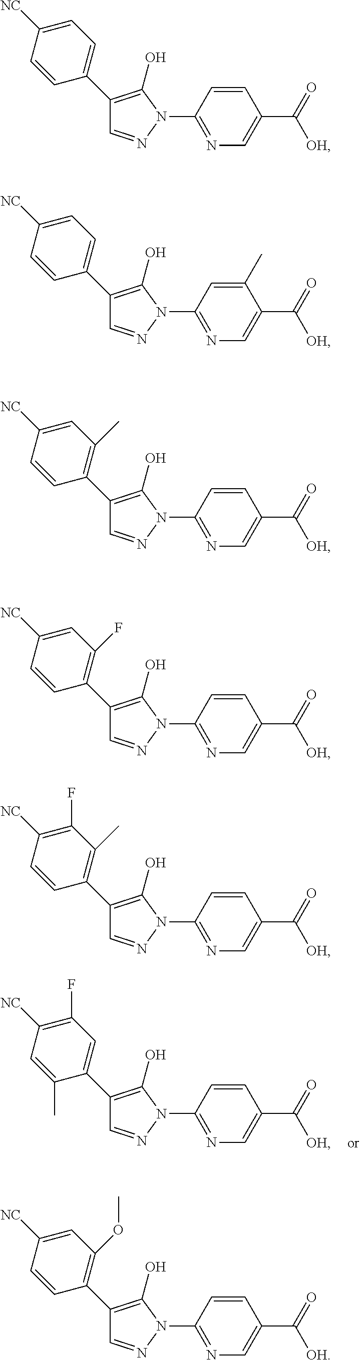

In embodiments of formulas described herein (e.g., Formula (IIIa)), a compound (e.g., a compound of Formula (IIIa)) excludes the following compounds

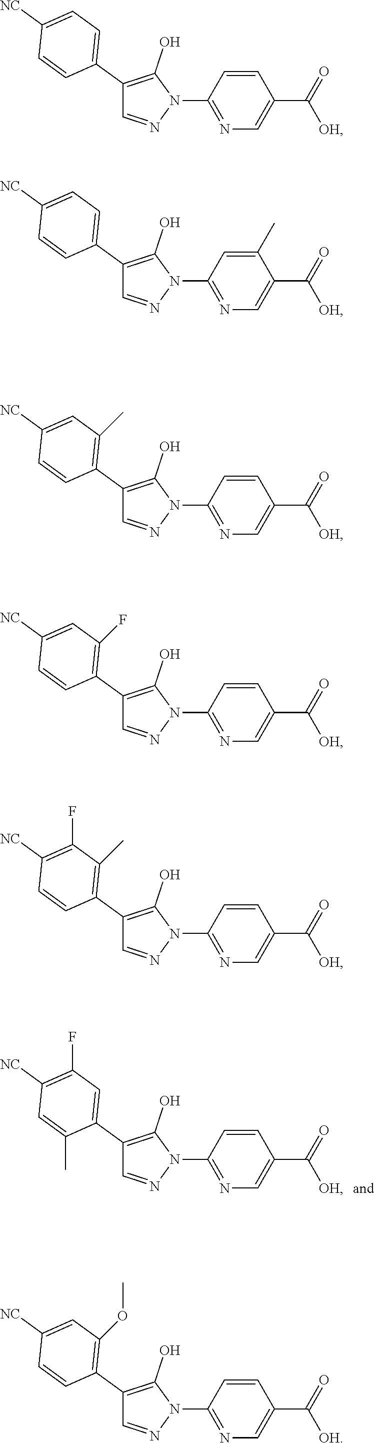

In embodiments, a compound of Formula (A), Formula (I), Formula (II), or Formula (III), has the following structure,

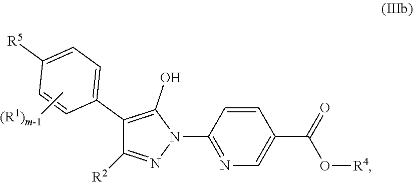

or a pharmaceutically acceptable salt thereof, wherein R¹, R², R⁴ and R⁵ are as defined anywhere herein.

In embodiments of formulas described herein (e.g., Formula (IIIb)), a compound (e.g., a compound of Formula (IIIb)) is not

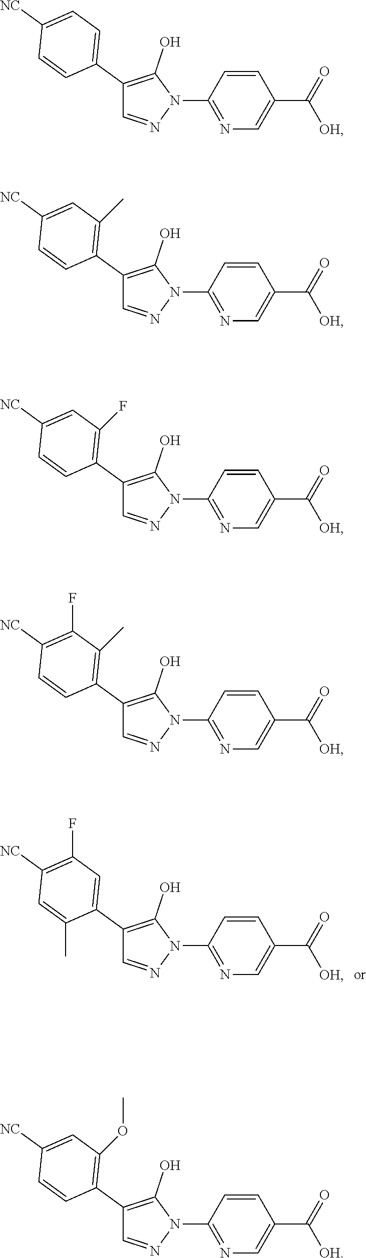

In embodiments of formulas described herein (e.g., Formula (IIIb)), a compound (e.g., a compound of Formula (IIIb)) excludes the following compounds

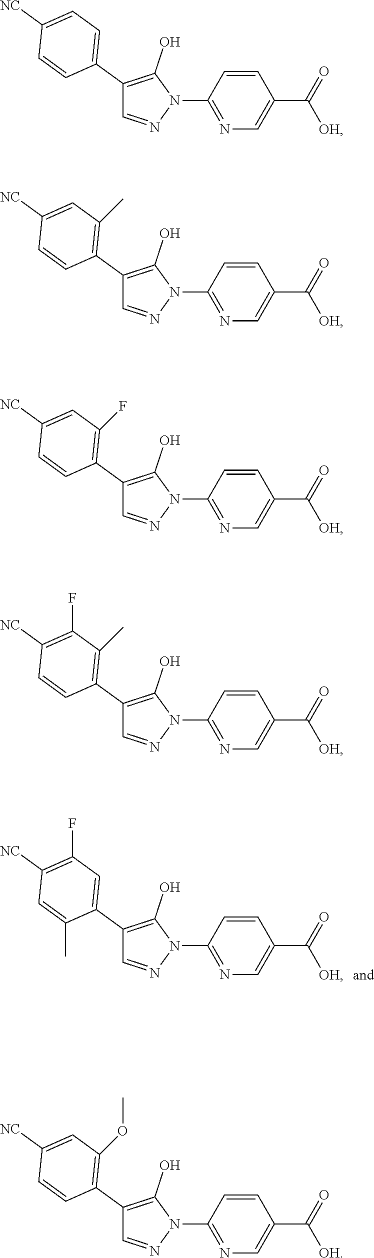

In embodiments, a compound of Formula (A), Formula (I), Formula (II), or Formula (III), has the following structure,

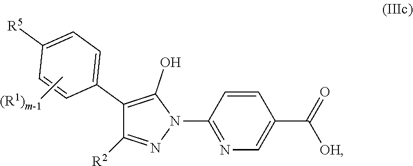

or a pharmaceutically acceptable salt thereof, wherein R¹, R², and R⁵ are as defined anywhere herein.

In embodiments of formulas described herein (e.g., Formula (IIIc)), a compound (e.g., a compound of Formula (IIIc)) is not

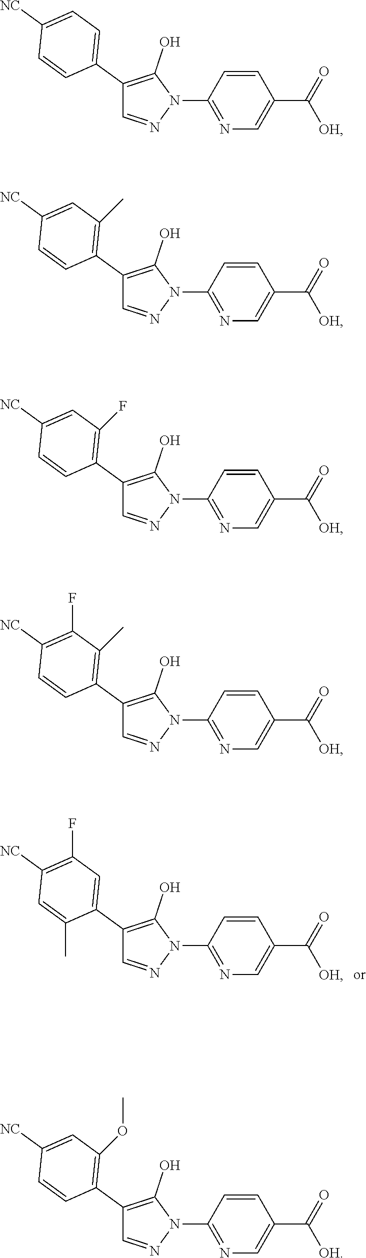

In embodiments of formulas described herein (e.g., Formula (IIIc)), a compound (e.g., a compound of Formula (IIIc)) excludes the following compounds

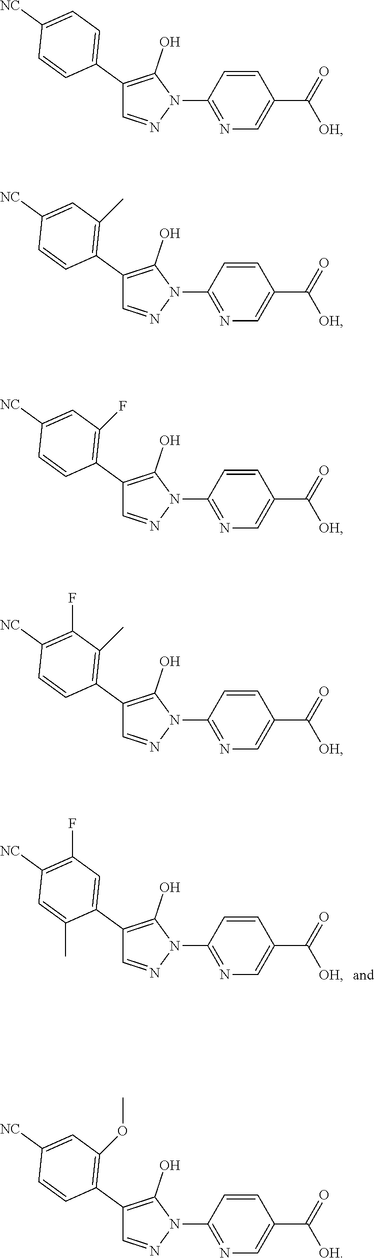

In embodiments, a compound of Formula (A), Formula (I), Formula (II), or Formula (III), has the following structure,

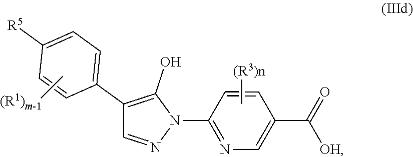

or a pharmaceutically acceptable salt thereof, wherein R¹, R³, and R⁵ are as defined anywhere herein.

In embodiments of formulas described herein (e.g., Formula (IIId)), a compound (e.g., a compound of Formula (IIId)) is not

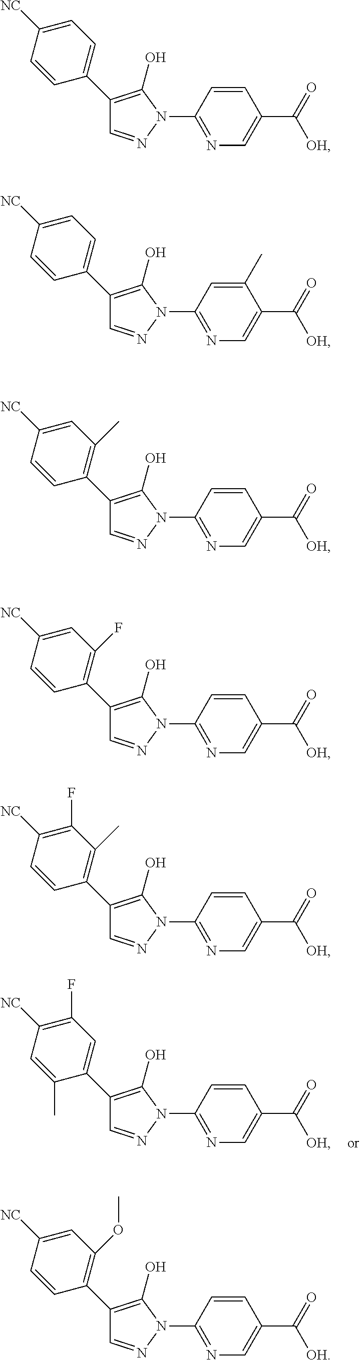

In embodiments of formulas described herein (e.g., Formula (IIId)), a compound (e.g., a compound of Formula (IIId)) excludes the following compounds

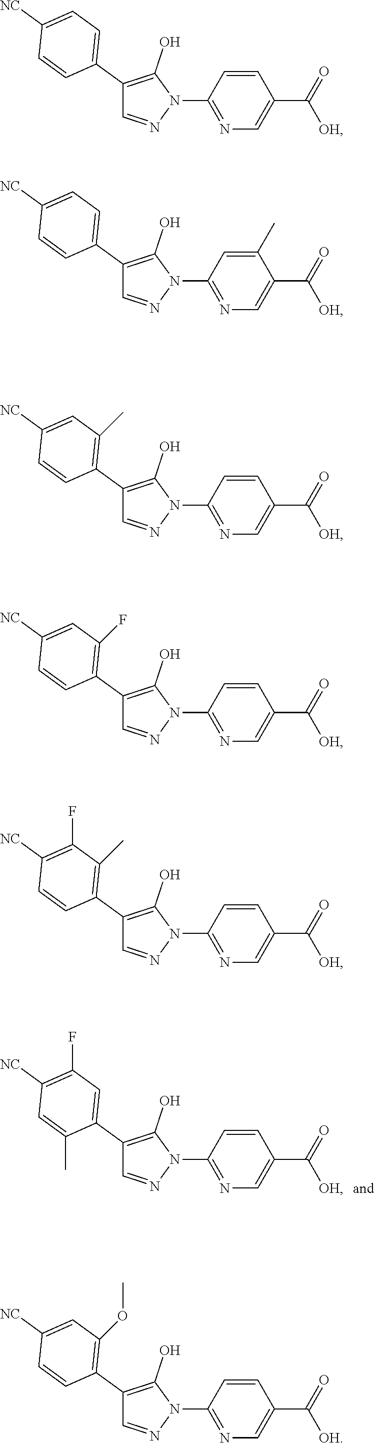

In embodiments, a compound of Formula (A), Formula (I), Formula (II), or Formula (III), has the following structure,

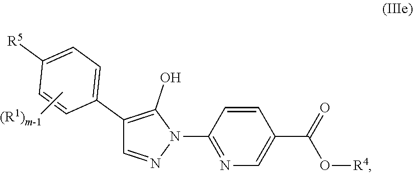

or a pharmaceutically acceptable salt thereof, wherein R¹, R⁴, and R⁵ are as defined anywhere herein.

In embodiments of formulas described herein (e.g., Formula (IIIe)), a compound (e.g., a compound of Formula (IIIe)) is not

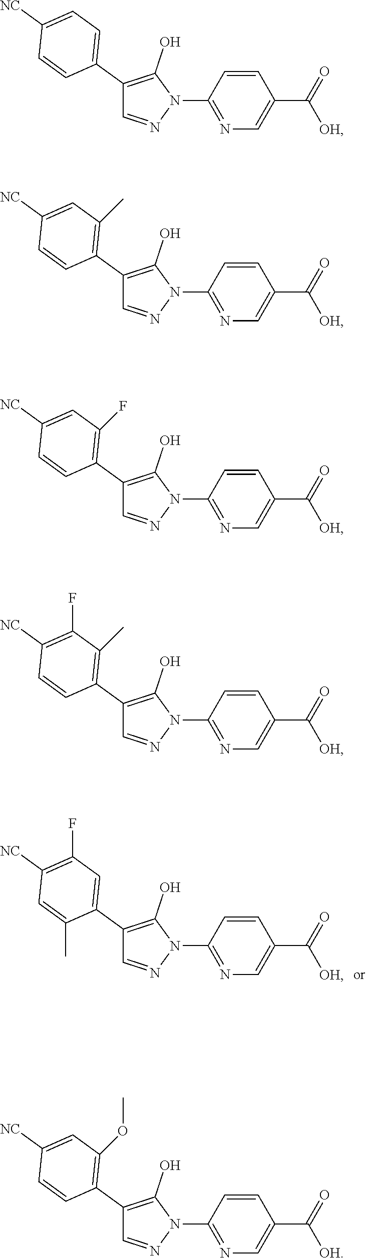

In embodiments of formulas described herein (e.g., Formula (IIIe)), a compound (e.g., a compound of Formula (IIIe)) excludes the following compounds

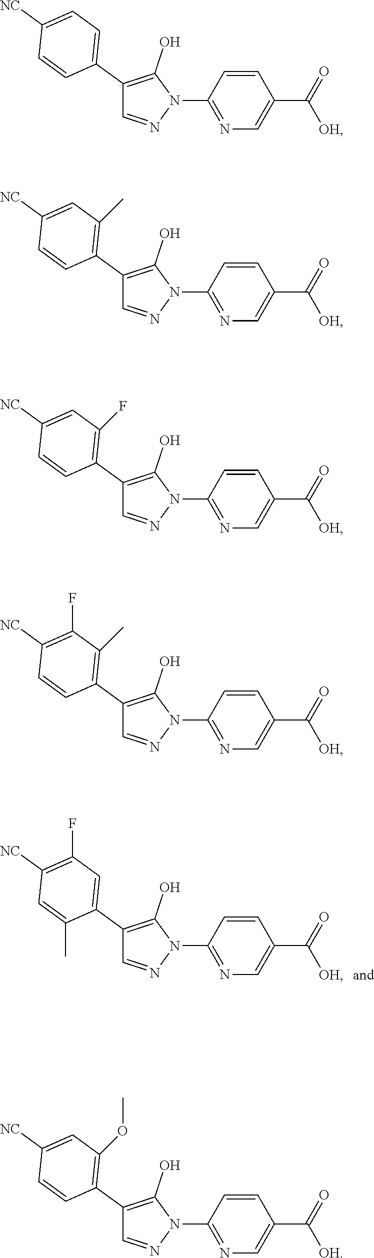

In embodiments, a compound of Formula (A), Formula (I), Formula (II), or Formula (III), has the following structure,

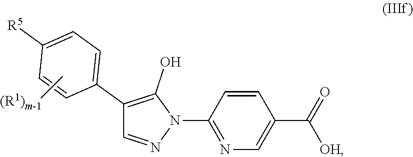

or a pharmaceutically acceptable salt thereof, wherein R¹ and R⁵ are as defined anywhere herein.

In embodiments of formulas described herein (e.g., Formula (IIIf)), a compound (e.g., a compound of Formula (IIIf)) is not

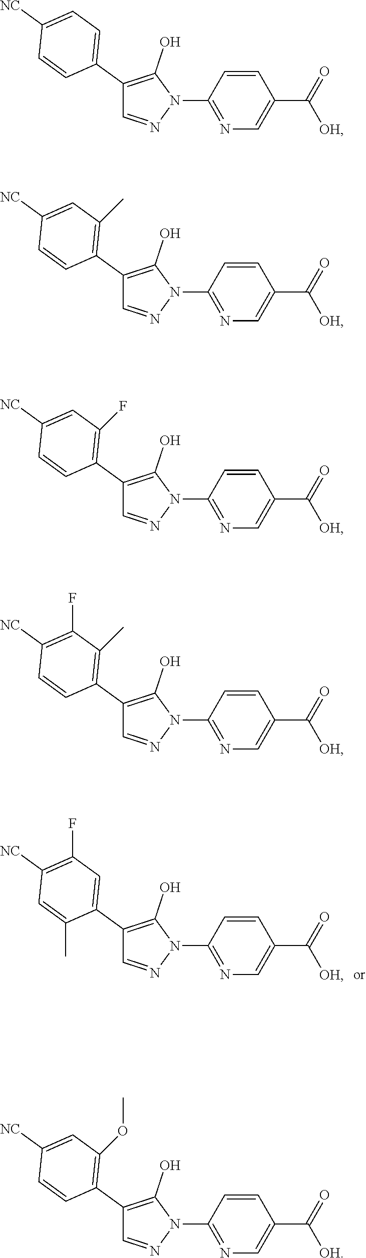

In embodiments of formulas described herein (e.g., Formula (IIIf)), a compound (e.g., a compound of Formula (IIIf)) excludes the following compounds

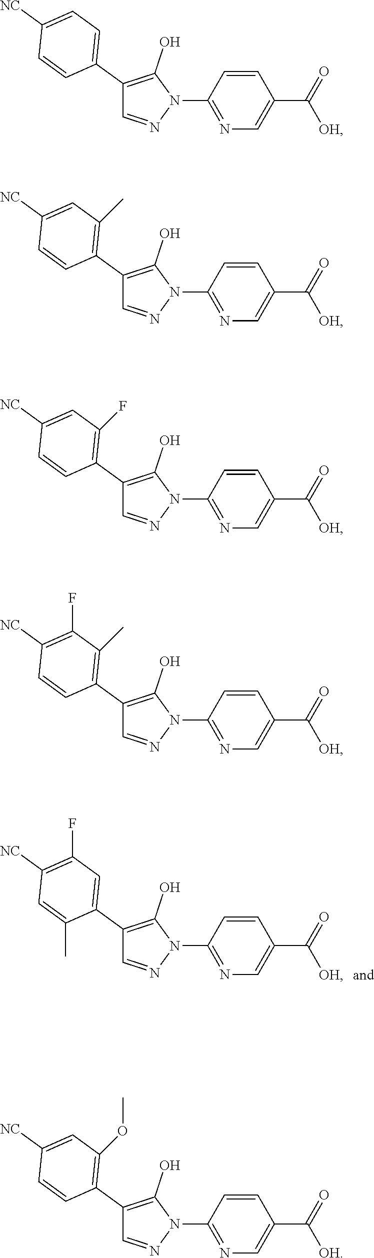

In embodiments, a compound of Formula (A), Formula (I), Formula (II), or Formula (III), has the following structure,

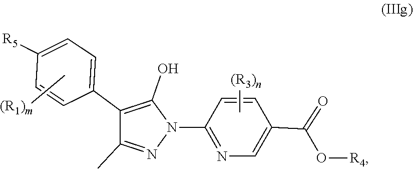

or a pharmaceutically acceptable salt thereof, wherein R¹, R³, R⁴ and R⁵ are as defined anywhere herein.

Exemplary Compounds

In some embodiments, the PHD inhibitor compounds is any one of Compounds 1-44 or a pharmaceutically acceptable salt thereof.

| Compd. | |
|---|---|
| No. | Structure |
| 1 | 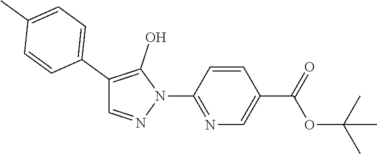 [SMILES: Cc1ccc(-c2cnn(-c3ccc(C(=O)OC(C)(C)C)cn3)c2O)cc1] |
| 2 | 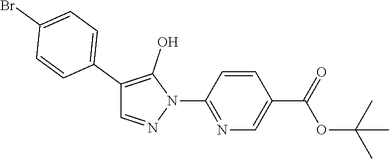 [SMILES: CC(C)(C)OC(=O)c1ccc(-n2ncc(-c3ccc(Br)cc3)c2O)nc1] |
| 3 | 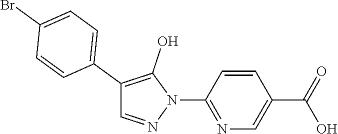 [SMILES: O=C(O)c1ccc(-n2ncc(-c3ccc(Br)cc3)c2O)nc1] |
| 4 | 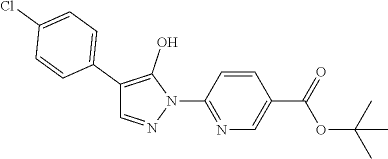 [SMILES: CC(C)(C)OC(=O)c1ccc(-n2ncc(-c3ccc(Cl)cc3)c2O)nc1] |
| 5 | 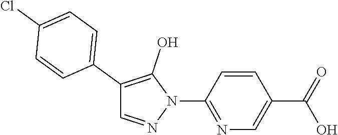 [SMILES: O=C(O)c1ccc(-n2ncc(-c3ccc(Cl)cc3)c2O)nc1] |
| 6 | 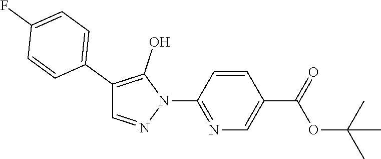 [SMILES: CC(C)(C)OC(=O)c1ccc(-n2ncc(-c3ccc(F)cc3)c2O)nc1] |
| 7 | 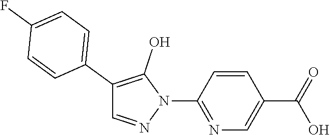 [SMILES: O=C(O)c1ccc(-n2ncc(-c3ccc(F)cc3)c2O)nc1] |
| 8 | 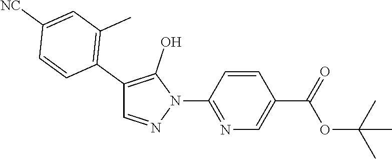 [SMILES: Cc1cc(C#N)ccc1-c1cnn(-c2ccc(C(=O)OC(C)(C)C)cn2)c1O] |
| 9 | 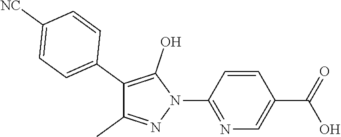 [SMILES: Cc1nn(-c2ccc(C(=O)O)cn2)c(O)c1-c1ccc(C#N)cc1] |
| 10 | 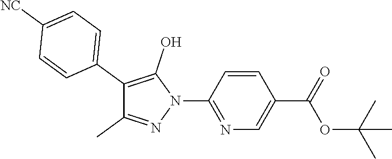 [SMILES: Cc1nn(-c2ccc(C(=O)OC(C)(C)C)cn2)c(O)c1-c1ccc(C#N)cc1] |
| 11 | 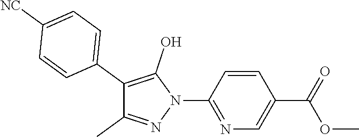 [SMILES: COC(=O)c1ccc(-n2nc(C)c(-c3ccc(C#N)cc3)c2O)nc1] |
| 12 | 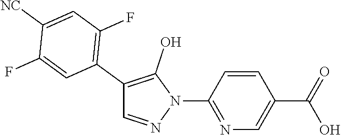 [SMILES: N#Cc1cc(F)c(-c2cnn(-c3ccc(C(=O)O)cn3)c2O)cc1F] |
| 13 | 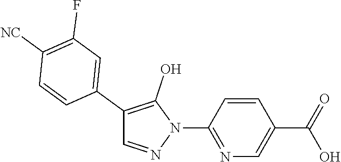 [SMILES: N#Cc1ccc(-c2cnn(-c3ccc(C(=O)O)cn3)c2O)cc1F] |
| 14 | 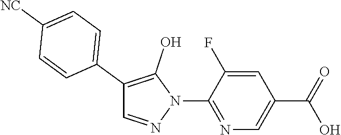 [SMILES: N#Cc1ccc(-c2cnn(-c3ncc(C(=O)O)cc3F)c2O)cc1] |
| 15 | 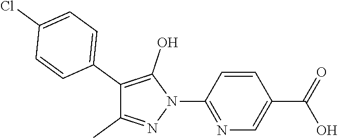 [SMILES: Cc1nn(-c2ccc(C(=O)O)cn2)c(O)c1-c1ccc(Cl)cc1] |
| 16 | 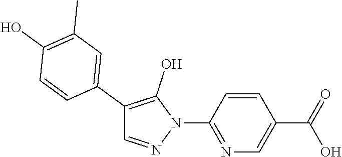 [SMILES: Cc1cc(-c2cnn(-c3ccc(C(=O)O)cn3)c2O)ccc1O] |
| 17 | 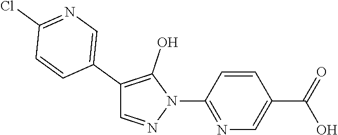 [SMILES: O=C(O)c1ccc(-n2ncc(-c3ccc(Cl)nc3)c2O)nc1] |
| 18 | 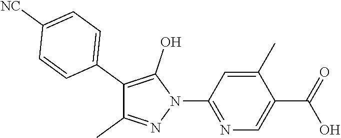 [SMILES: Cc1cc(-n2nc(C)c(-c3ccc(C#N)cc3)c2O)ncc1C(=O)O] |
| 19 | 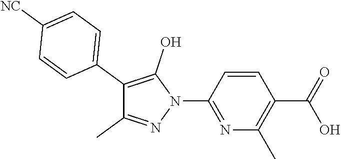 [SMILES: Cc1nc(-n2nc(C)c(-c3ccc(C#N)cc3)c2O)ccc1C(=O)O] |
| 20 | 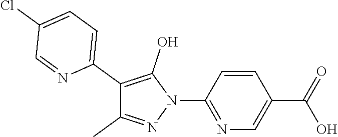 [SMILES: Cc1nn(-c2ccc(C(=O)O)cn2)c(O)c1-c1ccc(Cl)cn1] |
| 21 | 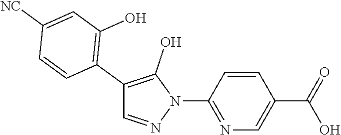 [SMILES: N#Cc1ccc(-c2cnn(-c3ccc(C(=O)O)cn3)c2O)c(O)c1] |
| 22 | 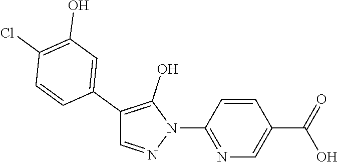 [SMILES: O=C(O)c1ccc(-n2ncc(-c3ccc(Cl)c(O)c3)c2O)nc1] |
| 23 | 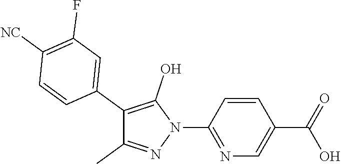 [SMILES: Cc1nn(-c2ccc(C(=O)O)cn2)c(O)c1-c1ccc(C#N)c(F)c1] |
| 24 | 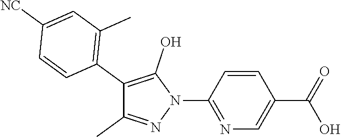 [SMILES: Cc1cc(C#N)ccc1-c1c(C)nn(-c2ccc(C(=O)O)cn2)c1O] |
| 25 | 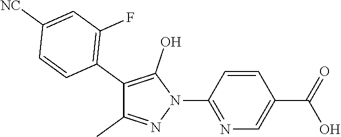 [SMILES: Cc1nn(-c2ccc(C(=O)O)cn2)c(O)c1-c1ccc(C#N)cc1F] |
| 26 | 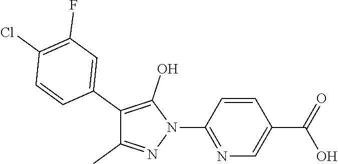 [SMILES: Cc1nn(-c2ccc(C(=O)O)cn2)c(O)c1-c1ccc(Cl)c(F)c1] |
| 27 | 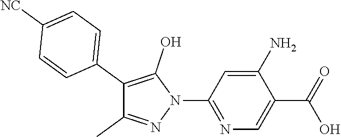 [SMILES: Cc1nn(-c2cc(N)c(C(=O)O)cn2)c(O)c1-c1ccc(C#N)cc1] |
| 28 | 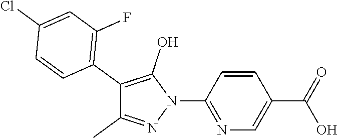 [SMILES: Cc1nn(-c2ccc(C(=O)O)cn2)c(O)c1-c1ccc(Cl)cc1F] |
| 29 | 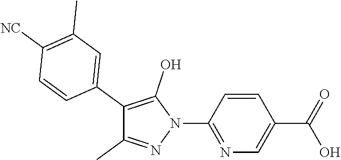 [SMILES: Cc1cc(-c2c(C)nn(-c3ccc(C(=O)O)cn3)c2O)ccc1C#N] |
| 30 | 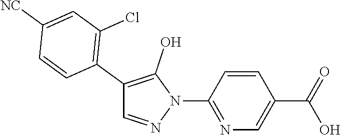 [SMILES: N#Cc1ccc(-c2cnn(-c3ccc(C(=O)O)cn3)c2O)c(Cl)c1] |
| 31 | 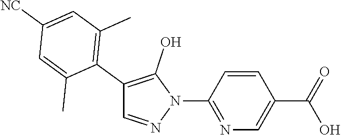 [SMILES: Cc1cc(C#N)cc(C)c1-c1cnn(-c2ccc(C(=O)O)cn2)c1O] |
| 32 | 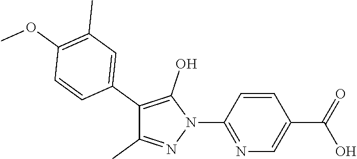 [SMILES: COc1ccc(-c2c(C)nn(-c3ccc(C(=O)O)cn3)c2O)cc1C] |
| 33 | 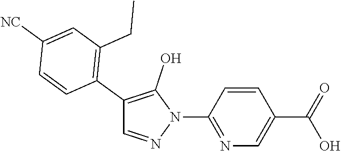 [SMILES: CCc1cc(C#N)ccc1-c1cnn(-c2ccc(C(=O)O)cn2)c1O] |
| 34 | 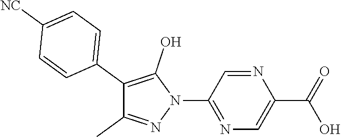 [SMILES: Cc1nn(-c2cnc(C(=O)O)cn2)c(O)c1-c1ccc(C#N)cc1] |
| 35 | 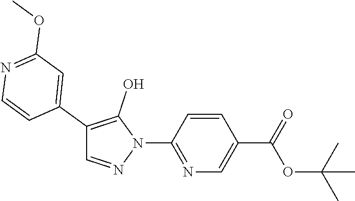 [SMILES: COc1cc(-c2cnn(-c3ccc(C(=O)OC(C)(C)C)cn3)c2O)ccn1] |
| 36 | 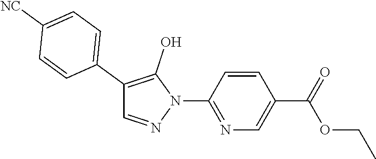 [SMILES: CCOC(=O)c1ccc(-n2ncc(-c3ccc(C#N)cc3)c2O)nc1] |
| 37 | 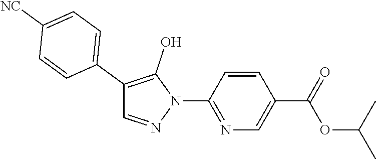 [SMILES: CC(C)OC(=O)c1ccc(-n2ncc(-c3ccc(C#N)cc3)c2O)nc1] |
| 38 | 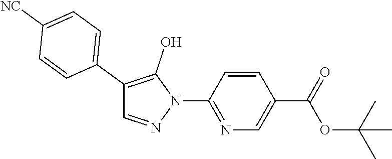 [SMILES: CC(C)(C)OC(=O)c1ccc(-n2ncc(-c3ccc(C#N)cc3)c2O)nc1] |
| 39 | 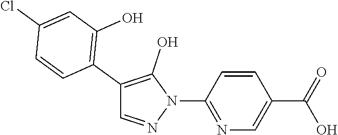 [SMILES: O=C(O)c1ccc(-n2ncc(-c3ccc(Cl)cc3O)c2O)nc1] |
| 40 | 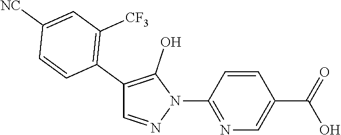 [SMILES: N#Cc1ccc(-c2cnn(-c3ccc(C(=O)O)cn3)c2O)c(C(F)(F)F)c1] |
| 41 | 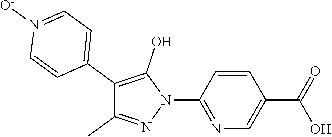 |
| 42 | 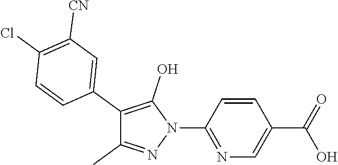 [SMILES: Cc1nn(-c2ccc(C(=O)O)cn2)c(O)c1-c1ccc(Cl)c(C#N)c1] |
| 43 | 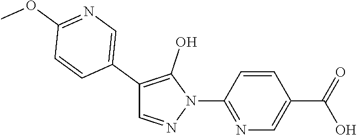 [SMILES: COc1ccc(-c2cnn(-c3ccc(C(=O)O)cn3)c2O)cn1] |
| 44 | 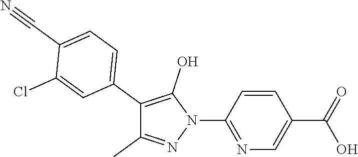 [SMILES: Cc1nn(-c2ccc(C(=O)O)cn2)c(O)c1-c1ccc(C#N)c(Cl)c1] |

Isotopologues

It should be understood that in the compounds described herein (e.g., a compound of any one of Formulas (A) and (I)-(III) such as any one of compounds 1-44), the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of the compounds described herein (e.g., a compound of any one of Formulas (A) and (I)-(III) such as any one of compounds 1-44). For example, different isotopic forms of hydrogen (H) include protium (¹H), deuterium (²H), and tritium (H). Protium is the predominant hydrogen isotope found in nature.

In some embodiments, one or more of the hydrogens of the compounds described herein (e.g., a compound of any one of Formulas (A) and (I)-(III) such as any one of compounds 1-44) is replaced by a deuterium. Enriching for deuterium may afford certain therapeutic advantages, such as increasing in vivo half-life or reducing dosage requirements, or may provide a compound useful as a standard for characterization of biological samples. In some embodiments, one or more of the hydrogens of the compounds described herein (e.g., a compound of any one of Formulas (A) and (I)-(III) such as any one of compounds 1-44) is replaced by tritium. Tritium is radioactive and may therefore provide for a radiolabeled compound, useful as a tracer in metabolic or kinetic studies.

Isotopic-enrichment of compounds disclosed herein (e.g., a compound of any one of Formulas (A) and (I)-(III) such as any one of compounds 1-44), may be achieved without undue experimentation by conventional techniques well known to those skilled in the art or by processes analogous to those described in the Schemes and Examples herein using appropriate isotopically-enriched reagents and/or intermediates.

The term “isotopologue” refers to a species that has the same chemical structure and formula as a specific compound provided herein, with the exception of the positions of isotopic substitution and/or level of isotopic enrichment at one or more positions, e.g., hydrogen vs. deuterium. Thus, the term “compound,” as used herein, encompasses a collection of molecules having identical chemical structure, but also having isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound provided depends upon a number of factors including, but not limited to, the isotopic purity of deuterated reagents used to make the compound and the efficiency of incorporation of deuterium in the various synthesis steps used to prepare the compound.

When a position is designated as “H” or “hydrogen”, the position is understood to have hydrogen at its natural abundance isotopic composition. When a position is designated as “D” or “deuterium”, the position is understood to have deuterium at an abundance that is at least 3340 times greater than the natural abundance of deuterium, which is 0.015% (i.e., the term “D” or “deuterium” indicates at least 50.1% incorporation of deuterium).

In embodiments, a compound provided herein may have an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Synthesis of Compounds of the Inventions

The compounds described herein (e.g., a compound of any one of Formulas A and I-III such as any one of compounds 1-44) can be prepared according to methods known in the art, including the exemplary syntheses of the Examples provided herein, such as the synthesis shown in Scheme A.

Purity of the compounds and their synthetic intermediates were determined by reverse phase HPLC using either one of the methods described below:

Method A: Mobile Phase: A: Water (0.01% TFA) B: Acetonitrile (0.01% TFA); Gradient Phase: 5% B increase to 95% B within 1.4 min, 95% B with 1.6 min (total run time:3 min); Flow Rate: 2.3 mL/min. Column: SunFire C18, 4.6*50 mm, 3.5 μm; Column Temperature: 50° C. Detectors: ADC ELSD, DAD (214 nm and 254 nm), ES-API.

Method B: Mobile Phase: A: Water (10 mM NH₄HCO₃) B: Acetonitrile; Gradient Phase: 5% to 95% B within 1.5 min, 95% B with 1.5 min (total run time:3 min); Flow Rate: 2.0 mL/min; Column: XBridge C18, 4.6*50 mm, 3.5 um; Column Temperature: 40° C. Detectors: ADC ELSD, DAD (214 nm and 254 nm), MSD (ES-API).

Abbreviations and acronyms used herein including the following:

| Term | Acronym |
|---|---|
| 4-Dimethylaminopyridine | DMAP |
| Acetyl | Ac |
| Aqueous | aq. |
| Benzyl | Bn |
| tert-Butyloxycarbonyl | Boc |
| Broad singlet | brs |
| Dichloromethane | DCM |
| Dimethylsulfoxide | DMSO |
| Doublet | d |
| Electrospray ionization | ESI |
| Equivalent | eq |
| Ethyl acetate | EtOAc |
| Gram | g |
| Hexanes | Hex |
| High performance liquid chromatography | HPLC |
| Hour | hr |
| Isopropyl | LPr |
| Liquid chromatography-mass spectrometry | LCMS |
| Megahertz | MHz |
| meta-Chloroperoxybenzoic acid | m-CPBA |
| Methanol | MeOH |
| Milligram | mg |
| Milliliter | mL |
| Minute | min |
| Multiplet | m |
| N,N-Diisopropylethylamine | DIPEA |
| N,N-Dimethylformamide | DMF |
| N,N-dimethylformamide dimethyl acetal | DMF-DMA |
| Normal | N |
| Nuclear magnetic resonance | NMR |
| Palladium on carbon | Pd/C |
| Pentet | P |
| Petroleum ether | PE |
| Phenyl | Ph |
| Quartet | q |
| Room temperature | RT |
| Singlet | s |
| Tetrahydrofuran | THF |
| Thin layer chromatography | TLC |
| Tri ethyl amine | TEA |
| Trifluoroacetic acid | TFA |
| Triplet | t |

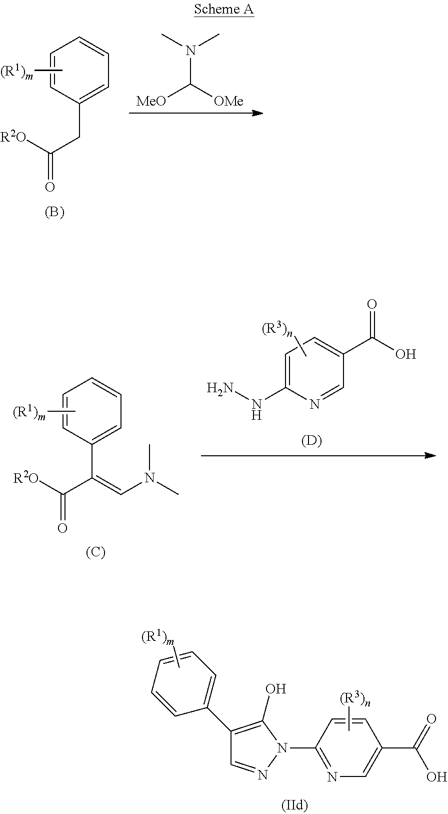

Compounds of Formula (IId) are prepared according to Scheme A using commercially available materials. Compounds of Formula (IId) are prepared according to Scheme A using commercially available materials. The reaction with esters of formula (B) with N,N-dimethylformamide dimethyl acetal yields enamine compounds with formula (C). Cyclization of (C) and hydrazide (D) furnishes pyrazole compounds with formula (IId).

### Compositions and Methods

The invention provides for use of a compound of any one of Formulas (A) and (I)-(III), or a pharmaceutically acceptable salt thereof, for the manufacture of a medicament for use in treating various conditions or disorders as described herein. In one embodiment, a pharmaceutical composition is provided comprising at least one compound of any one of Formulas (A) and (I)-(III), or a pharmaceutically acceptable salt thereof, and a pharmaceutically acceptable excipient or carrier. In various embodiments, the medicament or pharmaceutical composition can further comprise or be used in combination with at least one additional therapeutic agent.

The compounds of the present invention, or medicaments or compositions comprising the compounds, can be used to inhibit the activity of PHD. Inhibition of PHD may be of particular benefit in treating diseases including heart (e.g. ischemic heart disease, congestive heart failure, and valvular heart disease), lung (e.g., acute lung injury, pulmonary hypertension, pulmonary fibrosis, and chronic obstructive pulmonary disease), liver (e.g. acute liver failure and liver fibrosis and cirrhosis), and kidney (e.g. acute kidney injury and chronic kidney disease) disease. In one embodiment, the method of the invention comprises administering to a patient in need a therapeutically effective amount of a compound of any one of Formulas (A) and (I)-(III), or a pharmaceutically acceptable salt thereof, or a pharmaceutical composition comprising one or more compounds of any one of Formulas (A) and (I)-(III).

The invention is also directed to a method of inhibiting the activity of PHD. In one embodiment, the method comprises contacting PHD with an effective amount of one or more compounds selected from the group comprising compounds of any one of Formulas (A) and (I)-(III), or a pharmaceutically acceptable salt thereof.

In still other embodiments, the compounds disclosed herein (e.g., a compound of any one of Formulas (A) and (I)-(III) such as any one of compounds 1-44), or a pharmaceutically acceptable salt thereof, are useful for the treatment or prevention of anemia comprising treatment of anemic conditions associated with chronic kidney disease, polycystic kidney disease, aplastic anemia, autoimmune hemolytic anemia, bone marrow transplantation anemia, Churg-Strauss syndrome, Diamond Blackfan anemia, Fanconi's anemia, Felty syndrome, graft versus host disease, hematopoietic stem cell transplantation, hemolytic uremic syndrome, myelodysplastic syndrome, nocturnal paroxysmal hemoglobinuria, osteomyelofibrosis, pancytopenia, pure red-cell aplasia, purpura Schoenlein-Henoch, refractory anemia with excess of blasts, rheumatoid arthritis, Shwachman syndrome, sickle cell disease, thalassemia major, thalassemia minor, thrombocytopenic purpura, anemic or non-anemic patients undergoing surgery, anemia associated with or secondary to trauma, sideroblastic anemia, anemic secondary to other treatment including: reverse transcriptase inhibitors to treat HIV, corticosteroid hormones, cyclic cisplatin or non-cisplatin-containing chemotherapeutics, vinca alkaloids, mitotic inhibitors, topoisomerase II inhibitors, anthracyclines, alkylating agents, particularly anemia secondary to inflammatory, aging and/or chronic diseases. PHD1 inhibition may also be used to treat symptoms of anemia including chronic fatigue, pallor, and dizziness.

In other embodiments, the compounds disclosed herein (e.g., a compound of Formulas (A) and (I)-(III) such as any one of compounds 1-44), or a pharmaceutically acceptable salt thereof, are useful for the treatment or prevention of diseases of metabolic disorders, including but not limited to diabetes and obesity.

In yet other embodiments, the compounds disclosed herein (e.g., a compound of Formulas (A) and (I)-(III) such as any one of compounds 1-44), or a pharmaceutically acceptable salt thereof, are useful for the treatment or prevention of vascular disorders. These include but are not limited to hypoxic or wound healing related diseases requiring pro-angiogenic mediators for vasculogenesis, angiogenesis, and arteriogenesis

In still other embodiments, the compounds disclosed herein (e.g., a compound of Formulas (A) and (I)-(III) such as any one of compounds 1-44), or a pharmaceutically acceptable salt thereof, are useful for the treatment or prevention of ischemia reperfusion injury. These include but are not limited to stroke, myocardial infarction, and acute kidney injury.

In other embodiments, the compounds disclosed herein (e.g., a compound of Formulas (A) and (I)-(III) such as any one of compounds 1-44), or a pharmaceutically acceptable salt thereof, are useful in the treatment of inflammatory bowel disease. These include but are not limited to ulcerative colitis, and Crohn's disease.

In other embodiments, the compounds disclosed herein (e.g., a compound of Formulas (A) and (I)-(III) such as any one of compounds 1-44), or a pharmaceutically acceptable salt thereof, are useful in the treatment of cancers, such as colorectal cancer.

In other embodiments, the compounds disclosed herein (e.g., a compound of Formulas (A) and (I)-(III) such as any one of compounds 1-44), or a pharmaceutically acceptable salt thereof, are useful in the treatment of atherosclerosis.

In other embodiments, the compounds disclosed herein (e.g., a compound of Formulas (A) and (I)-(III) such as any one of compounds 1-44), or a pharmaceutically acceptable salt thereof, are useful in the treatment of cardiovascular disease.

In other embodiments, the compounds disclosed herein (e.g., a compound of Formulas (A) and (I)-(III) such as any one of compounds 1-44), or a pharmaceutically acceptable salt thereof, are useful in the treatment of a disease or condition of the eye. These include but are not limited to radiation retinopathy, retinopathy of prematurity, diabetic retinopathy, age-related macular degeneration, and ocular ischemia.

In other embodiments, the compounds disclosed herein (e.g., a compound of Formulas (A) and (I)-(III) such as any one of compounds 1-44), or a pharmaceutically acceptable salt thereof, are useful in the treatment of a disease that is associated with hyperoxia.

In other embodiments, the compounds disclosed herein (e.g., a compound of Formulas (A) and (I)-(III) such as any one of compounds 1-44), or a pharmaceutically acceptable salt thereof, are useful in the treatment of bronchopulmonary dysplasia (BPD).

In yet other embodiments, the compounds disclosed herein (e.g., a compound of Formulas (I)-(III) such as any one of compounds 1-44), or a pharmaceutically acceptable salt thereof, are useful in the treatment of heart diseases. The conditions include but are not limited to postoperative myocardial ischemia in pancreatic surgery, myocardial injury after percutaneous coronary intervention (PCI), myocardial injury after non-cardiac surgery, perioperative myocardial ischemia in elective operation of abdominal aortic aneurysm, myocardial injury after PCI, myocardial damage in patients undergoing coronary artery bypass graft (CABG) surgery, Minimally invasive mitral valve (MIMV) repair or replacement, adult patient undergoing open heart surgery, chronic heart failure, NYHA class II-IV.

In other embodiments, the compounds disclosed herein (e.g., a compound of Formulas (A) and ((I)-(III) such as any one of compounds 1-44), or a pharmaceutically acceptable salt thereof, are useful in the treatment of lung diseases. The conditions include but are not limited to lung injury during elective lung lobectomy, lung injury during CABG surgery, lung transplantation.

In other embodiments, the compounds disclosed herein (e.g., a compound of Formulas (A) and (I)-(III) such as any one of compounds 1-44), or a pharmaceutically acceptable salt thereof, are useful in the treatment of liver disease. The conditions include but are not limited to non-alcoholic steatohepatitis (NASH).

In other embodiments, the compounds disclosed herein (e.g., a compound of Formulas (A) and (I)-(III) such as any one of compounds 1-44), or a pharmaceutically acceptable salt thereof, are useful in the treatment of kidney disease. The conditions include but are not limited to contrast-induced acute kidney injury, stage III-IV chronic kidney disease undergoing planned coronary angiography, acute kidney injury in patients undergoing valvular heart surgery, non-dialysis dependent chronic kidney disease, chronic kidney disease patients initiating dialysis, non-dialysis dependent chronic kidney disease.

In addition, the compounds disclosed herein (e.g., a compound of any one of Formulas (A) and (I)-(III) such as any one of compounds 1-44), or a pharmaceutically acceptable salt thereof, may be used in combination with additional active ingredients in the treatment of the above conditions. The additional compounds may be co-administered separately with the compounds disclosed herein (e.g., a compound of any one of Formulas (A) and (I)-(III) such as any one of compounds 1-44), or a pharmaceutically acceptable salt thereof, or included with an additional active ingredient in a pharmaceutical composition according to the invention. In an exemplary embodiment, additional active ingredients are those that are known or discovered to be effective in the treatment of conditions, disorders, or diseases mediated by PHD enzyme or that are active against another targets associated with the particular condition, disorder, or disease, such as an alternate PHD modulator. The combination may serve to increase efficacy (e.g., by including in the combination a compound potentiating the potency or effectiveness of a compound according to the invention), decrease one or more side effects, or decrease the required dose of the compound according to the invention.

The compounds of the invention are used, alone or in combination with one or more other active ingredients, to formulate pharmaceutical compositions of the invention. A pharmaceutical composition of the invention comprises: (a) an effective amount of the compounds disclosed herein (e.g., a compound of any one of Formulas (A) and (I)-(III) such as any one of compounds 1-44), or a pharmaceutically acceptable salt, pharmaceutically acceptable prodrug, or pharmaceutically active metabolite thereof, and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. Suitable excipients may also include antioxidants. Such antioxidants may be used in a pharmaceutical composition or in a storage medium to prolong the shelf-life of the drug product.

### Pharmaceutical Formulations and Routes of Administration

The compounds and compositions of the present invention can be delivered directly or in pharmaceutical compositions or medicaments along with suitable carriers or excipients, as is well known in the art. Present methods of treatment can comprise administration of an effective amount of a compound of the invention to a subject in need. In a preferred embodiment, the subject is a mammalian subject, and in a most preferred embodiment, the subject is a human subject.

An effective amount of such compound, composition, or medicament can readily be determined by routine experimentation, as can the most effective and convenient route of administration, and the most appropriate formulation. Various formulations and drug delivery systems are available in the art. See, e.g., Gennaro, A. R., ed. (1995) Remington's Pharmaceutical Sciences, supra.

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art and include those listed in various pharmacopoeias. See, e.g., the U.S. Pharmacopeia (USP), Japanese Pharmacopoeia (JP), European Pharmacopoeia (EP), and British pharmacopeia (BP); the U.S. Food and Drug.

Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott NY; etc.) [0149] Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compounds and compositions formulated for parenteral administration by injection are usually sterile and can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the compounds of the invention are well-known in the art and include water-based solutions containing a base, such as, for example, sodium hydroxide, to form an ionized compound; sucrose or sodium chloride as a tonicity agent; and a buffer, for example, a buffer that contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays. In certain some embodiments, a compound of the disclosure is formulated for oral administration. An exemplary dose of a compound of the disclosure in a pharmaceutical formulation for oral administration is from about 0.5 to about 10 mg/kg body weight of subject. In some embodiments, a pharmaceutical formulation comprises from about 0.7 to about 5.0 mg/kg body weight of subject, or alternatively, from about 1.0 to about 2.5 mg/kg body weight of subject. A typical dosing regimen for oral administration would be administration of the pharmaceutical formulation for oral administration three times per week, two times per week, once per week or daily.

An effective amount or a therapeutically effective amount or dose of an agent, e.g., a compound of the invention, refers to that amount of the agent or compound that results in amelioration of symptoms or a prolongation of survival in a subject.

Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of compound or composition administered may be dependent on a variety of factors, including the sex, age, and weight of the subject being treated, the severity of the affliction, the manner of administration, and the judgment of the prescribing physician.

The present compounds and compositions may, if desired, be presented in a pack or dispenser device containing one or more unit dosage forms containing the active ingredient. Such a pack or device may, for example, comprise metal or plastic foil, such as a blister pack; or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein and are specifically contemplated.

### EXEMPLIFICATION

Synthesis for Exemplary Compounds

### Example 1: Preparation of Compound 1

### Tert-butyl 6-chloronicotinate

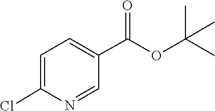

To a solution of 6-chloronicotinic acid (5.0 g, 6.37 mmol) and 4-dimethylaminopyridine (0.39 g, 0.64 mmol) in tetrahydrofuran (50.0 mL) was added di-tert-butyl dicarbonate (10.41 g, 47.77 mmol). The reaction mixture was stirred at reflux for 4 h and concentrated. The residue was purified by flash chromatography (petroleum ether/ethyl acetate=10/1) to afford tert-butyl 6-chloronicotinate (5.5 g, 81.12% yield) as yellow solid. LC-MS: m/z=214.0 (M+H)⁺, retention time 1.83 min (Method A).

### Tert-butyl 6-hydrazineylnicotinate

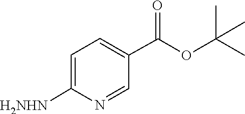

To a solution of tert-butyl 6-chloronicotinate (5.5 g, 25.82 mmol) in ethanol (25.0 mL) was added hydrazine hydrate (6.46 g, 129.11 mmol, 85% in water). The mixture was stirred at 100° C. for 2 h and concentrated to dryness. The residue was partitioned between ethyl acetate and water. The organic phase was washed with brine, dried over sodium sulfate and concentrated. The residue was triturated with petroleum ether and filtered to afford tert-butyl 6-hydrazineylnicotinate (5.0 g, 92.76% yield) as yellow solid. LC-MS: m/z=210.0 (M+H)⁺, retention time 1.19 min (Method A).

### Ethyl (E)-3-(dimethylamino)-2-(p-tolyl)acrylate

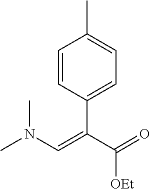 [SMILES: CCOC(=O)/C(=C/N(C)C)c1ccc(C)cc1]

To a solution of ethyl 2-(p-tolyl)acetate (1.00 g, 5.61 mmol) in N,N-dimethylformamide (10.0 mL) was added N,N-dimethylformamide diethyl acetal (3.34 g, 28.05 mmol, 3.73 mL). The reaction was stirred at 100° C. for 3 h and cooled to temperature. Ethyl acetate and water were added to the solution, and the layers were separated. The organic layer was washed with brine, dried over sodium sulfate and concentrated to give ethyl (E)-3-(dimethylamino)-2-(p-tolyl)acrylate (1.1 g, 4.71 mmol, 84% yield). LC-MS: m/z=234.0 [M+H]⁺, retention time 2.064 min (Method B). The product was pure enough and used directly to the next step.

### Tert-butyl 6-(5-hydroxy-4-(p-tolyl)-1H-pyrazol-1-yl)nicotinate

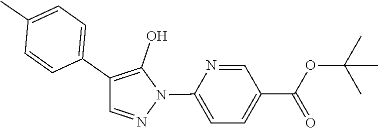 [SMILES: Cc1ccc(-c2cnn(-c3ccc(C(=O)OC(C)(C)C)cn3)c2O)cc1]

To a solution of ethyl (E)-3-(dimethylamino)-2-(p-tolyl)acrylate (50.00 mg, 2.14 mmol) and tert-butyl 6-hydrazineylnicotinate (448.4 mg, 2.14 mmol) in ethanol (10.0 mL) was added p-toluenesulfonic acid monohydrate (40 mg, 0.21 mmol). The reaction was stirred at 90° C. for 16 h and cooled to precipitate solid. The crude solid was purified by flash chromatography (dichloromethane/ethyl acetate=10/1) to afford tert-butyl 6-(5-hydroxy-4-(p-tolyl)-1H-pyrazol-1-yl)nicotinate (393 mg, 1.11 mmol, 52% yield) as yellow solid. LC-MS: m/z=352.0 [M+H]⁺, retention time 6.78 min (Method A). ¹HNMR (400 MHz, DMSO-d₆) (12.86 (s, 1H), 8.92 (s, 1H), 8.80-8.15 (m, 3H), 7.78 (s, 2H), 7.17 (d, J=8.0 Hz, 2H), 2.29 (s, 3H), 1.58 (s, 9H).

### Example 2: Preparation of Compound 2

### Ethyl (E)-2-(4-bromophenyl)-3-(dimethylamino)acrylate

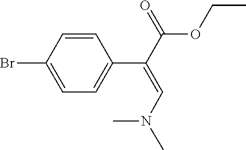 [SMILES: CCOC(=O)/C(=C/N(C)C)c1ccc(Br)cc1]

To a solution of ethyl 2-(4-bromophenyl) acetate (1.01 g, 4.15 mmol) in N,N-dimethylformamide (20.0 mL) was added N,N-dimethylformamide diethyl acetal (2.47 g, 20.7 mmol). The mixture was stirred at 100° C. for 16 h and cooled to temperature. Ethyl acetate and water were added to the solution, and the layers were separated. The organic layer was washed with brine, dried over sodium sulfate and concentrated to give ethyl (E)-2-(4-bromophenyl)-3-(dimethylamino)acrylate (980 mg, 3.32 mmol, 80% yield) as yellow oil. LC-MS: m/z=298.0 [M+H]⁺, retention time 1.710 min (Method B). The product was pure enough and used directly to the next step.

### Tert-butyl 6-(4-(4-bromophenyl)-5-hydroxy-1H-pyrazol-1-yl)nicotinate

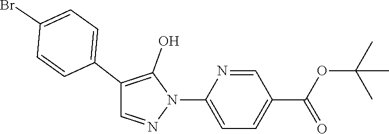 [SMILES: CC(C)(C)OC(=O)c1ccc(-n2ncc(-c3ccc(Br)cc3)c2O)nc1]

To a solution of ethyl (E)-2-(4-bromophenyl)-3-(dimethylamino)acrylate (300.0 mg, 1.01 mmol) and tert-butyl 6-hydrazineylnicotinate (210.5 mg, 1.01 mmol) in ethanol (10.0 mL) was added p-toluenesulfonic acid monohydrate (38 mg, 0.2 mmol). The mixture was stirred at 80° C. for 16 h and cooled to precipitate solid. The crude product was purified by flash chromatography (dichloromethane/methanol=100/3) to afford tert-butyl 6-(4-(4-bromophenyl)-5-hydroxy-1H-pyrazol-1-yl)nicotinate (286 mg, 0.69 mmol, 68% yield) as yellow solid. LC-MS: m/z=416.0 (M+H)⁺, retention time 6.76 min (Method A). ¹HNMR (400 MHz, DMSO-d₆) (13.13 (br, 1H), 8.91 (s, 1H), 8.80-8.25 (m, 3H), 7.89 (br, 2H), 7.53 (d, J=8.0 Hz, 2H), 1.58 (s, 9H).

### Example 3: Preparation of Compound 3

### 6-(4-(4-Bromophenyl)-5-hydroxy-1H-pyrazol-1-yl) nicotinic acid

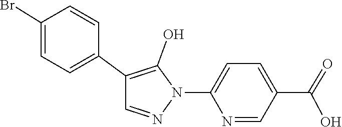 [SMILES: O=C(O)c1ccc(-n2ncc(-c3ccc(Br)cc3)c2O)nc1]

To a solution of tert-butyl 6-(4-(4-bromophenyl)-5-hydroxy-1H-pyrazol-1-yl)nicotinate (80.00 mg, 0.19 mmol) in dichloromethane (10.0 mL) was added trifluoroacetic acid (0.5 mL). The mixture was stirred at room temperature overnight and concentrated. The residue was triturated with ethyl acetate and filtered to afford 6-(4-(4-bromophenyl)-5-hydroxy-1H-pyrazol-1-yl) nicotinic acid (67 mg, 0.18 mmol, 97% yield) as yellow solid. LC-MS: m/z=360.0 (M+H)⁺, retention time 4.995 min (Method A). ¹HNMR (400 MHz, DMSO-d₆) (13.29 (br, 2H), 8.96 (s, 1H), 8.51-8.44 (m, 3H), 7.89 (d, J=7.5 Hz, 2H), 7.53 (d, J=8.5 Hz, 2H).

### Example 4: Preparation of Compound 4

### Ethyl (E)-3-(dimethylamino)-2-(4-chlorophenyl)acrylate

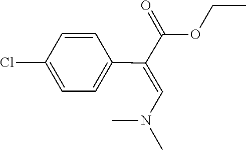 [SMILES: CCOC(=O)/C(=C/N(C)C)c1ccc(Cl)cc1]

To a solution of ethyl 2-(4-chlorophenyl)acetate (5.0 g, 25.25 mmol) in N,N-dimethylformamide (25.0 mL) was added N,N-dimethylformamide diethyl acetal (12.0 g, 101.01 mmol). The mixture was stirred at 100° C. overnight and cooled to room temperature. Ethyl acetate and water were added to the solution, and the layers were separated. The organic layer was washed with brine, dried over sodium sulfate and concentrated to give ethyl (E)-3-(dimethylamino)-2-(4-chlorophenyl)acrylate (3.5 g, 14.04 mmol, 55.60% yield) as colorless oil. LC-MS: m/z=254.1 [M+H]⁺, retention time 2.030 min (Method A). The product was pure enough and used directly to the next step.

### Tert-butyl 6-(4-(4-chlorophenyl)-5-hydroxy-1H-pyrazol-1-yl)nicotinate

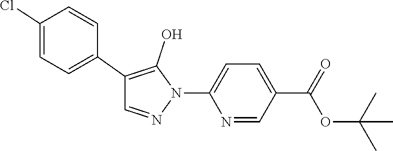 [SMILES: CC(C)(C)OC(=O)c1ccc(-n2ncc(-c3ccc(Cl)cc3)c2O)nc1]

To a solution of ethyl (E)-3-(dimethylamino)-2-(4-chlorophenyl)acrylate (0.83 g, 3.95 mmol) and tert-butyl 6-hydrazineylnicotinate (1.00 g, 3.95 mmol) in ethanol (10.0 mL) was added p-toluenesulfonic acid monohydrate (150 mg, 0.79 mmol). The mixture was stirred at reflux for 12 h and cooled to precipitate solid. The solid was filtered, washed with ethanol and dried to give tert-butyl 6-(4-(4-chlorophenyl)-5-hydroxy-1H-pyrazol-1-yl)nicotinate (800.0 mg, 2.16 mmol, 54.79% yield) as white solid. LC-MS: m/z=372.1 (M+H)⁺, retention time 6.645 min (Method A). ¹HNMR (400 MHz, DMSO-d₆) (9.04-8.86 (m, 1H), 8.62-8.33 (m, 3H), 7.95 (d, J=7.2 Hz, 2H), 7.41 (d, J=8.6 Hz, 2H), 1.58 (s, 9H).

### Example 5: Preparation of Compound 5

### 6-(4-(4-Chlorophenyl)-5-hydroxy-1H-pyrazol-1-yl)nicotinic acid

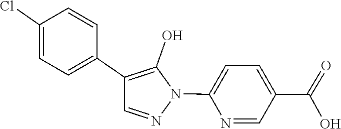 [SMILES: O=C(O)c1ccc(-n2ncc(-c3ccc(Cl)cc3)c2O)nc1]

To a solution of tert-butyl 6-(4-(4-chlorophenyl)-5-hydroxy-1H-pyrazol-1-yl)nicotinate (200.00 mg, 0.54 mmol) in dichloromethane (10.0 mL) was added trifluoroacetic acid (5.0 mL). The mixture was stirred at 40° C. for 2 h and concentrated. The residue was triturated with ethyl acetate and filtered to afford 6-(4-(4-chlorophenyl)-5-hydroxy-1H-pyrazol-1-yl) nicotinic acid (74.0 mg, 0.24 mmol, 43.52% yield) as white solid. LC-MS: m/z=316.0 (M+H)⁺, retention time 4.718 min (Method A). ¹HNMR (400 MHz, DMSO-d₆) (13.23 (s, 1H), 8.97 (s, 1H), 8.67-8.47 (m, 3H), 8.04-7.97 (m, 2H), 7.41 (d, J=3.8 Hz, 2H).

### Example 6: Preparation of Compound 6

### Ethyl (E)-3-(dimethylamino)-2-(4-fluorophenyl)acrylate

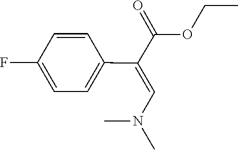 [SMILES: CCOC(=O)/C(=C/N(C)C)c1ccc(F)cc1]

To a solution of ethyl 2-(4-fluorophenyl)acetate (2.0 g, 10.98 mmol) in N,N-dimethylformamide (5.0 mL) was added N,N-dimethylformamide diethyl acetal (8.07 g, 54.8 mmol). The mixture was stirred at 100° C. overnight and cooled to room temperature. Ethyl acetate and water were added to the solution, and the layers were separated. The organic layer was washed with brine, dried over sodium sulfate and concentrated to give ethyl (E)-3-(dimethylamino)-2-(4-fluorophenyl)acrylate (2.0 g, 8.45 mmol, 77% yield) as colorless oil. LC-MS: m/z=238.0 [M+H]⁺, retention time 1.89 min (Method B). The product was pure enough and used directly to the next step

### Tert-butyl 6-(4-(4-fluorophenyl)-5-hydroxy-1H-pyrazol-1-yl)nicotinate

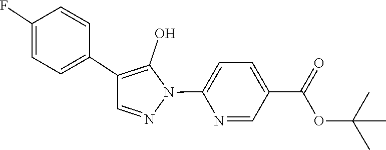 [SMILES: CC(C)(C)OC(=O)c1ccc(-n2ncc(-c3ccc(F)cc3)c2O)nc1]

To a solution of ethyl (E)-3-(dimethylamino)-2-(4-fluorophenyl)acrylate (567.0 mg, 2.39 mmol) and tert-butyl 6-hydrazineylnicotinate (500.0 mg, 2.39 mmol) in ethanol (5.0 mL) was added p-toluenesulfonic acid monohydrate (91.2 mg, 0.48 mmol). The mixture was stirred at reflux for 12 h and cooled to precipitate solid. The solid was filtered, washed with ethanol and dried to give tert-butyl 6-(4-(4-fluorophenyl)-5-hydroxy-1H-pyrazol-1-yl)nicotinate (350.0 mg, 0.98 mmol, 41% yield) as white solid. LC-MS: m/z=356.0 (M+H)⁺, retention time 6.23 min (Method A). ¹HNMR (400 MHz, DMSO-d₆) (8.91 (s, 1H), 8.41 (s, 3H), 7.93 (s, 2H), 7.19 (s, 2H), 1.57 (s, 9H).

### Example 7: Preparation of Compound 7

### 6-(4-(4-Fluorophenyl)-5-hydroxy-1H-pyrazol-1-yl)nicotinic acid

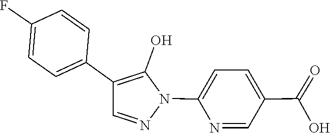 [SMILES: O=C(O)c1ccc(-n2ncc(-c3ccc(F)cc3)c2O)nc1]

To a solution of tert-butyl 6-(4-(4-fluorophenyl)-5-hydroxy-1H-pyrazol-1-yl)nicotinate (150.0 mg, 0.42 mmol) in dichloromethane (5.0 mL) was added trifluoroacetic acid (2.0 mL). The mixture was stirred at 40° C. for 2 h and concentrated. The residue was triturated with ethyl acetate and filtered to afford 6-(4-(4-fluorophenyl)-5-hydroxy-1H-pyrazol-1-yl) nicotinic acid (103.2 mg, 0.34 mmol, 81% yield) as white solid. LC-MS: m/z=300.0 (M+H)⁺, retention time 4.23 min (Method A). ¹HNMR (400 MHz, DMSO-d₆) (8.95 (s, 1H), 8.45 (s, 3H), 7.94 (s, 2H), 7.20-7.17 (m, 2H).

### Example 8: Preparation of Compound 8

### Ethyl 2-(4-cyano-2-methylphenyl) acetate

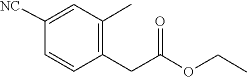

A mixture of 4-bromo-3-methylbenzonitrile (5.0 g, 25.6 mmol), diethyl malonate (27 g 168 mmol), tris(dibenzylideneacetone)dipalladium(0) (0.24 g, 0.26 mmol), tri-tert-butylphosphine tetrafluoroborate (0.08 g, 0.26 mmol), potassium carbonate (5.3 g, 38.4 mmol) and potassium hydrogen carbonate (3.84 g, 38.4 mmol) was stirred at 160° C. for 12 h. After cooling to room temperature, the reaction mixture was diluted with ethyl acetate and water. The organic layer was washed with brine, dried over sodium sulfate and concentrated. The residue was purified by flash chromatography (petroleum ether/ethyl acetate=1/1) to afford ethyl 2-(4-cyano-2-methylphenyl)acetate (2.0 g, 31.7% yield) as yellow oil. LC-MS: m/z 204.1 (M+H)⁺.

### Ethyl (E)-2-(4-cyano-2-methylphenyl)-3-(dimethylamino)acrylate

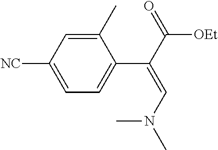 [SMILES: CCOC(=O)/C(=C/N(C)C)c1ccc(C#N)cc1C]

To a solution of ethyl 2-(4-cyano-2-methylphenyl)acetate (1.0 g, 5.0 mmol) in N,N-dimethylformamide (10.0 mL) was added N,N-dimethylformamide diethyl acetal (2.9 g, 25.0 mmol). The mixture was stirred at 100° C. overnight and cooled to room temperature. Ethyl acetate and water were added to the solution, and the layers were separated. The organic layer was washed with brine, dried over sodium sulfate and concentrated. The residue was purified by flash chromatography (dichloromethane/methanol=98/2) to give ethyl (E)-2-(4-cyano-2-methylphenyl)-3-(dimethylamino)acrylate (600 mg, 47.2% yield) as yellow oil. LC-MS: m/z 259.0 (M+H)⁺.

### Tert-butyl 6-chloronicotinate

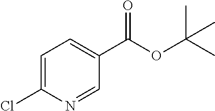

To a solution of 6-chloronicotinic acid (5.0 g, 6.37 mmol) and 4-dimethylaminopyridine (0.39 g, 0.64 mmol) in THF (50.0 mL) was added di-tert-butyl dicarbonate (10.41 g, 47.77 mmol). The reaction mixture was refluxed for 4 h and concentrated. The residue was purified by flash chromatography (petroleum ether/ethyl acetate=10/1) to afford tert-butyl 6-chloronicotinate (5.5 g, 81.12% yield) as yellow solid. LC-MS: m/z 214.0 (M+H)⁺.

### Tert-butyl 6-hydrazineylnicotinate

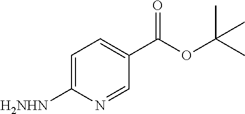

To a solution of tert-butyl 6-chloronicotinate (5.5 g, 25.82 mmol) in ethanol (25.0 mL) was added hydrazine hydrate (6.46 g, 129.11 mmol, 85% in water). The mixture was stirred at 100° C. for 2 h and concentrated. The residue was partitioned between ethyl acetate and water. The organic phase was washed with brine, dried over sodium sulfate and concentrated. The residue was triturated with petroleum ether and filtered to afford tert-butyl 6-hydrazineylnicotinate (5.0 g, 92.76% yield) as yellow solid. LC-MS: m/z 210.0 (M+H)⁺.

### Tert-butyl 6-(4-(4-cyano-2-methylphenyl)-5-hydroxy-1H-pyrazol-1-yl)nicotinate

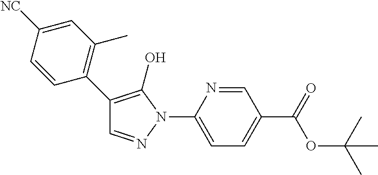 [SMILES: Cc1cc(C#N)ccc1-c1cnn(-c2ccc(C(=O)OC(C)(C)C)cn2)c1O]

To a solution of ethyl (E)-2-(4-cyano-2-methylphenyl)-3-(dimethylamino)acrylate (260 mg, 1.0 mmol) and tert-butyl 6-hydrazineylnicotinate (200 mg, 1.0 mmol) in ethanol (10.0 mL) was added 4-methylbenzenesulfonic acid (34.4 mg, 0.2 mmol). The mixture was stirred at reflux for 12 h and cooled to precipitate solid. The solid was filtered, washed with ethanol and dried to give tert-butyl 6-(4-(4-cyano-2-methylphenyl)-5-hydroxy-1H-pyrazol-1-yl)nicotinate (190 mg, 50% yield) as white solid. LC-MS: m/z 377.0 (M+H)⁺.

### Example 9: Preparation of Compound 9

### Ethyl-3-acetoxy-2-(4-cyanophenyl)but-2-enoate

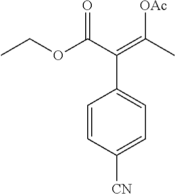 [SMILES: CCOC(=O)/C(=C(/C)OC(C)=O)c1ccc(C#N)cc1]

Under N₂ atmosphere, to a mixture of ethyl 2-(4-cyanophenyl)acetate (3.50 g, 18.50 mmol) in anhydrous THF (100 ml) at −30° C. was added LHMDS (28.00 mL, 27.75 mmol) dropwise over 20 min. The reaction was allowed to warm to 0° C. and stirred at 0° C. for 30 min. The reaction was then cooled to −30° C. again and to the solution was added a solution of acetyl chloride (2.18 g, 27.75 mmol) in THF (8 mL) dropwise over 15 min. The reaction was warmed to rt slowly and stirred for 4 hrs. After the reaction was completed by TLC, the mixture was quenched with a cold aqueous NH₄Cl solution (50 mL) and extracted with EtOAc (3×80 ml). The combined organic phases were dried with anhydrous Na₂SO₄ (30 g), filtered and concentrated in vacuo. The residue was purified by silica column chromatography (EA:Hex=1:50 to 1:5) to give the desired product (1.9 g) as yellow oil. ¹H NMR 6 ppm (300 MHz, CDCl₃) 7.68 (dd, J=6.6, 1.8 Hz, 2H), 7.41 (dd, J=6.6, 1.8 Hz, 2H), 4.11-4.18 (q, J=6.9 Hz, 2H), 2.25 (s, 3H), 1.88 (s, 3H), 1.20 (t, J=6.9 Hz, 3H).

### 6-(4-(4-Cyanophenyl)-5-hydroxy-3-methyl-1H-pyrazol-1-yl)nicotinic acid

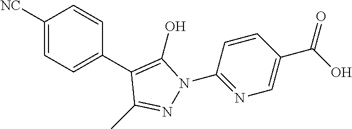 [SMILES: Cc1nn(-c2ccc(C(=O)O)cn2)c(O)c1-c1ccc(C#N)cc1]

To a mixture of ethyl 2-(4-cyanophenyl)-3-oxobutanoate (0.30 g, 1.38 mmol) in HOAc (24 ml) was added 4-hydrazinylbenzoic acid (1.06 g, 6.91 mmol). The mixture was stirred at 100° C. overnight. After the reaction was completed by TLC, the reaction was quenched water (20 mL) and a large amount of solid was precipitated. After filtration, the solid was dissolved in TEA (5 eq), water (20 mL) and MeOH (20 mL). The solution was extracted with EtOAc (3×10 mL) and the aqueous phase was adjusted pH to 3 with a diluted HCl solution. A large amount of solid was precipitated. The solid was collected by filtration and dried to give the desired product (128 mg) as yellow solid. LC-MS (ESI+): m/z 321 (M+H)⁺; HPLC purity was 97.3%; ¹H NMR (300 MHz, DMSO-d₆) δ ppm 13.25 (brs, 2H), 8.95 (d, J=1.5 Hz, 1H), 8.56 (d, J=8.7 Hz, 1H), 8.41 (dd, J=8.7 Hz, 1H), 7.89 (d, J=8.1 Hz, 2H), 7.81 (d, J=8.1 Hz, 2H), 2.51 (s, 3H).

### Example 10: Preparation of Compound 10

### Tert-butyl 6-hydrazinylnicotinate

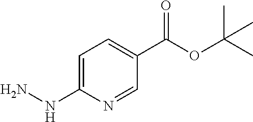

To a solution of tert-butyl 6-chloronicotinate (0.20 g, 0.94 mmol) in 1,4-dioxane (10 mL) was added hydrazine (0.35 g, 5.62 mmol) in one portion. The mixture was stirred at 80° C. overnight. After the reaction was completed by TLC analysis, the mixture was concentrated in vacuo. The residue was purified by preparative HPLC to give 180 mg of the title compound (180 mg) as oil. ¹H NMR (300 MHz, CDCl₃) δ ppm 8.71 (d, J=1.8 Hz, 1H), 8.01 (dd, J=8.1, 1.8 Hz, 1H), 6.70 (d, J=8.7 Hz, 1H), 6.24 (brs, 1H), 3.90 (brs, 2H), 1.60 (s, 9H).

### Tert-butyl 6-(4-(4-cyanophenyl)-5-hydroxy-3-methyl-1H-pyrazol-1-yl)nicotinate

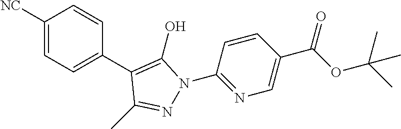 [SMILES: Cc1nn(-c2ccc(C(=O)OC(C)(C)C)cn2)c(O)c1-c1ccc(C#N)cc1]

The compound was synthesized according to the procedure for the preparation of 6-(4-(4-cyanophenyl)-5-hydroxy-3-methyl-1H-pyrazol-1-yl)nicotinic acid using tert-butyl 6-hydrazineylnicotinate. LC-MS (ESI−): m/z 375 (M−H)⁻; HPLC purity was 96.3%; ¹H NMR (300 MHz, CDCl₃) δ ppm 13.10 (brs, 1H), 8.88 (d, J=2.1 Hz, 1H), 8.42 (dd, J=8.7, 2.1 Hz, 1H), 7.96 (d, J=8.7 Hz, 1H), 7.69 (s, 4H), 2.46 (s, 3H), 1.63 (s, 9H).

### Example 1: Preparation of Compound 11

Methyl 6-(4-(4-cyanophenyl)-5-hydroxy-3-methyl-1H-pyrazol-1-yl)nicotinate

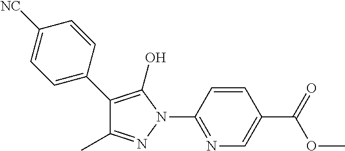 [SMILES: COC(=O)c1ccc(-n2nc(C)c(-c3ccc(C#N)cc3)c2O)nc1]

The compound was synthesized according to the procedure for the preparation of 6-(4-(4-cyanophenyl)-5-hydroxy-3-methyl-1H-pyrazol-1-yl)nicotinic acid using methyl 6-hydrazineylnicotinate. LC-MS: m/z=335 (M+H)⁺; 1H NMR (300 MHz, DMSO-d6) δ ppm 13.11 (s, 1H), 8.95 (d, J=1.6 Hz, 1H), 8.58 (d, J=8.8 Hz, 1H), 8.43 (dd, J=7.0, 2.3 Hz, 1H), 7.90 (dd, J=17.6, 8.3 Hz, 2H), 7.79 (dd, J=14.5, 8.4 Hz, 2H), 3.90 (s, 3H), 2.48 (s, 3H).

### Example 12: Preparation of Compound 12

### Di-tert-butyl 2-(4-cyano-2,5-difluorophenyl)malonate

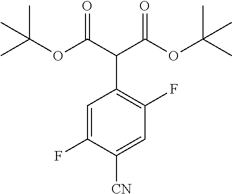 [SMILES: CC(C)(C)OC(=O)C(C(=O)OC(C)(C)C)c1cc(F)c(C#N)cc1F]

Under nitrogen atmosphere, to a solution of sodium hydride (60% dispersion in mineral oil) (5.09 g, 127.31 mmol) in anhydrous DMF (100 mL) at 0° C. was added Di-tert-butyl malonate (8.26 g, 31.19 mmol) dropwise over 15 min. After the reaction was stirred at 0° C. for 15 min, 2,4,5-Trifluorobenzonitrile (5.00 g, 31.83 mmol) was added to the reaction in one portion. The mixture was stirred at 0° C. for 15 min, followed by stirring at 60° C. overnight. After the reaction was completed by TLC analysis, the mixture was quenched with an aqueous saturated NH₄Cl solution (800 mL) and extracted with EtOAc (200 mL×3). The combined organic phase was washed with water (100 mL×2), dried with anhydrous Na₂SO₄ (50 g), filtered and concentrated in vacuo. 12.60 g of the crude title compound as yellow oil was obtained, which was used in the next step without further purification. LC-MS (ESI+): m/z 239 (M-(t-Bu)₂)⁺.

### 2-(4-Cyano-2,5-difluorophenyl)acetic acid

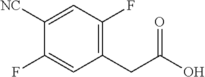 [SMILES: N#Cc1cc(F)c(CC(=O)O)cc1F]

To a solution of di-tert-butyl 2-(4-cyano-2,5-difluorophenyl)malonate (12.30 g, 34.80 mmol) in DCM (35 mL) was added TFA (35 mL) in one portion. The reaction was stirred at rt overnight. After the reaction was completed by TLC analysis, the mixture was concentrated in vacuo. The residue was added toluene (50 mL), stirred and concentrated to dryness again. 6.07 g of the crude title compound was obtained as white solid, which was used for the next step without further purification. LC-MS (ESI+): m/z 216 (M+H+H₂O)⁺.

### Methyl 2-(4-cyano-2,5-difluorophenyl)acetate

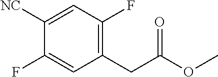

To a solution of 2-(4-cyano-2,5-difluorophenyl)acetic acid (4.00 g, 20.29 mmol) in MeOH (160 mL) at rt was added SOCl₂ (4.0 mL) over 5 min. The reaction was stirred at 70° C. for 3 hrs. After the reaction was completed by TLC analysis, 80% of the reaction solvent was removed by evaporation. The residue was quenched by ice water (180 mL) and extracted with EtOAc (50 mL×3). The combined organic phase was dried with anhydrous Na₂SO₄ (50 g), filtered and concentrated in vacuo. 4.30 g of the crude title compound was obtained as yellow oil, which was used for the next step without further purification. LC-MS (ESI+): m/z 230 (M+H+H₂O)⁺.

### Methyl (E)-2-(4-cyano-2,5-difluorophenyl)-3-(dimethylamino)acrylate

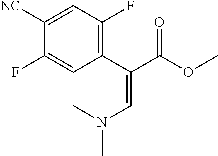 [SMILES: COC(=O)/C(=C/N(C)C)c1cc(F)c(C#N)cc1F]

The compound was synthesized according to the procedure for the preparation of ethyl (E)-2-(4-cyano-2-methylphenyl)-3-(dimethylamino)acrylate using methyl 2-(4-cyano-2,5-difluorophenyl)acetate. LC-MS (ESI+): m/z 267 (M+H)⁺.

### 6-(4-(4-Cyano-2,5-difluorophenyl)-5-hydroxy-1H-pyrazol-1-yl)nicotinic acid

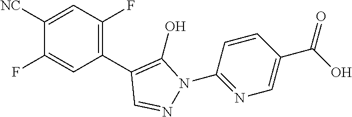 [SMILES: N#Cc1cc(F)c(-c2cnn(-c3ccc(C(=O)O)cn3)c2O)cc1F]

The compound was synthesized according to the procedure for the preparation of 6-(4-(4-Cyano-2-methylphenyl)-5-hydroxy-1H-pyrazol-1-yl)nicotinic acid using methyl (E)-2-(4-cyano-2,5-difluorophenyl)-3-(dimethylamino)acrylate. LC-MS (ESI−): m/z 341 (M−H)⁻, HPLC purity was 95.4%, ¹H NMR (300 MHz, DMSO-d₆) (ppm 13.41 (brs, 1H), 8.96 (d, J=2.1 Hz, 1H), 8.58-8.42 (m, 3H), 8.31 (d, J=3.0 Hz, 1H), 7.93 (dd, J=10.8, 5.4 Hz, 1H).

### Example 13: Preparation of Compound 13

### Methyl 2-(3-fluoro-4-hydroxyphenyl)acetate

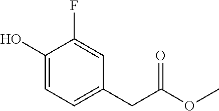

To a mixture of 2-(3-fluoro-4-hydroxyphenyl)acetic acid (2.0 g, 11.76 mmol) in MeOH (20 ml) was added conc. H₂SO₄ (0.2 mL). The reaction was stirred at 65° C. for 2 hrs. After the reaction was completed by TLC, the mixture was quenched with water (40 mL) and extracted with EtOAc (3×50 mL). The combined organic phase was dried, filtered and concentrated directly to give the title product (2.49 g) as oil. ¹H NMR (300 MHz, CDCl₃) δ ppm 7.03 (d, J=1.5 Hz, 1H), 6.91 (dd, J=3.9, 1.5 Hz, 2H), 3.70 (s, 3H), 3.54 (s, 2H).

### Methyl 2-(3-fluoro-4-(((trifluoromethyl)sulfonyl)oxy)phenyl)acetate

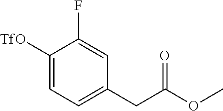 [SMILES: COC(=O)Cc1ccc(OS(=O)(=O)C(F)(F)F)c(F)c1]

Under N₂ atmosphere, to a mixture of methyl 2-(3-fluoro-4-hydroxyphenyl)acetate (2.49 g, 13.50 mmol) in DCM (52 mL) at 0° C. was added trifluoromethanesulfonic anhydride (5.71 g, 20.25 mmol) dropwise over 15 mins. TEA (4.10 g, 40.50 mmol) was added dropwise to the reaction over 10 min. The reaction was stirred at 0° C. for 4 hrs. After the reaction was completed by TLC, the mixture was quenched with an aqueous NaHCO₃ solution (30 mL) and extracted with DCM (3×50 mL). The combined organic phase was dried with anhydrous Na₂SO₄ (50 g), filtered and concentrated in vacuo. The residue was purified by silica column chromatography (EA:n-Hex=1:20) to give the desired product (2.18 g) as a yellow oil. ¹H NMR (300 MHz, CDCl₃) δ ppm 7.30 (d, J=8.1 Hz, 1H), 7.24 (dd, J=3.3, 1.11 Hz, 1H), 7.13 (d, J=8.1 Hz, 1H), 3.73 (s, 3H), 3.65 (s, 2H).

### Methyl 2-(4-cyano-3-fluorophenyl)acetate

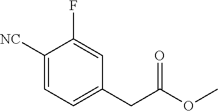

Under N₂ atmosphere, to a mixture of methyl 2-(3-fluoro-4-(((trifluoromethyl)sulfonyl)oxy)phenyl) acetate (2.18 g, 6.90 mmol) in anhydrous DMF (40 mL) was added Zn(CN)₂ (0.49 g, 4.14 mmol) and Pd(PPh₃)₄ (0.80 g, 0.69 mmol). The reaction was stirred at 80° C. for 4 hrs. After the reaction was completed by TLC, the mixture was quenched with water (50 mL) and extracted with EtOAc (3×50 mL). The combined organic phase was washed with water (2×20 mL), dried with anhydrous Na₂SO₄ (50 g), filtered and concentrated in vacuo. The residue was purified by silica column chromatography (EA:Hex=1:40 to 1:8) to give the desired product (1.27 g) as yellow oil. ¹H NMR (300 MHz, CDCl₃) δ ppm 7.59 (t, J=7.5 Hz, 1H), 7.20 (d, J=8.7 Hz, 2H), 3.73 (s, 3H), 3.70 (s, 2H).

### Methyl (E)-2-(4-cyano-3-fluorophenyl)-3-(dimethylamino)acrylate

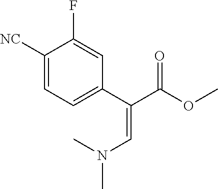 [SMILES: COC(=O)/C(=C/N(C)C)c1ccc(C#N)c(F)c1]

The compound was synthesized according to the procedure for the preparation of ethyl (E)-2-(4-cyano-2-methylphenyl)-3-(dimethylamino)acrylate using methyl 2-(4-cyano-3-fluorophenyl)acetate. LC-MS (ESI+): m/z 249 (M+H)⁺.

### 6-(4-(4-cyano-3-fluorophenyl)-5-hydroxy-1H-pyrazol-1-yl)nicotinic acid

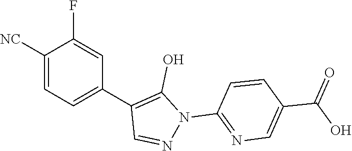 [SMILES: N#Cc1ccc(-c2cnn(-c3ccc(C(=O)O)cn3)c2O)cc1F]

The compound was synthesized according to the procedure for the preparation of 6-(4-(4-Cyano-2-methylphenyl)-5-hydroxy-1H-pyrazol-1-yl)nicotinic acid using methyl (E)-2-(4-cyano-3-fluorophenyl)-3-(dimethylamino)acrylate. LC-MS (ESI−): m/z 323 (M−H)⁻; ¹H-NMR (300 MHz, DMSO-d₆) δ ppm 13.32 (brs, 1H), 8.77 (d, J=1.2 Hz, 1H), 8.55 (s, 1H), 8.34 (d, J=8.4 Hz, 1H), 8.26 (dd, J=8.4, 2.1 Hz, 1H), 7.88 (d, J=12.3 Hz, 1H), 7.78 (d, J=8.1 Hz, 1H), 7.65 (t, J=7.5 Hz, 1H).

### Example 14: Preparation of Compound 14

### 5-Fluoro-6-hydrazinylnicotinic Acid

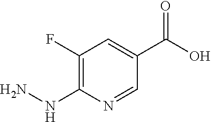 [SMILES: NNc1ncc(C(=O)O)cc1F]

To a solution of 6-chloro-5-fluoronicotinic acid (0.46 g, 2.59 mmol) in THF (22 mL) was added N₂H₄·H₂O (0.81 g, 12.95 mmol) dropwise over 1 min. After the mixture was stirred at 65° C. overnight, a large amount of solid was precipitated. The suspension was filtrated and the solid was slurried in MeOH (3 mL) for 1 hr. After filtration, 440 mg of the desired product was obtained as white solid. LC-MS (ESI+): m/z 172 (M+H)⁺.

### 6-(4-(4-cyanophenyl)-5-hydroxy-1H-pyrazol-1-yl)-5-fluoronicotinic acid

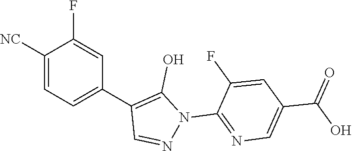 [SMILES: N#Cc1ccc(-c2cnn(-c3ncc(C(=O)O)cc3F)c2O)cc1F]

The compound was synthesized according to the procedure for the preparation of 6-(4-(4-Cyano-2-methylphenyl)-5-hydroxy-1H-pyrazol-1-yl)nicotinic acid using 5-fluoro-6-hydrazinylnicotinic acid and methyl (E)-2-(4-cyanophenyl)-3-(dimethylamino)acrylate. LC-MS (ESI+): m/z 325 (M+H)⁺; ¹H-NMR (300 MHz, DMSO-d₆) δ ppm 8.78 (s, 1H), 8.08 (d, J=9.9 Hz, 1H), 7.90 (d, J=8.4 Hz, 2H), 7.84 (s, 1H), 7.46 (d, J=8.4 Hz, 2H), 7.12 (brs, 2H).

### Example 15: Preparation of Compound 15

### Methyl 2-(4-chlorophenyl)-3-oxobutanoate

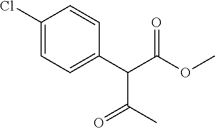

Under the nitrogen atmosphere, to a solution of methyl 2-(4-chlorophenyl)acetate (1.00 g, 5.41 mmol) in anhydrous THF (50 mL) at −40° C. was added LHMDS (8.1 mL, 8.11 mmol) dropwise over 10 min. After the resulting mixture was stirred at −40° C. for 1 hr, 1-(1H-imidazol-1-yl)ethan-1-one (0.89 g, 8.11 mmol) was added portion wise over 10 min. After addition, the mixture was warmed to rt and stirred for 1 hrs. After the reaction was completed as indicated by TLC analysis, the residue was diluted with water (50 mL) and extracted with ethyl acetate (50 mL×3). The combined organic phase was dried with Na₂SO₄ (30 g), filtered, and concentrated to dryness to afford the desired product (1.6 g) as oil. LC-MS (ESI+): m/z 249 (M+Na)⁺.

### 6-(4-(4-chlorophenyl)-5-hydroxy-1H-pyrazol-1-yl)nicotinic acid

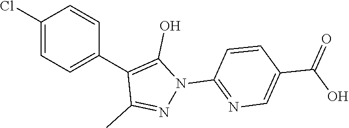 [SMILES: Cc1nn(-c2ccc(C(=O)O)cn2)c(O)c1-c1ccc(Cl)cc1]

The compound was synthesized according to the procedure for the preparation of 6-(4-(4-cyanophenyl)-5-hydroxy-3-methyl-1H-pyrazol-1-yl)nicotinic acid using methyl 2-(4-chlorophenyl)-3-oxobutanoate. LC-MS (ESI+): m/z 330 (M+H)⁺; ¹H-NMR (300 MHz, DMSO-d₆) δ ppm 12.77-13.27 (m, 2H), 8.95 (d, J=1.5 Hz, 1H), 8.55 (brs, 1H), 8.41 (dd, J=9.0 Hz, J=2.1 Hz, 1H), 7.66 (d, J=8.4 Hz, 2H), 7.44 (d, J=8.4 Hz, 2H), 2.42 (s, 3H).

### Example 16: Preparation of Compound 16

### methyl 2-(4-(benzyloxy)-3-methylphenyl)acetate

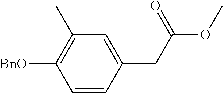 [SMILES: COC(=O)Cc1ccc(OCc2ccccc2)c(C)c1]

To a solution of methyl 2-(4-hydroxy-3-methylphenyl)acetate (300 mg, 1.70 mmol) in DMF (10 ml) was added benzyl bromide (292 mg, 1.70 mmol) and Cs₂CO₃ (1.66 g, 5.10 mmol). The mixture was stirred at rt for 1 hr. After the reaction was completed by TLC analysis, the mixture was quenched with water (50 mL) and extracted with EtOAc (50 mL×3). The combined organic phases were dried with anhydrous Na₂SO₄ (30 g), filtered and concentrated in vacuo. The residue was purified by silica column chromatography (PE:EtOAc=100:1˜50:1) to give 498 mg of the title compound. ¹H NMR (300 MHz, CDCl₃) δ ppm 7.29-7.45 (m, 5H), 7.03-7.08 (m, 2H), 6.82 (d, J=8.1 Hz, 1H), 5.06 (s, 2H), 3.68 (s, 3H), 3.54 (s, 2H), 2.27 (s, 3H).

### methyl (Z)-2-(4-(benzyloxy)-3-methylphenyl)-3-(dimethylamino)acrylate

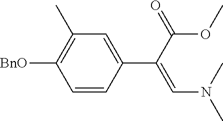 [SMILES: COC(=O)/C(=C\N(C)C)c1ccc(OCc2ccccc2)c(C)c1]

The compound was synthesized according to the procedure for the preparation of ethyl (E)-2-(4-cyano-2-methylphenyl)-3-(dimethylamino)acrylate using methyl 2-(4-(benzyloxy)-3-methylphenyl)acetate. LC-MS (ESI+): m/z 326 (M+H⁺).

### 6-(4-(4-(benzyloxy)-3-methylphenyl)-5-hydroxy-1H-pyrazol-1-yl)nicotinic acid

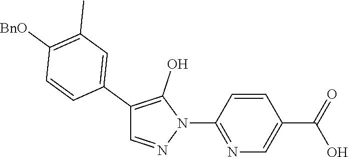 [SMILES: Cc1cc(-c2cnn(-c3ccc(C(=O)O)cn3)c2O)ccc1OCc1ccccc1]

The compound was synthesized according to the procedure for the preparation of 6-(4-(4-Cyano-2-methylphenyl)-5-hydroxy-1H-pyrazol-1-yl)nicotinic acid using methyl (Z)-2-(4-(benzyloxy)-3-methylphenyl)-3-(dimethylamino)acrylate. LC-MS (ESI+): m/z 402 (M+H⁺).

### 6-(5-hydroxy-4-(4-hydroxy-3-methylphenyl)-1H-pyrazol-1-yl)nicotinic acid

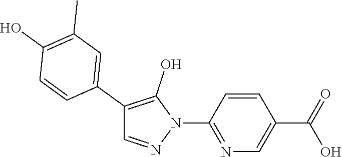 [SMILES: Cc1cc(-c2cnn(-c3ccc(C(=O)O)cn3)c2O)ccc1O]

A solution of 6-(4-(4-(benzyloxy)-3-methylphenyl)-5-hydroxy-1H-pyrazol-1-yl)nicotinic acid (60 mg, 0.12 mmol) and Pd/C (10 mg) in MeOH (6.5 mL) was stirred under hydrogen atmosphere from a balloon for 7 hrs. After the reaction was completed as indicated by TLC analysis, the suspension was filtered through a package of Celite and the filtered cake was washed with MeOH (5 mL). The combined filtrate was concentrated and purified with preparative HPLC to give 9 mg of the title compound as solid. LC-MS (ESI−): m/z 310 (M−H⁻), ¹H NMR (300 MHz, CD₃OD) δ ppm 8.94 (s, 1H), 8.45 (d, J=8.7 Hz, 1H), 8.11 (d, J=8.7 Hz, 1H), 7.90 (s, 1H), 7.48 (s, 1H), 7.41 (d, J=8.7 Hz, 1H), 6.75 (s, 1H), 6.65 (d, J=6.9 Hz, 1H), 2.22 (s, 3H).

### Example 17: Preparation of Compound 17

ethyl (Z)-2-(6-chloropyridin-3-yl)-3-(dimethylamino)acrylate

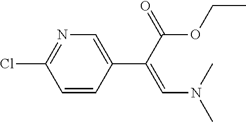 [SMILES: CCOC(=O)/C(=C\N(C)C)c1ccc(Cl)nc1]

The compound was synthesized according to the procedure for the preparation of ethyl (E)-2-(4-cyano-2-methylphenyl)-3-(dimethylamino)acrylate using ethyl 2-(6-chloropyridin-3-yl)acetate. LC-MS (ESI+): m/z 241 (M+H⁺), ¹H NMR (300 MHz, CDCl₃) δ ppm 8.18 (d, J=2.4 Hz, 1H), 7.66 (s, 1H), 7.51 (dd, J=2.4, 8.1 Hz, 1H), 7.26 (d, J=8.4 Hz, 1H), 3.64 (s, 3H), 2.75 (s, 6H).

### 6-(4-(6-chloropyridin-3-yl)-5-hydroxy-1H-pyrazol-1-yl)nicotinic acid

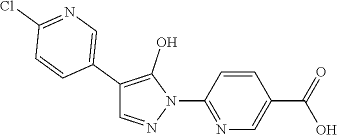 [SMILES: O=C(O)c1ccc(-n2ncc(-c3ccc(Cl)nc3)c2O)nc1]

The compound was synthesized according to the procedure for the preparation of 6-(4-(4-Cyano-2-methylphenyl)-5-hydroxy-1H-pyrazol-1-yl)nicotinic acid using ethyl (Z)-2-(6-chloropyridin-3-yl)-3-(dimethylamino)acrylate. LC-MS (ESI+): m/z 317 (M+H⁺), ¹H NMR (300 MHz, DMSO-d₆) δ13.44 (brs, 2H), 8.94 (d, J=16.8 Hz, 2H), 8.67 (s, 1H), 8.55 (s, 1H), 8.44-8.48 (m, 1H), 8.38 (d, J=7.8 Hz, 1H), 7.51 (d, J=8.4 Hz, 1H).

### Example 18: Preparation of Compound 18

### 6-hydrazinyl-4-methylnicotinic acid

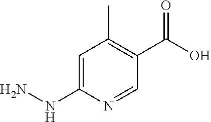

To a solution of 6-fluoro-4-methylnicotinic acid (0.50 g, 3.23 mmol) in THF (50 ml) was added hydrazine (2.00 g, 32.26 mmol). The reaction was stirred at 66° C. for 2 hrs. After the reaction was completed as indicated by LC-MS, the mixture was diluted with ethanol (6 mL) and a large amount of solid was precipitated. After filtration, 725 mg of the desired product was obtained. LC-MS (ESI+): m/z 168 (M+H⁺), ¹H NMR (300 MHz, DMSO-d₆) δ 8.44 (s, 1H), 7.56 (brs, 1H), 6.43 (s, 1H), 5.90 (brs, 2H), 2.44 (s, 3H)

### 6-(4-(4-cyanophenyl)-5-hydroxy-3-methyl-1H-pyrazol-1-yl)-4-methylnicotinic acid

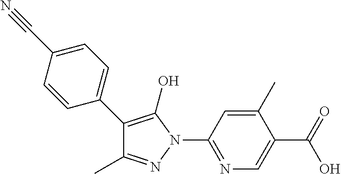 [SMILES: Cc1cc(-n2nc(C)c(-c3ccc(C#N)cc3)c2O)ncc1C(=O)O]

To a solution of 6-hydrazinyl-4-methylnicotinic acid (0.30 g, 1.80 mmol) in acetic acid (20 ml) was added ethyl 2-(4-cyanophenyl)-3-oxobutanoate (0.43 g, 1.98 mmol). The reaction was stirred at 100° C. overnight. After the reaction was completed as indicated by LC-MS analysis, the mixture was cooled to rt and a large amount of solid was precipitated. After filtration, the filter cake was further purified by preparative HPLC to give 36 mg of the desired product was obtained. LC-MS (ESI+): m/z 335 (M+H⁺), ¹H-NMR (300 MHz, DMSO-d₆) δ12.97-13.21 (brs, 2H), 8.87 (s, 1H), 8.45 (brs, 1H), 8.01 (d, J=8.1 Hz, 2H), 7.82 (d, J=8.1 Hz, 2H), 2.78 (s, 3H), 2.52 (s, 3H).

### Example 19: Preparation of Compound 19

### 6-hydrazineyl-2-methylnicotinic acid

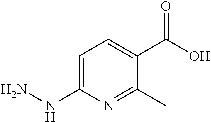

The compound was synthesized according to the procedure for the preparation of 6-hydrazinyl-4-methylnicotinic acid using 6-fluoro-2-methylnicotinic acid. ¹H-NMR (300 MHz, D₂O) δ 7.63 (d, J=8.7 Hz, 1H), 6.55 (d, J=8.7 Hz, 1H), 2.37 (s, 3H).

### 6-(4-(4-cyanophenyl)-5-hydroxy-3-methyl-1H-pyrazol-1-yl)-2-methylnicotinic acid

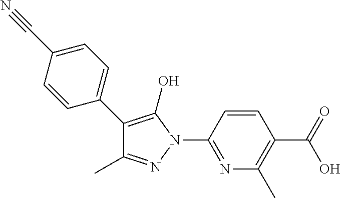 [SMILES: Cc1nc(-n2nc(C)c(-c3ccc(C#N)cc3)c2O)ccc1C(=O)O]

The compound was synthesized according to the procedure for the preparation of 6-(4-(4-cyanophenyl)-5-hydroxy-3-methyl-1H-pyrazol-1-yl)-4-methylnicotinic acid using 6-hydrazineyl-2-methylnicotinic acid. LC-MS (ESI+): m/z 335 (M+H)⁺; ¹H-NMR (300 MHz, DMSO-d₆) δ12.95 (brs, 1H), 8.28-8.35 (m, 2H), 7.89 (d, J=8.7 Hz, 2H), 7.79 (d, J=8.7 Hz, 2H), 2.80 (s, 3H), 2.58 (s, 3H).

### Example 20: Preparation of Compound 20

### ethyl 2-(5-chloropyridin-2-yl)-3-oxobutanoate

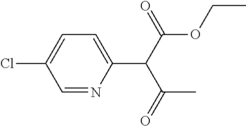 [SMILES: CCOC(=O)C(C(C)=O)c1ccc(Cl)cn1]

Under N₂ atmosphere, to a mixture of ethyl 2-(5-chloropyridin-2-yl)acetate (500 mg, 2.5 mmol) in anhydrous THF (15 mL) at −55° C. was added LHMDS (5.0 mL, 5.0 mmol) dropwise over 20 min. After the reaction was stirred at 0° C. for 1 hr, a solution of 1-(1H-imidazol-1-yl)ethan-1-one (412 mg, 3.75 mmol) in THF (15 mL) at −55° C. was added dropwise over 15 min. The reaction was warmed to rt slowly and stirred at rt for 1 hr. After the reaction was completed as indicated by TLC analysis, the mixture was quenched with an aqueous NH₄Cl solution (50 mL) and extracted with EtOAc (3×20 mL). The combined organic phase was dried with anhydrous Na₂SO₄ (20 g), filtered and concentrated in vacuo. The residue was purified by silica column chromatography (EtOAc:Hex=1:100) to give the title compound (152 mg) as solid. LC-MS (ESI+): m/z 242 (M+H)⁺.

### 6-(4-(5-chloropyridin-2-yl)-5-hydroxy-3-methyl-1H-pyrazol-1-yl)nicotinic acid

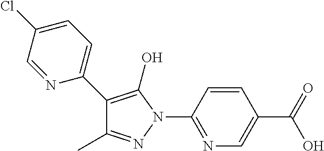 [SMILES: Cc1nn(-c2ccc(C(=O)O)cn2)c(O)c1-c1ccc(Cl)cn1]

The compound was synthesized according to the procedure for the preparation of 6-(4-(4-cyanophenyl)-5-hydroxy-3-methyl-1H-pyrazol-1-yl)nicotinic acid using ethyl 2-(5-chloropyridin-2-yl)-3-oxobutanoate. LC-MS (ESI+): m/z 331 (M+H)⁺; ¹H NMR (300 MHz, DMSO-d₆)(513.20 (brs, 2H), 8.95 (d, J=1.8 Hz, 1H), 8.53-8.61 (m, 1H), 8.52 (s, 1H), 8.41 (dd, J=8.7 Hz, J=2.1 Hz, 1H), 8.32 (d, J=5.1 Hz, 1H), 7.87 (dd, J=8.7 Hz, J=2.7 Hz, 1H), 2.62 (s, 3H).

### Example 21: Preparation of Compound 21

### methyl 2-(4-bromo-2-methoxyphenyl)acetate

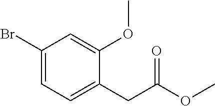

To a solution of 2-(4-bromo-2-methoxyphenyl)acetic acid (2.00 g, 8.16 mmol) in MeOH (35 ml) at rt was added in SOCl₂ (6 mL, 82.71 mmol) dropwise over 5 min. After addition, the mixture was stirred at 55° C. overnight. After the reaction was completed as indicated by TLC analysis, the reaction mixture was concentrated to dryness directly. The residue was diluted with an aqueous NaHCO₃ solution (50 mL) and extracted with EtOAc (100 mL×3). The combined organic phase was dried with Na₂SO₄, filtered and concentrated to get the desired product (2.05 g) as yellow oil. LC-MS (ESI+): m/z 281 (M+Na)⁺; ¹H-NMR (300 MHz, CDCl₃) δ 7.02-7.08 (m, 2H), 7.00 (s, 1H), 3.93 (s, 3H), 3.81 (s, 3H), 3.57 (s, 2H).

### methyl 2-(4-cyano-2-methoxyphenyl)acetate

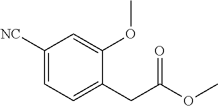

Under nitrogen protection, to a solution of methyl 2-(4-bromo-2-methoxyphenyl)acetate (3.02 g, 11.66 mmol) in DMF (20 ml) was added in Zn(CN)₂ (2.74 g, 23.32 mmol), Pd₂(dba)₃ (0.11 g, 0.12 mmol) and S-Phos (0.48 g, 1.17 mmol). The reaction was stirred at 110° C. for 4 hrs. After the reaction was completed as indicated by TLC analysis, the reaction was filtered to remove the un-dissolved solid. The filtrate was concentrated to dryness directly. The residue was purified by flash silica chromatography (EtOAc/PE=1/50 to 1/10) to give the desired product (2.04 g) as white solid. LC-MS (ESI+): m/z 206 (M+H)⁺; ¹H-NMR (300 MHz, CDCl₃) δ 7.23-7.30 (m, 3H), 3.86 (s, 3H), 3.71 (s, 3H), 3.67 (s, 2H).

### methyl (Z)-2-(4-cyano-2-methoxyphenyl)-3-(dimethylamino)acrylate

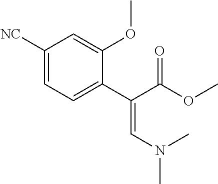 [SMILES: COC(=O)/C(=C\N(C)C)c1ccc(C#N)cc1OC]

A solution of methyl 2-(4-cyano-2-methoxyphenyl)acetate (0.94 g, 4.57 mmol) in DMF-DMA (27.4 g) was stirred at 100° C. overnight. After the reaction was completed as indicated by TLC analysis, the mixture was diluted with ice water (80 mL) and extracted with EtOAc (20 mL×3). The combined organic phase was dried with Na₂SO₄, filtered and concentrated to give the desired product (1.23 g) as brown oil. LC-MS (ESI+): m/z 261 (M+H)⁺;

### 6-(4-(4-cyano-2-methoxyphenyl)-5-hydroxy-1H-pyrazol-1-yl)nicotinic acid

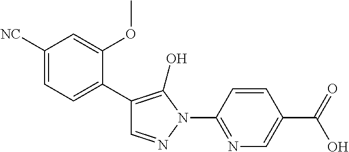 [SMILES: COc1cc(C#N)ccc1-c1cnn(-c2ccc(C(=O)O)cn2)c1O]

To a suspension of methyl (Z)-2-(4-cyano-2-methoxyphenyl)-3-(dimethylamino)acrylate (0.32 g, 1.24 mmol) in i-PrOH (6 mL) was added in 6-hydrazinylnicotinic acid (0.19 g, 1.24 mmol) and HCl (1.25 mL, 1M, 1.24 mmol). After the reaction was stirred at rt for 6 hr, a large amount of solid was precipitated. The suspension was filtrated. The filter cake was dissolved in i-PrOH (6 mL), water (1 mL) and DIEA (320 mg). The resulting mixture was stirred at 50° C. overnight. A diluted HCl solution (1M, 5 mL) was added to the reaction and the reaction was continued to stir at rt for additional 20 min. A large amount of solid was precipitated. After filtration, the filter cake was slurried in methanol (5 mL) overnight. After filtration and drying, 185 mg of the title compound was obtained. LC-MS (ESI+): m/z 335 (M−H)⁻; ¹H-NMR (300 MHz, DMSO-d₆) δ13.5 (brs, 1H), 8.97 (s, 1H), 8.56-8.68 (brs, 2H), 8.45-8.47 (m, 2H), 7.44-7.49 (m, 2H), 3.96 (s, 3H).

### 6-(4-(4-cyano-2-hydroxyphenyl)-5-hydroxy-1H-pyrazol-1-yl)nicotinic acid

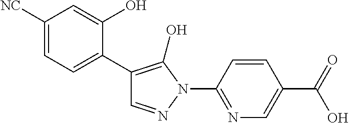 [SMILES: N#Cc1ccc(-c2cnn(-c3ccc(C(=O)O)cn3)c2O)c(O)c1]

Under nitrogen protection, a solution of 6-(4-(4-cyano-2-methoxyphenyl)-5-hydroxy-1H-pyrazol-1-yl)nicotinic acid (0.15 g, 0.45 mmol) in DMF (8 mL) was added in EtSNa (0.11 g, 1.34 mmol) in one portion. The reaction was stirred at 150° C. for 48 hrs. After the reaction was completed as indicated by TLC analysis, the mixture was diluted with water (80 mL) and adjusted pH to 2 with a diluted HCl solution. A large amount of solid was precipitated. After filtration, the solid was purified by preparative HPLC purification to give 20 mg of the title compound as yellow solid. LC-MS (ESI+): m/z 321 (M−H)⁻; ¹H-NMR (300 MHz, CD₃OD) δ 9.10 (s, 1H), 8.30-8.50 (m, 2H), 8.20 (m, 1H), 7.82 (m, 1H), 7.12 (m, 1H), 7.02 (s, 1H)

### Example 22: Preparation of Compound 22

### 1-chloro-2-methoxy-4-methylbenzene

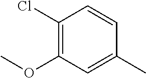

To a solution of 2-chloro-5-methylphenol (1 g, 6.99 mmol) in DMF (4 mL) was added K₂CO₃ (2.4 g, 17.48 mmol) and Mel (1.04 g, 7.34 mmol). The reaction was stirred at rt for about 6 hr. After the reaction was completed as indicated by TLC analysis, the reaction was diluted with water (20 mL) and extracted with EtOAc (20 mL×3). The combined organic phase was washed with water (10 mL), dried with Na₂SO₄ and concentrated to dryness to give 1.05 g of the crude desired product. GC-MS (EI+): 156; ¹H-NMR (300 MHz, CDCl₃) δ 7.22 (d, J=8.1 Hz, 1H), 6.69-6.74 (m, 2H), 3.88 (s, 3H), 2.33 (s, 3H).

### 4-(bromomethyl)-1-chloro-2-methoxybenzene

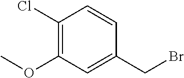

To a solution of 1-chloro-2-methoxy-4-methylbenzene (20.98 g, 0.13 mol) in CCl₄ (200 mL) was added in NBS (26.24 g, 0.15 mol) and BPO (1.62 g, 6.7 mmol). The resulting mixture was stirred at 80° C. overnight. After the reaction was completed by TLC analysis, the mixture was filtered and the filtrate was diluted with a diluted HCl solution (20 mL, 1N) and extracted with DCM (200 mL×3). The combined organic phase was washed with a saturated NaHCO₃ solution (40 mL), dried with Na₂SO₄ (60 g), filtered and concentrated to give 41.44 g of the crude product. The crude product was further purified by silica column chromatography (PE/EtOAc=100/1 to 60/1) to give 32.6 g of the title compound. ¹H-NMR (300 MHz, CDCl₃) δ 7.32 (d, J=8.1 Hz, 1H), 6.90-6.96 (m, 2H), 4.54 (s, 2H), 3.93 (s, 3H).

### 2-(4-chloro-3-methoxyphenyl)acetonitrile

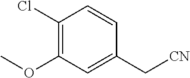

To a solution of 4-(bromomethyl)-1-chloro-2-methoxybenzene (10 g, 42.7 mmol) in EtOH (50 mL) and H₂O (10 mL) was added in NaCN (3.14 g, 64.05 mmol) in one portion. The resulting mixture was stirred at 80° C. overnight. After the reaction was completed by TLC analysis, the reaction was diluted with water (100 mL) and extracted with DCM (200 mL×2). The combined organic phase was washed with a saturate NaHCO₃ solution (50 mL) and brine (50 mL), dried and concentrated to afford 7.58 g of the crude product. The crude product was purified by column chromatography (PE:EtOAc=60/1 to 15/1) to give 5.58 g of the title compound. ¹H-NMR (300 MHz, CDCl₃) δ 7.34 (d, J=8.1 Hz, 1H), 6.83-6.88 (m, 2H), 3.91 (s, 3H), 3.73 (s, 2H).

### 2-(4-chloro-3-methoxyphenyl)acetic acid

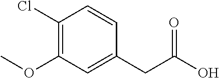

To a solution of 2-(4-chloro-3-methoxyphenyl)acetonitrile (5.58 g, 31 mmol) in ethanol (120 mL) and water (40 mL) was added KOH (8.68 g, 155 mol) in one portion. The reaction was stirred at reflux for about 3 hr. After the reaction was completed as indicated by TLC analysis, the reaction was concentrated to remove the most of ethanol. The residue was adjusted pH to 3 with a diluted HCl solution and extracted with EtOAc (200 mL×3). The combined organic phase was washed with water (30 mL), dried and concentrated to dryness to give 7.29 g of the crude product. ¹H-NMR (300 MHz, DMSO-d₆) δ12.40 (brs, 1H), 7.34 (d, J=7.8 Hz, 1H), 7.05 (d, J=1.5 Hz, 1H), 6.84 (d, J=7.8, 1.5 Hz, 1H), 3.92 (s, 3H), 3.72 (s, 2H).

### methyl 2-(4-chloro-3-methoxyphenyl)acetate

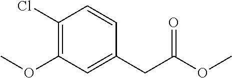

A solution of 2-(4-chloro-3-methoxyphenyl)acetic acid (7.29 g, 36.45 mmol) in methanol (100 mL) was added conc. H₂SO₄ (3.578 g, 36.45 mmol) in one portion. The reaction was stirred at 70° C. for 2 hr. After the reaction was completed as indicated by TLC analysis, the reaction was cooled to rt and concentrated to remove the most of methanol. The residue was diluted with water (20 mL) and extracted with EtOAc (50 mL×2). The combined organic phase was dried and concentrated. The residue was purified by column chromatography (PE/EtOAc=60/1 to 20/1) to give 4.68 g of the title compound. ¹H-NMR (300 MHz, CDCl₃) δ 7.29 (d, J=8.1 Hz, 1H), 6.86 (d, J=1.5 Hz, 1H), 6.80 (d, J=7.8, 1.5 Hz, 1H), 3.90 (s, 3H), 3.72 (s, 3H), 3.60 (s, 2H).

### methyl (Z)-2-(4-chloro-3-methoxyphenyl)-3-(dimethylamino)acrylate

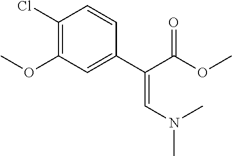

The compound was synthesized according to the procedure for the preparation of methyl (Z)-2-(4-cyano-2-methoxyphenyl)-3-(dimethylamino)acrylate using methyl 2-(4-chloro-3-methoxyphenyl)acetate. LC-MS (ESI+): m/z 270 (M+H)⁺;

### 6-(4-(4-chloro-3-methoxyphenyl)-5-hydroxy-1H-pyrazol-1-yl)nicotinic acid

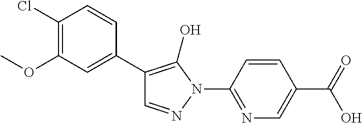 [SMILES: COc1cc(-c2cnn(-c3ccc(C(=O)O)cn3)c2O)ccc1Cl]

The compound was synthesized according to the procedure for the preparation of 6-(4-(4-cyano-2-methoxyphenyl)-5-hydroxy-1H-pyrazol-1-yl)nicotinic acid using methyl (Z)-2-(4-chloro-3-methoxyphenyl)-3-(dimethylamino)acrylate. LC-MS (ESI+): m/z 346 (M+H)⁺;

### 6-(4-(4-chloro-3-hydroxyphenyl)-5-hydroxy-1H-pyrazol-1-yl)nicotinic acid

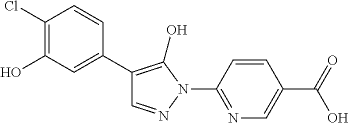 [SMILES: O=C(O)c1ccc(-n2ncc(-c3ccc(Cl)c(O)c3)c2O)nc1]

The compound was synthesized according to the procedure for the preparation of 6-(4-(4-cyano-2-hydroxyphenyl)-5-hydroxy-1H-pyrazol-1-yl)nicotinic acid using 6-(4-(4-chloro-3-methoxyphenyl)-5-hydroxy-1H-pyrazol-1-yl)nicotinic acid. LC-MS (ESI+): m/z 332 (M+H)⁺; ¹H-NMR (300 MHz, CDCl₃) δδ 13.30 (brs, 1H), 10.07 (s, 1H), 8.95 (s, 1H), 8.42-8.52 (m, 2H), 8.33 (s, 1H), 7.70 (s, 1H), 7.25 (s, 2H).

### Example 23: Preparation of Compound 23

### methyl 2-(4-bromo-3-fluorophenyl)acetate

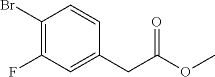

The compound was synthesized according to the procedure for the preparation of methyl 2-(4-bromo-2-methoxyphenyl)acetate using 2-(4-bromo-3-fluoro)acetic acid. ¹H-NMR (300 MHz, CDCl₃) δ 7.49 (t, J=8.1 Hz, 1H), 7.08 (d, J=9.0 Hz, 1H), 6.95 (d, J=8.1 Hz, 1H), 3.71 (s, 3H), 3.59 (s, 2H).

### methyl 2-(4-cyano-3-fluorophenyl)acetate

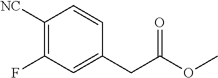

The compound was synthesized according to the procedure for the preparation of methyl 2-(4-cyano-2-methoxyphenyl)acetate using methyl 2-(4-bromo-3-fluorophenyl)acetate. ¹H-NMR (300 MHz, CDCl₃) δ 7.59 (t, J=8.1 Hz, 1H), 7.19 (d, J=8.7 Hz, 2H), 3.73 (s, 3H), 3.70 (s, 2H).

### methyl 2-(4-cyano-3-fluorophenyl)-3-oxobutanoate

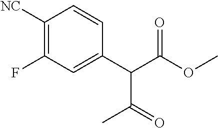 [SMILES: COC(=O)C(C(C)=O)c1ccc(C#N)c(F)c1]

Under nitrogen protection, a solution methyl 2-(4-cyano-3-fluorophenyl)acetate (363 mg, 1.88 mmol) in THF (15 mL) at −78° C. was added LiHMDS (2.9 mmol, 2.9 mL) dropwise over 10 mins. After the resulting mixture was stirred at −78° C. for 30 mins, a solution of 1-acetylimidazole (249 mg, 2.25 mmol) in THF (5 mL) was added dropwise to the reaction over 10 mins. The resulting mixture was continued to stir at −78° C. for 1 hr. After the reaction was completed as indicated by TLC analysis, the reaction was quenched with a saturated NH₄Cl solution (20 mL) and extracted with EtOAc (30 mL×2). The combined organic phase was dried and concentrated to dryness to afford 410 mg of the crude title compound, which was used for next step without further purification. LC-MS (ESI−): m/z 234 (M−H)⁻;

### 6-(4-(4-cyano-3-fluorophenyl)-5-hydroxy-3-methyl-1H-pyrazol-1-yl)nicotinic acid

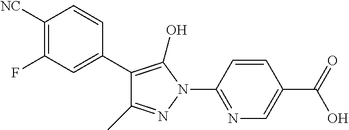 [SMILES: Cc1nn(-c2ccc(C(=O)O)cn2)c(O)c1-c1ccc(C#N)c(F)c1]

The compound was synthesized according to the procedure for the preparation of 6-(4-(4-cyanophenyl)-5-hydroxy-3-methyl-1H-pyrazol-1-yl)-4-methylnicotinic acid using methyl 2-(4-cyano-3-fluorophenyl)-3-oxobutanoate. LC-MS (ESI+): m/z 337 (M+H⁺), ¹H NMR (300 MHz, DMSO-d₆) δ 8.99 (d, J=1.5 Hz, 1H), 8.34 (m, 1H), 8.26 (d, J=8.1 Hz, 2H), 7.86 (d, J=11.7 Hz, 1H), 7.68 (m, 1H), 7.57 (m, 1H), 2.44 (s, 3H).

### Example 24: Preparation of Compound 24

### 3-methyl-4-vinylbenzonitrile

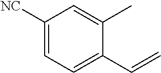

Under nitrogen protection, a solution of 4-bromo-3-methylbenzonitrile (5 g, 25.51 mmol) and vinyl trifluoro-potassium borate (6.84 g, 51.02 mmol) in THF/water (250 mL/25 mL) was added Cs₂CO₃ (33.3 g, 102.04 mmol) and Pd(dppf)Cl₂ (1.8 g, 2.55 mmol). The resulting mixture was stirred at 70° C. overnight. After the reaction was completed as indicated by TLC analysis, the reaction was quenched with water (100 mL) and extracted with EtOAc (50 mL×2). The combined organic phase was dried and concentrated. The residue was purified by column chromatography (PE:EtOAc=20/1 to 10/1) to give 3.2 g of the title compound as light yellow oil. GC-MS (EI+): m/z 143 (M⁺).

### 2-(4-cyano-2-methylphenyl)acetic acid

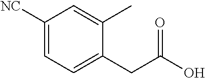

A solution of 3-methyl-4-vinylbenzonitrile (3.2 g, 22.38 mmol) in DME (200 mL) and water (48 mL) was added 12 (0.57 g, 2.24 mmol) in one portion. The resulting mixture at rt was added Oxone (27.52 g, 44.76 mmol) portion wise over 10 mins. After addition, the reaction was stirred at rt overnight. After the reaction was completed as indicated by TLC analysis, the suspension was filtered to remove the un-dissolved solid, the filtrate was concentrated under vacuum to remove the most of organic solvent. The residue was diluted with a saturated Na₂S₂O₃ (50 mL) solution and extracted with EtOAc (50 mL×3). The combined organic phase was dried and concentrated. The residue was slurred in n-hexane (30 mL) for 30 mins and filtered. After the n-hexane slurry and filtration purification was repeated twice, 3.56 g of the crude product was obtained, which was used for next step directly without further purification.

### methyl 2-(4-cyano-2-methylphenyl)acetate

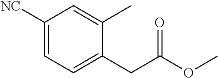

The compound was synthesized according to the procedure for the preparation of methyl 2-(4-bromo-2-methoxyphenyl)acetate using 2-(4-bromo-2-methyl)acetic acid.

¹H NMR (300 MHz, CDCl₃) δ 7.45-7.48 (m, 2H), 7.30 (d, J=8.1 Hz, 1H), 3.71 (s, 3H), 3.69 (s, 2H), 2.34 (s, 3H).

### methyl 2-(4-cyano-2-methylphenyl)-3-oxobutanoate

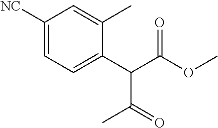 [SMILES: COC(=O)C(C(C)=O)c1ccc(C#N)cc1C]

The compound was synthesized according to the procedure for the preparation of methyl 2-(4-cyano-3-fluorophenyl)-3-oxobutanoate using methyl 2-(4-cyano-2-methylphenyl)acetate. LC-MS (ESI+): m/z 232 (M+H)⁺;

### 6-(4-(4-cyano-2-methylphenyl)-5-hydroxy-3-methyl-1H-pyrazol-1-yl)nicotinic acid

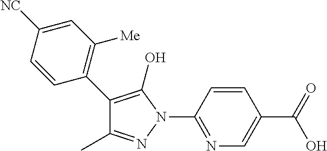 [SMILES: Cc1cc(C#N)ccc1-c1c(C)nn(-c2ccc(C(=O)O)cn2)c1O]

The compound was synthesized according to the procedure for the preparation of 6-(4-(4-cyano-3-fluorophenyl)-5-hydroxy-3-methyl-1H-pyrazol-1-yl)nicotinic acid using methyl 2-(4-cyano-2-methylphenyl)-3-oxobutanoate. LC-MS (ESI+): m/z 332 (M+H)⁺; ¹H-NMR (300 MHz, DMSO-d₆) δ12.91 (brs, 2H), 8.95 (s, 1H), 8.52 (d, J=8.7 Hz, 1H), 8.39 (d, J=8.7 Hz, 1H), 8.23 (s, 1H), 7.80 (s, 1H), 6.78 (s, 1H).

### Example 25: Preparation of Compound 25

### methyl 2-(4-bromo-2-fluorophenyl)acetate

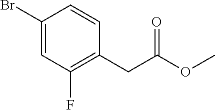

The compound was synthesized according to the procedure for the preparation of methyl 2-(4-chloro-3-methoxyphenyl)acetate using 2-(4-bromo-2-fluorophenyl)acetic acid. ¹H NMR (300 MHz, CDCl₃) δ 7.24-7.27 (m, 2H), 7.14 (t, J=8.4 Hz, 1H), 3.71 (s, 3H), 3.63 (s, 2H).

### methyl 2-(4-cyano-2-fluorophenyl)acetate

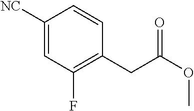

The compound was synthesized according to the procedure for the preparation of methyl 2-(4-cyano-3-fluorophenyl)acetate using methyl 2-(4-bromo-2-fluorophenyl)acetate. ¹H NMR (300 MHz, CDCl₃) δ 7.86 (dd, J=8.7, 1.2 Hz, 1H), 7.70 (d, J=8.1 Hz, 1H), 7.60 (t, J=7.5 Hz, 1H), 3.87 (s, 2H), 3.64 (s, 3H).

### methyl 2-(4-cyano-2-fluorophenyl)-3-oxobutanoate

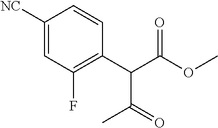 [SMILES: COC(=O)C(C(C)=O)c1ccc(C#N)cc1F]

The compound was synthesized according to the procedure for the preparation of methyl 2-(4-cyano-3-fluorophenyl)-3-oxobutanoate using methyl 2-(4-cyano-2-fluorophenyl)acetate. LC-MS (ESI+): m/z 258 (M+Na)⁺.

### 6-(4-(4-cyano-2-fluorophenyl)-5-hydroxy-3-methyl-1H-pyrazol-1-yl)nicotinic acid

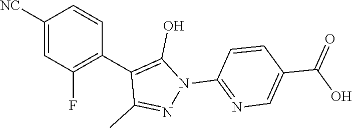 [SMILES: Cc1nn(-c2ccc(C(=O)O)cn2)c(O)c1-c1ccc(C#N)cc1F]

The compound was synthesized according to the procedure for the preparation of 6-(4-(4-cyano-3-fluorophenyl)-5-hydroxy-3-methyl-1H-pyrazol-1-yl)nicotinic acid using methyl 2-(4-cyano-2-fluorophenyl)-3-oxobutanoate. LC-MS (ESI+): m/z 339 (M+H⁺), ¹H NMR (300 MHz, DMSO-d₆) δ13.22 (brs, 1H), 8.94 (d, J=1.8 Hz, 1H), 8.59 (d, J=9.3 Hz, 1H), 8.38 (d, J=9.3 Hz, 1H), 7.69-7.94 (m, 3H), 2.23 (s, 3H).

### Example 26: Preparation of Compound 26

### methyl 2-(4-chloro-3-fluorophenyl)acetate

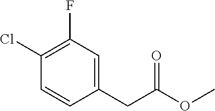

The compound was synthesized according to the procedure for the preparation of methyl 2-(4-chloro-3-methoxyphenyl)acetate using 2-(4-chloro-3-fluorophenyl)acetic acid. ¹H NMR (300 MHz, CDCl₃) δ 7.34 (t, J=7.8 Hz, 1H), 7.10 (dd, J=8.1, 1.8 Hz, 1H), 7.01 (d, J=8.1 Hz, 1H), 3.71 (s, 3H), 3.6 (s, 2H).

### methyl 2-(4-chloro-3-fluorophenyl)-3-oxobutanoate

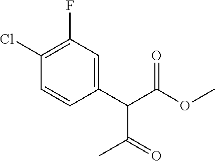 [SMILES: COC(=O)C(C(C)=O)c1ccc(Cl)c(F)c1]

The compound was synthesized according to the procedure for the preparation of methyl 2-(4-cyano-2-fluorophenyl)-3-oxobutanoate using methyl 2-(4-chloro-3-fluorophenyl)acetate. LC-MS (ESI+): m/z 267 (M+Na)⁺;

### 6-(4-(4-chloro-3-fluorophenyl)-5-hydroxy-3-methyl-1H-pyrazol-1-yl)nicotinic acid

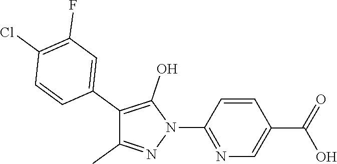 [SMILES: Cc1nn(-c2ccc(C(=O)O)cn2)c(O)c1-c1ccc(Cl)c(F)c1]

The compound was synthesized according to the procedure for the preparation of 6-(4-(4-cyano-2-fluorophenyl)-5-hydroxy-3-methyl-1H-pyrazol-1-yl)nicotinic acid using methyl 2-(4-chloro-3-fluorophenyl)-3-oxobutanoate. LC-MS (ESI+): m/z 348 (M+Na)⁺; ¹H-NMR (300 MHz, DMSO-d₆) δ13.30 (brs, 1H), 13.01 (brs, 1H), 8.95 (s, 1H), 8.56 (brs, 1H), 8.41 (dd, J=9.0, 2.1 Hz, 1H), 7.74 (d, J=11.7 Hz, 1H), 7.55-7.58 (m, 2H), 2.47 (s, 3H).

### Example 27: Preparation of Compound 27

### 4-amino-6-hydrazineylnicotinic acid

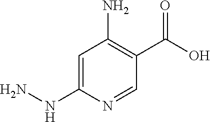 [SMILES: NNc1cc(N)c(C(=O)O)cn1]

The compound was synthesized according to the procedure for the preparation of 6-hydrazinyl-4-methylnicotinic acid using 4-amino-6-fluoronicotinic acid. LC-MS (ESI+): m/z 169 (M+H)⁺;

### 4-amino-6-(4-(4-cyanophenyl)-5-hydroxy-3-methyl-1H-pyrazol-1-yl)nicotinic acid

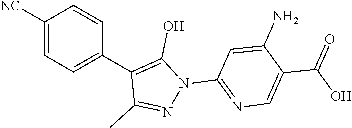 [SMILES: Cc1nn(-c2cc(N)c(C(=O)O)cn2)c(O)c1-c1ccc(C#N)cc1]

The compound was synthesized according to the procedure for the preparation of 6-(4-(4-cyano-3-fluorophenyl)-5-hydroxy-3-methyl-1H-pyrazol-1-yl)nicotinic acid using 4-amino-6-hydrazineylnicotinic acid. LC-MS (ESI+): m/z 336 (M+H)⁺; ¹H-NMR (300 MHz, DMSO-d₆) δ8.63 (s, 1H), 8.08 (s, 2H), 7.88 (d, J=8.4 Hz, 2H), 7.76 (d, J=8.4 Hz, 2H), 7.37 (brs, 1H), 2.39 (s, 3H).

### Example 28: Preparation of Compound 28

### 6-(4-(4-chloro-2-fluorophenyl)-5-hydroxy-3-methyl-1H-pyrazol-1-yl)nicotinic acid

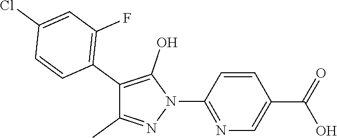 [SMILES: Cc1nn(-c2ccc(C(=O)O)cn2)c(O)c1-c1ccc(Cl)cc1F]

The compound was synthesized according to the procedure for the preparation of 6-(4-(4-chloro-3-fluorophenyl)-5-hydroxy-3-methyl-1H-pyrazol-1-yl)nicotinic acid. LC-MS (ESI+): m/z 348 (M+Na)⁺; ¹H-NMR (300 MHz, DMSO-d₆) δ13.36 (brs, 1H), 12.82 (brs, 1H), 8.95 (d, J=1.5 Hz, 1H), 8.58 (brs, 1H), 8.40 (m, 1H), 7.48-7.56 (m, 2H), 7.33 (m, 1H), 2.21 (s, 3H).

### Example 29: Preparation of Compound 29

### 2-methyl-4-vinylbenzonitrile

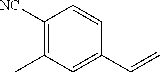

The compound was synthesized according to the procedure for the preparation of 3-methyl-4-vinylbenzonitrile using 4-bromo-2-methylbenzonitrile. ¹H NMR (300 MHz, CDCl₃) δ 7.55 (d, J=7.8 Hz, 1H), 7.26-7.32 (m, 2H), 6.70 (m, 1H), 5.85 (d, J=17.4 Hz, 1H), 5.42 (d, J=10.8 Hz, 1), 2.54 (s, 3H).

### methyl 2-(4-cyano-3-methylphenyl)acetate

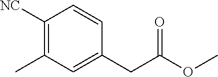

The compound was synthesized according to the procedure for the preparation of methyl 2-(4-cyano-2-methylphenyl)acetate using 2-methyl-4-vinylbenzonitrile. ¹H NMR (300 MHz, CDCl₃) δ 7.56 (d, J=7.8 Hz, 1H), 7.18-7.25 (m, 2H), 3.71 (s, 3H), 3.64 (s, 2H), 2.54 (s, 3H).

### methyl 2-(4-cyano-3-methylphenyl)-3-oxobutanoate

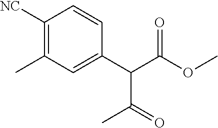

The compound was synthesized according to the procedure for the preparation of methyl 2-(4-cyano-3-fluorophenyl)-3-oxobutanoate using methyl 2-(4-cyano-3-methylphenyl)acetate. LC-MS (ESI−): m/z 230 (M−H)⁻;

### 6-(4-(4-cyano-3-methylphenyl)-5-hydroxy-3-methyl-1H-pyrazol-1-yl)nicotinic acid

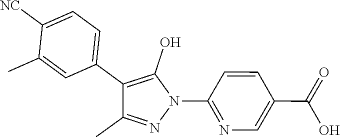 [SMILES: Cc1cc(-c2c(C)nn(-c3ccc(C(=O)O)cn3)c2O)ccc1C#N]

The compound was synthesized according to the procedure for the preparation of 6-(4-(4-cyanophenyl)-5-hydroxy-3-methyl-1H-pyrazol-1-yl)-4-methylnicotinic acid using methyl 2-(4-cyano-3-methylphenyl)-3-oxobutanoate. LC-MS (ESI+): m/z 333 (M−H)⁻; ¹H-NMR (300 MHz, CD₃OD) δ8.96 (s, 1H), 8.40 (d, J=8.7, 2.1 Hz, 1H), 8.39 (d, J=8.7 Hz, 1H), 7.71 (s, 1H), 7.58-7.66 (m, 2H), 2.55 (s, 3H), 2.45 (s, 3H).

### Example 30: Preparation of Compound 30

### methyl 2-(4-bromo-2-chlorophenyl)acetate

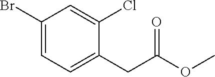

The compound was synthesized according to the procedure for the preparation of methyl 2-(4-bromo-2-fluorophenyl)acetate using 2-(4-bromo-2-fluorophenyl)acetic acid. ¹H-NMR (300 MHz, CDCl₃) δ 7.55 (d, J=1.8 Hz, 1H), 7.37 (dd, J=8.1, 1.8 Hz, 1H), 7.16 (d, J=1.8 Hz, 1H), 3.71 (s, 3H), 3.67 (s, 2H).

### methyl 2-(2-chloro-4-cyanophenyl)acetate

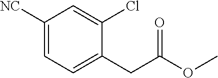

The compound was synthesized according to the procedure for the preparation of methyl 2-(4-cyano-2-fluorophenyl)acetate using methyl 2-(4-bromo-2-chlorophenyl)acetate. GC-MS (EI+): m/z 209.

### methyl (Z)-2-(2-chloro-4-cyanophenyl)-3-(dimethylamino)acrylate

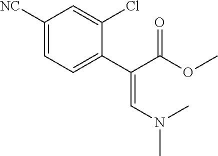 [SMILES: COC(=O)/C(=C\N(C)C)c1ccc(C#N)cc1Cl]

The compound was synthesized according to the procedure for the preparation of methyl (Z)-2-(4-cyano-2-methoxyphenyl)-3-(dimethylamino)acrylate using methyl 2-(2-chloro-4-cyanophenyl)acetate. LC-MS (ESI+): m/z 265 (M+H)⁺

### 6-(4-(2-chloro-4-cyanophenyl)-5-hydroxy-1H-pyrazol-1-yl)nicotinic acid

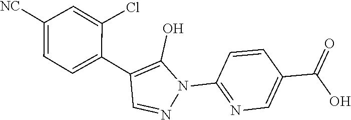 [SMILES: N#Cc1ccc(-c2cnn(-c3ccc(C(=O)O)cn3)c2O)c(Cl)c1]

The compound was synthesized according to the procedure for the preparation of 6-(4-(4-cyano-2-methoxyphenyl)-5-hydroxy-1H-pyrazol-1-yl)nicotinic acid using methyl (Z)-2-(2-chloro-4-cyanophenyl)-3-(dimethylamino)acrylate. LC-MS (ESI+): m/z 341 (M+H)⁺; ¹H-NMR (300 MHz, DMSO-d₆) δ 13.42 (brs, 2H), 8.97 (s, 1H), 8.44-8.60 (m, 3H), 8.28 (d, J=8.1 Hz, 1H), 8.06 (s, 1H), 7.81 (d, J=8.1 Hz, 1H).

### Example 31: Preparation of Compound 31

### 3,5-dimethyl-4-vinylbenzonitrile

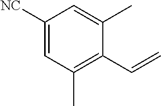

The compound was synthesized according to the procedure for the preparation of 3-methyl-4-vinylbenzonitrile using 4-bromo-3,5-dimethylbenzonitrile. ¹H-NMR (300 MHz, CDCl₃) δ 7.32 (s, 2H), 6.64 (m, 1H), 5.54 (dd, J=11.7, 1.5 Hz, 1H), 5.32 (dd, J 17.7, 1.5 Hz, 1H), 2.32 (s, 6H).

### methyl 2-(4-cyano-2,6-dimethylphenyl)acetate

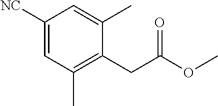

The compound was synthesized according to the procedure for the preparation of methyl 2-(4-cyano-2-methylphenyl)acetate using 3,5-dimethyl-4-vinylbenzonitrile. ¹H-NMR (300 MHz, CDCl₃) δ 7.33 (s, 2H), 3.72 (s, 2H), 3.70 (s, 3H), 2.35 (s, 6H).

### methyl (Z)-2-(4-cyano-2,6-dimethylphenyl)-3-(dimethylamino)acrylate

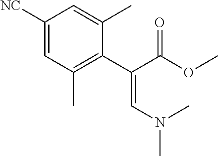 [SMILES: COC(=O)/C(=C\N(C)C)c1c(C)cc(C#N)cc1C]

The compound was synthesized according to the procedure for the preparation of methyl (Z)-2-(4-cyano-2-methoxyphenyl)-3-(dimethylamino)acrylate using methyl 2-(4-cyano-2,6-dimethylphenyl)acetate. ¹H-NMR (300 MHz, CDCl₃) δ 7.62 (s, 1H), 7.28 (s, 2H), 3.59 (s, 3H), 2.64 (s, 6H), 2.20 (s, 6H).

### 6-(4-(4-cyano-2,6-dimethylphenyl)-5-hydroxy-1H-pyrazol-1-yl)nicotinic acid

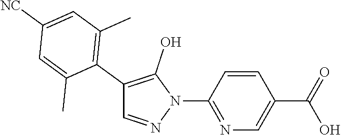 [SMILES: Cc1cc(C#N)cc(C)c1-c1cnn(-c2ccc(C(=O)O)cn2)c1O]

The compound was synthesized according to the procedure for the preparation of 6-(4-(4-cyano-2-methoxyphenyl)-5-hydroxy-1H-pyrazol-1-yl)nicotinic acid using methyl (Z)-2-(4-cyano-2,6-dimethylphenyl)-3-(dimethylamino)acrylate. LC-MS (ESI+): m/z 335 (M+H)⁺; ¹H-NMR (300 MHz, CD₃OD) δ8.03 (s, 1H), 8.52 (m, 2H), 7.75 (s, 1H), 7.40-7.47 (m, 2H), 2.30 (s, 6H).

### Example 32: Preparation of Compound 32

### methyl 2-(4-methoxy-3-methylphenyl)acetate

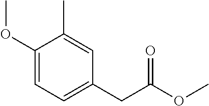

The compound was synthesized according to the procedure for the preparation of methyl 2-(4-bromo-2-methoxyphenyl)acetate using 2-(4-methoxy-3-methylphenyl)acetic acid. ¹H-NMR (300 MHz, CD₃OD) δ 7.05-7.07 (m, 2H), 6.77 (d, J=7.8 Hz, 1H), 3.81 (1, 3H), 3.76 (s, 3H), 3.54 (s, 2H), 2.20 (s, 3H).

### methyl 2-(4-methoxy-3-methylphenyl)-3-oxobutanoate

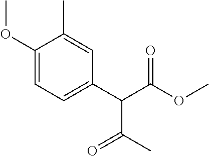 [SMILES: COC(=O)C(C(C)=O)c1ccc(OC)c(C)c1]

The compound was synthesized according to the procedure for the preparation of methyl 2-(4-cyano-3-fluorophenyl)-3-oxobutanoate using methyl 2-(4-methoxy-3-methylphenyl)acetate. LC-MS (ESI+): m/z 259 (M+Na)⁺;

### 6-(5-hydroxy-4-(4-methoxy-3-methylphenyl)-3-methyl-1H-pyrazol-1-yl)nicotinic acid

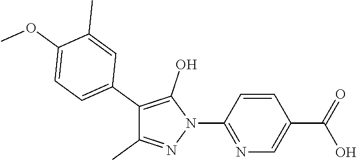 [SMILES: COc1ccc(-c2c(C)nn(-c3ccc(C(=O)O)cn3)c2O)cc1C]

The compound was synthesized according to the procedure for the preparation of 6-(4-(4-cyano-3-fluorophenyl)-5-hydroxy-3-methyl-1H-pyrazol-1-yl)nicotinic acid using methyl 2-(4-methoxy-3-methylphenyl)-3-oxobutanoate. LC-MS (ESI+): m/z 340 (M+H)⁺; ¹H-NMR (300 MHz, DMSO-d₆) δ13.30 (brs, 1H), 12.35 (brs, 1H), 8.93 (s, 1H), 8.58 (s, 1H), 8.39 (d, J=6.9 Hz, 1H), 7.34-7.36 (m, 2H), 6.96 (d, J=9.0 Hz, 1H), 3.80 (s, 3H), 2.35 (s, 3H), 2.18 (s, 3H).

### Example 33: Preparation of Compound 33

### diethyl 2-(2-bromo-4-cyanophenyl)malonate

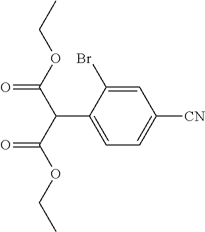 [SMILES: CCOC(=O)C(C(=O)OCC)c1ccc(C#N)cc1Br]

Under nitrogen protection, a solution of diethyl malonate (2 g, 12.5 mmol) in DMF at 0° C. was added NaH (600 mg, 15 mmol) portion wise over 5 min. After the reaction was stirred at 0° C. for 30 min, 3-bromo-4-fluorobenzonitrile (2.08 g, 10 mmol) was added to the reaction in one portion and the reaction was stirred at 80° C. for 1.5 hr. After the reaction was completed as indicated by TLC analysis, the reaction was quenched with water (20 mL) and adjusted pH to 5 with a diluted HCl solution. The resulting mixture was then extracted with EtOAc (50 mL×2). The combined organic phase was washed with water (20 mL) and brine (20 mL), dried and concentrated to dryness. The residue was purified by column chromatography (PE/EtOAc=20/1 to 10/1) to give 1.8 g of the title compound. LC-MS (ESI+): m/z 340, 342 (M+H)⁺. ¹H-NMR (300 MHz, CDCl₃) δ 7.90 (s, 2H), 7.64 (s, 2H), 5.24 (s, 1H), 4.21-4.32 (m, 4H), 1.29 (t, J=7.2 Hz, 6H).

### ethyl 2-(2-bromo-4-cyanophenyl)acetate

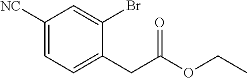

To a solution of diethyl 2-(2-bromo-4-cyanophenyl)malonate (1.8 g, 5.3 mmol) in DMSO (20 mL) and water (96 mg, 5.3 mmol) was added LiCl (339 mg, 8 mmol) in one portion. The resulting mixture was stirred at 120° C. overnight. After the reaction was completed as indicated by TLC analysis, the reaction was quenched by water (20 mL), adjusted pH to 6 with a diluted HCl solution and extracted with EtOAc (20 mL×2). The combined organic phase was washed with water (20 mL) and brine (20 mL), dried and concentrated. The residue was purified by column chromatography (PE/EtOAc=20/1 to 10/1) to give 780 mg of the title compound. ¹H-NMR (300 MHz, CDCl₃) δ 7.87 (s, 1H), 7.59 (d, J=7.8 Hz, 1H), 7.42 (d, J=7.8 Hz, 1H), 4.20 (q, J=6.9 Hz, 2H), 3.84 (s, 2H), 1.27 (t, J=6.9 Hz, 3H)

### ethyl 2-(4-cyano-2-vinylphenyl)acetate

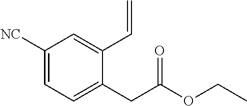 [SMILES: C=Cc1cc(C#N)ccc1CC(=O)OCC]

The compound was synthesized according to the procedure for the preparation of 3-methyl-4-vinylbenzonitrile using ethyl 2-(2-bromo-4-cyanophenyl)acetate. ¹H NMR (300 MHz, CDCl₃) δ 7.7 (s, 1H), 7.52 (d, J=7.8 Hz, 1H), 7.33 (d, J=7.8 Hz, 1H), 6.89 (m, 1H), 5.70 (d, J=17.4 Hz, 1H), 5.42 (d, J=10.8 Hz, 1), 4.15 (q, J=7.2 Hz, 1H), 3.73 (s, 3H), 1.24 (q, J=7.2 Hz, 1H).

### ethyl 2-(4-cyano-2-ethylphenyl)acetate

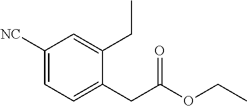 [SMILES: CCOC(=O)Cc1ccc(C#N)cc1CC]

To a solution of methyl 2-(4-cyano-2-vinylphenyl)acetate (560 mg, 2.6 mmol) in methanol (10 mL) was added Pd/C (56 mg). The suspension was stirred under hydrogen atmosphere from a balloon for about 1 hr. After the reaction was completed as indicated by TLC analysis, the suspension was filtered through a package of Celite and the filter cake was washed with methanol (10 mL). The filtrate was concentrated to dryness to give 540 mg of the crude title compound. ¹H-NMR (300 MHz, CDCl₃) δ 7.61 (s, 1H), 7.49 (d, J=8.1 Hz, 1H), 7.31 (d, J=8.1 Hz, 1H), 4.16 (q, J=7.2 Hz, 2H), 3.69 (s, 2H), 2.68 (q, J=7.5 Hz, 2H), 1.21-1.28 (m, 6H).

### ethyl (Z)-2-(4-cyano-2-ethylphenyl)-3-(dimethylamino)acrylate

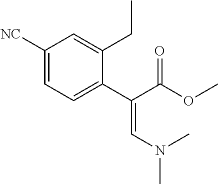 [SMILES: CCc1cc(C#N)ccc1/C(=C/N(C)C)C(=O)OC]

The compound was synthesized according to the procedure for the preparation of methyl (Z)-2-(4-cyano-2-methoxyphenyl)-3-(dimethylamino)acrylate using ethyl 2-(4-cyano-2-ethylphenyl)acetate. ¹H-NMR (300 MHz, CDCl₃) δ 7.60 (s, 1H), 7.49 (s, 1H), 7.41 (d, J=8.1 Hz, 1H), 7.23 (m, 1H), 4.12 (q, J=6.9 Hz, 2H), 3.60 (s, 3H), 2.67 (s, 6H), 1.14 (t, J=6.9 Hz, 3H)

### 6-(4-(4-cyano-2-ethylphenyl)-5-hydroxy-1H-pyrazol-1-yl)nicotinic acid

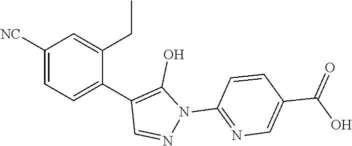 [SMILES: CCc1cc(C#N)ccc1-c1cnn(-c2ccc(C(=O)O)cn2)c1O]

The compound was synthesized according to the procedure for the preparation of 6-(4-(4-cyano-2-methoxyphenyl)-5-hydroxy-1H-pyrazol-1-yl)nicotinic acid using ethyl (Z)-2-(4-cyano-2-ethylphenyl)-3-(dimethylamino)acrylate. LC-MS (ESI+): m/z 335 (M+H)⁺; ¹H-NMR (300 MHz, DMSO-d₆) δ8.97 (s, 1H), 8.52 (m, 2H), 8.12 (s, 1H), 7.75 (s, 1H), 7.60-7.69 (m, 2H), 2.78 (q, J=7.5 Hz, 2H), 1.17 (q, J=7.5 Hz, 3H)

### Example 34: Preparation of Compound 34

### 5-hydrazineylpyrazine-2-carboxylic acid

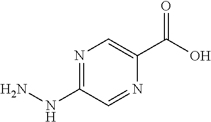 [SMILES: NNc1cnc(C(=O)O)cn1]

The compound was synthesized according to the procedure for the preparation of 6-hydrazinyl-4-methylnicotinic acid using 5-fluoropyrazine-2-carboxylic acid. LC-MS (ESI+): m/z 155 (M+H)⁺;

### 5-(4-(4-cyanophenyl)-5-hydroxy-3-methyl-1H-pyrazol-1-yl)pyrazine-2-carboxylic acid

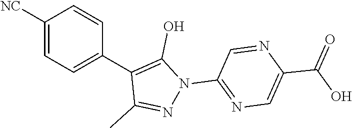 [SMILES: Cc1nn(-c2cnc(C(=O)O)cn2)c(O)c1-c1ccc(C#N)cc1]

The compound was synthesized according to the procedure for the preparation of 6-(4-(4-cyano-3-fluorophenyl)-5-hydroxy-3-methyl-1H-pyrazol-1-yl)nicotinic acid using 5-hydrazineylpyrazine-2-carboxylic acid. LC-MS (ESI+): m/z 322 (M+H)⁺; ¹H-NMR (300 MHz, DMSO-d₆) δ9.80 (s, 1H), 9.04 (s, 1H), 7.91 (d, J=8.4 Hz, 2H), 7.83 (d, J=8.4 Hz, 2H), 2.51 (s, 3H).

### Example 35: Preparation of Compound 35

### Ethyl 2-(2-methoxypyridin-4-yl)acetate

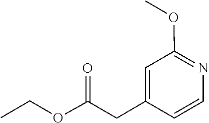

To a solution of 2-methoxy-4-methylpyridine (2.0 g, 16.2 mmol) in anhydrous tetrahydrofuran (50.0 mL) was added lithium diisopropylamide (16.0 mL, 32.0 mmol, 2.0 M in n-heptane) at −78° C. under nitrogen. The mixture was stirred at −78° C. for 10 min and diethyl carbonate (3.78 g, 32.0 mmol) was added. The mixture was allowed to warm up to room temperature and left stirring for 2.0 h. The reaction was quenched with water and extracted with ethyl acetate twice. The organic layer was washed with brine, dried over sodium sulfate and concentrated under reduced pressure. The crude product was purified by flash chromatography (petroleum ether/ethyl acetate=10/1) to obtain ethyl 2-(2-methoxypyridin-4-yl)acetate (2.0 g, 10.2 mmol, 63.4% yield) as yellow oil. LC-MS: m/z=196.1 (M+H)⁺, retention time 1.97 min (Method A).

### Ethyl (E)-3-(dimethylamino)-2-(2-methoxypyridin-4-yl)acrylate

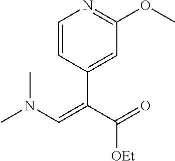

To a solution of ethyl 2-(2-methoxypyridin-4-yl)acetate (1.95 g, 10 mmol) in N,N-dimethylformamide (3.0 mL) was added N,N-dimethylformamide diethyl acetal (5.95 g, 50 mmol). The mixture was stirred at 100° C. for 12.0 h and cooled. Ethyl acetate and water were added to the solution, and the layers were separated. The organic layer was washed with brine, dried over sodium sulfate and concentrated under reduced pressure. The crude product was purified by flash chromatography (dichloromethane/methanol=98/2) to obtain ethyl (E)-3-(dimethylamino)-2-(2-methoxypyridin-4-yl)acrylate (1.1 g, 4.4 mmol, 44% yield) as colurless oil. LC-MS: m/z=251.0 [M+H]⁺, retention time 1.68 min (Method B).

### Tert-butyl 6-(5-hydroxy-4-(2-methoxypyridin-4-yl)-1H-pyrazol-1-yl)nicotinate

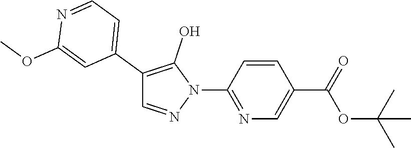 [SMILES: COc1cc(-c2cnn(-c3ccc(C(=O)OC(C)(C)C)cn3)c2O)ccn1]

To a solution of ethyl (E)-3-(dimethylamino)-2-(2-methoxypyridin-4-yl)acrylate (0.25 g, 1.0 mmol) and tert-butyl 6-hydrazineylnicotinate (0.21 g, 1.0 mmol) in ethanol (5.0 mL) was added p-toluenesulfonic acid monohydrate (19 mg, 0.1 mmol). The mixture was stirred at refluxing temperature for 12.0 h and cooled to precipitate solid. The solid was filtered, washed with ethanol and dried to give tert-butyl 6-(5-hydroxy-4-(2-methoxypyridin-4-yl)-1H-pyrazol-1-yl)nicotinate (210 mg, 0.57 mmol, 57% yield) as white solid. LC-MS: m/z=369.0 (M+H)⁺, retention time 4.38 min (Method A). ¹HNMR (400 MHz, DMSO-d₆) (13.53 (m, 1H), 8.91 (s, 1H), 8.39-8.67 (m, 2H), 8.04-8.06 (d, J=5.0 Hz, 1H), 7.39-7.50 (m, 2H), 3.86 (m, 3H), 1.58 (s, 9H).

### Example 36: Preparation of Compound 36

### Ethyl 6-hydrazineylnicotinate

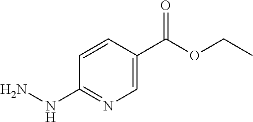

To a solution of ethyl 6-chloronicotinate (1.0 g, 5.40 mmol) in tetrahydrofuran (6.0 mL) was added hydrazine hydrate (300 mg, 5.94 mmol, 85% in water). The mixture was stirred at refluxing temperature overnight. The mixture was cooled and concentrated to dryness. The residue was partitioned between ethyl acetate and water. The organic phase was washed with brine, dried over sodium sulfate and concentrated. The crude product was obtained (600 mg, 3.31 mmol, 61.4% yield) as yellow oil. LC-MS: m/z=182.1 (M+H)⁺, retention time 0.37 min (Method A). The product was pure enough and used directly to the next step.

### Ethyl 6-(4-(4-cyanophenyl)-5-hydroxy-1H-pyrazol-1-yl)nicotinate

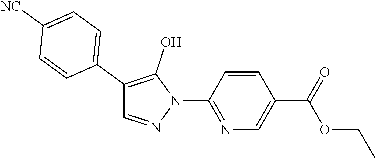 [SMILES: CCOC(=O)c1ccc(-n2ncc(-c3ccc(C#N)cc3)c2O)nc1]

To a solution of methyl (E)-2-(4-cyanophenyl)-3-(dimethylamino)acrylate (300 mg, 1.30 mmol) and ethyl 6-hydrazineylnicotinate (235 mg, 1.30 mmol) in ethanol (5.0 mL) was added p-toluenesulfonic acid monohydrate (25 mg, 0.13 mmol). The reaction was stirred at 90° C. overnight and cooled to precipitate solid. The solid was filtered, washed with ethanol and dried to obtain ethyl 6-(4-(4-cyanophenyl)-5-hydroxy-1H-pyrazol-1-yl)nicotinate (51 mg, 0.15 mmol, 11.7% yield) as yellow solid. LC-MS: m/z=335.1 [M+H]⁺, retention time 5.05 min (Method A). ¹HNMR (400 MHz, DMSO-d₆) (13.58 (s, 1H), 8.97 (s, 1H), 8.70-8.47 (m, 3H), 8.15 (s, 2H), 7.79-7.77 (m, 2H), 4.39-4.34 (m, 2H), 1.37-1.22 (m, 3H).

### Example 37: Preparation of Compound 37

### Isopropyl 6-chloronicotinate

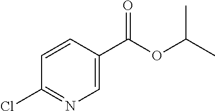

To a solution of 6-chloronicotinic acid (3.0 g, 19.2 mmol) in dichloromethane (50.0 mL) was added carbonyl diimidazole (3.42 g, 21.1 mmol) at room temperature. The mixture was stirred for 1.0 h and isopropyl alcohol (3.78 g, 32.0 mmol) was added. The dichloromethane was removed under vacuum. A catalytic amount of sodium isopropoxide (164 mg, 2.0 mmol) was added and the mixture was heated at 90° C. for 1.0 h. The solution was concentrated under vacuum. The resulting residue was slurried with water and extracted with ethyl acetate. The organic layer was washed with brine, dried over sodium sulfate and concentrated to give isopropyl 6-chloronicotinate (3.4 g, 17.1 mmol, 89% yield) as yellow solid. LC-MS: m/z=200.0 [M+H]⁺, retention time 2.04 min (Method A). The product was pure enough and used directly to the next step

### Isopropyl 6-hydrazineylnicotinate

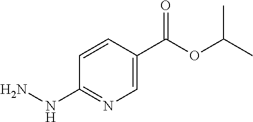

To a solution of isopropyl 6-chloronicotinate (1.0 g, 5.03 mmol) in ethanol (10.0 mL) was added hydrazine hydrate (1.0 g, 20.1 mmol, 85% in water). The mixture was stirred at 80° C. overnight. The mixture was cooled and concentrated to give dryness. The residue was partitioned between ethyl acetate and water. The organic phase was washed with brine, dried over sodium sulfate and concentrated. The residue was triturated with petroleum ether and filtered to afford isopropyl 6-hydrazineylnicotinate (800 mg, 4.1 mmol, 81.6% yield) as yellow solid. LC-MS: m/z=196.0 (M+H)⁺, retention time 0.39 min (Method A).

### Isopropyl 6-(4-(4-cyanophenyl)-5-hydroxy-1H-pyrazol-1-yl)nicotinate

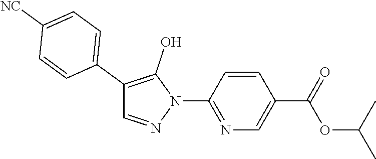 [SMILES: CC(C)OC(=O)c1ccc(-n2ncc(-c3ccc(C#N)cc3)c2O)nc1]

To a solution of methyl (E)-2-(4-cyanophenyl)-3-(dimethylamino)acrylate (300 mg, 1.30 mmol) and isopropyl 6-hydrazineylnicotinate (254 mg, 1.30 mmol) in ethanol (5.0 mL) was added p-toluenesulfonic acid monohydrate (25 mg, 0.13 mmol). The reaction was stirred at 90° C. overnight and cooled to precipitate solid. The solid was filtered, washed with ethanol and dried to obtain isopropyl 6-(4-(4-cyanophenyl)-5-hydroxy-1H-pyrazol-1-yl)nicotinate (53 mg, 0.15 mmol, 11.7% yield) as yellow solid. LC-MS: m/z=349.0 [M+H]⁺, retention time 5.44 min (Method A). ¹HNMR (400 MHz, DMSO-d₆) δ 13.55 (s, 1H), 8.95 (s, 1H), 8.69 (s, 1H), 8.53-8.44 (m, 2H), 8.14-8.12 (m, 2H), 8.79-8.77 (m, 2H), 5.21-5.15 (m, 1H), 1.36-1.34 (m, 6H).

### Example 38: Preparation of Compound 38

### Tert-butyl 6-chloronicotinate

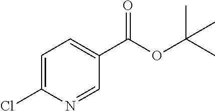

To a solution of 6-chloronicotinic acid (5.0 g, 6.37 mmol) and 4-dimethylaminopyridine (0.39 g, 0.64 mmol) in tetrahydrofuran (50.0 mL) was added di-tert-butyl dicarbonate (10.41 g, 47.77 mmol). The reaction mixture was refluxed for 4.0 h and concentrated. The residue was purified by flash chromatography (petroleum ether/ethyl acetate=10/1) to afford tert-butyl 6-chloronicotinate (5.5 g, 81.12% yield) as yellow solid. LC-MS: m/z=214.0 (M+H)⁺, retention time 1.83 min (Method A).

### Tert-butyl 6-hydrazineylnicotinate

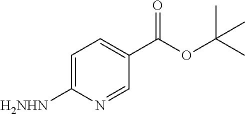

To a solution of tert-butyl 6-chloronicotinate (5.5 g, 25.82 mmol) in ethanol (25.0 mL) was added hydrazine hydrate (6.46 g, 129.11 mmol, 85% in water). The mixture was stirred at 100° C. for 2.0 h. The mixture was cooled and concentrated to dryness. The residue was partitioned between ethyl acetate and water. The organic phase was washed with brine, dried over sodium sulfate and concentrated. The residue was triturated with petroleum ether and filtered to afford tert-butyl 6-hydrazineylnicotinate (5.0 g, 92.76% yield) as yellow solid. LC-MS: m/z=210.0 (M+H)⁺, retention time 1.19 min (Method A).

### Methyl 2-(4-cyanophenyl)acetate

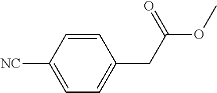

To a mixture of 2-(4-cyanophenyl)acetic acid (5.0 g, 31.0 mmol) in methanol (10.0 mL) was added hydrochloric acid in methanol (20.0 mL, 3.0 M) at 0° C. The mixture was stirred at 70° C. for 3.0 h and cooled to precipitate solid. The solid was filtered, washed with methanol and dried to give methyl 2-(4-cyanophenyl)acetate (5.0 g, 28.4 mmol, 92% yield) as yellow solid. LC-MS: m/z=176.0 [M+H]⁺, retention time 1.54 min (Method A).

### Methyl (E)-2-(4-cyanophenyl)-3-(dimethylamino)acrylate

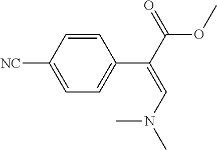 [SMILES: COC(=O)/C(=C/N(C)C)c1ccc(C#N)cc1]

To a solution of methyl 2-(4-cyanophenyl)acetate (5.0 g, 28.5 mmol) in N,N-dimethylformamide (25.0 mL) was added N,N-dimethylformamide diethyl acetal (14.0 g, 114.16 mmol). The mixture was stirred at 100° C. for 16.0 h and cooled. Ethyl acetate and water were added to the solution, and the layers were separated. The organic layer was washed with brine, dried over sodium sulfate and concentrated to give methyl (E)-2-(4-cyanophenyl)-3-(dimethylamino)acrylate (5.20 g, 25.4 mmol, 89% yield) as yellow solid. LC-MS: m/z=231.0 [M+H]⁺, retention time 1.70 min (Method A). The product was pure enough and used directly to the next step

### Tert-butyl 6-(4-(4-cyanophenyl)-5-hydroxy-1H-pyrazol-1-yl)nicotinate

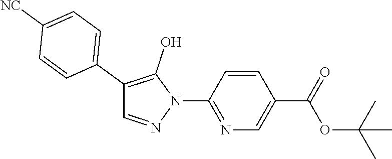 [SMILES: CC(C)(C)OC(=O)c1ccc(-n2ncc(-c3ccc(C#N)cc3)c2O)nc1]

To a solution of methyl (E)-2-(4-cyanophenyl)-3-(dimethylamino)acrylate (2.0 g, 8.70 mmol) and tert-butyl 6-hydrazineylnicotinate (1.82 g, 8.70 mmol) in ethanol (20.0 mL) was added p-toluenesulfonic acid monohydrate (171 mg, 0.9 mmol). The reaction was stirred at 90° C. for 16.0 h and cooled to precipitate solid. The solid was filtered, washed with ethanol and dried to obtain tert-butyl 6-(4-(4-cyanophenyl)-5-hydroxy-1H-pyrazol-1-yl)nicotinate (2.3 g, 6.35 mmol, 73% yield) as yellow solid. LC-MS: m/z=363.1 (M+H)⁺, retention time 5.82 min (Method A). ¹HNMR (400 MHz, DMSO-d₆) (13.58 (s, 1H), 8.92 (s, 1H), 8.69 (s, 1H), 8.51 (s, 1H), 8.44-8.41 (m, 1H), 8.15-8.13 (m, 2H), 7.81-7.78 (m, 2H), 1.58 (s, 9H).

### Example 39: Preparation of Compound 39

### methyl 2-(4-chloro-2-methoxyphenyl)acetate

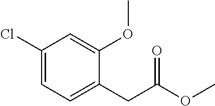

The compound was synthesized according to the procedure for the preparation of methyl 2-(4-bromo-2-methoxyphenyl)acetate using 2-(4-chloro-2-methoxyphenyl)acetic acid. LC-MS (ESI+): m/z 237 (M+Na)⁺; ¹H-NMR (300 MHz, CDCl₃) δ 7.10 (d, J=8.1 Hz, 1H), 6.85-6.92 (m, 2H), 3.86 (s, 3H), 3.71 (s, 3H), 3.67 (s, 2H).

### methyl (Z)-2-(4-chloro-2-methoxyphenyl)-3-(dimethylamino)acrylate

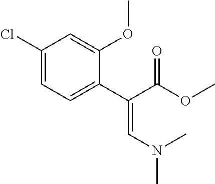 [SMILES: COC(=O)/C(=C\N(C)C)c1ccc(Cl)cc1OC]

The compound was synthesized according to the procedure for the preparation of methyl (Z)-2-(4-cyano-2-methoxyphenyl)-3-(dimethylamino)acrylate using methyl 2-(4-chloro-2-methoxyphenyl)acetate. LC-MS (ESI+): m/z 270 (M+H)⁺.

### 6-(4-(4-chloro-2-methoxyphenyl)-5-hydroxy-1H-pyrazol-1-yl)nicotinic acid

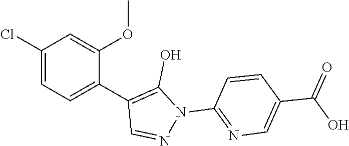 [SMILES: COc1cc(Cl)ccc1-c1cnn(-c2ccc(C(=O)O)cn2)c1O]

The compound was synthesized according to the procedure for the preparation of 6-(4-(4-cyano-2-methoxyphenyl)-5-hydroxy-1H-pyrazol-1-yl)nicotinic acid using methyl (Z)-2-(4-chloro-2-methoxyphenyl)-3-(dimethylamino)acrylate. LC-MS (ESI+): m/z 346 (M+H)⁺;

### 6-(4-(4-chloro-2-hydroxyphenyl)-5-hydroxy-1H-pyrazol-1-yl)nicotinic acid

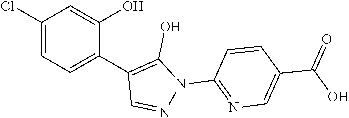 [SMILES: O=C(O)c1ccc(-n2ncc(-c3ccc(Cl)cc3O)c2O)nc1]

The compound was synthesized according to the procedure for the preparation of 6-(4-(4-cyano-2-hydroxyphenyl)-5-hydroxy-1H-pyrazol-1-yl)nicotinic acid using 6-(4-(4-chloro-2-methoxyphenyl)-5-hydroxy-1H-pyrazol-1-yl)nicotinic acid. LC-MS (ESI+): m/z 332 (M+H)⁺; ¹H-NMR (300 MHz, DMSO-d₆) δ12.91 (brs, 2H), 8.95 (s, 1H), 8.52 (d, J=8.7 Hz, 1H), 8.39 (d, J=8.7 Hz, 1H), 8.23 (s, 1H), 7.80 (s, 1H), 6.78 (s, 2H).

### Example 40: Preparation of Compound 40

### diethyl 2-(4-cyano-2-(trifluoromethyl)phenyl)malonate

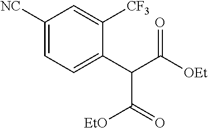 [SMILES: CCOC(=O)C(C(=O)OCC)c1ccc(C#N)cc1C(F)(F)F]

Under nitrogen protection, a solution of ethyl 3-(ethylperoxy)-3-oxopropanoate (10.20 g, 63.50 mmol) in DMF (100 ml) was added in Cs₂CO₃ (45.50 g, 139.70 mmol). After the reaction was stirred at 70° C. for 10 mins, 4-fluoro-3-(trifluoromethyl)benzonitrile (12.00 g, 63.50 mmol) was added to the reaction. The resulting mixture was stirred at 70° C. for 2 hrs. After the reaction was completed as indicated by TLC, the reaction was quenched with water (300 mL) and extracted with EtOAc (500 mL×2). The combined organic phase was washed with water (50 mL), dried with Na₂SO₄, filtered and concentrated to get the desired product (20.8 g) as oil. ¹H-NMR (300 MHz, CDCl₃) δ 7.99 (s, 1H), 7.86-7.92 (m, 2H), 5.11 (s, 1H), 4.20-4.33 (m, 4H), 1.28 (t, J=7.2 Hz, 6H).

### ethyl 2-(4-cyano-2-(trifluoromethyl)phenyl)acetate

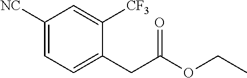 [SMILES: CCOC(=O)Cc1ccc(C#N)cc1C(F)(F)F]

To a solution of diethyl 2-(4-cyano-2-(trifluoromethyl)phenyl)malonate (15.00 g, 45.60 mmol) in DMSO (150 ml) was added in LiCl (2.90 g, 68.40 mmol) and water (0.80 mL, 45.6 mmol). The mixture was stirred at 120° C. overnight. After the reaction was completed as indicated by TLC, the reaction was quenched with water (300 mL) and extracted with EtOAc (150 mL×3). The combined organic phase was washed with water (100 mL), dried with Na₂SO₄, filtered and concentrated. The residue was purified by silica chromatography (EA/PE=1/25) to give the desired product (8.28 g) as white solid. ¹H-NMR (300 MHz, CDCl₃) δ 7.97 (d, J=1.8 Hz, 1H), 7.83 (d, J=7.8 Hz, 1H), 7.57 (d, J=7.8 Hz, 1H), 4.18 (q, J=7.2 Hz, 2H), 3.88 (s, 2H), 1.26 (t, J=7.2 Hz, 3H).

### ethyl (Z)-2-(4-cyano-2-(trifluoromethyl)phenyl)-3-(dimethylamino)acrylate

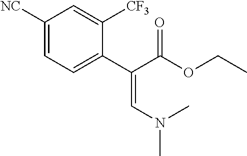 [SMILES: CCOC(=O)/C(=C\N(C)C)c1ccc(C#N)cc1C(F)(F)F]

To a solution of ethyl 2-(4-cyano-2-(trifluoromethyl)phenyl)acetate (4.00 g, 15.56 mmol) and DME-DMA (15 mL) in a seal tube was stirred at 150° C. overnight. After the reaction was completed as indicated by TLC, the mixture was diluted with water (100 mL) and extracted with EtOAc (150 mL×3). The combined organic phase was dried with Na₂SO₄, filtered and concentrated. The residue was purified by silica chromatography (EA/PE=1/9) to get the desired product (3.2 g) as brown oil. ¹H-NMR (300 MHz, CDCl₃) δ 7.94 (s, 1H), 7.75 (d, J=7.8 Hz, 1H), 7.59 (s, 1H), 7.46 (d, J=7.8 Hz, 1H), 3.95-4.08 (m, 2H), 2.66 (brs, 6H), 1.10 (t, J=7.2 Hz, 3H).

### 6-(4-(4-cyano-2-(trifluoromethyl)phenyl)-5-hydroxy-1H-pyrazol-1-yl)nicotinic acid

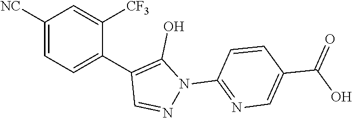 [SMILES: N#Cc1ccc(-c2cnn(-c3ccc(C(=O)O)cn3)c2O)c(C(F)(F)F)c1]

To a solution of ethyl (Z)-2-(4-cyano-2-(trifluoromethyl)phenyl)-3-(dimethylamino)acrylate (1.00 g, 3.20 mmol) in i-PrOH (38 mL) was added in 6-hydrazinylnicotinic acid (0.59 g, 0.38 mmol) and a diluted HCl solution (38 mL, 1M). The reaction was stirred at rt for 24 hrs. After the reaction was completed as indicated by LCMS analysis, DIEA (15 eq) was added and the mixture was stirred at rt for 2 days. After the mixture was adjusted pH to 3, a large amount of solid was precipitated. After filtration and slurry purification, 90 mg of the desired product was obtained as solid. HPLC purity was 93.1%. LC-MS (ESI+): m/z 375 (M+H)⁺; ¹H-NMR (300 MHz, DMSO-d₆) δ8.98 (s, 1H), 8.47-8.57 (m, 2H), 8.34 (s, 1H), 8.17 (d, J=8.4 Hz, 1H), 7.97-8.02 (m, 2H).

### Example 41: Preparation of Compound 41

### ethyl 3-oxo-2-(pyridin-4-yl)butanoate

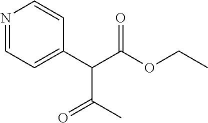

The compound was synthesized according to the procedure for the preparation of methyl 2-(4-cyano-2-fluorophenyl)-3-oxobutanoate using ethyl 3-oxo-2-(pyridin-4-yl)butanoate. LC-MS (ESI+): m/z 208 (M+H)⁺

### 6-(5-hydroxy-3-methyl-4-(pyridin-4-yl)-1H-pyrazol-1-yl)nicotinic acid

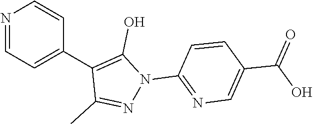 [SMILES: Cc1nn(-c2ccc(C(=O)O)cn2)c(O)c1-c1ccncc1]

The compound was synthesized according to the procedure for the preparation of 6-(4-(4-cyano-3-fluorophenyl)-5-hydroxy-3-methyl-1H-pyrazol-1-yl)nicotinic acid using ethyl 3-oxo-2-(pyridin-4-yl)butanoate. LC-MS (ESI+): m/z 297 (M+H)⁺; ¹H-NMR (300 MHz, CD₃OD) δ 9.00 (s, 1H), 8.24-8.33 (m, 4H), 7.84 (d, J=6.0 Hz, 2H), 2.44 (s, 3H)

### 4-(1-(5-carboxypyridin-2-yl)-5-hydroxy-3-methyl-1H-pyrazol-4-yl)pyridine 1-oxide

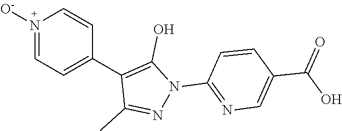

A solution of 6-(5-hydroxy-3-methyl-4-(pyridin-4-yl)-1H-pyrazol-1-yl)nicotinic acid (190 mg, 0.64 mmol) in DMF (10 mL) at rt was added m-CPBA (166 mg, 096 mmol) in one portion. The resulting mixture was stirred at rt for about 2 hrs. After the reaction was completed as indicated by HPLC analysis, the reaction mixture was directly purified by preparative HPLC to give 50 mg of the title compound. LC-MS (ESI+): m/z 313 (M+H)⁺; ¹H-NMR (300 MHz, CD₃OD) δ9.03 (d, J=2.1 Hz, 1H), 8.61 (d, J=6.3 Hz, 1H), 8.45 (m, 1H), 8.07 (d, J=8.7 Hz, 1H), 7.92 (d, J=6.3 Hz, 1H), 2.07 (s, 3H).

### Example 42: Preparation of Compound 42

### methyl 2-(3-bromo-4-chlorophenyl)acetate

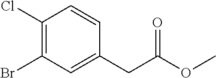

The compound was synthesized according to the procedure for the preparation of methyl 2-(4-bromo-2-methoxyphenyl)acetate using 2-(3-bromo-4-chlorophenyl)acetic acid. ¹H-NMR (300 MHz, CDCl₃) δ 7.55 (d, J=1.8 Hz, 1H), 7.40 (d, J=8.1 Hz, 1H), 7.17 (dd, J=8.1, 1.8 Hz, 1H), 3.71 (s, 3H), 3.58 (s, 2H).

### methyl 2-(4-chloro-3-cyanophenyl)acetate

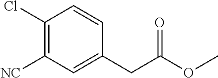

The compound was synthesized according to the procedure for the preparation of methyl 2-(4-cyano-2-fluorophenyl)acetate using methyl 2-(3-bromo-4-chlorophenyl)acetate. ¹H-NMR (300 MHz, CDCl₃) δ 7.61 (s, 1H), 7.48 (s, 2H), 3.73 (s, 3H), 3.64 (s, 2H).

### methyl 2-(4-chloro-3-cyanophenyl)-3-oxobutanoate

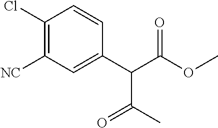 [SMILES: COC(=O)C(C(C)=O)c1ccc(Cl)c(C#N)c1]

The compound was synthesized according to the procedure for the preparation of methyl 2-(4-cyano-3-fluorophenyl)-3-oxobutanoate using methyl 2-(4-chloro-3-cyanophenyl)acetate. The crude product was used for next step directly without further treatment.

### 6-(4-(4-chloro-3-cyanophenyl)-5-hydroxy-3-methyl-1H-pyrazol-1-yl)nicotinic acid

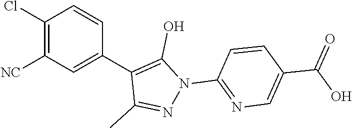 [SMILES: Cc1nn(-c2ccc(C(=O)O)cn2)c(O)c1-c1ccc(Cl)c(C#N)c1]

The compound was synthesized according to the procedure for the preparation of 6-(4-(4-cyano-3-fluorophenyl)-5-hydroxy-3-methyl-1H-pyrazol-1-yl)nicotinic acid using methyl 2-(4-chloro-3-cyanophenyl)-3-oxobutanoate. LC-MS (ESI+): m/z 355 (M+H)⁺; ¹H-NMR (300 MHz, DMSO-d₆) δ8.95 (s, 1H), 8.57 (d, J=6.9 Hz, 1H), 8.43 (m, 1H), 8.22 (s, 1H), 8.03 (d, J=9.0 Hz, 1H), 7.75 (d, J=9.0 Hz, 1H), 1.91 (s, 3H).

### Example 43: Preparation of Compound 43

### 2-(6-Oxo-1,6-dihydropyridin-3-yl)acetic acid

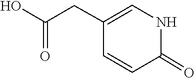 [SMILES: O=C(O)Cc1ccc(=O)[nH]c1]

A mixture of 2-(6-chloropyridin-3-yl)acetic acid (1.0 g, 6.4 mmol) in glacial acetic acid (13.0 mL) and water (3.0 mL) was stirred at 150° C. in a sealed tube for 3.0 d. The solution was cooled and evaporated to give a residue. The residue was re-evaporated from toluene to obtain 2-(6-oxo-1,6-dihydropyridin-3-yl)acetic acid (1.1 g, crude) as brown solid. LC-MS: m/z=154.1 [M+H]⁺, retention time 1.63 min (Method A). The crude product was used to the next step.

### Methyl 2-(6-oxo-1,6-dihydropyridin-3-yl)acetate

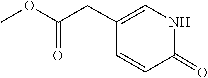

To a solution of 2-(6-oxo-1,6-dihydropyridin-3-yl)acetic acid (1.1 g, crude) in methanol (20.0 mL) was added concentrated sulfuric acid (0.5 mL). The mixture was stirred at room temperature for 15 h and concentrated. The residue was partitioned between ethyl acetate and saturated sodium hydrocarbonate solution. The organic layer was washed with brine, dried over sodium sulfate and concentrated under reduced pressure to provide methyl 2-(6-oxo-1,6-dihydropyridin-3-yl)acetate (800 mg, 4.79 mmol, 74.8% yield) as white solid. LC-MS: m/z=168.1 [M+H]⁺, retention time 1.35 min (Method A).

### Methyl 2-(6-methoxypyridin-3 yl)acetate

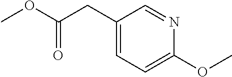

To a solution of methyl 2-(6-oxo-1,6-dihydropyridin-3-yl)acetate (450 mg, 2.69 mmol) and cesium carbonate (1.05 g, 3.23 mmol) in anhydrous tetrahydrofuran (20.0 mL) was added iodomethane (580 mg, 4.20 mmol). The mixture was stirred at room temperature for 18 h. The reaction solution was diluted with ethyl acetate and water. The organic layer was washed with brine, dried over sodium sulfate and concentrated. The residue was purified by flash chromatography (petroleum ether/ethyl acetate=3/1) to afford a mixture of methyl 2-(1-methyl-6-oxo-1,6-dihydropyridin-3-yl)acetate and methyl 2-(6-methoxypyridin-3-yl)acetate (400 mg, 2.21 mmol, 82.1% yield) as yellow oil. LC-MS: m/z=182.1 (M+H)⁺, retention time 1.33 min (Method A).

### Methyl (E)-3-(dimethylamino)-2-(6-methoxypyridin-3-yl)acrylate

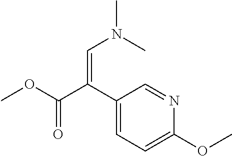

To a solution of methyl 2-(1-methyl-6-oxo-1,6-dihydropyridin-3-yl)acetate and methyl 2-(6-methoxypyridin-3-yl)acetate (400 mg, 2.21 mmol) in N,N-dimethylformamide (5.0 mL) was added N,N-dimethylformamide diethyl acetal (1.63 g, 11.1 mmol). The mixture was stirred at 100° C. overnight and cooled. Ethyl acetate and water were added to the solution, and the layers were separated. The organic layer was washed with brine, dried over sodium sulfate and concentrated to give a mixture of methyl (E)-3-(dimethylamino)-2-(1-methyl-6-oxo-1,6-dihydropyridin-3-yl)acrylate and methyl (E)-3-(dimethylamino)-2-(6-methoxypyridin-3-yl)acrylate (400 mg, 1.69 mmol, 76.7% yield) as yellow oil. LC-MS: m/z=237.1 [M+H]⁺, retention time 1.08 min, 1.51 min (Method B). The mixture was used to the next step.

### Tert-butyl 6-(5-hydroxy-4-(6-methoxypyridin-3-yl)-1H-pyrazol-1-yl)nicotinate

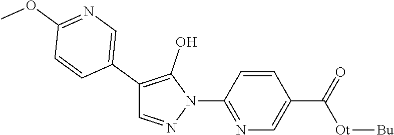

A mixture of methyl (E)-3-(dimethylamino)-2-(1-methyl-6-oxo-1,6-dihydropyridin-3-yl)acrylate and methyl (E)-3-(dimethylamino)-2-(6-methoxypyridin-3-yl)acrylate (350 mg, 1.48 mmol) in ethanol (5.0 mL) was stirred at 90° C. in a sealed tube for 16.0 h and cooled. The insoluble solid was filtered and the filtrate was concentrated to dryness. The two isomers were separated by reverse prep-HPLC as white solid. Tert-butyl 6-(5-hydroxy-4-(6-methoxypyridin-3-yl)-1H-pyrazol-1-yl)nicotinate (76 mg, 0.21 mmol, 27.9% yield). LC-MS: m/z=369.0 [M+H]⁺, retention time 2.29 min (Method A). ¹HNMR (500 MHz, DMSO-d₆) δ 12.97 (br, 1H), 8.91 (d, J=1.5 Hz, 1H), 8.70 (s, 1H), 8.47-8.39 (m, 2H), 8.19-8.14 (m, 1H), 6.85-6.83 (m, 1H), 3.85 (s, 3H), 1.58 (s, 9H).

### 6-(5-Hydroxy-4-(6-methoxypyridin-3-yl)-1H-pyrazol-1-yl)nicotinic acid

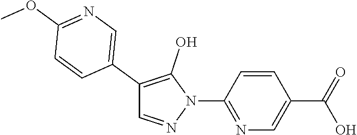 [SMILES: COc1ccc(-c2cnn(-c3ccc(C(=O)O)cn3)c2O)cn1]

To a solution of tert-butyl 6-(5-hydroxy-4-(6-methoxypyridin-3-yl)-1H-pyrazol-1-yl)nicotinate (76 mg, 0.21 mmol) in dichloromethane (6.0 mL) was added trifluoroacetic acid (3.0 mL). The mixture was stirred at room temperature overnight and concentrated. The residue was triturated with ethyl acetate and filtered to afford 6-(5-hydroxy-4-(6-methoxypyridin-3-yl)-1H-pyrazol-1-yl)nicotinic acid (5.4 mg, 0.017 mmol, 8.2% yield) as white solid. LC-MS: m/z=313.0 [M+H]⁺, retention time 3.73 min (Method A). ¹HNMR (400 MHz, DMSO-d₆) (13.47 (br, 1H), 13.06 (br, 1H), 8.96 (s, 1H), 8.73-8.44 (m, 4H), 8.21-8.18 (m, 1H), 6.84 (d, J=8.8 Hz, 1H), 3.85 (s, 3H).

### Example 44: Preparation of Compound 44

### diethyl 2-(3-chloro-4-cyanophenyl)malonate

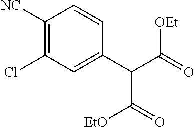 [SMILES: CCOC(=O)C(C(=O)OCC)c1ccc(C#N)c(Cl)c1]

The compound was synthesized according to the procedure for the preparation of diethyl 2-(2-bromo-4-cyanophenyl)malonate using 2-chloro-4-fluorobenzonitrile. ¹H-NMR (300 MHz, CDCl₃) δ 7.68 (d, J=8.1 Hz, 1H), 7.61 (d, J=1.2 Hz, 1H), 7.45 (dd, J=8.1, 1.2 Hz, 1H), 4.63 (s, 1H), 4.30 (q, J=6.9 Hz, 4H), 1.26 (t, J=6.9 Hz, 6H).

### ethyl 2-(3-chloro-4-cyanophenyl)acetate

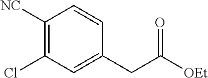 [SMILES: CCOC(=O)Cc1ccc(C#N)c(Cl)c1]

The compound was synthesized according to the procedure for the preparation of ethyl 2-(2-bromo-4-cyanophenyl)acetate using diethyl 2-(3-chloro-4-cyanophenyl)malonate. ¹H-NMR (300 MHz, CDCl₃) δ 7.63 (d, J=8.1 Hz, 1H), 7.47 (d, J=1.2 Hz, 1H), 7.27 (dd, J=8.1, 1.2 Hz, 1H), 4.22 (q, J=6.9 Hz, 2H), 3.66 (s, 2H), 1.26 (t, J=6.9 Hz, 3H).

### ethyl (E)-3-acetoxy-2-(3-chloro-4-cyanophenyl)but-2-enoate

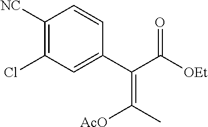 [SMILES: CCOC(=O)/C(=C(\C)OC(C)=O)c1ccc(C#N)c(Cl)c1]

The compound was synthesized according to the procedure for the preparation of 6-(4-(4-cyano-2-fluorophenyl)-5-hydroxy-3-methyl-1H-pyrazol-1-yl)nicotinic acid using ethyl 2-(3-chloro-4-cyanophenyl)acetate. LC-MS (ESI+): m/z 330 (M+Na)⁺.

### 6-(4-(3-chloro-4-cyanophenyl)-5-hydroxy-3-methyl-1H-pyrazol-1-yl)nicotinic acid

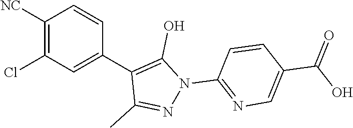 [SMILES: Cc1nn(-c2ccc(C(=O)O)cn2)c(O)c1-c1ccc(C#N)c(Cl)c1]

The compound was synthesized according to the procedure for the preparation of 6-(4-(4-cyano-3-fluorophenyl)-5-hydroxy-3-methyl-1H-pyrazol-1-yl)nicotinic acid using ethyl (E)-3-acetoxy-2-(3-chloro-4-cyanophenyl)but-2-enoate. LC-MS (ESI+): m/z 355 (M+H)⁺; ¹H-NMR (300 MHz, DMSO-d₆) δ13.35 (brs, 2H), 8.95 (s, 1H), 8.57 (d, J=9.0 Hz, 1H), 8.42 (d, J=9.0, 1.8 Hz, 1H), 8.14 (s, 1H), 7.92 (d, J=8.4 Hz, 1H), 7.82 (d, J=8.4 Hz, 1H), 2.53 (s, 3H).

### Example 45: In Vitro Assays Demonstrate PHD Inhibition

Enzymatic half maximal inhibitory concentration (IC₅₀) values were determined on selected compounds of the invention.

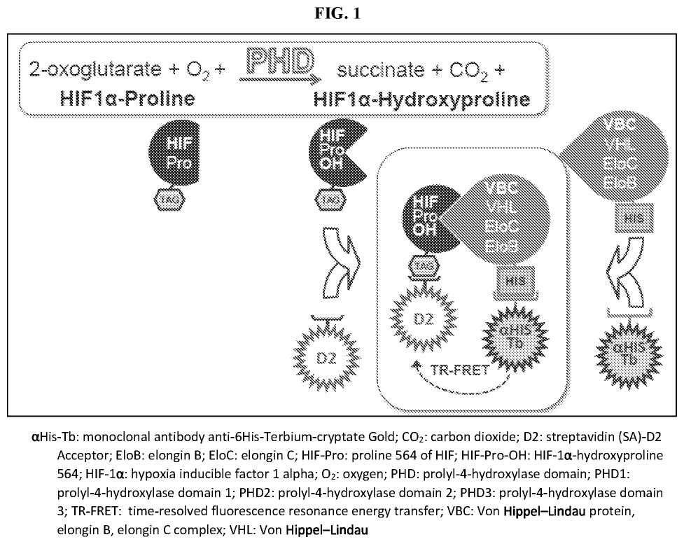

Time-resolved fluorescence resonance energy transfer (TR-FRET) assay was utilized to determine the enzymatic half maximal inhibitory concentration (IC₅₀) value of PHD inhibitors against the full-length human prolyl-4-hydroxylase domain (PHD) enzymes, PHD1, PHD2, and PHD3. The TR-FRET assay was developed based on the specific binding of hydroxylated HIF-1α peptide with the complex formed by VHL, EloB and EloC (VBC), to generate a fluorescent signal. Terbium (Tb)-Donor (monoclonal antibody anti-6His-Tb-cryptate Gold) and D2-acceptor (streptavidin [SA]-D2) of TR-FRET are linked to the VBC complex and to HIF-1α peptide, respectively. The VBC complex binds specifically to the HIF-1α peptide when it is hydroxylated, allowing energy transfer from TR-FRET donor to acceptor (FIG. 1).

### Materials and Methods

All chemicals and materials unless otherwise noted were of standard laboratory grade and were purchased from Sigma-Aldrich (St. Louis, Mo., USA).

### Reagents

TR-FRET Reagents

Monoclonal antibody anti-6His-Tb-cryptate Gold (catalog #61HI2TLA) and streptavidin (SA)-D2 (catalog #610SADLA) were purchased from CisBio International (Bedford, Mass., USA).

N-terminus biotinylated HIF-1a C35 synthetic peptide representing amino acids 547 to 581 and including the proline 564 PHD2 hydroxylation site was purchased from California Peptide Research (Salt Lake City, Utah, USA).

### Recombinant Proteins

VBC complex

His-tagged recombinant VHL protein, EloB, EloC complex (His-VBC) was supplied by Axxam (Milan, Italy). Recombinant human VHL (National Center for Biotechnology Information [NCBI] accession number NP_00542.1) contained a His tag at the C-terminus of amino acids 55 to 213 and is referred to as VHL-His. VHL-His was co-expressed in E. coli with full-length human EloB (NCBI accession number Q15370.1) and full-length human EloC (NCBI accession number Q15369.1) and purified by affinity chromatography on a nickel-nitrilotriacetic acid (Ni-NTA) column as the His-VBC complex. Purity (˜80%) was assessed by sodium dodecyl sulfate polyacrylamide gel electrophoresis (SDS-PAGE).

PHD1

Recombinant human PHD1 protein (catalog #81064, Lot #24717001) was purchased from Active Motif (Carlsbad, Calif., USA). PHD1 was expressed in a baculovirus expression system as the full-length protein (NCBI accession number NP_542770.2) with an N-terminal FLAG tag (molecular weight 44.9 kDa). Purity (>90%) was assessed by SDS-PAGE.

PHD2

The full-length human PHD2 enzyme was produced with a baculovirus infected insect cell (BIIC) expression system by Beryllium (Bedford, Mass., USA). The PHD2 construct contained amino acids 1 to 426 of PHD2 (UniProt Knowledgebase[UniProtKB]/Swiss-Prot accession number Q9GZT9.1), and a His tag and a Tobacco Etch Virus (TEV) protease cleavage site at the N-terminus. The construct was expressed in Sf9 insect cells, purified by Ni-NTA column and digested with TEV protease to remove the His tag. The purity of final cleaved protein was assessed by SDS-PAGE and was found to be >94% pure.

PHD3

Recombinant human PHD3 protein (molecular weight 31.1 kDa) was purchased from Active Motif (Carlsbad, Calif., USA). It was expressed in E. coli as the full-length protein (NCBI accession number NP_071356.1) with an N-terminal 6-His tag (catalog #81033, Lot #24417001). Purity was assessed by SDS-PAGE and was found to be >75% pure.

PHD Inhibitors.

Small molecule PHD inhibitors were synthesized and their identities were confirmed as described herein.

TR-FRET Assay Procedure

PHD inhibitor compound was preincubated with PHD enzyme in a 10 μL reaction volume in white 384-well Optiplate microplates (catalog #6007290, Perkin Elmer, Waltham, Mass., USA). For this, 5 μL PHD inhibitor compound was serially diluted with dilution buffer (50 mM HEPES [4-(2-hydroxyethyl)-1-piperazineethanesulfonic acid] pH 7.5, 50 mM sodium chloride [NaCl], 0.01% Tween-20, 0.01% purified bovine serum albumin [BSA]) and mixed with 5 μL PHD enzyme mix prepared as a 4× concentrate in the dilution buffer containing PHD enzyme (60 nM PHD1, 20 nM PHD2, 140 nM PHD3), 40 μM ferrous ammonium sulfate (FAS), 4 mM sodium (Na) ascorbate. The plates were incubated for 30 minutes at room temperature without rotation.

Five microliters of the VBC/anti-6His-Tb-cryptate Gold mix prepared as a 4× concentrate in dilution buffer containing 20 nM His-VBC, 1.32 nM monoclonal antibody anti-6His-Tb-cryptate Gold was then added. This step was followed immediately by the addition of 5 μL of the HIF-1α C35 substrate mix prepared as a 4× concentrate in the dilution buffer containing 120 nM biotin-labeled HIF-1α C35, 132 nM SA-D2, 4 μM 2-oxoglutarate (2-OG) to reach a final reaction volume of 20 μL.

The final assay reaction contained 50 mM HEPES, pH 7.5, 50 mM NaCl, 1 μM 2-OG, 10 μM FAS, 1 mM Na ascorbate, 0.01% Tween-20, 0.01% purified BSA, 30 nM biotin-labeled HIF-1α C35, 5 nM His-VBC, 0.33 nM monoclonal antibody anti-6His-Tb-cryptate Gold, 33 nM SA-D2 and PHD enzyme (15 nM PHD1, 5 nM PHD2, or 35 nM PHD3) with the diluted compound.

For the measurement of the IC₅₀ of PHD inhibitor compound, reactions were incubated for 10 minutes at room temperature and then read on a Perkin Elmer EnVision (Waltham, Mass., USA) at an excitation wavelength of 340 nm and at emission wavelengths of 615 nm and 665 nm. The data represent the quotient of the signal intensity at 665 nm and 615 nm, automatically calculated by Envision Manager software (Perkin Elmer, Waltham, Mass., USA). The IC₅₀ values (mean, standard deviation, standard error of the mean, geometric mean and 95% confidence interval) were determined using a four-parameter curve-fit using GraphPad Prism 7.0 (GraphPad, La Jolla, Calif., USA) and represent the compound concentration plotted against the calculated ratio of 665 nm and 615 nm. TR-FRET assays were performed in triplicate at each concentration of compound and the assays were repeated independently three times.

Kis were calculated from IC₅₀s based on the Cheng Prussoff equation:

Ki=IC50/(1+[2-OG]/Km)

The final concentration of 2-OG in both the PHD1 and PHD2 assays is 1 uM. The Km of 2-OG for PHD1 was determined to be 12.7 nM, while the Km of 2-OG for PHD2 was determined to be 22.6 nM.

Exemplary Compounds

| | | PHD1 | | |
|---|---|---|---|---|
| Cmpd. | | IC50 | PHD2 | PHD3 |
| No. | Structure | (nM) | IC5 0(nM) | IC50 (nM) |
| 1 | 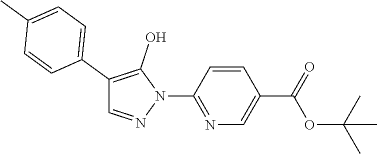 [SMILES: Cc1ccc(-c2cnn(-c3ccc(C(=O)OC(C)(C)C)cn3)c2O)cc1] | B | C | — |
| 2 | 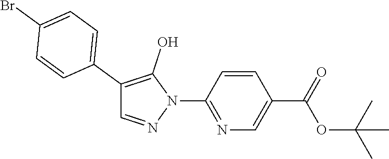 [SMILES: CC(C)(C)OC(=O)c1ccc(-n2ncc(-c3ccc(Br)cc3)c2O)nc1] | B | D | — |
| 3 | 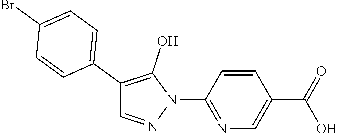 [SMILES: O=C(O)c1ccc(-n2ncc(-c3ccc(Br)cc3)c2O)nc1] | A | A | B |
| 4 | 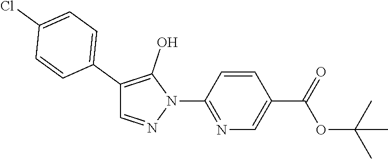 [SMILES: CC(C)(C)OC(=O)c1ccc(-n2ncc(-c3ccc(Cl)cc3)c2O)nc1] | B | C | — |
| 5 | 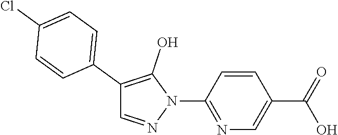 [SMILES: O=C(O)c1ccc(-n2ncc(-c3ccc(Cl)cc3)c2O)nc1] | A | A | B |
| 6 | 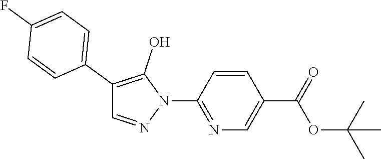 [SMILES: CC(C)(C)OC(=O)c1ccc(-n2ncc(-c3ccc(F)cc3)c2O)nc1] | A | C | — |
| 7 | 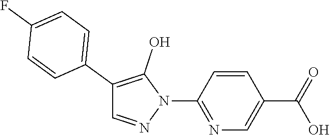 [SMILES: O=C(O)c1ccc(-n2ncc(-c3ccc(F)cc3)c2O)nc1] | A | A | C |
| 8 | 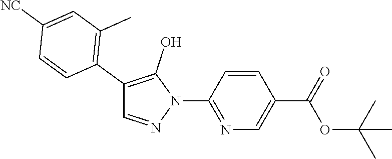 [SMILES: Cc1cc(C#N)ccc1-c1cnn(-c2ccc(C(=O)OC(C)(C)C)cn2)c1O] | A | A | A |
| 9 | 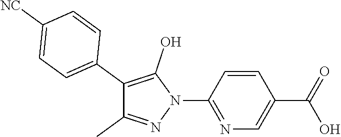 [SMILES: Cc1nn(-c2ccc(C(=O)O)cn2)c(O)c1-c1ccc(C#N)cc1] | A | A | A |
| 10 | 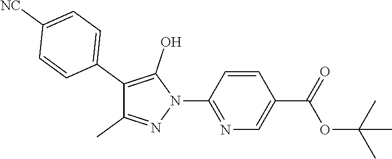 [SMILES: Cc1nn(-c2ccc(C(=O)OC(C)(C)C)cn2)c(O)c1-c1ccc(C#N)cc1] | A | A | D |
| 11 | 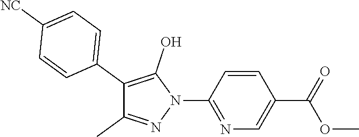 [SMILES: COC(=O)c1ccc(-n2nc(C)c(-c3ccc(C#N)cc3)c2O)nc1] | A | A | B |
| 12 | 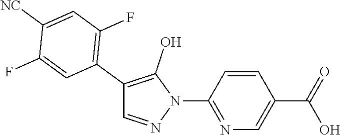 [SMILES: N#Cc1cc(F)c(-c2cnn(-c3ccc(C(=O)O)cn3)c2O)cc1F] | A | A | B |
| 13 | 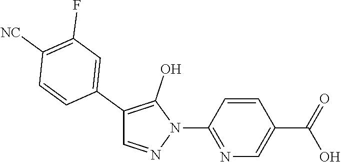 [SMILES: N#Cc1ccc(-c2cnn(-c3ccc(C(=O)O)cn3)c2O)cc1F] | A | A | A |
| 14 | 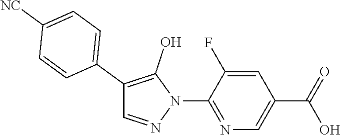 [SMILES: N#Cc1ccc(-c2cnn(-c3ncc(C(=O)O)cc3F)c2O)cc1] | C | C | — |
| 15 | 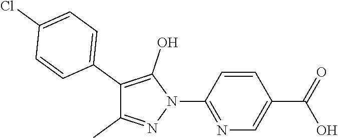 [SMILES: Cc1nn(-c2ccc(C(=O)O)cn2)c(O)c1-c1ccc(Cl)cc1] | A | A | — |
| 16 | 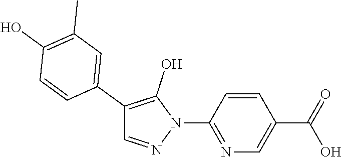 [SMILES: Cc1cc(-c2cnn(-c3ccc(C(=O)O)cn3)c2O)ccc1O] | B | B | C |
| 17 | 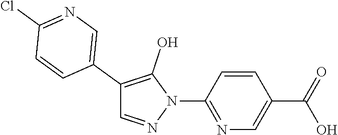 [SMILES: O=C(O)c1ccc(-n2ncc(-c3ccc(Cl)nc3)c2O)nc1] | A | A | A |
| 18 | 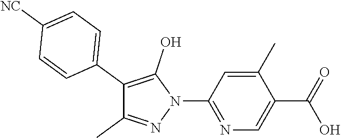 [SMILES: Cc1cc(-n2nc(C)c(-c3ccc(C#N)cc3)c2O)ncc1C(=O)O] | A | A | A |
| 19 | 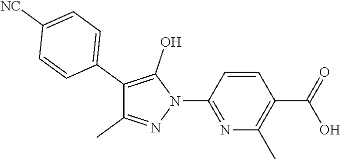 [SMILES: Cc1nc(-n2nc(C)c(-c3ccc(C#N)cc3)c2O)ccc1C(=O)O] | C | C | — |
| 20 | 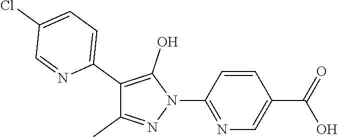 [SMILES: Cc1nn(-c2ccc(C(=O)O)cn2)c(O)c1-c1ccc(Cl)cn1] | A | B | — |
| 21 | 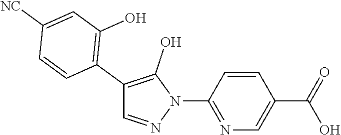 [SMILES: N#Cc1ccc(-c2cnn(-c3ccc(C(=O)O)cn3)c2O)c(O)c1] | A | A | B |
| 22 | 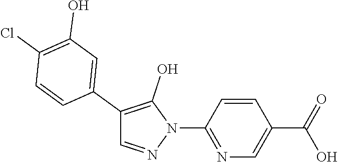 [SMILES: O=C(O)c1ccc(-n2ncc(-c3ccc(Cl)c(O)c3)c2O)nc1] | A | A | — |
| 23 | 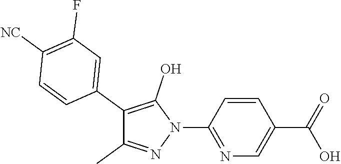 [SMILES: Cc1nn(-c2ccc(C(=O)O)cn2)c(O)c1-c1ccc(C#N)c(F)c1] | A | A | — |
| 24 | 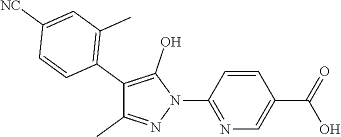 [SMILES: Cc1cc(C#N)ccc1-c1c(C)nn(-c2ccc(C(=O)O)cn2)c1O] | A | A | A |
| 25 | 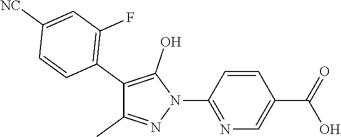 [SMILES: Cc1nn(-c2ccc(C(=O)O)cn2)c(O)c1-c1ccc(C#N)cc1F] | A | A | — |
| 26 | 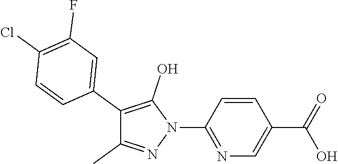 [SMILES: Cc1nn(-c2ccc(C(=O)O)cn2)c(O)c1-c1ccc(Cl)c(F)c1] | A | A | B |
| 27 | 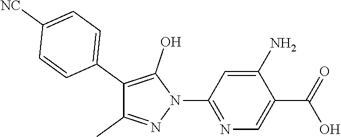 [SMILES: Cc1nn(-c2cc(N)c(C(=O)O)cn2)c(O)c1-c1ccc(C#N)cc1] | A | A | — |
| 28 | 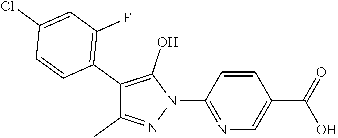 [SMILES: Cc1nn(-c2ccc(C(=O)O)cn2)c(O)c1-c1ccc(Cl)cc1F] | A | B | B |
| 29 | 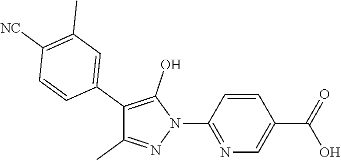 [SMILES: Cc1cc(-c2c(C)nn(-c3ccc(C(=O)O)cn3)c2O)ccc1C#N] | A | A | — |
| 30 | 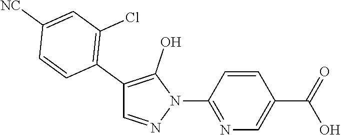 [SMILES: N#Cc1ccc(-c2cnn(-c3ccc(C(=O)O)cn3)c2O)c(Cl)c1] | A | A | — |
| 31 | 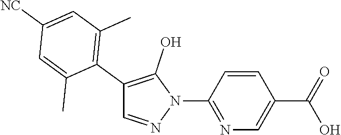 [SMILES: Cc1cc(C#N)cc(C)c1-c1cnn(-c2ccc(C(=O)O)cn2)c1O] | A | B | — |
| 32 | 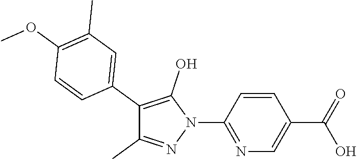 [SMILES: COc1ccc(-c2c(C)nn(-c3ccc(C(=O)O)cn3)c2O)cc1C] | B | B | — |
| 33 | 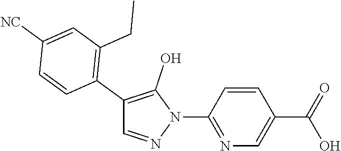 [SMILES: CCc1cc(C#N)ccc1-c1cnn(-c2ccc(C(=O)O)cn2)c1O] | A | A | — |
| 34 | 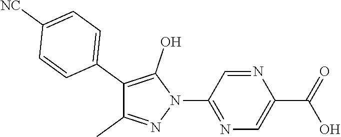 [SMILES: Cc1nn(-c2cnc(C(=O)O)cn2)c(O)c1-c1ccc(C#N)cc1] | A | A | — |
| 35 | 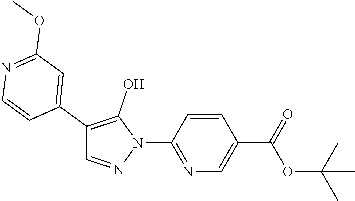 [SMILES: COc1cc(-c2cnn(-c3ccc(C(=O)OC(C)(C)C)cn3)c2O)ccn1] | A | A | A |
| 36 | 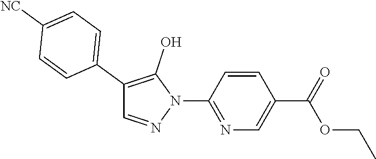 [SMILES: CCOC(=O)c1ccc(-n2ncc(-c3ccc(C#N)cc3)c2O)nc1] | A | A | A |
| 37 | 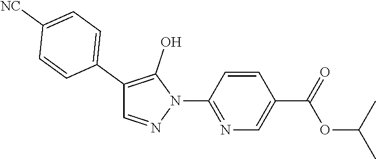 [SMILES: CC(C)OC(=O)c1ccc(-n2ncc(-c3ccc(C#N)cc3)c2O)nc1] | A | A | A |
| 38 | 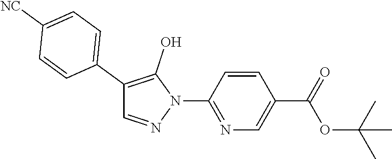 [SMILES: CC(C)(C)OC(=O)c1ccc(-n2ncc(-c3ccc(C#N)cc3)c2O)nc1] | A | A | C |
| 39 | 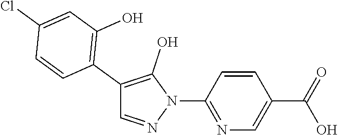 [SMILES: O=C(O)c1ccc(-n2ncc(-c3ccc(Cl)cc3O)c2O)nc1] | B | A | C |
| 40 | 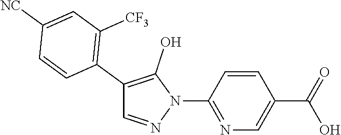 [SMILES: N#Cc1ccc(-c2cnn(-c3ccc(C(=O)O)cn3)c2O)c(C(F)(F)F)c1] | A | A | — |
| 41 | 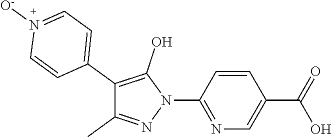 | D | D | — |
| 42 | 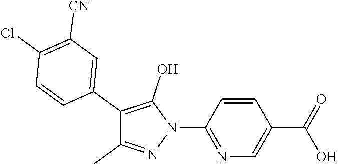 [SMILES: Cc1nn(-c2ccc(C(=O)O)cn2)c(O)c1-c1ccc(Cl)c(C#N)c1] | A | A | — |
| 43 | 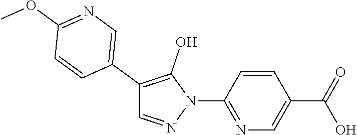 [SMILES: COc1ccc(-c2cnn(-c3ccc(C(=O)O)cn3)c2O)cn1] | A | A | — |
| 44 | 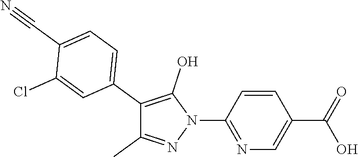 [SMILES: Cc1nn(-c2ccc(C(=O)O)cn2)c(O)c1-c1ccc(C#N)c(Cl)c1] | A | A | — |

Legend: A = IC50 < 100 nM
B = 100 nM ≤ IC50 < 1,000 nM
C = 1,000 nM ≤ IC50 < 10,000 nM
D = IC50 ≥ 10,000 nM

From the ongoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

1. A compound of Formula A: 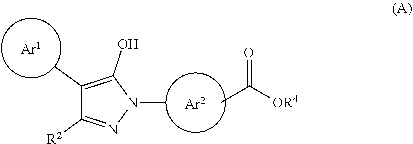
or a pharmaceutically acceptable salt thereof, wherein:
Ar¹ is phenyl or a six-membered nitrogen-containing heteroaryl, wherein said phenyl or heteroaryl is optionally substituted with halogen, CN, OH, C₁₋₃ alkyl optionally substituted with one or more halogens, or C₁₋₃ alkoxy;
R² is H or C₁₋₃ alkyl;
Ar² is a six-membered nitrogen-containing heteroaryl, optionally substituted with halogen, OH, amine, or C₁₋₃ alkyl;
R⁴ is hydrogen or C₁₋₄ alkyl; and
wherein Formula (A) excludes the following compounds:

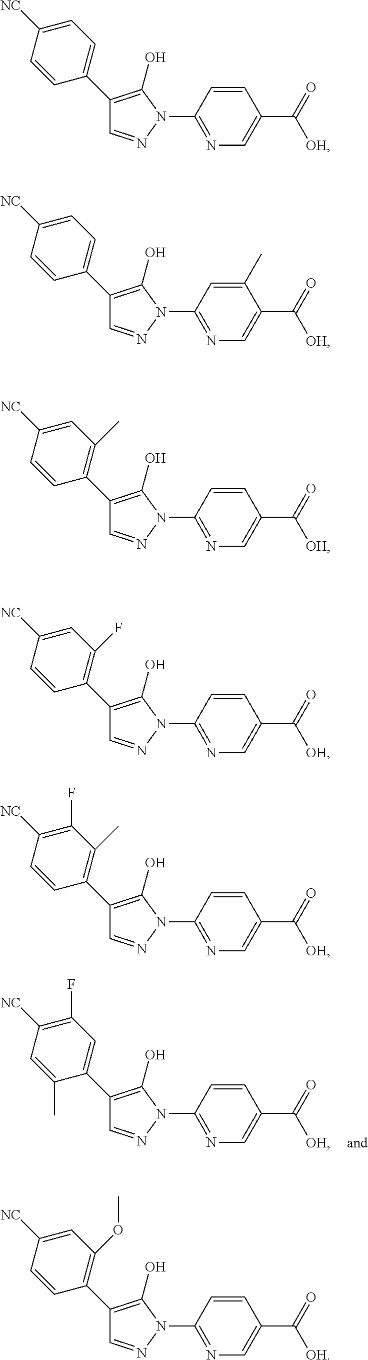

2. The compound of claim 1, wherein Ar¹ is 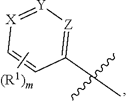
wherein
X, Y, and Z are independently CH or N, wherein N is optionally oxidized;
each R¹ is independently selected from the group consisting of hydrogen, halogen, CN, OH, C₁₋₃ alkyl optionally substituted with one or more halogens, and C₁₋₃ alkoxy; and
m is 1, 2, 3, or 4.

3. The compound of claim 1 or 2, wherein Ar¹ is a phenyl substituted by at least one R¹ that is CN or halogen.

4. The compound of claim 3, wherein Ar¹ is substituted by one or two R¹ groups independently selected from C₁₋₃ alkyl optionally substituted with one or more halogens, halogen, CN or OH.

5. The compound of claim 1 or 2, wherein Ar¹ is a pyridyl N-oxide or is a pyridyl optionally substituted by at least one R¹ that is C₁₋₃ alkoxy or halogen.

6. The compound of any one of claims 1-5, wherein Ar² is 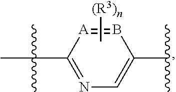
wherein
A and B are independently CH or N, wherein N is optionally oxidized;
each R³ is independently selected from the group consisting of hydrogen, halogen, OH, amine, and C₁₋₃ alkyl; and
n is 0, 1, or 2.

7. The compound of any one of claims 1-6, wherein Ar² is a group that is pyridyl or pyrazinyl, and wherein said group is unsubstituted or comprises a substituent that is halogen, C₁₋₃ alkyl, or OH.

8. The compound of any one of claims 1-7, wherein R² is H or CH₃.

9. The compound of any one of claims 1-8, wherein R⁴ is H.

10. The compound of any one of claims 1-8, wherein R⁴ is C₁₋₄ alkyl.

11. The compound of claim 1, having a structure according to Formula (I): 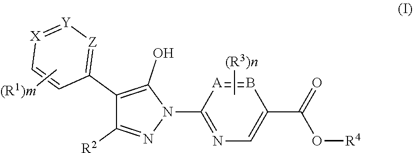
or a pharmaceutically acceptable salt thereof, wherein:
X, Y, Z, A and B are independently CH or N, wherein N is optionally oxidized;
m is 1, 2, 3, or 4;
n is 0, 1, or 2;
each R¹ is independently selected from the group consisting of hydrogen, halogen, CN, OH, C₁₋₃ alkyl optionally substituted with one or more halogens, and C₁₋₃ alkoxy;
R² is hydrogen or C₁₋₃ alkyl;
each R³ is independently selected from the group consisting of hydrogen, halogen, OH, amine, and C₁₋₃ alkyl; and
R⁴ is hydrogen or C₁₋₄ alkyl.

12. The compound of claim 11, having the structure of Formula (Ia): 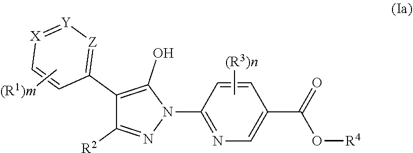
or a pharmaceutically acceptable salt thereof.

13. The compound of claim 11, having the structure of Formula (Ib): 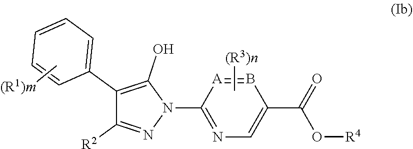
or a pharmaceutically acceptable salt thereof.

14. The compound of claim 11, having the structure of Formula (Ic): 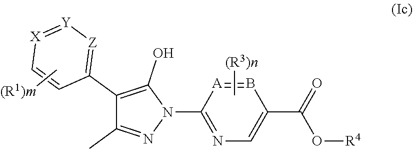
or a pharmaceutically acceptable salt thereof.

15. The compound of claim 1, having a structure according to Formula (II): 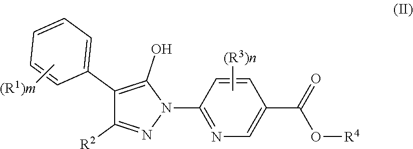
or a pharmaceutically acceptable salt thereof, wherein:
m is 1, 2, 3, or 4;
n is 0, 1, or 2;
R¹, each time taken, is independently selected from the group consisting of hydrogen, halogen, CN, OH, C₁₋₃ alkyl optionally substituted with one or more halogens, and C₁₋₃ alkoxy;
R² is hydrogen or C₁₋₃ alkyl;
R³, each time taken, is independently selected from the group consisting of hydrogen, halogen, OH, amine, or C₁₋₃ alkyl; and
R⁴ is hydrogen or C₁₋₄ alkyl.

16. The compound of claim 15, having the structure of Formula (IIa): 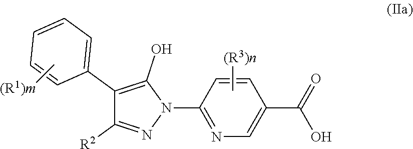
or a pharmaceutically acceptable salt thereof

17. The compound of claim 15, having the structure of Formula (IIb): 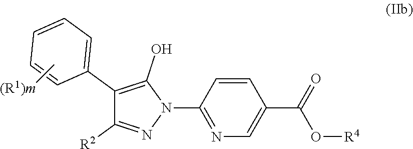
or a pharmaceutically acceptable salt thereof.

18. The compound of claim 15, having the structure of Formula (IIc): 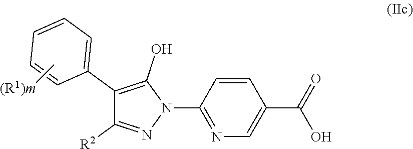
or a pharmaceutically acceptable salt thereof.

19. The compound of claim 15, having the structure of Formula (IId): 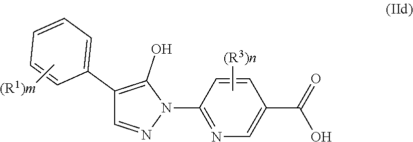
or a pharmaceutically acceptable salt thereof.

20. The compound of claim 15, having the structure of Formula (IIe): 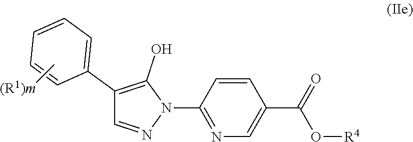
or a pharmaceutically acceptable salt thereof.

21. The compound of claim 15, having the structure of Formula (IIf): 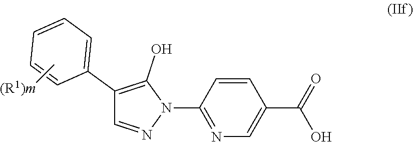
or a pharmaceutically acceptable salt thereof.

22. The compound of claim 15, having the structure of Formula (IIg): 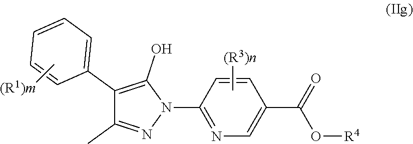
or a pharmaceutically acceptable salt thereof.

23. The compound of claim 1, having a structure according to Formula III: 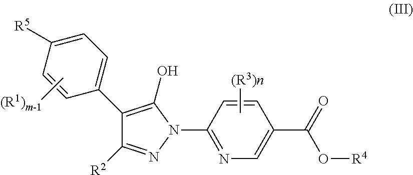
or a pharmaceutically acceptable salt thereof, wherein:
m is 1, 2, 3, or 4;
n is 0, 1, or 2;
R¹, each time taken, is independently selected from the group consisting of hydrogen, halogen, OH, and C₁₋₃ alkyl optionally substituted with one or more halogens;
R² is hydrogen or C₁₋₃ alkyl;
R³, each time taken, is independently selected from the group consisting of hydrogen, halogen, OH, amine, and C₁₋₃ alkyl;
R⁴ is hydrogen or C₁₋₄ alkyl;
R⁵ is CN or halogen.

24. The compound of claim 23, having the structure of Formula (IIIa): 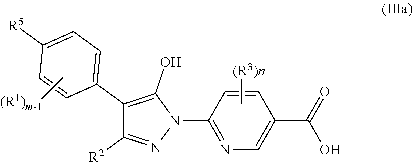
or a pharmaceutically acceptable salt thereof.

25. The compound of claim 23, having the structure of Formula (IIIb): 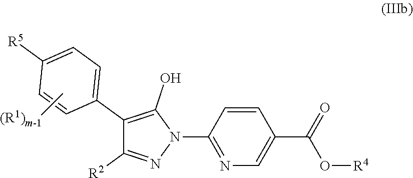
or a pharmaceutically acceptable salt thereof.

26. The compound of claim 23, having the structure of Formula (IIIc): 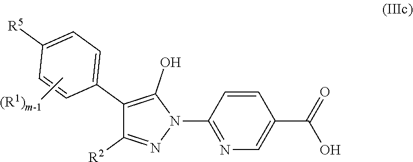
or a pharmaceutically acceptable salt thereof.

27. The compound of claim 23, having the structure of Formula (IIId) 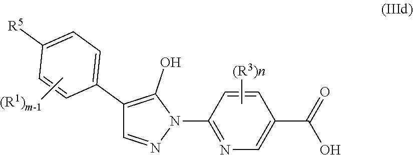
or a pharmaceutically acceptable salt thereof.

28. The compound of claim 23, having the structure of Formula (IIIe): 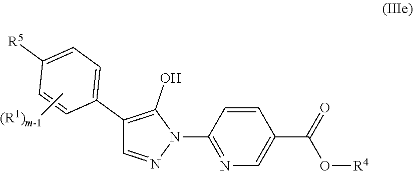
or a pharmaceutically acceptable salt thereof.

29. The compound of claim 23, having the structure of Formula (IIIf): 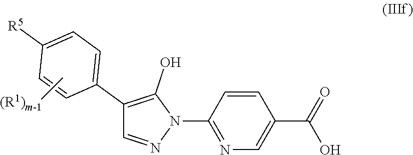
or a pharmaceutically acceptable salt thereof.

30. The compound of claim 23, having the structure of Formula III(g): 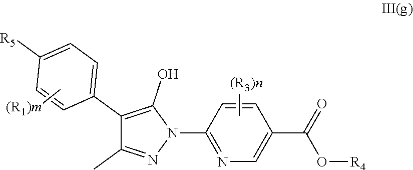
or a pharmaceutically acceptable salt thereof.

31. The compound of claim 11, 12, or 14, wherein X is CH.

32. The compound of claim 11, 12, or 14, wherein X is N.

33. The compound of claim 32, wherein N is optionally oxidized.

34. The compound of claim 11, 12, or 14, wherein Y is CH.

35. The compound of claim 11, 12, or 14, wherein Y is N.

36. The compound of claim 11, 12, or 14, wherein Z is CH.

37. The compound of claim 11, 12, or 14, wherein Z is N.

38. The compound of claim 11, 12, or 14, wherein A is CH.

39. The compound of claim 11, 12, or 14, wherein A is N.

40. The compound of claim 11, 12, or 14, wherein B is CH.

41. The compound of claim 11, 12, or 14, wherein B is N.

42. The compound of any one of claims 11-30, wherein m is 1.

43. The compound of any one of claims 11-30, wherein m is 2.

44. The compound of any one of claims 11-30, wherein m is 3.

45. The compound of any one of claims 11-22, wherein m is 4.

46. The compound of any one of claims 11-16, 19, 22-24, 27 and 30, wherein n is 0.

47. The compound of any one of claims 11-16, 19, 22-24, 27 and 30, wherein n is 1.

48. The compound of any one of claims 11-16, 19, 22-24, 27 and 30, wherein n is 2.

49. The compound of any one of claims 11-30, wherein R¹ is hydrogen.

50. The compound of any one of claims 11-30, wherein R¹ is halogen.

51. The compound of claim 50, wherein R¹ is F.

52. The compound of claim 50, wherein R¹ is Cl.

53. The compound of claim 50, wherein R¹ is Br.

54. The compound of any one of claims 11-30, wherein R¹ is CN.

55. The compound of any one of claims 11-30, wherein R¹ is OH.

56. The compound of any one of claims 11-30, wherein R¹ is C₁₋₃ alkyl optionally substituted with one or more halogens.

57. The compound of claim 56, wherein R¹ is C₁₋₃ alkyl.

58. The compound of claim 56, wherein R¹ is methyl.

59. The compound of claim 56 wherein R¹ is ethyl.

60. The compound of claim 56, wherein R¹ is CF₃.

61. The compound of any one of claims 11-30, wherein R¹ is C₁₋₃ alkoxy.

62. The compound of claim 61, wherein R¹ is methoxy.

63. The compound of any one of claims 11-18 and 23-26, wherein R² is hydrogen.

64. The compound of any one of claims 11-18 and 23-26, wherein R² is C₁₋₃ alkyl.

65. The compound of claim 64, wherein R² is methyl.

66. The compound of any one of claims 11-16, 19, 22-24, 27 and 30, wherein R³ is hydrogen.

67. The compound of any one of claims 11-16, 19, 22-24, 27 and 30, wherein R³ is halogen.

68. The compound of claim 56, wherein R³ is F.

69. The compound of any one of claims 11-16, 19, 22-24, 27 and 30, wherein R³ is OH.

70. The compound of any one of claims 11-16, 19, 22-24, 27 and 30, wherein R³ is amine.

71. The compound of claim 70, wherein R³ is NH₂.

72. The compound of any one of claims 11-16, 19, 22-24, 27 and 30, wherein R³ is C₁₋₃ alkyl.

73. The compound of claim 72, wherein R³ is methyl.

74. The compound of any one of claims 11-15, 17, 20, 22, 25, 28, and 30, wherein R⁴ is hydrogen.

75. The compound of any one of claims 11-15, 17, 20, 22, 25, and 30, wherein R⁴ is C₁₋₄ alkyl.

76. The compound of claim 75, wherein R⁴ is methyl.

77. The compound of claim 75, wherein R⁴ is ethyl.

78. The compound of claim 75, wherein R⁴ is isopropyl.

79. The compound of claim 75, wherein R⁴ is tert-butyl.

80. The compound of any one of claims 23-30, wherein R⁵ is F.

81. The compound of any one of claims 23-30, wherein R⁵ is Cl.

82. The compound of any one of claims 23-30, wherein R⁵ is Br.

83. The compound of any one of claims 23-30, wherein R⁵ is CN.

84. The compound of claim 1, which is selected from the group consisting of:

| Compd. | |
|---|---|
| No. | Structure |
| 1 | 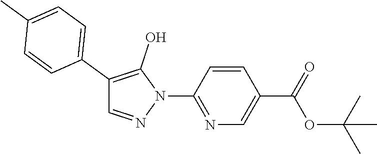 [SMILES: Cc1ccc(-c2cnn(-c3ccc(C(=O)OC(C)(C)C)cn3)c2O)cc1] |
| 2 | 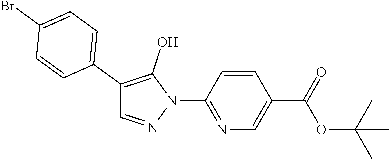 [SMILES: CC(C)(C)OC(=O)c1ccc(-n2ncc(-c3ccc(Br)cc3)c2O)nc1] |
| 3 | 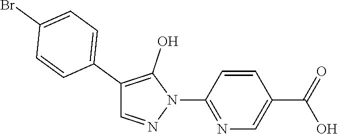 [SMILES: O=C(O)c1ccc(-n2ncc(-c3ccc(Br)cc3)c2O)nc1] |
| 4 | 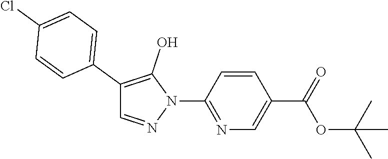 [SMILES: CC(C)(C)OC(=O)c1ccc(-n2ncc(-c3ccc(Cl)cc3)c2O)nc1] |
| 5 | 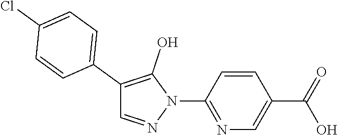 [SMILES: O=C(O)c1ccc(-n2ncc(-c3ccc(Cl)cc3)c2O)nc1] |
| 6 | 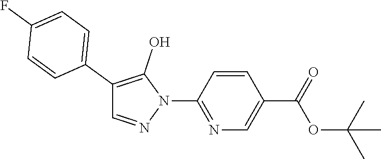 [SMILES: CC(C)(C)OC(=O)c1ccc(-n2ncc(-c3ccc(F)cc3)c2O)nc1] |
| 7 | 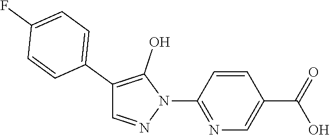 [SMILES: O=C(O)c1ccc(-n2ncc(-c3ccc(F)cc3)c2O)nc1] |
| 8 | 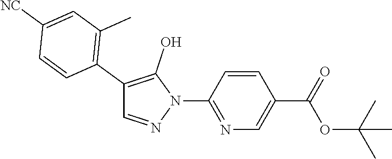 [SMILES: Cc1cc(C#N)ccc1-c1cnn(-c2ccc(C(=O)OC(C)(C)C)cn2)c1O] |
| 9 | 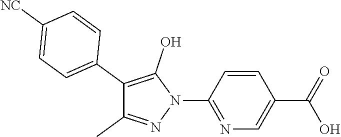 [SMILES: Cc1nn(-c2ccc(C(=O)O)cn2)c(O)c1-c1ccc(C#N)cc1] |
| 10 | 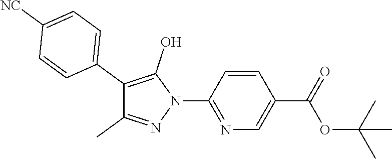 [SMILES: Cc1nn(-c2ccc(C(=O)OC(C)(C)C)cn2)c(O)c1-c1ccc(C#N)cc1] |
| 11 | 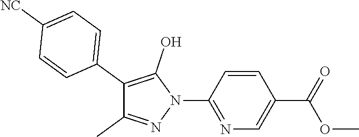 [SMILES: COC(=O)c1ccc(-n2nc(C)c(-c3ccc(C#N)cc3)c2O)nc1] |
| 12 | 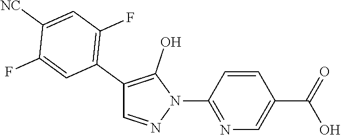 [SMILES: N#Cc1cc(F)c(-c2cnn(-c3ccc(C(=O)O)cn3)c2O)cc1F] |
| 13 | 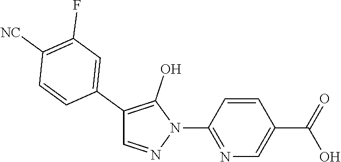 [SMILES: N#Cc1ccc(-c2cnn(-c3ccc(C(=O)O)cn3)c2O)cc1F] |
| 14 | 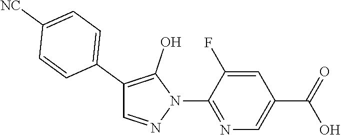 [SMILES: N#Cc1ccc(-c2cnn(-c3ncc(C(=O)O)cc3F)c2O)cc1] |
| 15 | 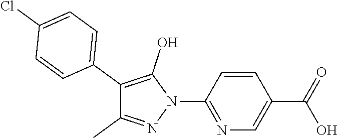 [SMILES: Cc1nn(-c2ccc(C(=O)O)cn2)c(O)c1-c1ccc(Cl)cc1] |
| 16 | 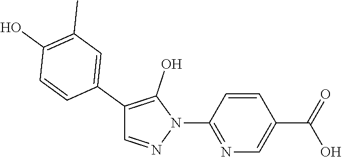 [SMILES: Cc1cc(-c2cnn(-c3ccc(C(=O)O)cn3)c2O)ccc1O] |
| 17 | 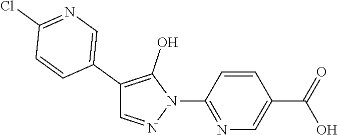 [SMILES: O=C(O)c1ccc(-n2ncc(-c3ccc(Cl)nc3)c2O)nc1] |
| 18 | 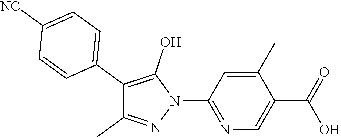 [SMILES: Cc1cc(-n2nc(C)c(-c3ccc(C#N)cc3)c2O)ncc1C(=O)O] |
| 19 | 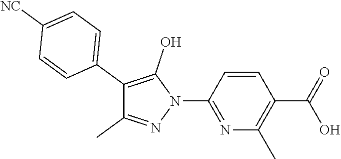 [SMILES: Cc1nc(-n2nc(C)c(-c3ccc(C#N)cc3)c2O)ccc1C(=O)O] |
| 20 | 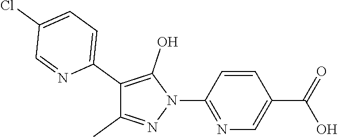 [SMILES: Cc1nn(-c2ccc(C(=O)O)cn2)c(O)c1-c1ccc(Cl)cn1] |
| 21 | 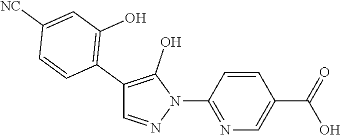 [SMILES: N#Cc1ccc(-c2cnn(-c3ccc(C(=O)O)cn3)c2O)c(O)c1] |
| 22 | 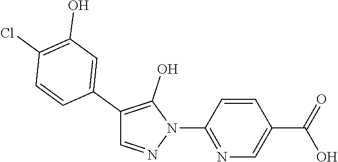 [SMILES: O=C(O)c1ccc(-n2ncc(-c3ccc(Cl)c(O)c3)c2O)nc1] |
| 23 | 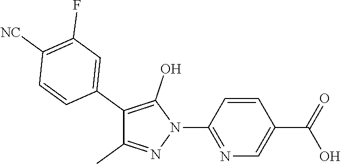 [SMILES: Cc1nn(-c2ccc(C(=O)O)cn2)c(O)c1-c1ccc(C#N)c(F)c1] |
| 24 | 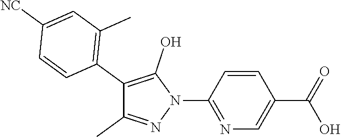 [SMILES: Cc1cc(C#N)ccc1-c1c(C)nn(-c2ccc(C(=O)O)cn2)c1O] |
| 25 | 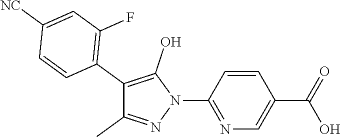 [SMILES: Cc1nn(-c2ccc(C(=O)O)cn2)c(O)c1-c1ccc(C#N)cc1F] |
| 26 | 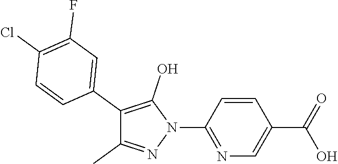 [SMILES: Cc1nn(-c2ccc(C(=O)O)cn2)c(O)c1-c1ccc(Cl)c(F)c1] |
| 27 | 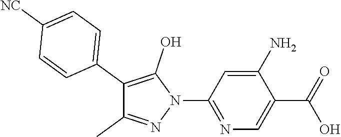 [SMILES: Cc1nn(-c2cc(N)c(C(=O)O)cn2)c(O)c1-c1ccc(C#N)cc1] |
| 28 | 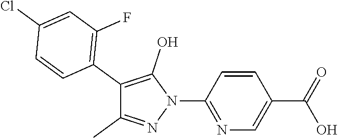 [SMILES: Cc1nn(-c2ccc(C(=O)O)cn2)c(O)c1-c1ccc(Cl)cc1F] |
| 29 | 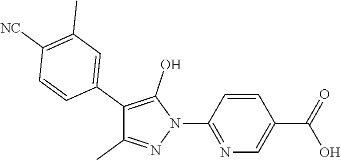 [SMILES: Cc1cc(-c2c(C)nn(-c3ccc(C(=O)O)cn3)c2O)ccc1C#N] |
| 30 | 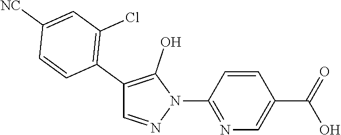 [SMILES: N#Cc1ccc(-c2cnn(-c3ccc(C(=O)O)cn3)c2O)c(Cl)c1] |
| 31 | 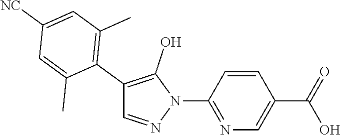 [SMILES: Cc1cc(C#N)cc(C)c1-c1cnn(-c2ccc(C(=O)O)cn2)c1O] |
| 32 | 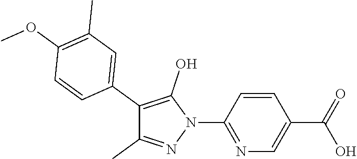 [SMILES: COc1ccc(-c2c(C)nn(-c3ccc(C(=O)O)cn3)c2O)cc1C] |
| 33 | 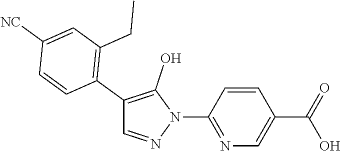 [SMILES: CCc1cc(C#N)ccc1-c1cnn(-c2ccc(C(=O)O)cn2)c1O] |
| 34 | 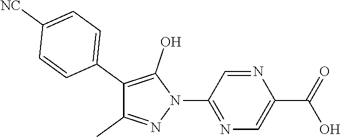 [SMILES: Cc1nn(-c2cnc(C(=O)O)cn2)c(O)c1-c1ccc(C#N)cc1] |
| 35 | 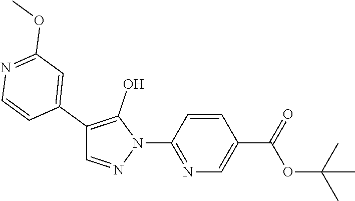 [SMILES: COc1cc(-c2cnn(-c3ccc(C(=O)OC(C)(C)C)cn3)c2O)ccn1] |
| 36 | 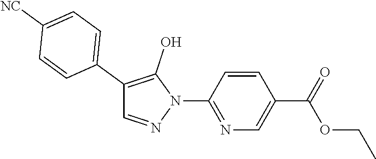 [SMILES: CCOC(=O)c1ccc(-n2ncc(-c3ccc(C#N)cc3)c2O)nc1] |
| 37 | 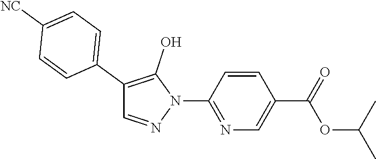 [SMILES: CC(C)OC(=O)c1ccc(-n2ncc(-c3ccc(C#N)cc3)c2O)nc1] |
| 38 | 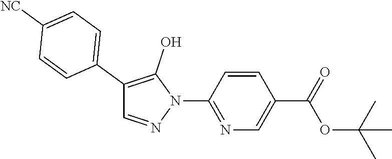 [SMILES: CC(C)(C)OC(=O)c1ccc(-n2ncc(-c3ccc(C#N)cc3)c2O)nc1] |
| 39 | 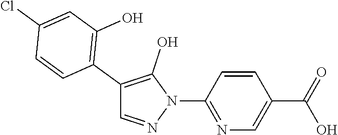 [SMILES: O=C(O)c1ccc(-n2ncc(-c3ccc(Cl)cc3O)c2O)nc1] |
| 40 | 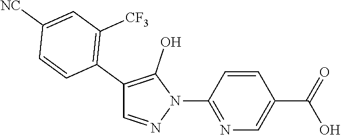 [SMILES: N#Cc1ccc(-c2cnn(-c3ccc(C(=O)O)cn3)c2O)c(C(F)(F)F)c1] |
| 41 | 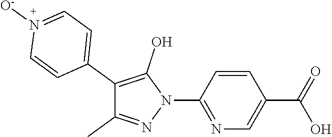 |
| 42 | 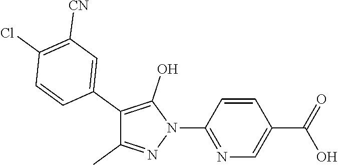 [SMILES: Cc1nn(-c2ccc(C(=O)O)cn2)c(O)c1-c1ccc(Cl)c(C#N)c1] |
| 43 | 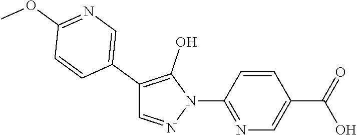 [SMILES: COc1ccc(-c2cnn(-c3ccc(C(=O)O)cn3)c2O)cn1] |
| 44 | 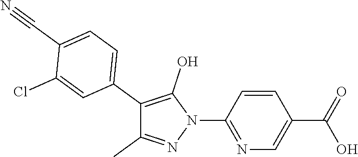 [SMILES: Cc1nn(-c2ccc(C(=O)O)cn2)c(O)c1-c1ccc(C#N)c(Cl)c1] |

or a pharmaceutically acceptable salt thereof

85. The compound of any one of claims 1-84, or a pharmaceutically acceptable salt thereof wherein at least one hydrogen atom is replaced with a deuterium atom.

86. A pharmaceutical composition comprising the compound of any one of claims 1-85, or a pharmaceutically acceptable salt thereof, and a pharmaceutically acceptable excipient.

87. A method for treating a disease mediated by PHD activity comprising administering to a subject the compound of any one of claims 1-85.

88. The method of claim 87, wherein the disease mediated by PHD activity is an ischemic reperfusion injury.

89. The method of claim 88, wherein the ischemic reperfusion injury is selected from stroke, myocardial infarction, and acute kidney injury.

90. The method of claim 87, wherein the disease mediated by PHD activity is inflammatory bowel disease.

91. The method of claim 90, wherein the inflammatory bowel disease is ulcerative colitis.

92. The method of claim 90, wherein the inflammatory bowel disease is Crohn's disease.

93. The method of claim 87, wherein the disease mediated by PHD activity is cancer.

94. The method of claim 93, wherein the cancer is colorectal cancer.

95. The method of claim 87, wherein the disease mediated by PHD activity is liver disease.

96. The method of claim 87, wherein the disease mediated by PHD activity is atherosclerosis.

97. The method of claim 87, wherein the disease mediated by PHD activity is cardiovascular disease.

98. The method of claim 87, wherein the disease mediated by PHD activity is a disease or condition of the eye.

99. The method of claim 98, wherein the disease or condition of the eye is selected from radiation retinopathy, retinopathy of prematurity, diabetic retinopathy, age-related macular degeneration, and ocular ischemia.

100. The method of claim 87, wherein the disease is anemia.

101. The method of claim 100, wherein the anemia is anemia associated with chronic kidney disease.

102. The method of claim 87, wherein the disease is chronic kidney disease.

103. The method of claim 87, wherein the disease is associated with hyperoxia.

104. The method of claim 103, wherein the disease is retinopathy of prematurity.

105. The method of claim 103, wherein the disease is bronchopulmonary dysplasia (BPD).

106. The method of claim 87, wherein the disease is selected from ischemic heart disease, valvular heart disease, congestive heart failure, acute lung injury, pulmonary fibrosis, pulmonary hypertension, chronic obstructive pulmonary disease (COPD), acute liver failure, liver fibrosis, and cirrhosis.
